(19) United States
(12) Patent Application Publication
(10) Pub. No.: US 2026/0125346 A1
(43) Pub. Date: May 7, 2026

(54) Isoquinolinone Derivatives and 4H-Quinolizinone Derivatives and Pharmaceutical Compositions Thereof for the Treatment of Disease

(71) Applicant: BeOne Medicines I GmbH, Basel (CH)

(72) Inventors: Ling Qin, Beijing (CN); Yunhang Guo, Beijing (CN); Zhiwei Wang, Beijing (CN)

(73) Assignee: BeOne Medicines I GmbH, Basel (CH)

(21) Appl. No.: 19/381,476

(22) Filed: Nov. 6, 2025

Related U.S. Application Data
(63) Continuation of application No. PCT/CN2024/092225, filed on May 10, 2024.

(30) Foreign Application Priority Data
May 11, 2023 (WO) PCT/CN2023/093652
Jul. 20, 2023 (WO) PCT/CN2023/108473

(51) Int. Cl. C07D 217/24; A61K 31/4375; A61K 31/472; A61K 31/4725; A61K 31/496; A61K 31/5025; A61K 31/506; A61K 31/519; A61K 31/5377; A61K 31/675; C07B 59/00; C07D 401/04; C07D 401/06; C07D 401/10; C07D 401/12; C07D 401/14; C07D 405/04; C07D 405/10; C07D 405/12; C07D 405/14; C07D 409/12; C07D 409/14; C07D 413/14; C07D 417/04; C07D 471/04; C07D 487/04; C07D 491/056; C07D 491/107; C07D 495/04; C07D 513/04; C07F 9/62

(52) U.S. Cl. CPC C07D 217/24; A61K 31/4375; A61K 31/472; A61K 31/4725; A61K 31/496; A61K 31/5025; A61K 31/506; A61K 31/519; A61K 31/5377; A61K 31/675; C07B 59/002; C07D 401/04; C07D 401/06; C07D 401/10; C07D 401/12; C07D 401/14; C07D 405/04; C07D 405/10; C07D 405/12; C07D 405/14; C07D 409/12; C07D 409/14; C07D 413/14; C07D 417/04; C07D 471/04; C07D 487/04; C07D 491/056; C07D 491/107; C07D 495/04; C07D 513/04; C07F 9/62

(57) ABSTRACT

Disclosed herein is an isoquinolin-1(2H)-one derivative of Formula (I):

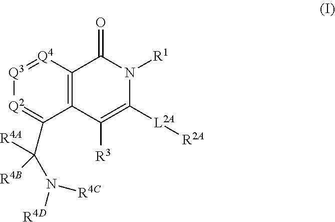

or a stereoisomer, tautomer, or pharmaceutically acceptable salt thereof, wherein values for the variables (e.g., L^{2A}, R¹, R^{2A}, R³, R^{4A}, R^{4B}, R^{4C}, R^{4D}, Q², Q³, Q⁴) are as described herein. Also disclosed is a pharmaceutical composition comprising such derivative, and a method for treating or preventing a disorder or a disease responsive to the inhibition of PI3Kα activity in a subject using such derivative.

### CROSS REFERENCE TO RELATED APPLICATIONS

This application is a continuation of International Application No. PCT/CN2024/092225, filed May 10, 2024, which claims priority to International Application No. PCT/CN2023/093652, filed May 11, 2023, and International Application No. PCT/CN2023/108473, filed Jul. 20, 2023, the disclosures of each of which are hereby incorporated by reference in their entireties.

### FIELD OF THE DISCLOSURE

Disclosed herein are an isoquinolin-1(2H)-one derivative and a 4H-quinolizin-4-one derivative, or a stereoisomer thereof, or a pharmaceutically acceptable salt thereof useful as a PI3Kα inhibitor, and a pharmaceutical composition comprising the same. Also disclosed herein is a method of treating cancer using an isoquinolin-1(2H)-one derivative, or a stereoisomer thereof, or a pharmaceutically acceptable salt thereof as a PI3Kα inhibitor. Further disclosed herein is a method of treating cancer using a 4H-quinolizin-4-one derivative, or a stereoisomer thereof, or a pharmaceutically acceptable salt thereof as a PI3Kα inhibitor.

### BACKGROUND OF THE DISCLOSURE

Phosphoinositide 3-kinase (PI3K) signaling is one of the most frequently aberrantly activated pathways in human cancers. Under physiological conditions, the PI3K pathway is activated in response to insulin, growth factors and cytokines. Activated PI3K regulates key metabolic processes, including glucose metabolism, biosynthesis of macromolecules and maintenance of redox balance, to support both systemic metabolic homeostasis and the growth of individual cells. Oncogenic activation of the PI3K pathway in cancer cells reprograms cellular metabolism by augmenting the activity of nutrient transporters and metabolic enzymes, thereby supporting the anabolic demands of aberrantly growing cells.

The PI3K signaling network is activated downstream of receptor tyrosine kinases (RTKs), cytokine receptors, integrins and G protein-coupled receptors (GPCRs) and plays a central role in promoting cell survival and growth (Fruman et al., 2017). Class IA PI3K exists as heterodimers of a catalytic subunit (p110α, p110β or p110δ) associated with a regulatory subunit (p85α or p85β, or shorter variants thereof)(Thorpe, Yuzugullu, & Zhao, 2015). Activation of PI3K at the plasma membrane stimulates phosphorylation of its phospholipid substrate phosphatidylinositol 4,5-bisphosphate (PtdIns(4,5)P2) to produce the second messenger, phosphatidylinositol 3,4,5-trisphosphate (PtdIns(3,4,5)P3). PtdIns(3,4,5)P3 accumulation at the plasma membrane, and perhaps at other intracellular membranes, creates docking sites to recruit downstream effector proteins that contain a subclass of pleckstrin homology (PH) domain, including protein kinase (such as AKT and BTK), adaptor proteins and regulators of GTPase, to regulate their activities.

Genetic events leading to growth factor-independent activation of the PI3K pathway are one of the most frequently occurring drivers of human cancer. Among the genetic alternations, PIK3CA mutation is one of the most frequently mutated kinase genes in solid tumors, with mutation rate of ˜14% across all cancers (Zhang et al., 2017). It is now evident that cancers of the endometrium, breast, colon, lung, as well as benign tumors of the skin are among the tumor types with the highest frequencies of PIK3CA mutations (Samuels & Waldman, 2010). Oncogenic mutation of PIK3CA is highly enriched at hotspot mutations in the helical (E542K, E545K) and kinase (H1047R) domains (Arafeh & Samuels, 2019).

Given the oncogenic functions of PIK3CA mutation in cancer progression, several inhibitors of PI3Kα have been developed, and one p110α-selective PI3K inhibitor Alpelisib (BYL719) has shown improved clinical responses when used in combination with estrogen receptor antagonist (Andre et al., 2019), which was thus approved by FDA to treat HR+Her2− metastasis breast cancer harboring PIK3CA mutation.

Though there is drug approved and also several inhibitors being continuously investigated in clinical trials, the efficacy of p110α-selective PI3K inhibitors are modest, in part due to the on-target toxicity of hyperglycemia and/or hyperinsulinemia. Targeted inhibition of PI3Kα disrupts glucose metabolism in multiple tissues, which prevents glucose uptake in the skeletal muscle and adipose tissue, resulting in hyperglycemia. Hyperglycemia is usually transient at the early stage of PI3Kα inhibition, due to compensatory insulin release from the pancreas. However, the hyperglycemia may be exacerbated or prolonged in patients with any degree of insulin resistance and, in these cases, requires discontinuation of therapy (Mayer et al., 2017). In addition, it is reported in pre-clinical study that high level of insulin in circulation would activate PI3K-mTOR signaling axis in tumors, compromising the effectiveness of PI3Kα inhibitors (Hopkins et al., 2018).

Hence, there is a need to improve the therapeutic index of PI3Kα inhibitors by identifying compounds with increased selectivity for mutant PI3Kα over wild-type PI3Kα. This is expected to specifically inhibit the mutant PI3Kα in cancer cell, sparing the wild-type PI3Kα in the host tissues, thus creating a larger therapeutic window, enabling sufficient target inhibition and achieving improved anti-tumor efficacy.

### SUMMARY OF THE DISCLOSURE

Disclosed herein are isoquinolin-1(2H)-one derivatives of Formula (I) and 4H-quinolizin-4-one derivatives of Formula (VI), and their methods of use.

Aspect 1. A compound of Formula (I) or Formula (VI),

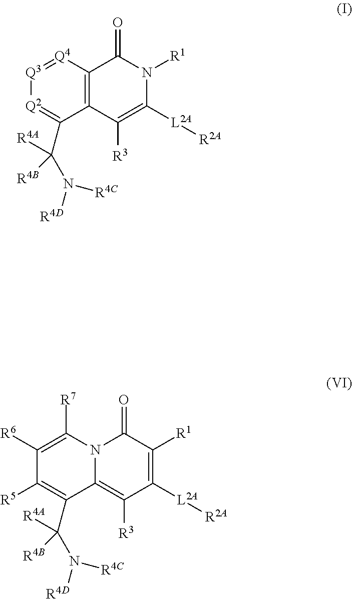

or a stereoisomer thereof, or a tautomer thereof, or a pharmaceutically acceptable salt thereof, wherein
Q² is C(R⁵) or N;
Q³ is C(R⁶) or N;
Q⁴ is C(R⁷) or N;
at most two of Q², Q³, and Q⁴ is N;
R¹ is hydrogen, —CN, —C₁₋₆alkyl, —C₂₋₆alkenyl, —C₂₋₆alkynyl, cycloalkyl, cycloalkenyl, cycloalkynyl, heterocyclyl, aryl, or heteroaryl, wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{11a};
each R^{11a} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{111a}, —COR^{111a}, —CO₂R^{111a}, —CONR^{111a}R^{111b}, —NR^{111a}R^{111b}, —NR^{111a}COR^{111b}, —NR^{111a}CONR^{111b}R^{111c}, —NR^{111a}CO₂R^{111b}, —P(O)R^{111b}R^{111c}, or —SO₂R^{111b}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{111d};
R^{111a}, R^{111b}, and R^{111c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{111d};
each R^{111d} is independently hydrogen, halogen, —CN, —NO₂, —C₁₋₆alkyl, —C₂₋₆alkenyl, —C₂₋₆alkynyl, —C₁₋₆ haloalkyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl;
*-L^{2A}-** is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or

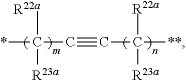

wherein one or more methylene units of *—(CH₂)ₘ—C(R^{22a})═C(R^{23a})—(CH₂)ₙ—**, and

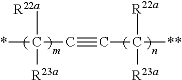

are optionally and independently replaced by —CH(R^{22a})—, —C(R^{22a}R^{23a})—, cycloalkylene, heterocycloalkylene, —N(R^{21a})—, —N(R^{21a})C(O)—, —N(R^{21a})C(NR^{21b})—, —N(R^{21a})C(NOR^{21b})—, —N(R^{21a})C(NCN)—, —C(O)N(R^{21a})—, —N(R^{21a})S(O)₂—, —S(O)₂N(R^{21a})—, —O—, —C(O)—, —OC(O)—, —C(O)O—, —S—, —S(O)—, or —S(O)₂—; wherein * refers to the position attached to the

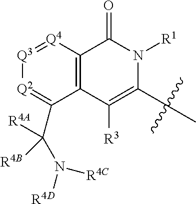

moiety (e.g., the isoquinolin-1(2H)-one, as for example, in formula (IV)), and ** refers to the position attached to R^{2A};
m, and n are each independently 0, 1, 2, 3, 4, 5 or 6;
R^{21a} and R^{21b} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{211b};
each R^{211b} is independently hydrogen, halogen, —CN, —NO₂, —C₁₋₆alkyl, —C₂₋₆alkenyl, —C₂₋₆alkynyl, cycloalkyl, cycloalkenyl, cycloalkynyl, heterocyclyl, aryl, heteroaryl, —C₁₋₆haloalkyl, —OC₁₋₆alkyl, —COC₁₋₆alkyl, or —CO₂C₁₋₆alkyl;
R^{22a}, and R^{23a} are each independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{222a}, —COR^{222a}, —CO₂R^{222a}, —CONR^{222a}R^{222b}, —NR^{222a}R^{222b}, —NR^{222a}COR^{222b}, —NR^{222a}CONR^{222b}R^{222c}, or —NR^{222a}CO₂R^{222b}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{223a};
R^{222a}, R^{222b}, and R^{222c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{223a};
each R^{223a} is independently hydrogen, halogen, —CN, —NO₂, —C₁₋₆alkyl, —C₂₋₆alkenyl, —C₂₋₆alkynyl, —C₁₋₆ haloalkyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl;
R^{2A} is selected from hydrogen, —C₁₋₆alkyl, —C₂₋₆alkenyl, —C₂₋₆alkynyl, cycloalkyl, cycloalkenyl, cycloalkynyl, unsaturated heterocyclic ring comprising at least one carbon-carbon double bond, aryl, or heteroaryl; wherein said —C₁₋₆alkyl, —C₂₋₆alkenyl, —C₂₋₆alkynyl, cycloalkyl, cycloalkenyl, cycloalkynyl, unsaturated heterocyclic ring, aryl, or heteroaryl are each independently optionally substituted with at least one substituent R^{24a};
each R^{24a} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —P(O)R^{241b}R^{241c}, —BR^{241b}R^{241c}, —SiR^{241a}R^{241b}R^{241c}, —SeR^{241a}, —OR^{241a}, —SR^{241a}, —COR^{241a}, —CO₂R^{241a}, —CONR^{241a}R^{241b}, —NR^{241a}R^{241b}, NR^{241a}COR^{241b}, —NR^{241a}CONR^{241b}R^{241c}, or —NR^{241a}CO₂R^{241b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{242a};
each R^{242a} is independently hydrogen, halogen, oxo, —OH, —CN, —NO₂, —NR^{241a}R^{241b}, —COR^{241a}, —CONR^{241a}R^{241b}, —C₁₋₆alkyl, —OC₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{243a};
R^{241a}, R^{241b}, and R^{241c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{243a};
each R^{243a} is independently hydrogen, halogen, —CN, —NO₂, —C₁₋₆alkyl, —C₂₋₆alkenyl, —C₂₋₆alkynyl, —C₁₋₆ haloalkyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl;
R³ is hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{3a}, —COR^{3a}, —CO₂R^{3a}, —CONR^{3a}R^{3b}, —NR^{3a}R^{3b}, —NR^{3a}COR^{3b}, —NR^{3a}CONR^{3b}R^{3c}, or —NR^{3a}CO₂R^{3b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{33a};
each R^{33a} is independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆haloalkyl, —COC₁₋₆alkyl, or —CO₂C₁₋₆alkyl;
R^{3a}, R^{3b}, and R^{3c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{333a};
each R^{333a} is independently hydrogen, halogen, —CN, —NO₂, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl;
R^{4A} and R^{4B}, which may be the same or different, are each independently hydrogen, halogen, —C₁₋₆ alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{4a}, —COR^{4a}, —CO₂R^{4a}, —CONR^{4a}R^{4b}, —NR^{4a}R^{4b}, —NR^{4a}COR^{4b}, —NR^{4a}CONR^{4b}R^{4d}, or —NR^{4a}CO₂R^{4b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{44a}.
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl;
R^{4a}, R^{4b}, and R^{4d} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{M};
each R^{M} is independently hydrogen, halogen, —CN, —NO₂, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl;
R^{4C} is hydrogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —COC₁₋₆alkyl, or —CO₂C₁₋₆alkyl; wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —COC₁₋₆alkyl, or —CO₂C₁₋₆alkyl are each independently optionally substituted with at least one substituent R^{44d};
each R^{44d} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl;
R^{4D} is cycloalkyl, cycloalkenyl, cycloalkynyl, heterocyclyl, aryl, or heteroaryl; wherein said cycloalkyl, cycloalkenyl, cycloalkynyl, heterocyclyl, aryl, or heteroaryl are each independently optionally substituted with at least one substituent R^{44e};
each R^{44e} is independently halogen, —C₁₋₆alkyl, —C₂₋₆alkenyl, —C₂₋₆alkynyl, —CN, —NO₂, —OR^{444d}, —SO₂R^{444d}, —COR^{444d}, —CO₂R^{444d}, —CONR^{444d}R^{444e}, —C(═NR^{444d})NR^{444e}R^{444f}, —NR^{444d}R^{444e}, —NR^{444d}COR^{444e}, —NR^{444d}CONR^{444e}R^{444f}, —NR^{444d}CO₂R^{444e}, —NR^{444d}SONR^{444e}R^{444f}, —SO₂NR^{444e}R^{444f}, —SO₂NR^{444e}COR^{444f}, or —NR^{444d}SO₂R^{444e}; each of said —C₁₋₆alkyl, —C₂₋₆alkenyl, —C₂₋₆alkynyl, cycloalkyl, cycloalkenyl, cycloalkynyl, heterocyclyl, aryl, heteroaryl are each independently optionally substituted with at least one substituent R^{N};
R^{444d}, R^{444e}, and R^{444f} are each independently selected from hydrogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —C₁₋₆haloalkyl, —COC₁₋₆ alkyl, or —CO₂C₁₋₆alkyl;
R^{N} is each independently selected from hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆ haloalkyl, —COC₁₋₆alkyl, or —CO₂C₁₋₆alkyl;
R⁵, if present, is hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{5a}, —COR^{5a}, —CO₂R^{5a}, —CONR^{5a}R^{5b}, —NR^{5a}R^{5b}, —NR^{5a}COR^{5b}, —NR^{5a}CONR^{5b}R^{5c}, or —NR^{5a}CO₂R^{5b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{55a};
each R^{55a} is independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆haloalkyl, —COC₁₋₆alkyl, or —CO₂C₁₋₆alkyl;
R^{5a}, R^{5b}, and R^{5c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{555a};
each R^{555a} is independently hydrogen, halogen, —CN, —NO₂, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl;
R⁶, if present, is hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR⁶a, —COR⁶a, —CO₂R^{6a}, —CONR^{6a}R^{6b}, —NR^{6a}R^{6b}, —NR^{6a}COR^{6b}, —NR^{6a}CONR^{6b}R^{6c}, or —NR^{6a}CO₂R^{6b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{66a};
each R^{66a} is independently hydrogen, halogen, —CN, —NO₂, —C₁₋₆alkyl, —C₂₋₆alkenyl, —C₂₋₆alkynyl, cycloalkyl, cycloalkenyl, cycloalkynyl, heterocyclyl, aryl, heteroaryl, —C₁₋₆haloalkyl, —OC₁₋₆alkyl, —COC₁₋₆alkyl, or —CO₂C₁₋₆alkyl; R^{6a}, R^{6b}, and R^{6c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{666a};
each R^{666a} is independently hydrogen, halogen, —CN, —NO₂, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl;
R⁷, if present, is hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{7a}, —COR^{7a}, —CO₂R^{7a}, —CONR^{7a}R^{7b}, —NR^{7a}R^{7b}, —NR^{7a}COR^{7b}, —NR^{7a}CONR^{7b}R^{7c}, or —NR^{7a}CO₂R^{7b}; wherein said —C₁₋₆alkyl, —C₂₋₆alkenyl, —C₂₋₆alkynyl, cycloalkyl, cycloalkenyl, cycloalkynyl, heterocyclyl, aryl or heteroaryl are each independently optionally substituted with at least one substituent R^{77a};
each R^{77a} is independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆haloalkyl, —COC₁₋₆alkyl, or —CO₂C₁₋₆alkyl;
R^{7a}, R^{7b}, and R^{7c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{777a};
each R^{777a} is independently hydrogen, halogen, —CN, —NO₂, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl.

In embodiments of Aspect 1, Q² is C(R⁵); Q³ is C(R⁶); and Q⁴ is C(R⁷) or N.

Aspect 2. The compound according to aspect 1, having formula (II):

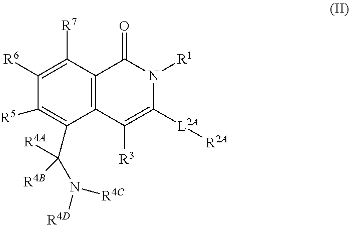

wherein R¹, L^{2A}, R^{2A}, R³, R^{4A}, R^{4B}, R^{4C}, R^{4D}, R⁵, R⁶, and R⁷, are defined as Aspect 1.

Aspect 3. The compound according to aspect 1 or 2, having formula (III-1)-(III-9):

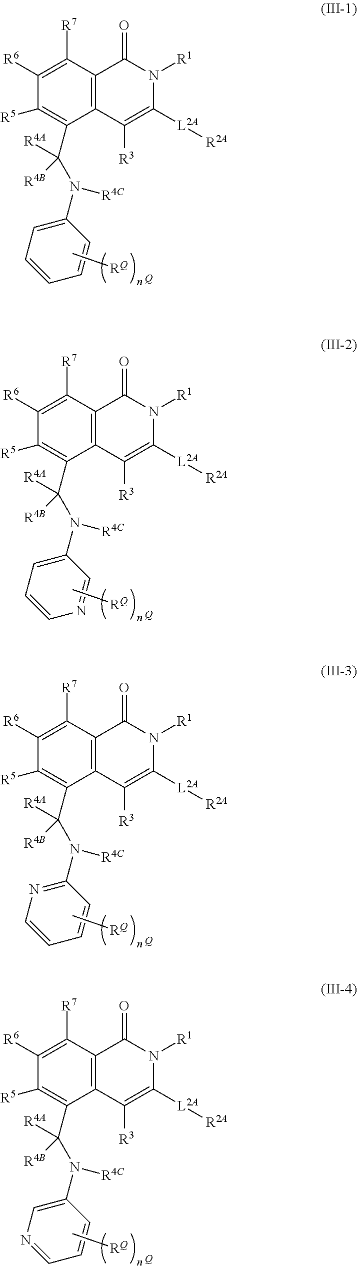

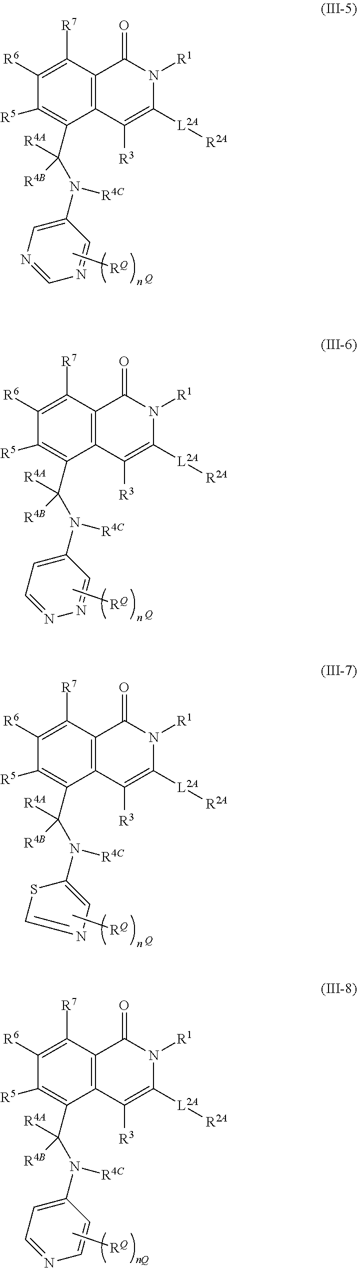

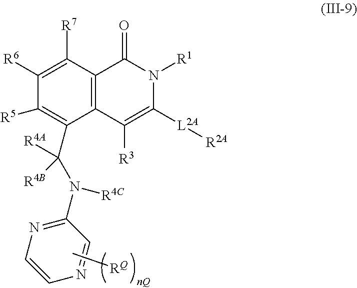

wherein each R^{Q} is independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{Q1}, —SO₂R^{Q1}, —SO₂NR^{Q2}R^{Q3}, —COR^{Q1}, —CO₂R^{Q1}, —CONR^{Q1}R^{Q2}, —NR^{Q1}R^{Q2}, —NR^{Q1}COR^{Q2}, —NR^{Q1}CONR^{Q2}R^{Q3}, or —NR^{Q1}CO₂R^{Q2}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent
R^{Q1}, R^{Q2}, and R^{Q3} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R¹¹;
R^{Q11} is hydrogen, halogen, —CN, —NO₂, —C₁₋₆alkyl, —C₂₋₆alkenyl, —C₂₋₆alkynyl, —C₁₋₆haloalkyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl;
n^{Q} is 0, 1, 2, 3, 4, or 5.

Aspect 4a. The compound according to aspect 3, having formula (IV-a):

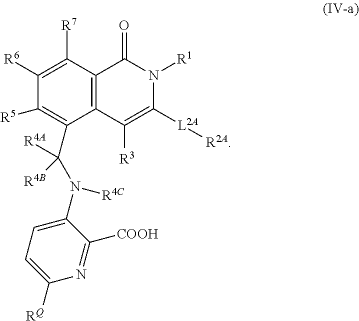

Aspect 4. The compound according to aspect 3, having formula (IV):

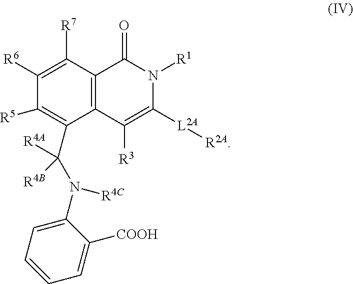

In embodiments of Aspect 4, R¹ is hydrogen, deuterium, —C₁₋₆alkyl, deuterated —C₁₋₆alkyl, heterocyclyl, heteroaryl, or aryl; wherein said —C₁₋₆alkyl, deuterated —C₁₋₆alkyl, heterocyclyl, heteroaryl, or aryl are each independently optionally substituted with at least one substituent R^{11a};
each R^{11a} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{1a}, —COR^{1a}, —CO₂R^{1a}, —CONR^{1a}R^{1b}, —SO₂R^{1a}, —NR^{1a}R^{1b}, —NR^{1a}COR^{1b}, —NR^{1a}CONR^{1b}R^{1c}, or —NR^{1a}CO₂R^{1b}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{1d}.

R^{1a}, R^{1b}, and R^{1c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent Rid;
each R^{1d} is independently hydrogen, halogen, oxo, —CN, —NO₂, —C₁₋₆alkyl, —C₂₋₆alkenyl, —C₂₋₆alkynyl, —C₁₋₆haloalkyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl.

R³ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
R^{4A} and R^{4B}, which may be the same or different, are each independently hydrogen, deuterium, halogen, —C₁₋₆alkyl, or deuterated —C₁₋₆alkyl; wherein said —C₁₋₆alkyl or deuterated —C₁₋₆alkyl is each independently optionally substituted with at least one substituent selected from halogen, —C₁₋₆alkyl, —C₁₋₆haloalkyl, —CN, —NO₂, —OC₁₋₆alkyl, —COC₁₋₆alkyl, and —CO₂C₁₋₆alkyl;
R^{4C} is hydrogen, halogen, —C₁₋₆alkyl or deuterated —C₁₋₆alkyl; wherein said —C₁₋₆alkyl or deuterated —C₁₋₆alkyl is optionally substituted with at least one substituent selected from hydrogen, halogen, —C₁₋₆ alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, and —CO₂C₁₋₆alkyl;
R⁵ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
R⁶ is hydrogen, halogen, —C₁₋₆alkyl, —OC₁₋₆alkyl, —CN, or —NO₂; wherein said —C₁₋₆alkyl or —OC₁₋₆alkyl is each independently optionally substituted with at least one substituent selected from hydrogen, deuterium, halogen, —C₁₋₆alkyl, —CN, and —NO₂;
R⁷ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
-L^{2A}- is a covalent bond, *—N(R^{21a})C(O)—**, **—N(R^{21a})C(O)—*, *—(CR^{22a}R^{23a})ₘ—C(R^{22a})═C(R^{23a})—

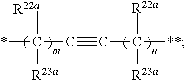

(CR^{22a}R^{23a})—**, or
* refers to the position attached to the isoquinolin-1(2H)-one;
** refers to the position attached to R^{2A};
m and n are each independently 0, 1, 2, or 3;
R^{21a} is hydrogen or —C₁₋₆alkyl; wherein said —C₁₋₆alkyl is optionally substituted with at least one substituent R^{211b};
each R^{211b} is independently hydrogen or halogen;
R^{22a} and R^{23a} are each independently hydrogen, halogen, or —C₁₋₆alkyl; wherein said —C₁₋₆alkyl is each independently optionally substituted with at least one substituent R^{223a};
   each R^{223a} is independently hydrogen or halogen;
R^{2A} is —C₁₋₆alkyl, 3- to 15-membered cycloalkyl, unsaturated 3- to 15-membered heterocyclyl comprising at least one carbon-carbon double bond, 5- to 12-membered aryl, or 5- to 15-membered heteroaryl; wherein each of said —C₁₋₆alkyl, 3- to 15-membered cycloalkyl, unsaturated 3- to 7-membered heterocyclyl comprising at least one carbon-carbon double bond, 5- to 12-membered aryl, or 5- to 15-membered heteroaryl is optionally substituted with at least one R^{Y1};
each R^{Y1} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆ alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, or —SC₁₋₆alkyl are each independently optionally substituted with at least one substituent R^{Y1a};
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂.

Aspect 5. The compound according to aspect 1, having formula (V):

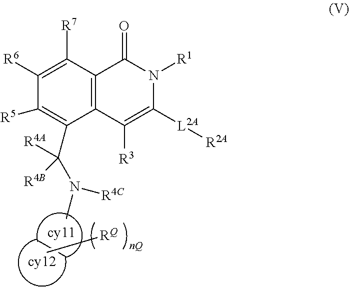

wherein cyl1 and cyl2 are each independently 5-, or 6-, membered cycloalkyl ring, cycloalkenyl ring, heterocyclyl ring, aryl ring, or heteroaryl ring;
at least one of cyl1 and cyl2 is an aromatic ring;
cyl1 and cyl2 are each independently optionally substituted by R^{Q}.

Aspect 6. The compound according to aspect 5, having formula (V-1)-(V-11):

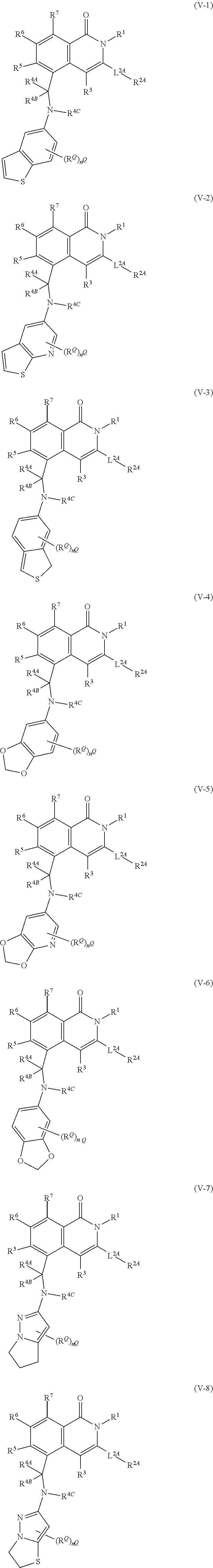

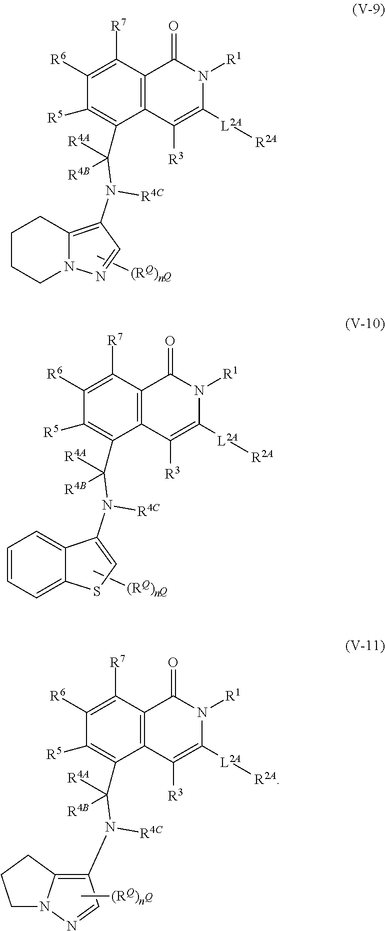

Aspect 7. The compound according to any one of aspects 1 to 6, wherein:
-L²A-** is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or

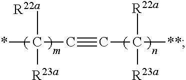

m, and n is each independently 0, 1, 2, 3, 4, 5 or 6;
R^{21a} is hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{211b};
each R^{211b} is independently hydrogen, halogen, —CN, —NO₂, —C₁₋₆alkyl, —C₂₋₆alkenyl, —C₂₋₆alkynyl, cycloalkyl, cycloalkenyl, cycloalkynyl, heterocyclyl, aryl, heteroaryl, —C₁₋₆haloalkyl, —OC₁₋₆alkyl, —COC₁₋₆alkyl, or —CO₂C₁₋₆alkyl;
R^{22a}, and R^{23a} are each independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{222a}, —COR^{222a}, —CO₂R^{222a}, —CONR^{222a}R^{222b}, —NR^{222a}R^{222b}, —NR^{222a}COR^{222b}, —NR^{222a}CONR^{222b}R^{222c}, or —NR^{222a}CO₂R^{222b}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{223a};
R^{222a}, R^{222b}, and R^{222c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{223a};
R^{223a} is hydrogen, halogen, —CN, —NO₂, —C₁₋₆alkyl, —C₂₋₆alkenyl, —C₂₋₆alkynyl, —C₁₋₆haloalkyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl.

Aspect 8. The compound according to aspect 7, wherein:
*-L²A-** is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or

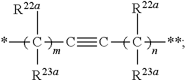

m, and n is each independently 0, 1, 2, 3, 4, 5 or 6;
R^{21a} is hydrogen, —C₁₋₆alkyl, —C₂₋₆alkenyl, —C₂₋₆alkynyl, or cycloalkyl; wherein said —C₁₋₆alkyl, —C₂₋₆alkenyl, —C₂₋₆alkynyl, or cycloalkyl are each independently optionally substituted with at least one substituent R^{211b};
each R^{211b} is independently hydrogen, halogen, —CN, —NO₂, —C₁₋₆alkyl, —C₂₋₆alkenyl, —C₂₋₆alkynyl, cycloalkyl, cycloalkenyl, cycloalkynyl, heterocyclyl, aryl, heteroaryl, —C₁₋₆haloalkyl, —OC₁₋₆alkyl, —COC₁₋₆alkyl, or —CO₂C₁₋₆alkyl;
R^{22a}, and R^{23a} are each independently hydrogen, halogen, —C₁₋₆alkyl, —C₂₋₆alkenyl, —C₂₋₆alkynyl, cycloalkyl, —CN, —NO₂, —OR^{222a}, —COR^{222a}, —CO₂R^{222a}, —CONR^{222a}R^{222b}, —NR^{222a}R^{222b}, —NR^{222a}COR^{222b}, —NR^{222a}CONR^{222b}R^{222c}, or —NR^{222a}CO₂R^{222b}; wherein said —C₁₋₆alkyl, —C₂₋₆alkenyl, —C₂₋₆alkynyl, or cycloalkyl are each independently optionally substituted with at least one substituent R^{223a};
R^{222a}, R^{222b}, and R^{222c} are each independently hydrogen, —C₁₋₆alkyl, —C₂₋₆alkenyl, —C₂₋₆alkynyl, or cycloalkyl; wherein said —C₁₋₆alkyl, —C₂₋₆alkenyl, —C₂₋₆alkynyl, or cycloalkyl are each independently optionally substituted with at least one substituent R^{223a};
each R^{223a} is independently hydrogen, halogen, —CN, —NO₂, —C₁₋₆alkyl, —C₂₋₆alkenyl, —C₂₋₆alkynyl, —C₁₋₆ haloalkyl, cycloalkyl, cycloalkenyl, cycloalkynyl, heterocyclyl, aryl, heteroaryl, —OC₁₋₆alkyl, —COC₁₋₆ alkyl, or —CO₂C₁₋₆alkyl.

Aspect 9. The compound according to aspect 8, wherein:
*-L²A-** is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or

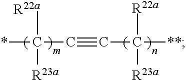

m, and n is each independently 0, 1, 2, 3, 4, 5 or 6;
R^{21a} is hydrogen, —C₁₋₆alkyl; wherein said —C₁₋₆alkyl is each independently optionally substituted with at least one substituent R^{211b};
each R^{211b} is independently hydrogen, halogen, —CN, —NO₂, —C₁₋₆alkyl, —C₁₋₆haloalkyl, —OC₁₋₆alkyl, —COC₁₋₆alkyl, or —CO₂C₁₋₆alkyl;
R^{22a}, and R^{23a} are each independently hydrogen, halogen, —C₁₋₆alkyl, —CN, —NO₂, or —OR^{222a}; wherein said —C₁₋₆alkyl is each independently optionally substituted with at least one substituent R^{223a};
each R^{223a} is independently hydrogen, halogen, —CN, —NO₂, —C₁₋₆alkyl, —C₁₋₆haloalkyl, —OC₁₋₆alkyl, —COC₁₋₆alkyl, or —CO₂C₁₋₆alkyl.

Aspect 10. The compound according to aspect 9, wherein:
*-L²A-** is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or

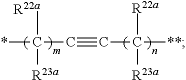

m, and n is each independently 0, 1, 2, or 3;
R^{21a} is hydrogen, or —C₁₋₆alkyl; wherein said —C₁₋₆alkyl is each independently optionally substituted with at least one substituent R^{211b};
each R^{211b} is independently hydrogen, or halogen;
R^{22a}, and R^{23a} are each independently hydrogen, halogen, or —C₁₋₆alkyl; wherein said —C₁₋₆alkyl is each independently optionally substituted with at least one substituent R^{223a};
each R^{223a} is independently hydrogen, or halogen.

Aspect 11. The compound according to aspect 10, wherein:
L^{2A}- is a covalent bond, *—NHC(O)—**, **—NHC(O)—*, *—CH═CH—**, or *—C≡C—**.

In embodiments of Aspect 11, -L^{2A}- is a covalent bond.

Aspect 12. The compound according to any one of aspects 1 to 11, wherein:
R^{2A} is selected from —C₁₋₆alkyl, cycloalkyl, unsaturated heterocyclyl comprising at least one carbon-carbon double bond, aryl, or heteroaryl; wherein said —C₁₋₆alkyl, cycloalkyl, unsaturated heterocyclyl, aryl, or heteroaryl are each independently optionally substituted with at least one substituent R^{24a}; each R^{24a} is independently hydrogen, halogen, oxo, —C₁₋₆alkyl, —SC₁₋₆alkyl, cycloalkyl, cycloalkenyl, cycloalkynyl, heterocyclyl, aryl, heteroaryl, —CN, —COOH, —P(O)R^{241b}R^{241c}, or —OR^{241a}; wherein said —C₁₋₆ alkyl, cycloalkyl, cycloalkenyl, cycloalkynyl, heterocyclyl, aryl or heteroaryl are each independently optionally substituted with at least one substituent R^{242a};
each R^{242a} is independently hydrogen, halogen, —CN, —C₁₋₆alkyl, —NR^{241a}R^{241b}, —COR^{241a}, —CONR^{241a}R^{241b}, —OR^{241a}, heterocyclyl, aryl, or heteroaryl; wherein said —C₁₋₆alkyl, heterocyclyl, aryl, or heteroaryl are each independently optionally substituted with at least one substituent R^{243a};
R^{241a}, R^{241b}, and R^{241c} are each independently hydrogen, cycloalkyl, heterocyclyl, or —C₁₋₆alkyl; wherein said —C₁₋₆alkyl is each independently optionally substituted with at least one substituent R^{243a};
each R^{243a} is independently hydrogen, halogen, —CN, —OH, —NO₂, —C₁₋₆alkyl, —OC₁₋₆alkyl, or —C(O)C₁₋₆ alkyl.

In embodiments, R^{2A} is selected from —C₁₋₆alkyl, cycloalkyl, unsaturated heterocyclic ring comprising at least one carbon-carbon double bond, aryl, or heteroaryl; wherein said —C₁₋₆alkyl, cycloalkyl, unsaturated heterocyclic ring, aryl, or heteroaryl are each independently optionally substituted with at least one substituent R^{24a};
each R^{24a} is independently hydrogen, halogen, oxo, —C₁₋₆alkyl, —SC₁₋₆alkyl, cycloalkyl, cycloalkenyl, cycloalkynyl, heterocyclyl, aryl, heteroaryl, —CN, —P(O)R^{241b}R^{241c}, or —OR^{241a}; wherein said —C₁₋₆alkyl, cycloalkyl, cycloalkenyl, cycloalkynyl, heterocyclyl, aryl or heteroaryl are each independently optionally substituted with at least one substituent R^{242a};
each R^{242a} is independently hydrogen, halogen, —CN, —C₁₋₆alkyl, —NR^{241a}R^{241b}, —COR^{241a}, —CONR^{241a}R^{241b}, —OC₁₋₆alkyl, heterocyclyl, aryl, or heteroaryl; wherein said —C₁₋₆alkyl, heterocyclyl, aryl, or heteroaryl are each independently optionally substituted with at least one substituent R^{243a};
R^{241a}, R^{241b}, and R^{241c} are each independently hydrogen, cycloalkyl, heterocyclyl, or —C₁₋₆alkyl; wherein said —C₁₋₆alkyl is each independently optionally substituted with at least one substituent R^{243a};
each R^{243a} is independently hydrogen, halogen, —CN, —OH, —NO₂, or —OC₁₋₆alkyl.

Aspect 13. The compound according to any one of aspects 1 to 11, wherein:
R^{2A} is selected from:
   —C₁₋₆alkyl;
   3- to 7-membered cycloalkyl;
   unsaturated 3- to 7-membered heterocyclic ring comprising at least one carbon-carbon double bond, optionally substituted with one or more independently selected F, —C₁₋₆alkyl, —CN, or aryl;
   5- to 12-membered aryl optionally substituted with one or more independently selected F, —C₁₋₆ alkyl, —C₁₋₆alkynyl, —SC₁₋₆alkyl, —OC₁₋₆alkyl, —PO(C₁₋₆alkyl)₂, —CN, —COOH, —CONH₂, cycloalkyl, —COcycloalkyl, heterocyclyl, —COheterocyclyl, aryl or heteroaryl; wherein said —C₁₋₆alkyl, cycloalkyl, heterocyclyl, or heteroaryl are each independently optionally substituted with —OH, —CN, F, —NHC₁₋₆alkyl, —C₁₋₆alkyl or —OC₁₋₆alkyl, or
   5- to 15-membered heteroaryl optionally substituted with one or more independently selected F, —C₁₋₆alkyl, —OC₁₋₆alkyl, —CN, oxo, cycloalkyl, heterocyclyl, aryl or heteroaryl; wherein said —C₁₋₆alkyl, cycloalkyl, heterocyclyl, aryl or heteroaryl are each independently optionally substituted with cycloalkyl, heterocyclyl, aryl, heteroaryl, —CN, F, Cl, —OH, —CONH₂, —C₁₋₆alkyl or —OC₁₋₆alkyl.

Aspect 14. The compound according to any one of aspects 1 to 11, wherein:

R^{2A} is selected from —C₁₋₆alkyl,

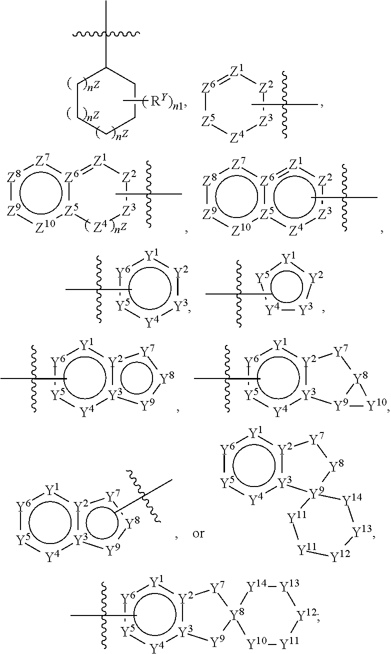

Z¹, Z², Z³, Z⁴, Z⁵, Z⁶, Z⁷, Z⁸, Z⁹, Z¹⁰, Y¹, Y², Y³, Y⁴, Y⁵, Y⁶, Y⁷, Y⁸, Y⁹, Y¹⁰, Y¹¹, Y¹², Y¹³ and Y¹⁴ are each independently C(R^{Y}), C(R^{Y′}R^{Y}), N(R^{Y}), N, O, or S;
n^{Z} is each independently selected from 0, 1, 2, or 3;
n1 is each independently selected from 0, 1, 2, or 3;
R^{Y′} and R^{Y} are each independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —OC₁₋₆alkyl, —SC₁₋₆alkyl, —P(O)(C₁₋₆alkyl)₂, —OH, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl or —SC₁₋₆alkyl are each independently optionally substituted with at least one substituent R^{Ya};
each R^{Ya} is independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —OC₁₋₆alkyl, or —NO₂.

Aspect 15. The compound according to any one of aspects 1 to 11, wherein:
R^{2A} is selected from methyl,

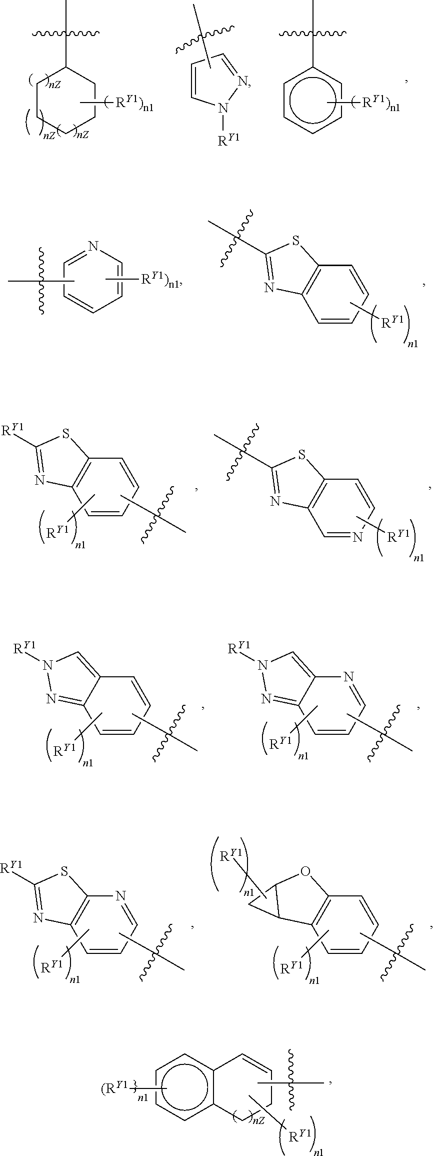

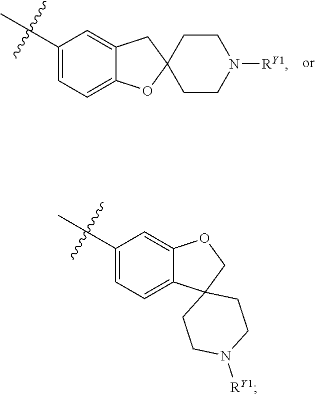

each R¹ is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆ alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl or —SC₁₋₆alkyl are each independently optionally substituted with at least one substituent R^{Y1a};
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂;
n^{Z} is each independently selected from 0, 1, 2, or 3;
n1 is each independently selected from 0, 1, 2, or 3.

In embodiments of Aspect 15, R^{2A} is

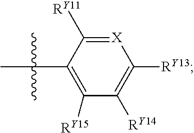

X is C(R¹²) or N;
R^{Y11}, R^{Y12}, R^{Y13}, R^{Y14}, and R^{Y15} are each independently hydrogen, halogen, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆ alkyl or —SC₁₋₆alkyl are each independently optionally substituted with at least one substituent R^{Y1a}; or
(R^{Y11} and R^{Y12}), (R^{Y12} and R^{Y13}), (R^{Y13} and R^{Y14}), or (R^{Y14} and R^{Y15}), together with the atoms to which they are attached, form a C₃₋₁₀cycloalkyl, C₃₋₁₀cycloalkenyl, 4- to 12-membered heterocyclyl ring containing 1, 2 or 3 heteroatoms independently selected from nitrogen, oxygen and optionally oxidized sulfur, 6- to 12-membered aryl, or 4- to 12-membered heteroaryl ring containing 1, 2 or 3 heteroatoms independently selected from nitrogen, oxygen and optionally oxidized sulfur, and optionally substituted with at least one substituent R^{Y1a};
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂.

In some embodiments, R^{Y11} and R^{Y15} are hydrogen.

In some embodiments, 1, 2, or 3 of R^{Y12}, R^{Y13}, and R^{Y14} is hydrogen. In further embodiments, R^{Y12}, R^{Y13}, and R^{Y14} are selected from hydrogen, halogen, —C₁₋₆alkyl, —P(O)(C₁₋₆alkyl)₂, —OH, —CN, —OC₁₋₆alkyl, —SC₁₋₆alkyl, or —NO₂. In alternative further embodiments, (R^{Y12} and R^{Y13}), together with the atoms to which they are attached, form a 4- to 12-membered heterocyclyl ring containing 1, 2 or 3 heteroatoms independently selected from nitrogen, oxygen and optionally oxidized sulfur, or 4- to 12-membered heteroaryl ring containing 1, 2 or 3 heteroatoms independently selected from nitrogen, oxygen and optionally oxidized sulfur, and optionally substituted with at least one substituent R^{Y1a}.

In some embodiments, R^{Y12} and R^{Y14} are hydrogen; and R^{Y13} is halogen, —C₁₋₆alkyl, —C₂₋₆alkenyl, —C₂₋₆alkynyl, cycloalkyl, cycloalkenyl, or cycloalkynyl, heterocyclyl, —O-heterocyclyl, —CO-heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆ alkyl or —SC₁₋₆alkyl are each independently optionally substituted with at least one substituent R^{Y1a}.

In embodiments of Aspect 15, R^{2A} is

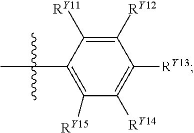

R^{Y11}, R^{Y12}, R^{Y13}, R^{Y14}, and R^{Y15} are each independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO— heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl or —SC₁₋₆alkyl are each independently optionally substituted with at least one substituent R^{Y1a};
(R^{Y11} and R^{Y12}), (R^{Y12} and R^{Y13}), (R^{Y13} and R^{Y14}), or (R^{Y14} and R^{Y15}), together with the atoms to which they are attached, form a C₃₋₁₀ cycloalkyl, C₃₋₁₀ cycloalkenyl, 4- to 12-membered heterocyclyl ring containing 1, 2 or 3 heteroatoms independently selected from nitrogen, oxygen and optionally oxidized sulfur, 6- to 12-membered aryl, or 4- to 12-membered heteroaryl ring containing 1, 2 or 3 heteroatoms independently selected from nitrogen, oxygen and optionally oxidized sulfur, and optionally substituted with at least one substituent R^{Y1a};
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂.

In some embodiments, R^{Y11}, R^{Y12}, R^{Y13}, R^{Y14}, and R^{Y15} are each independently hydrogen, halogen, —C₁₋₆alkyl, cycloalkyl, heterocyclyl, aryl, heteroaryl, —OH, —CN, —OC₁₋₆alkyl, —SC₁₋₆alkyl, or —NO₂, wherein said —C₁₋₆alkyl, cycloalkyl, heterocyclyl, aryl, heteroaryl are each independently optionally substituted with at least one substituent R^{Y1a};
(R^{Y11} and R^{Y12}), (R^{Y12} and R^{Y13}), (R^{Y13} and R^{Y14}), or (R^{Y14} and R^{Y15}), together with the atoms to which they are attached, form a 5- to 6-membered heterocyclyl ring containing 1, 2 or 3 heteroatoms independently selected from nitrogen, oxygen and optionally oxidized sulfur, 6- to 12-membered aryl, or 5- to 6-membered heteroaryl ring containing 1, 2 or 3 heteroatoms independently selected from nitrogen, oxygen and optionally oxidized sulfur, and optionally substituted with at least one substituent R^{Y1a};
each R^{Y1a} is independently hydrogen, —CN, halogen, —C₁₋₆alkyl, —OC₁₋₆alkyl, or —OH.

In some embodiments, R^{Y11}, R^{Y12}, R^{Y13}, R^{Y14}, and R^{Y15} are each independently hydrogen, F, —CN, pyrazolyl, —C₁₋₆alkyl, cyclopropanyl, piperidinyl, or tetrahydro-pyranyl; wherein said pyrazolyl, —C₁₋₆ alkyl, cyclopropanyl, piperidinyl, or tetrahydro-pyranyl are each independently optionally substituted with at least one substituent R^{Y1a};
each R^{Y1a} is independently hydrogen, —CN, F, —C₁₋₆alkyl, —OC₁₋₆alkyl, or —OH.

In embodiments of Aspect 15, R^{2A} is

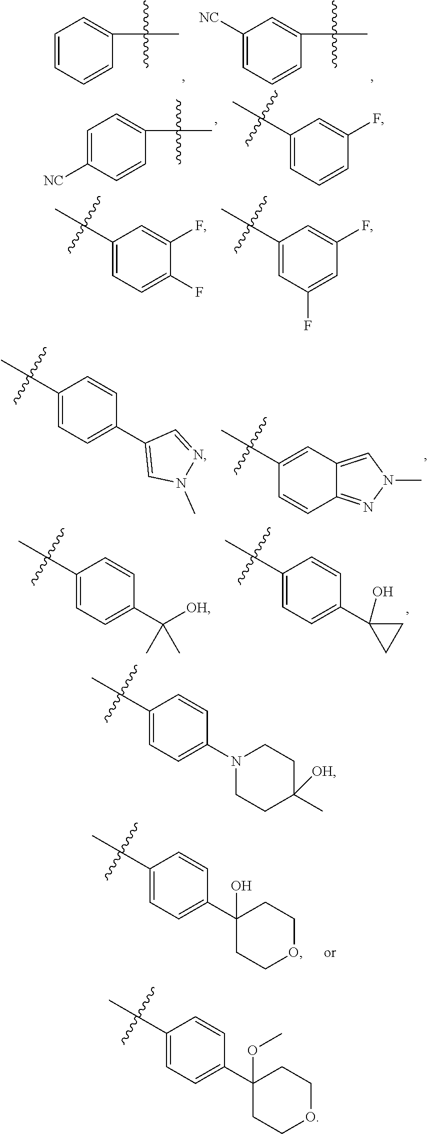

In embodiments of Aspect 15, R^{2A} is

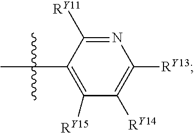

R^{Y11}, R^{Y13}, R^{Y14}, and R^{Y15} are each independently hydrogen, halogen, —C₁₋₆alkyl, cycloalkyl, heterocyclyl, aryl, heteroaryl, —OH, —CN, —OC₁₋₆alkyl, —SC₁₋₆alkyl, or —NO₂, wherein said —C₁₋₆alkyl, cycloalkyl, heterocyclyl, aryl, heteroaryl are each independently optionally substituted with at least one substituent R^{Y1a};
(R^{Y13} and R^{Y14}), or (R^{Y14} and R^{Y15}), together with the atoms to which they are attached, form a 5- to 6-membered heterocyclyl ring containing 1, 2 or 3 heteroatoms independently selected from nitrogen, oxygen and optionally oxidized sulfur, 6- to 12-membered aryl, or 5- to 6-membered heteroaryl ring containing 1, 2 or 3 heteroatoms independently selected from nitrogen, oxygen and optionally oxidized sulfur, and optionally substituted with at least one substituent R^{Y1a};
each R^{Y1a} is independently hydrogen, —CN, halogen, —C₁₋₆alkyl, —OC₁₋₆alkyl, or —OH.

In some embodiments, R^{Y11}, R^{Y13}, R^{Y14}, and R^{Y15} are each independently hydrogen, F, —CN, pyrazolyl, —C₁₋₆alkyl, cyclopropanyl, piperidinyl, or tetrahydro-pyranyl; wherein said pyrazolyl, —C₁₋₆ alkyl, cyclopropanyl, piperidinyl, or tetrahydro-pyranyl are each independently optionally substituted with at least one substituent R^{Y1a};
each R^{Y1a} is independently hydrogen, —CN, F, —C₁₋₆alkyl, —OC₁₋₆alkyl, or —OH.

In some embodiments, R^{Y11}, R^{Y13}, R^{Y14}, and R^{Y15} are each independently hydrogen, F, —CN, pyrazolyl, —C₁₋₆alkyl, cyclopropanyl, piperidinyl, or tetrahydro-pyranyl; wherein said pyrazolyl, —C₁₋₆ alkyl, cyclopropanyl, piperidinyl, or tetrahydro-pyranyl are each independently optionally substituted with at least one substituent R^{Y1a};
each R^{Y1a} is independently hydrogen, —CN, F, —C₁₋₆alkyl, —OC₁₋₆alkyl, or —OH.

In embodiments of Aspect 15, R^{2A} is selected from

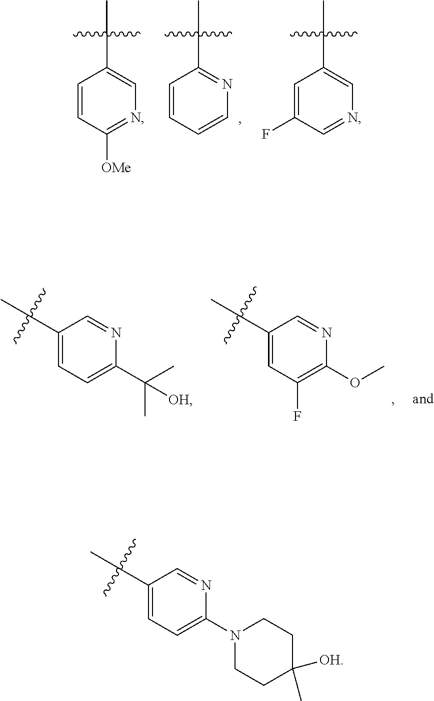

Aspect 16. The compound according to aspect 15, wherein:
each R^{Y1} is independently F, —C₁₋₆alkyl, aryl, heteroaryl, —P(O)(CH₃)₂, —OH, —CN, or —OC₁₋₆alkyl, wherein said —C₁₋₆alkyl, aryl, heteroaryl, or —OC₁₋₆alkyl are each independently optionally substituted with at least one substituent R^{Y1a};
each R^{Y1a} is independently F, —C₁₋₆alkyl, —OH, —CN, or —OC₁₋₆alkyl.

Aspect 17. The compound according to any one of aspects 1 to 11, wherein:
R^{2A} is selected from methyl,

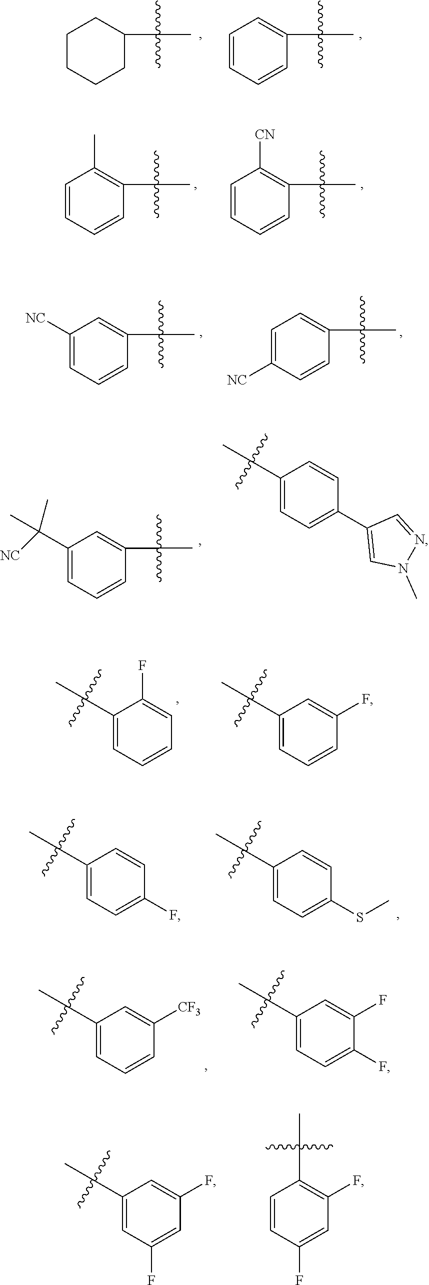

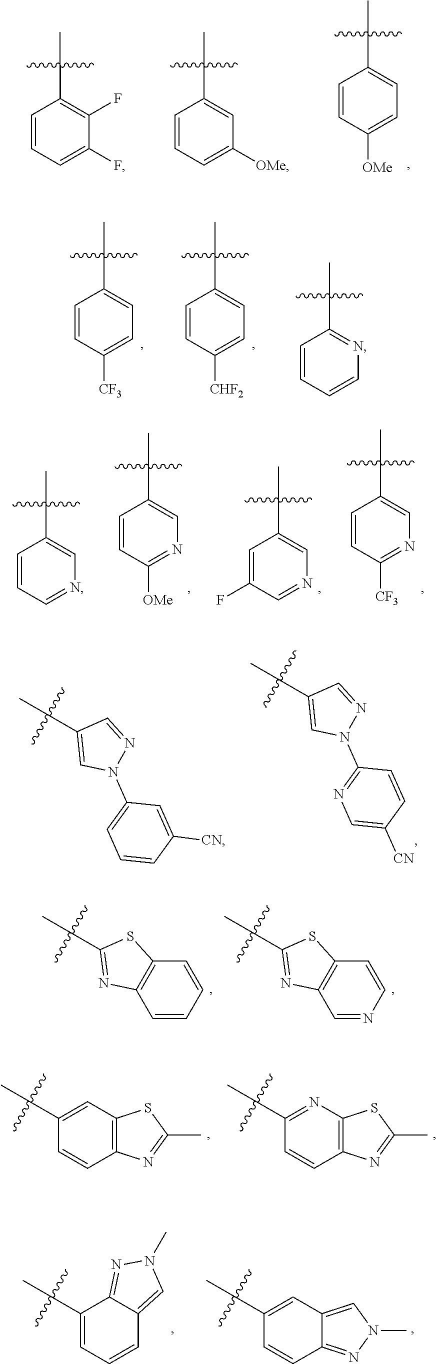

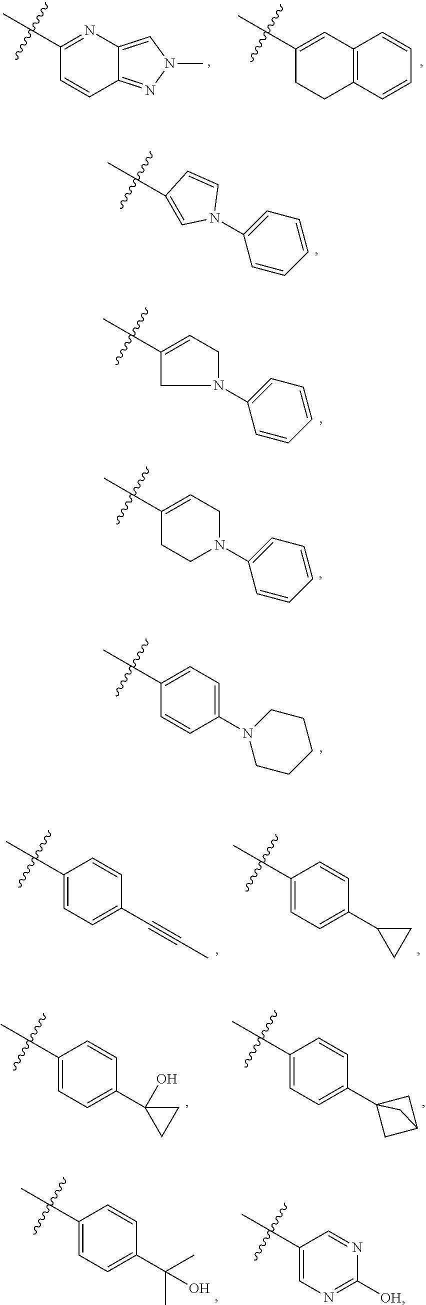

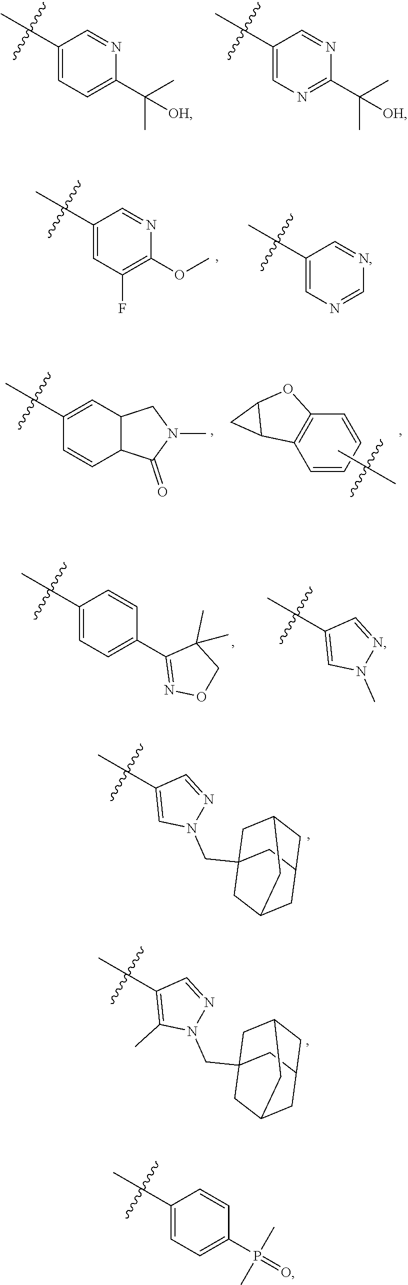

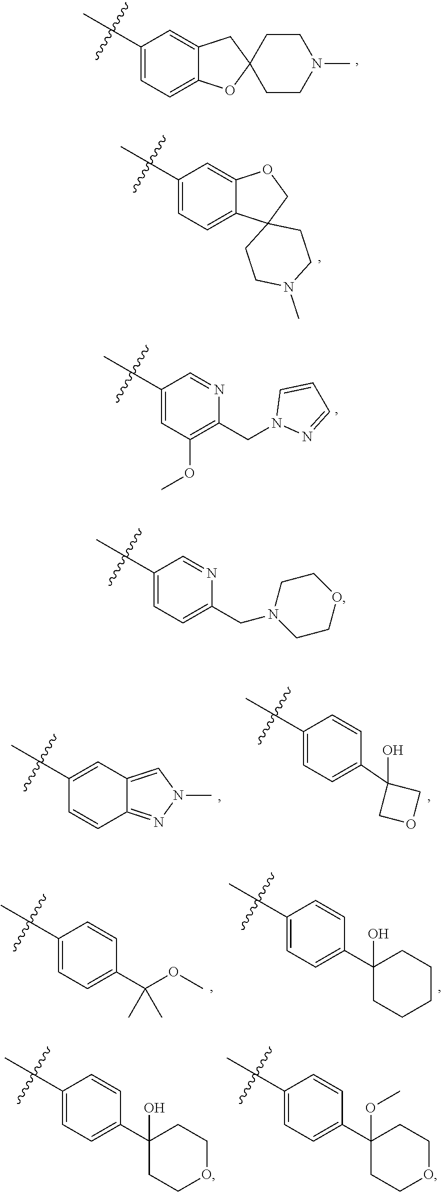

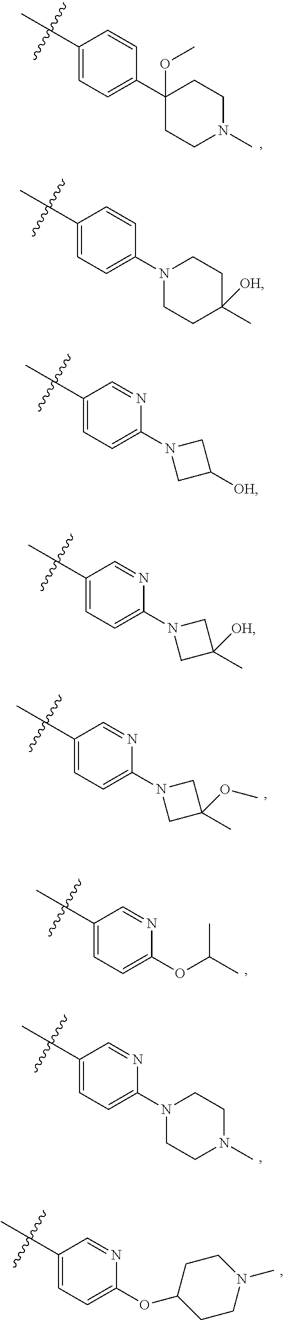

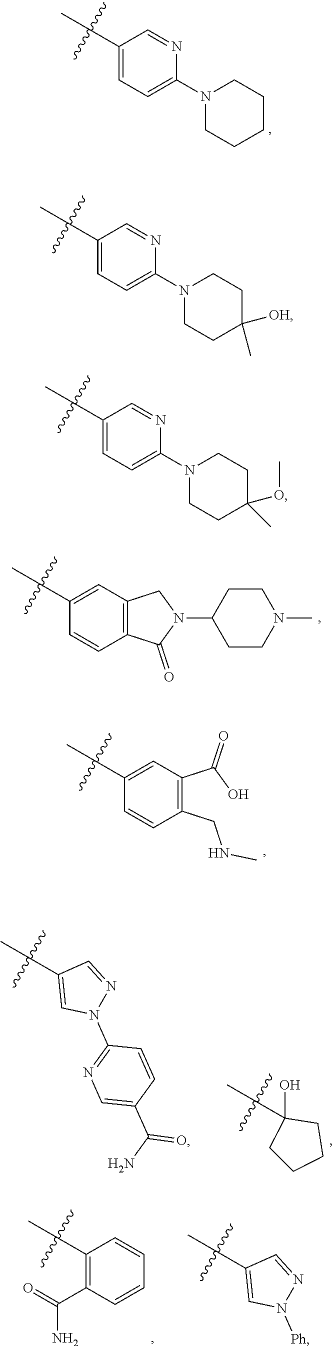

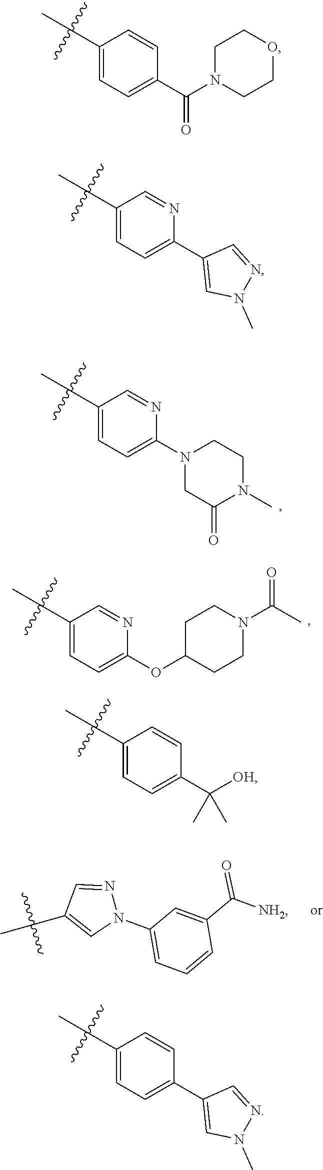

Aspect 18(a). The compound according to any one of aspects 1 to 17, wherein R^{4A} and R^{4B}, which are different, are each independently hydrogen, deuterium, halogen, —C₁₋₆alkyl, or deuterated —C₁₋₆alkyl, the carbon atom R^{4A} and R^{4B} attached to form S or R configuration.

In embodiments, R^{4A} is hydrogen or deuterium, and R^{4B} is methyl or —CD₃. In embodiments, R^{4A} is hydrogen and R^{4B} is methyl.

In embodiments, R^{4A} is hydrogen or deuterium, and R^{4B} is methyl or —CD₃, the carbon atom R^{4A} and R^{4B} attached to form S configuration. In embodiments, R^{4A} is hydrogen and R^{4B} is methyl, the carbon atom R^{4A} and R^{4B} attached to form S configuration.

In embodiments, R^{4A} is hydrogen or deuterium, and R^{4B} is methyl or —CD₃, the carbon atom R^{4A} and R^{4B} attached to form R configuration. In embodiments, R^{4A} is hydrogen and R^{4B} is methyl, the carbon atom R^{4A} and R^{4B} attached to form R configuration.

Aspect 18. The compound according to any one of aspects 1 to 17, wherein R^{4A} and R^{4B}, which may be the same or different, are each independently hydrogen, halogen, or —C₁₋₆alkyl; wherein said —C₁₋₆ alkyl is each independently optionally substituted with at least one substituent R^{44a};
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 19. The compound according to any one of aspects 1 to 17, wherein:
R^{4A} and R^{4B}, which are different, are each independently hydrogen, halogen, or —C₁₋₆alkyl; wherein said —C₁₋₆alkyl is each independently optionally substituted with at least one substituent R^{44a};
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 20. The compound according to any one of aspects 1 to 17, wherein:
R^{4A} and R^{4B}, which are different, are each independently hydrogen, or —C₁₋₆alkyl; wherein said —C₁₋₆ alkyl is each independently optionally substituted with at least one substituent R^{44a};
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 21. The compound according to any one of aspects 1 to 17, wherein:
R^{4A} and R^{4B}, which are different, are each independently hydrogen, or —C₁₋₆alkyl; wherein said —C₁₋₆ alkyl is each independently optionally substituted with at least one substituent R^{44a}; the carbon atom R^{4A} and R^{4B} attached to form S or R configuration;
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 22. The compound according to any one of aspects 1 to 17, wherein:
R^{4A} is hydrogen, and R^{4B} is —C₁₋₆alkyl; wherein said —C₁₋₆alkyl is each independently optionally substituted with at least one substituent R^{44a}; the carbon atom R^{4A} and R^{4B} attached to form S configuration;
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 23. The compound according to any one of aspects 1 to 17, wherein:
R^{4B} is hydrogen, and R^{4A} is —C₁₋₆alkyl; wherein said —C₁₋₆alkyl is each independently optionally substituted with at least one substituent R^{44a}; the carbon atom R^{4A} and R^{4B} attached to form R configuration;
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

In embodiments of Aspect 23, the compound has formula (IV-1)

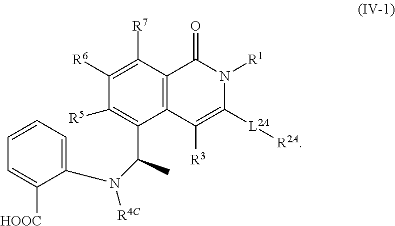

Aspect 24. The compound according to any one of aspects 1 to 17, wherein:
R^{4A} is hydrogen, and R^{4B} is —C₁₋₆alkyl; wherein said —C₁₋₆alkyl is each independently optionally substituted with at least one substituent R^{44a};
each R^{44a} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 25. The compound according to any one of aspects 1 to 24, wherein:
R^{4C} is hydrogen, halogen, or —C₁₋₆alkyl; wherein said —C₁₋₆alkyl is each independently optionally substituted with at least one substituent R^{44d};
each R^{44d} is independently hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, or —CO₂C₁₋₆alkyl.

Aspect 26. The compound according to any one of aspects 1 to 24, wherein R^{4C} is hydrogen.

Aspect 27. The compound according to any one of aspects 1 to 26, wherein:
R^{4D} is aryl, or heteroaryl; wherein said aryl, or heteroaryl are each independently optionally substituted with at least one substituent R^{44e};
each R^{44e} is independently halogen, —C₁₋₆alkyl, —C₂₋₆alkenyl, —C₂₋₆alkynyl, —CN, —NO₂, —OR^{444d}, —SO₂R^{444d}, —COR^{444d}, —CO₂R^{444d}, —CONR^{444d}R^{444e}, —C(═NR^{444d})NR^{444e}R^{444f}, —NR^{444d}R^{444e}, —NR^{444d}COR^{444e}, —NR^{444d}CONR^{444e}R^{444f}, —NR^{444d}CO₂R^{444e}, —NR^{444d}SONR^{444e}R^{444f}, —SO₂NR^{444e}R^{444f}, —SO₂NR^{444e}COR^{444f}, or —NR^{444d}SO₂R^{444e}; each of said —C₁₋₆alkyl, —C₂₋₆alkenyl, —C₂₋₆alkynyl, cycloalkyl, cycloalkenyl, cycloalkynyl, heterocyclyl, aryl, heteroaryl are each independently optionally substituted with at least one substituent R^{N}.

R^{444d}, R^{444e}, and R^{444f} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, heteroaryl —C₁₋₆haloalkyl, —COC₁₋₆alkyl, or —CO₂C₁₋₆alkyl;
R^{N} is each independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆haloalkyl, —COC₁₋₆alkyl, or —CO₂C₁₋₆alkyl.

Aspect 28. The compound according to any one of aspects 1 to 26, wherein:
R^{4D} is aryl; wherein said aryl, or heteroaryl are each independently optionally substituted with at least one substituent R^{44e};
each R^{44e} is independently halogen, —C₁₋₆alkyl, —CN, —NO₂, —OR^{444d}, —SO₂R^{444d}, —COR^{444d}, —CO₂R^{444d}, —CONR^{444d}R^{444e}, —NR^{444d}R^{444e}, or —SO₂NR^{444e}R^{444f}; each of said —C₁₋₆alkyl is each independently optionally substituted with at least one substituent R^{N};
R^{444d}, R^{444e}, and R^{444f} are each independently hydrogen, halogen, —C₁₋₆alkyl, —CN, —NO₂, —OC₁₋₆alkyl, —C₁₋₆haloalkyl, —COC₁₋₆alkyl, or —CO₂C₁₋₆alkyl;
R^{N} is each independently hydrogen, halogen, —C₁₋₆alkyl, —CN, —NO₂, —OC₁₋₆alkyl, —C₁₋₆haloalkyl, —COC₁₋₆alkyl, or —CO₂C₁₋₆alkyl.

Aspect 29. The compound according to any one of aspects 1 to 26, wherein: R^{4D} is phenyl; wherein said phenyl is optionally substituted with at least one substituent R^{44e};
each R^{44e} is independently halogen, —C₁₋₆alkyl, —CN, —NO₂, —OR^{444d}, —SO₂R^{444d}, —COR^{444d}, —CO₂R^{444d}, —CONR^{444d}R^{444e}, —NR^{444d}R^{444e}, —SO₂R^{444d}, or —SO₂NR^{444e}R^{444f}; each of said —C₁₋₆alkyl is each independently optionally substituted with at least one substituent R^{N};
R^{444d}, R^{444e}, and R^{444f} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, heteroaryl —C₁₋₆haloalkyl, —COC₁₋₆alkyl, or —CO₂C₁₋₆alkyl;
R^{N} is each independently hydrogen, halogen, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —C₁₋₆haloalkyl, —COC₁₋₆alkyl, or —CO₂C₁₋₆alkyl.

Aspect 30. The compound according to any one of aspects 1 to 26, wherein R^{4D} is

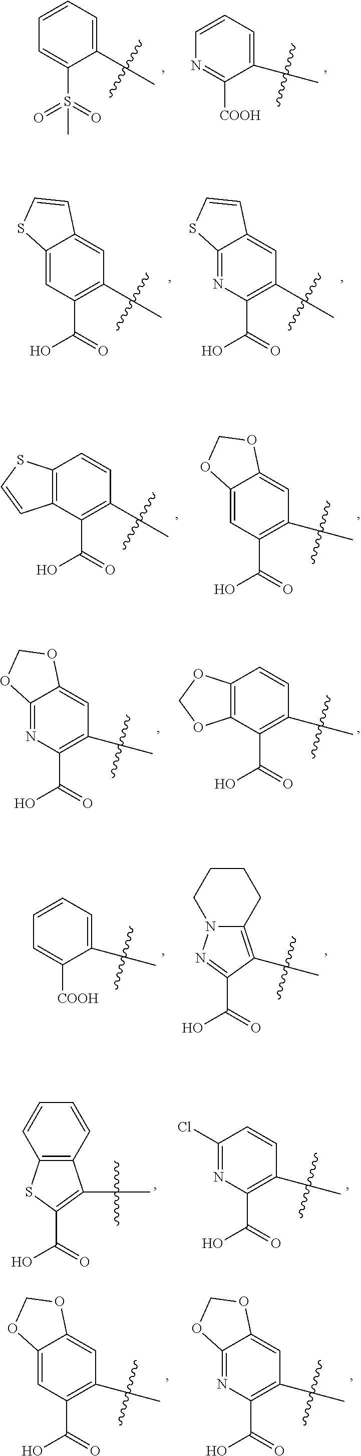

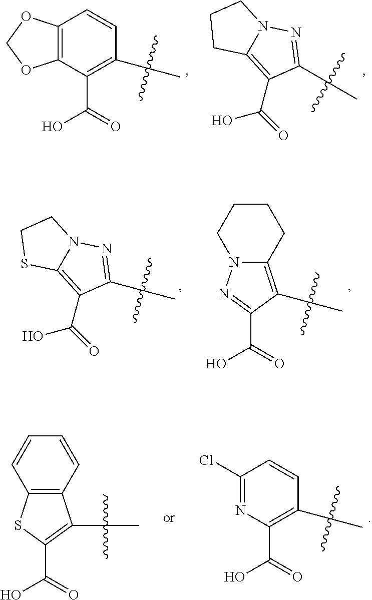 [SMILES: *c1ccc2c(c1C(=O)O)OCO2 ; *c1nn2c(c1C(=O)O)CCC2 ; *c1nn2c(c1C(=O)O)SCC2 ; *c1c(C(=O)O)nn2c1CCCC2 ; *c1c(C(=O)O)sc2ccccc12 ; *c1ccc(Cl)nc1C(=O)O]

Aspect 31. The compound according to any one of aspects 1-30, wherein
R¹ is hydrogen, —C₁₋₆alkyl, heterocyclyl, heteroaryl, or aryl; wherein said —C₁₋₆alkyl, heterocyclyl, heteroaryl, or aryl are each independently optionally substituted with at least one substituent R^{1a};
each R^{11a} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{1a}, —COR^{1a}, —CO₂R^{1a}, —CONR^{1a}R^{1b}, —NR^{1a}R^{1b}, —NR^{1a}COR^{1b}, —NR^{1a}CONR^{1b}R^{1c}, or —NR^{1a}CO₂R^{1b}; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{1d};
R^{1a}, R^{1b}, and R^{1c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{1d};
each R^{1d} is independently hydrogen, halogen, oxo, —CN, —NO₂, —C₁₋₆alkyl, —C₂₋₆alkenyl, —C₂₋₆alkynyl, —C₁₋₆haloalkyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl.

In embodiments of Aspect 31, R¹ is —C₁₋₆alkyl, deuterated —C₁₋₆alkyl, oxetanyl, tetrahydrofuranyl, tetrahydro-pyranyl, pyridinyl, thietane 1,1-dioxidyl, pyrazolyl, isoxazolyl, oxazolyl, or thiazolyl; wherein said —C₁₋₆alkyl, or deuterated —C₁₋₆alkyl are each independently optionally substituted with at least one substituent selected from hydrogen, deuterium, —CN, oxetanyl, tetrahydrofuranyl, tetrahydro-pyranyl, pyridinyl, thietane 1,1-dioxidyl, pyrazolyl, isoxazolyl, oxazolyl, and thiazolyl; wherein said oxetanyl, tetrahydrofuranyl, tetrahydro-pyranyl, pyridinyl, thietane 1,1-dioxidyl, pyrazolyl, isoxazolyl, oxazolyl, or thiazolyl are each optionally substituted with at least one substituent selected from —C₁₋₆alkyl.

Aspect 32. The compound according to any one of aspects 1-30, wherein R¹ is hydrogen, methyl, —CH₂CN, —CH₂OCH₃, —CH₂CH₃, —CH₂CH₃CN, —CH₂SO₂CH₃, —COCH₃, —CH₂OH, —CH₂CH₂OCH₃,

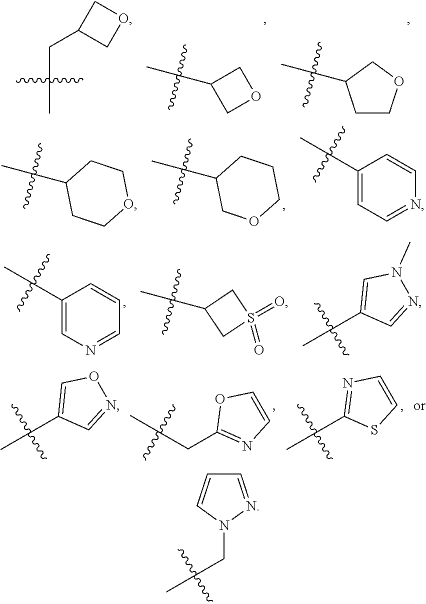

In embodiments of Aspect 32, R¹ is methyl, —CD₃, or —CH₂CN.

In embodiments of Aspect 32, R¹ is methyl.

In embodiments of Aspect 32, R¹ is —CD₃.

Aspect 33. The compound according to any one of aspects 1-32, wherein R³ is hydrogen.

Aspect 34. The compound according to any one of aspects 1-33, wherein R⁵ is hydrogen.

Aspect 35(a). The compound according to any one of aspects 1-34, wherein R⁶ is hydrogen, F, —C₁₋₆alkyl optionally substituted with at least one halogen, or deuterated methyl; preferably, R⁶ is hydrogen, F, methyl, —CD₃, —CF₃, —CHF₂ or —CH₂F.

Aspect 35. The compound according to any one of aspects 1-34, wherein R⁶ is hydrogen, F, or —C₁₋₆alkyl optionally substituted with at least one halogen; preferably, R⁶ is F, —CH₃, —CH₂F, —CHF₂, or —CF₃; more preferably, R⁶ is —CH₃ or —CF₃.

Aspect 36. The compound according to any one of aspects 1-34, wherein R⁶ is deuterated methyl; preferably, R⁶ is —CD₃.

In embodiments of Aspect 36, R⁶ is methyl.

In embodiments of Aspect 36, R⁶ is —CD₃.

Aspect 37. The compound according to any one of aspects 1-36, wherein R⁷ is hydrogen.

Aspect 38. The compound according to any one of aspects 1-37, wherein R¹ is deuterated C₁₋₆ alkyl; R^{4A}, R^{4B} are each independently deuterium or deuterated C₁₋₆alkyl.

Aspect 39. The compound according to any one of aspects 1-37, wherein R¹ is —CD₃; and R^{4A}, R^{4B} are each independently deuterium or —CD₃.

Aspect 40. The compound according to any one of aspects 1-37, wherein R¹ is —CD₃; R^{4A} is D; and R^{4B} is —CD₃.

Aspect 41. The compound according to any one of aspects 1-40, wherein the compound of Formula (I) is selected from:

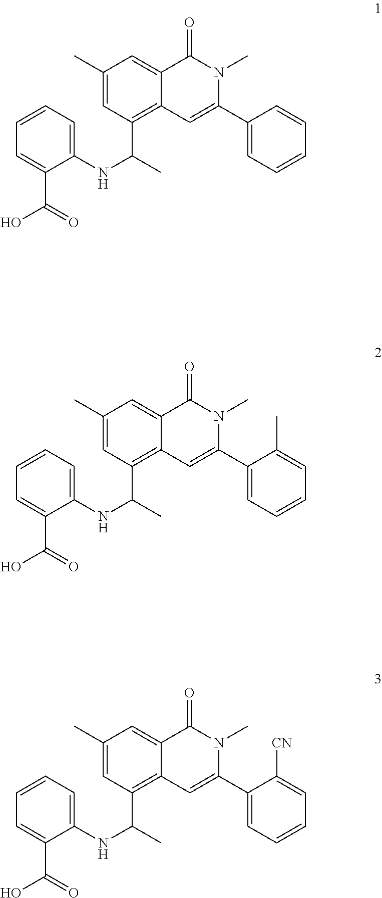 [SMILES: Cc1cc(C(C)Nc2ccccc2C(=O)O)c2cc(-c3ccccc3)n(C)c(=O)c2c1 ; Cc1cc(C(C)Nc2ccccc2C(=O)O)c2cc(-c3ccccc3C)n(C)c(=O)c2c1 ; Cc1cc(C(C)Nc2ccccc2C(=O)O)c2cc(-c3ccccc3C#N)n(C)c(=O)c2c1]

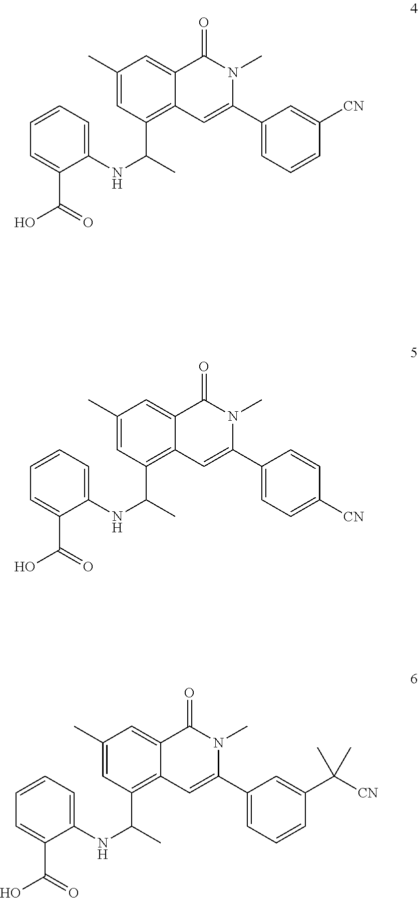 [SMILES: Cc1cc(C(C)Nc2ccccc2C(=O)O)c2cc(-c3cccc(C#N)c3)n(C)c(=O)c2c1 ; Cc1cc(C(C)Nc2ccccc2C(=O)O)c2cc(-c3ccc(C#N)cc3)n(C)c(=O)c2c1 ; Cc1cc(C(C)Nc2ccccc2C(=O)O)c2cc(-c3cccc(C(C)(C)C#N)c3)n(C)c(=O)c2c1]

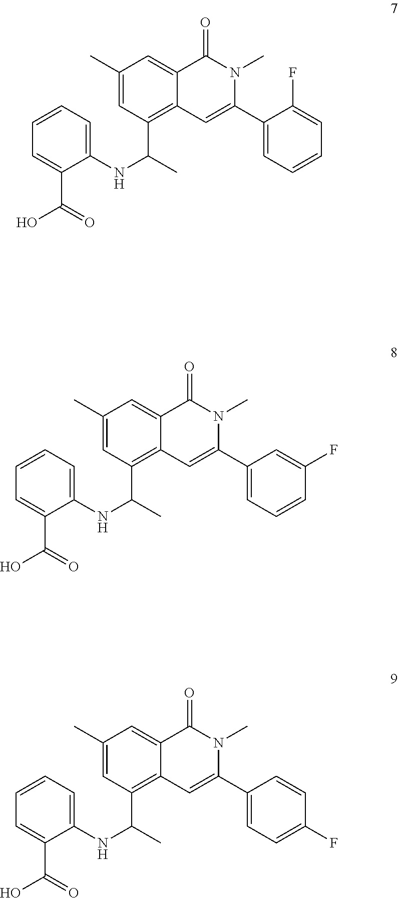 [SMILES: Cc1cc(C(C)Nc2ccccc2C(=O)O)c2cc(-c3ccccc3F)n(C)c(=O)c2c1 ; Cc1cc(C(C)Nc2ccccc2C(=O)O)c2cc(-c3cccc(F)c3)n(C)c(=O)c2c1 ; Cc1cc(C(C)Nc2ccccc2C(=O)O)c2cc(-c3ccc(F)cc3)n(C)c(=O)c2c1]

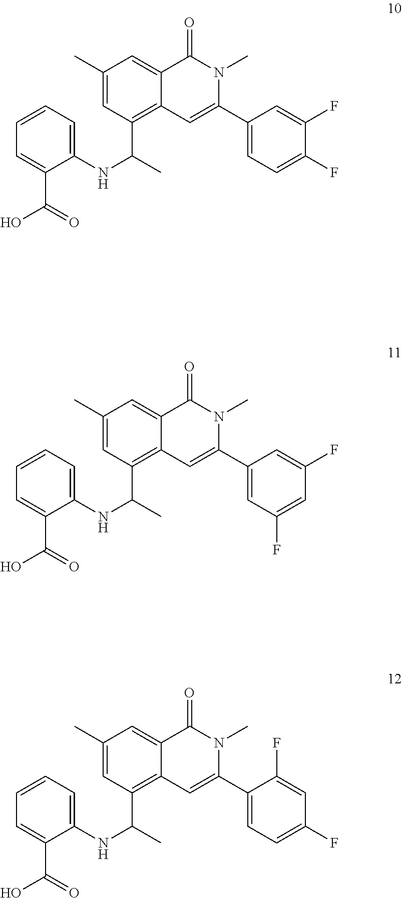 [SMILES: Cc1cc(C(C)Nc2ccccc2C(=O)O)c2cc(-c3ccc(F)c(F)c3)n(C)c(=O)c2c1 ; Cc1cc(C(C)Nc2ccccc2C(=O)O)c2cc(-c3cc(F)cc(F)c3)n(C)c(=O)c2c1 ; Cc1cc(C(C)Nc2ccccc2C(=O)O)c2cc(-c3ccc(F)cc3F)n(C)c(=O)c2c1]

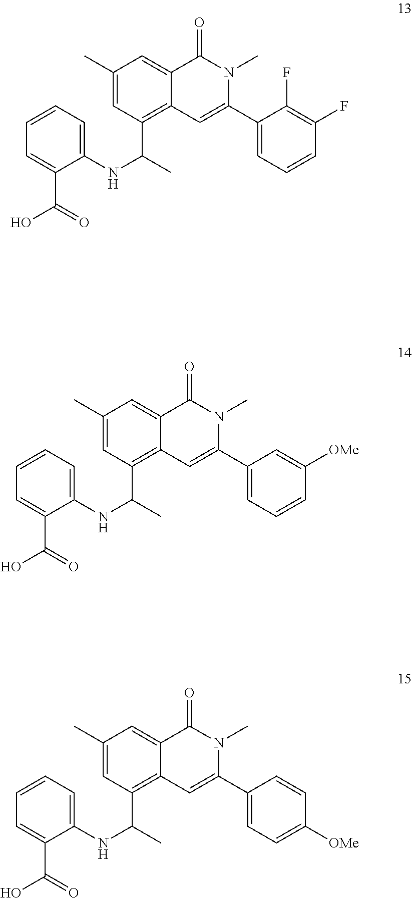 [SMILES: Cc1cc(C(C)Nc2ccccc2C(=O)O)c2cc(-c3cccc(F)c3F)n(C)c(=O)c2c1 ; COc1cccc(-c2cc3c(C(C)Nc4ccccc4C(=O)O)cc(C)cc3c(=O)n2C)c1 ; COc1ccc(-c2cc3c(C(C)Nc4ccccc4C(=O)O)cc(C)cc3c(=O)n2C)cc1]

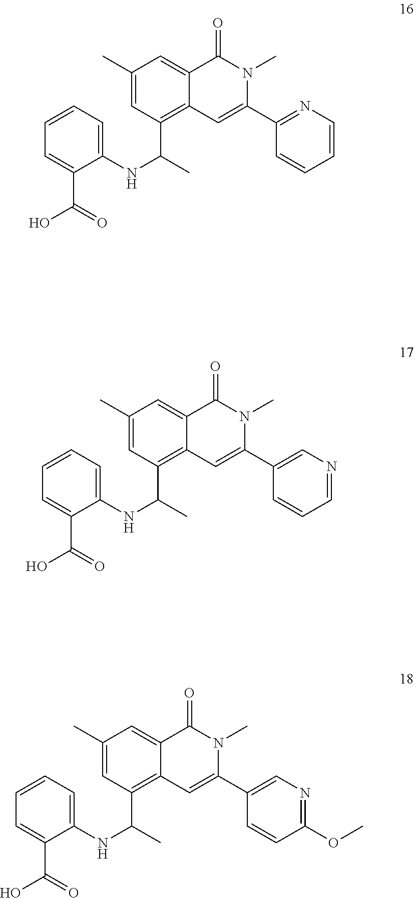 [SMILES: Cc1cc(C(C)Nc2ccccc2C(=O)O)c2cc(-c3ccccn3)n(C)c(=O)c2c1 ; Cc1cc(C(C)Nc2ccccc2C(=O)O)c2cc(-c3cccnc3)n(C)c(=O)c2c1 ; COc1ccc(-c2cc3c(C(C)Nc4ccccc4C(=O)O)cc(C)cc3c(=O)n2C)cn1]

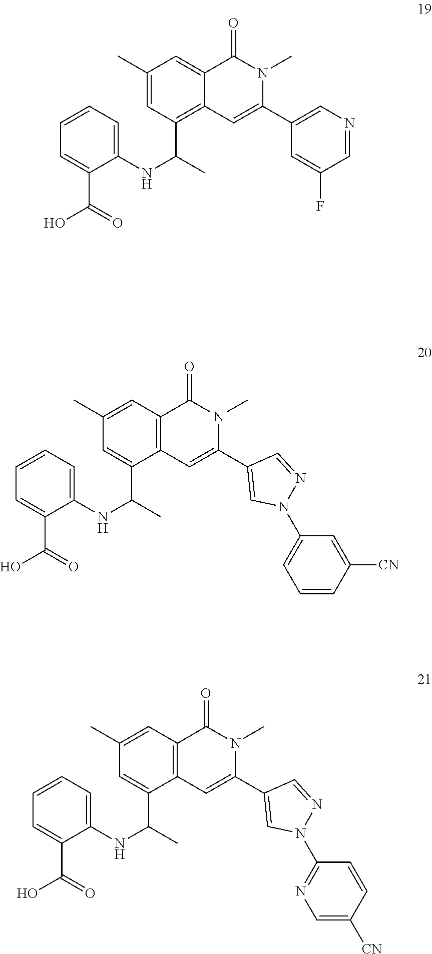 [SMILES: Cc1cc(C(C)Nc2ccccc2C(=O)O)c2cc(-c3cncc(F)c3)n(C)c(=O)c2c1 ; Cc1cc(C(C)Nc2ccccc2C(=O)O)c2cc(-c3cnn(-c4cccc(C#N)c4)c3)n(C)c(=O)c2c1 ; Cc1cc(C(C)Nc2ccccc2C(=O)O)c2cc(-c3cnn(-c4ccc(C#N)cn4)c3)n(C)c(=O)c2c1]

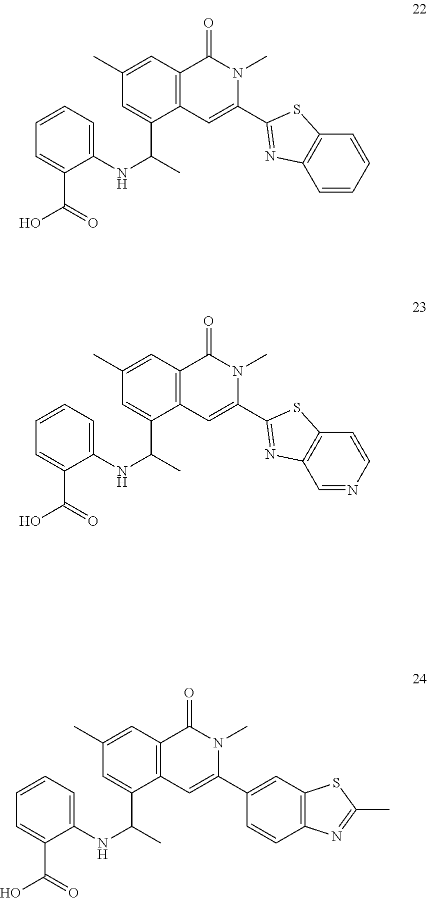 [SMILES: Cc1cc(C(C)Nc2ccccc2C(=O)O)c2cc(-c3nc4ccccc4s3)n(C)c(=O)c2c1 ; Cc1cc(C(C)Nc2ccccc2C(=O)O)c2cc(-c3nc4cnccc4s3)n(C)c(=O)c2c1 ; Cc1cc(C(C)Nc2ccccc2C(=O)O)c2cc(-c3ccc4nc(C)sc4c3)n(C)c(=O)c2c1]

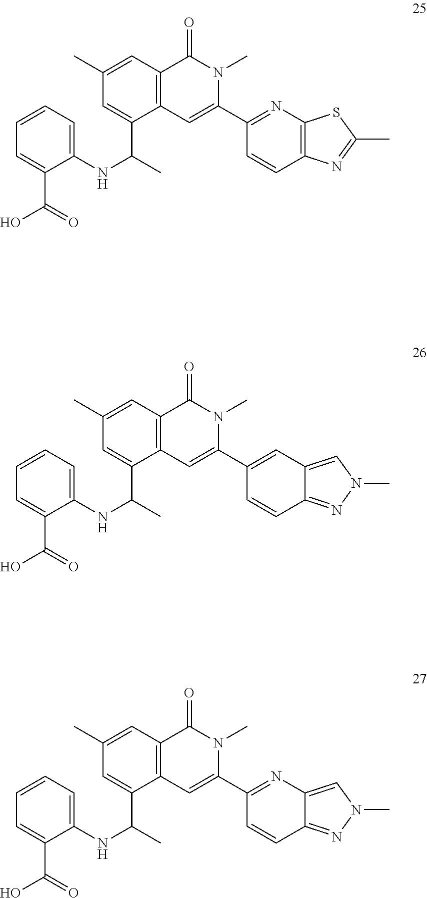 [SMILES: Cc1cc(C(C)Nc2ccccc2C(=O)O)c2cc(-c3ccc4nc(C)sc4n3)n(C)c(=O)c2c1 ; Cc1cc(C(C)Nc2ccccc2C(=O)O)c2cc(-c3ccc4nn(C)cc4c3)n(C)c(=O)c2c1 ; Cc1cc(C(C)Nc2ccccc2C(=O)O)c2cc(-c3ccc4nn(C)cc4n3)n(C)c(=O)c2c1]

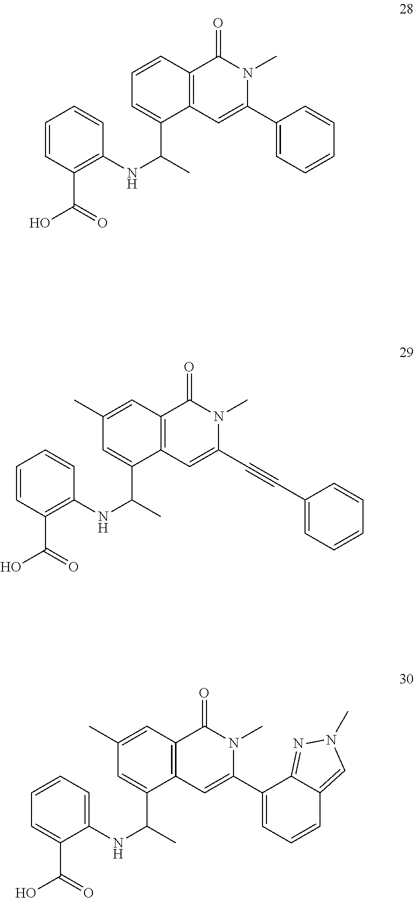 [SMILES: CC(Nc1ccccc1C(=O)O)c1cccc2c(=O)n(C)c(-c3ccccc3)cc12 ; Cc1cc(C(C)Nc2ccccc2C(=O)O)c2cc(C#Cc3ccccc3)n(C)c(=O)c2c1 ; Cc1cc(C(C)Nc2ccccc2C(=O)O)c2cc(-c3cccc4cn(C)nc34)n(C)c(=O)c2c1]

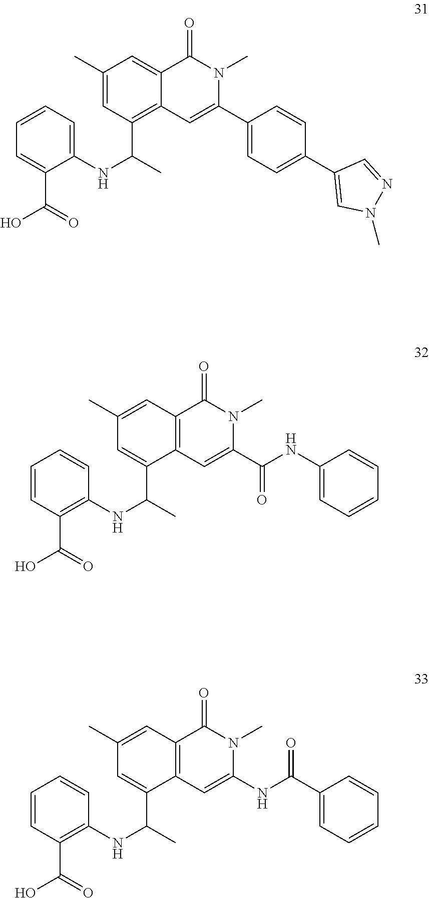 [SMILES: Cc1cc(C(C)Nc2ccccc2C(=O)O)c2cc(-c3ccc(-c4cnn(C)c4)cc3)n(C)c(=O)c2c1 ; Cc1cc(C(C)Nc2ccccc2C(=O)O)c2cc(C(=O)Nc3ccccc3)n(C)c(=O)c2c1 ; Cc1cc(C(C)Nc2ccccc2C(=O)O)c2cc(NC(=O)c3ccccc3)n(C)c(=O)c2c1]

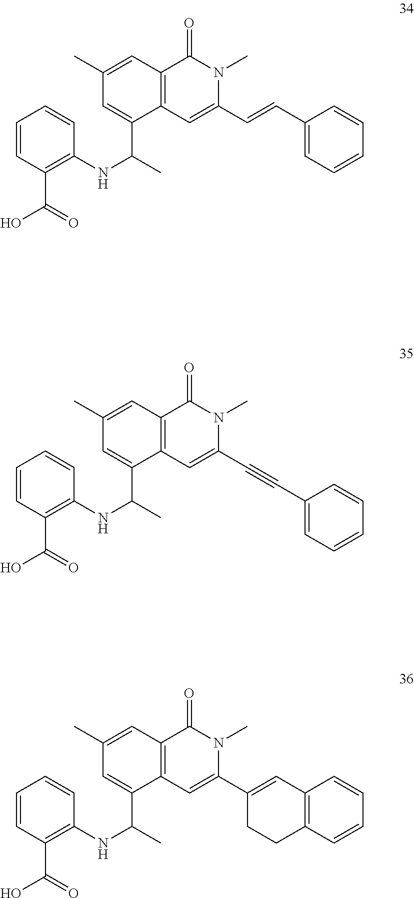 [SMILES: Cc1cc(C(C)Nc2ccccc2C(=O)O)c2cc(/C=C/c3ccccc3)n(C)c(=O)c2c1 ; Cc1cc(C(C)Nc2ccccc2C(=O)O)c2cc(C#Cc3ccccc3)n(C)c(=O)c2c1 ; Cc1cc(C(C)Nc2ccccc2C(=O)O)c2cc(C3=Cc4ccccc4CC3)n(C)c(=O)c2c1]

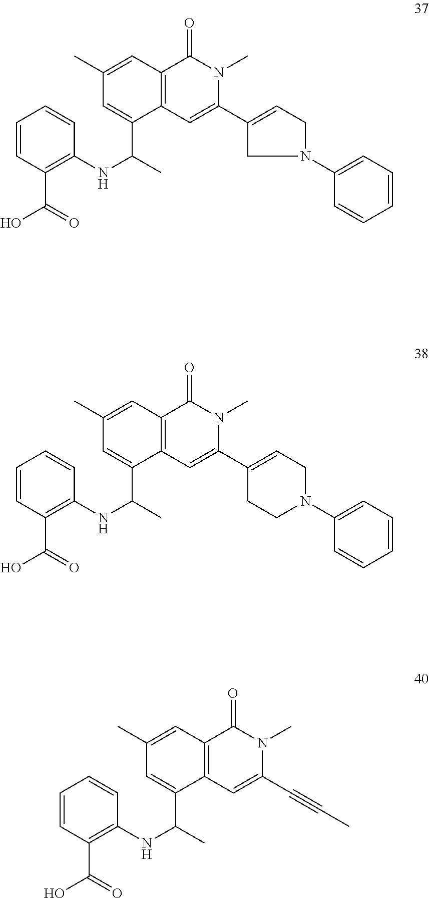 [SMILES: Cc1cc(C(C)Nc2ccccc2C(=O)O)c2cc(C3=CCN(c4ccccc4)C3)n(C)c(=O)c2c1 ; Cc1cc(C(C)Nc2ccccc2C(=O)O)c2cc(C3=CCN(c4ccccc4)CC3)n(C)c(=O)c2c1 ; CC#Cc1cc2c(C(C)Nc3ccccc3C(=O)O)cc(C)cc2c(=O)n1C]

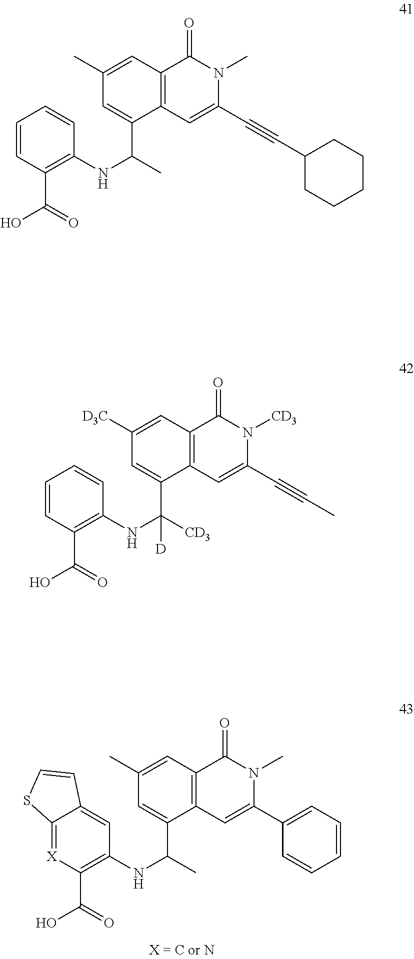

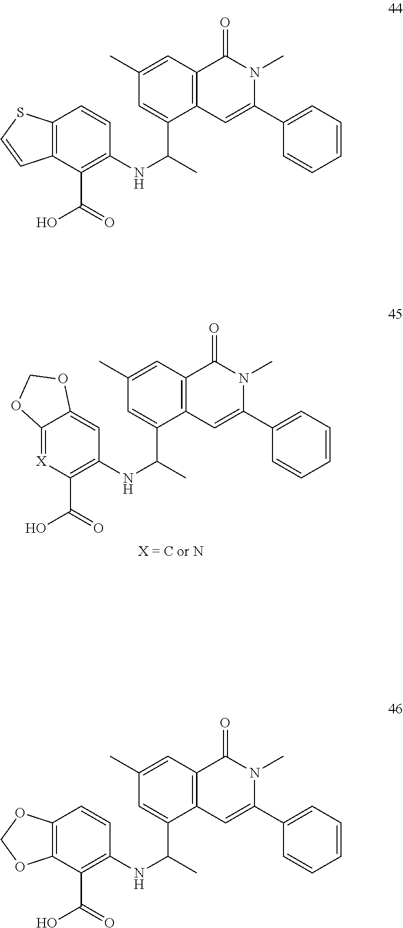

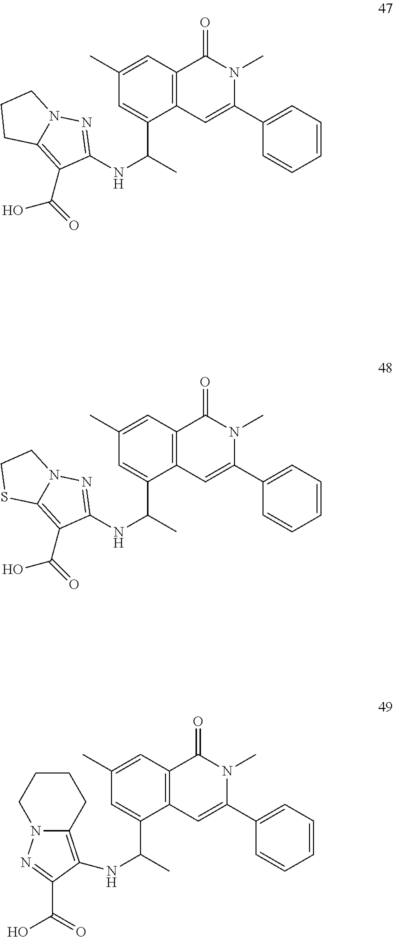 [SMILES: Cc1cc(C(C)Nc2nn3c(c2C(=O)O)CCC3)c2cc(-c3ccccc3)n(C)c(=O)c2c1 ; Cc1cc(C(C)Nc2nn3c(c2C(=O)O)SCC3)c2cc(-c3ccccc3)n(C)c(=O)c2c1 ; Cc1cc(C(C)Nc2c(C(=O)O)nn3c2CCCC3)c2cc(-c3ccccc3)n(C)c(=O)c2c1]

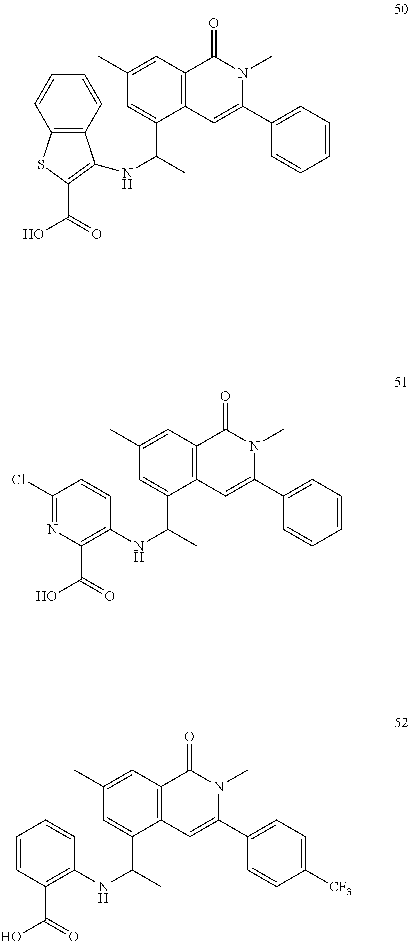 [SMILES: Cc1cc(C(C)Nc2c(C(=O)O)sc3ccccc23)c2cc(-c3ccccc3)n(C)c(=O)c2c1 ; Cc1cc(C(C)Nc2ccc(Cl)nc2C(=O)O)c2cc(-c3ccccc3)n(C)c(=O)c2c1 ; Cc1cc(C(C)Nc2ccccc2C(=O)O)c2cc(-c3ccc(C(F)(F)F)cc3)n(C)c(=O)c2c1]

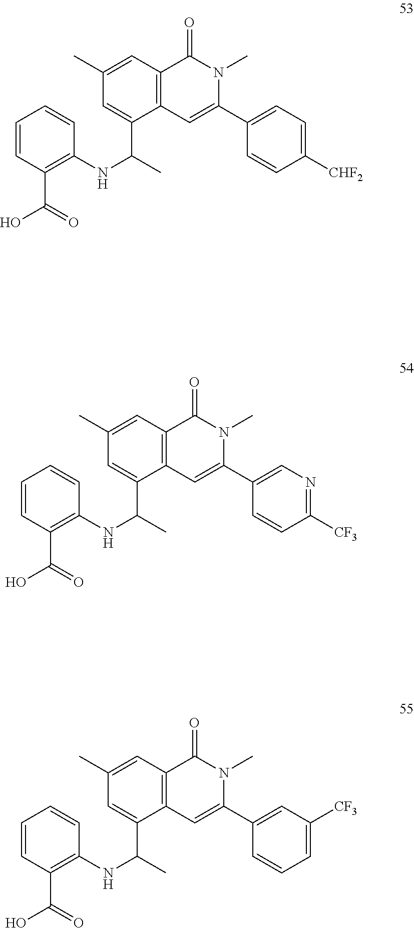 [SMILES: Cc1cc(C(C)Nc2ccccc2C(=O)O)c2cc(-c3ccc(C(F)F)cc3)n(C)c(=O)c2c1 ; Cc1cc(C(C)Nc2ccccc2C(=O)O)c2cc(-c3ccc(C(F)(F)F)nc3)n(C)c(=O)c2c1 ; Cc1cc(C(C)Nc2ccccc2C(=O)O)c2cc(-c3cccc(C(F)(F)F)c3)n(C)c(=O)c2c1]

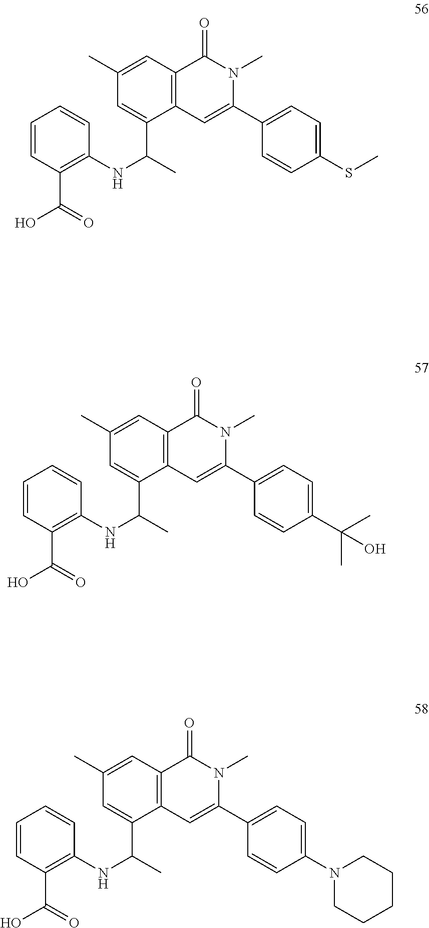 [SMILES: CSc1ccc(-c2cc3c(C(C)Nc4ccccc4C(=O)O)cc(C)cc3c(=O)n2C)cc1 ; Cc1cc(C(C)Nc2ccccc2C(=O)O)c2cc(-c3ccc(C(C)(C)O)cc3)n(C)c(=O)c2c1 ; Cc1cc(C(C)Nc2ccccc2C(=O)O)c2cc(-c3ccc(N4CCCCC4)cc3)n(C)c(=O)c2c1]

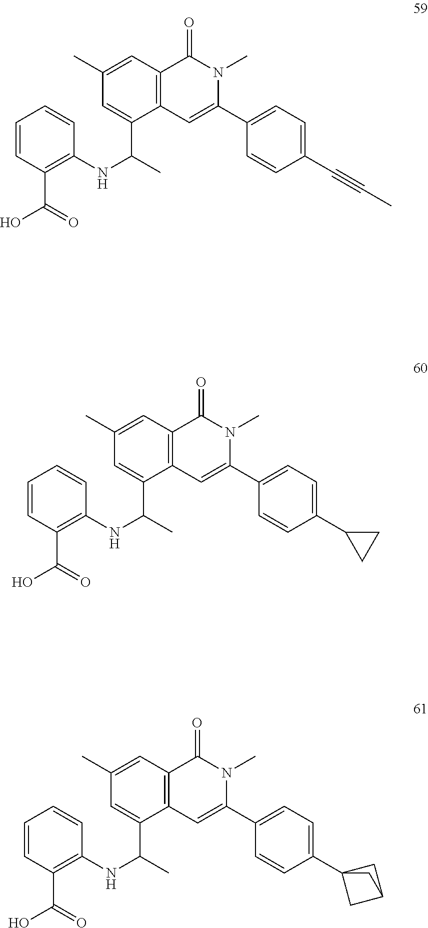 [SMILES: CC#Cc1ccc(-c2cc3c(C(C)Nc4ccccc4C(=O)O)cc(C)cc3c(=O)n2C)cc1 ; Cc1cc(C(C)Nc2ccccc2C(=O)O)c2cc(-c3ccc(C4CC4)cc3)n(C)c(=O)c2c1 ; Cc1cc(C(C)Nc2ccccc2C(=O)O)c2cc(-c3ccc(C45CC(C4)C5)cc3)n(C)c(=O)c2c1]

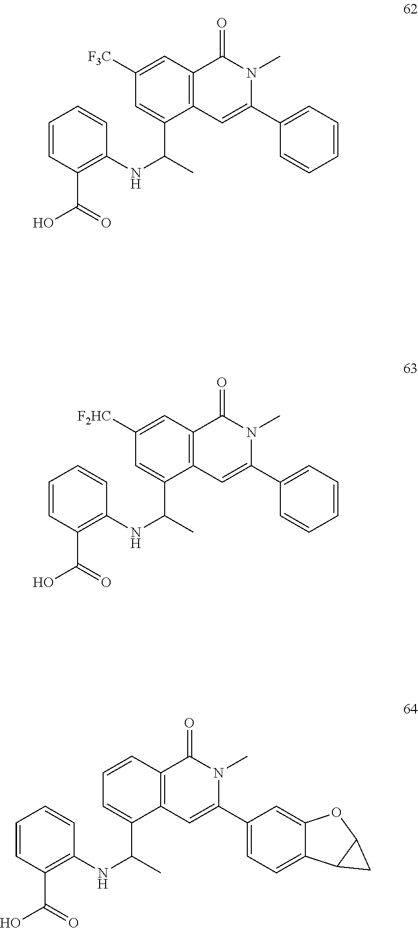 [SMILES: CC(Nc1ccccc1C(=O)O)c1cc(C(F)(F)F)cc2c(=O)n(C)c(-c3ccccc3)cc12 ; CC(Nc1ccccc1C(=O)O)c1cc(C(F)F)cc2c(=O)n(C)c(-c3ccccc3)cc12 ; CC(Nc1ccccc1C(=O)O)c1cccc2c(=O)n(C)c(-c3ccc4c(c3)OC3CC43)cc12]

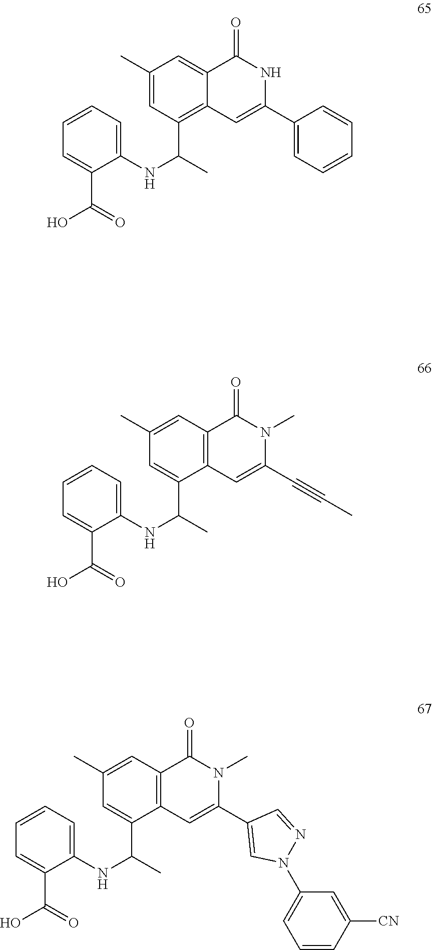 [SMILES: Cc1cc(C(C)Nc2ccccc2C(=O)O)c2cc(-c3ccccc3)[nH]c(=O)c2c1 ; CC#Cc1cc2c(C(C)Nc3ccccc3C(=O)O)cc(C)cc2c(=O)n1C ; Cc1cc(C(C)Nc2ccccc2C(=O)O)c2cc(-c3cnn(-c4cccc(C#N)c4)c3)n(C)c(=O)c2c1]

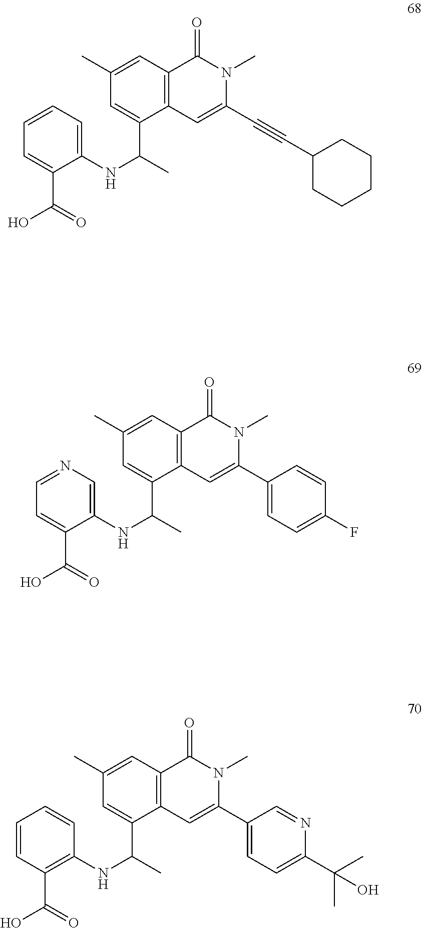 [SMILES: Cc1cc(C(C)Nc2ccccc2C(=O)O)c2cc(C#CC3CCCCC3)n(C)c(=O)c2c1 ; Cc1cc(C(C)Nc2cnccc2C(=O)O)c2cc(-c3ccc(F)cc3)n(C)c(=O)c2c1 ; Cc1cc(C(C)Nc2ccccc2C(=O)O)c2cc(-c3ccc(C(C)(C)O)nc3)n(C)c(=O)c2c1]

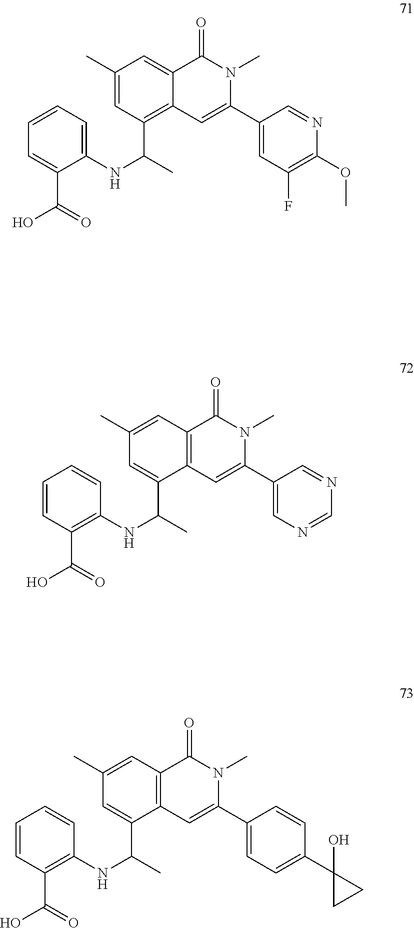 [SMILES: COc1ncc(-c2cc3c(C(C)Nc4ccccc4C(=O)O)cc(C)cc3c(=O)n2C)cc1F ; Cc1cc(C(C)Nc2ccccc2C(=O)O)c2cc(-c3cncnc3)n(C)c(=O)c2c1 ; Cc1cc(C(C)Nc2ccccc2C(=O)O)c2cc(-c3ccc(C4(O)CC4)cc3)n(C)c(=O)c2c1]

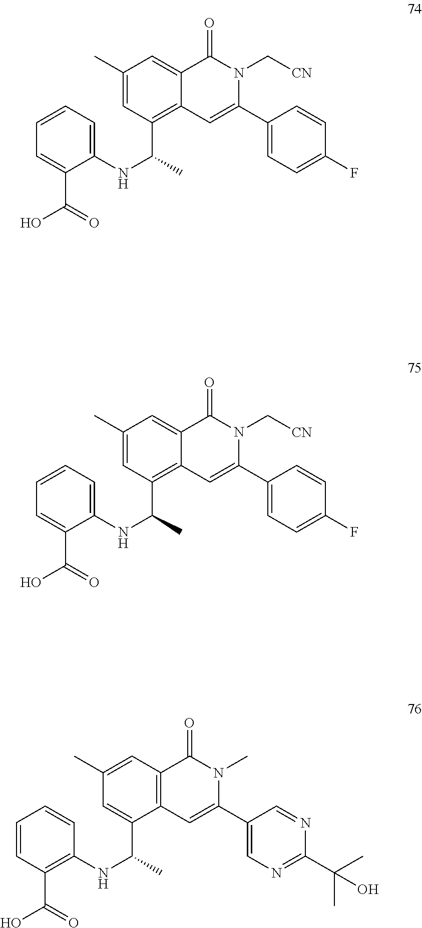 [SMILES: Cc1cc([C@H](C)Nc2ccccc2C(=O)O)c2cc(-c3ccc(F)cc3)n(CC#N)c(=O)c2c1 ; Cc1cc([C@@H](C)Nc2ccccc2C(=O)O)c2cc(-c3ccc(F)cc3)n(CC#N)c(=O)c2c1 ; Cc1cc([C@H](C)Nc2ccccc2C(=O)O)c2cc(-c3cnc(C(C)(C)O)nc3)n(C)c(=O)c2c1]

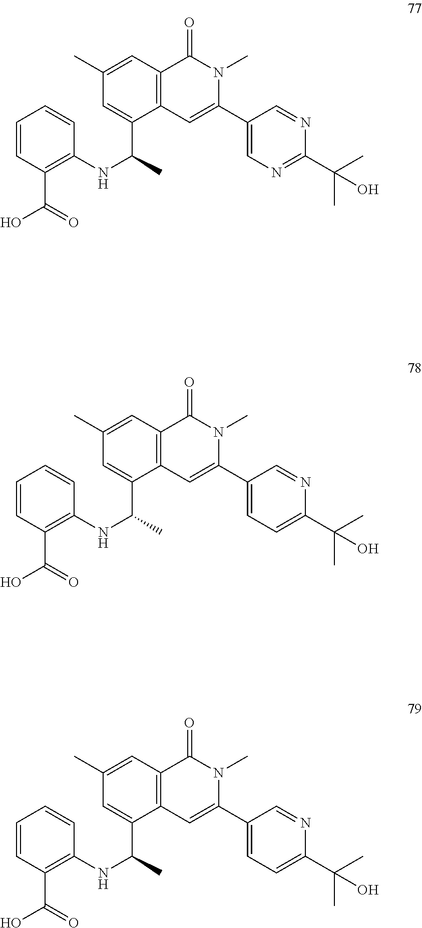 [SMILES: Cc1cc([C@@H](C)Nc2ccccc2C(=O)O)c2cc(-c3cnc(C(C)(C)O)nc3)n(C)c(=O)c2c1 ; Cc1cc([C@H](C)Nc2ccccc2C(=O)O)c2cc(-c3ccc(C(C)(C)O)nc3)n(C)c(=O)c2c1 ; Cc1cc([C@@H](C)Nc2ccccc2C(=O)O)c2cc(-c3ccc(C(C)(C)O)nc3)n(C)c(=O)c2c1]

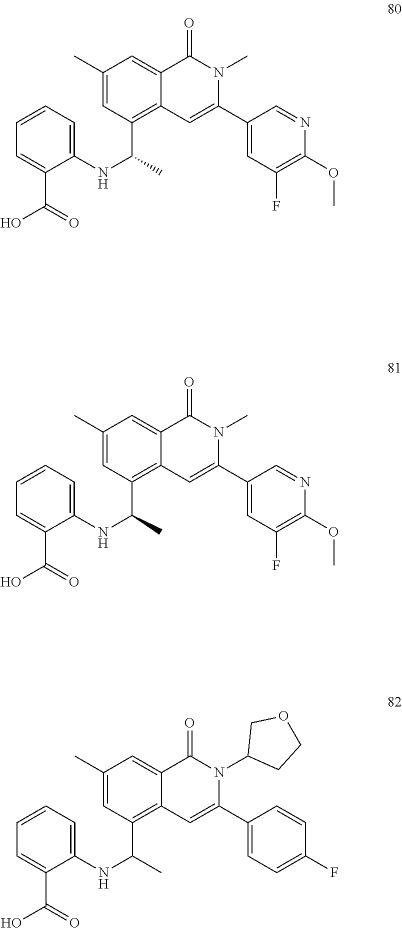 [SMILES: COc1ncc(-c2cc3c([C@H](C)Nc4ccccc4C(=O)O)cc(C)cc3c(=O)n2C)cc1F ; COc1ncc(-c2cc3c([C@@H](C)Nc4ccccc4C(=O)O)cc(C)cc3c(=O)n2C)cc1F ; Cc1cc(C(C)Nc2ccccc2C(=O)O)c2cc(-c3ccc(F)cc3)n(C3CCOC3)c(=O)c2c1]

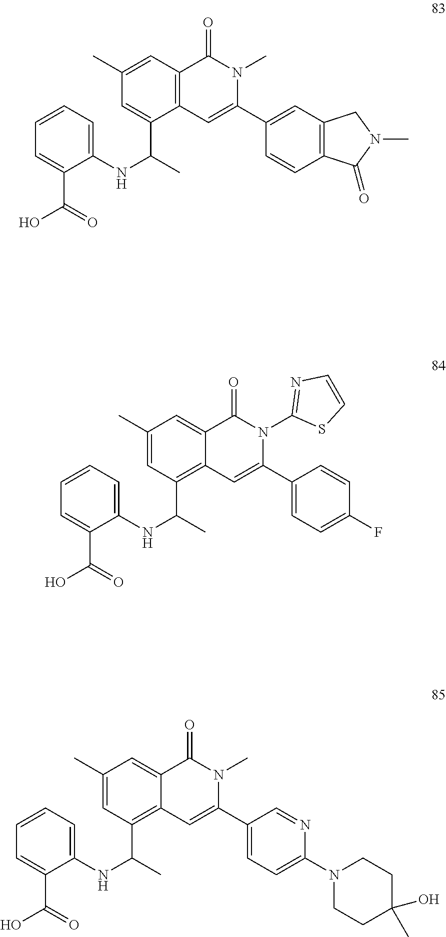 [SMILES: Cc1cc(C(C)Nc2ccccc2C(=O)O)c2cc(-c3ccc4c(c3)CN(C)C4=O)n(C)c(=O)c2c1 ; Cc1cc(C(C)Nc2ccccc2C(=O)O)c2cc(-c3ccc(F)cc3)n(-c3nccs3)c(=O)c2c1 ; Cc1cc(C(C)Nc2ccccc2C(=O)O)c2cc(-c3ccc(N4CCC(C)(O)CC4)nc3)n(C)c(=O)c2c1]

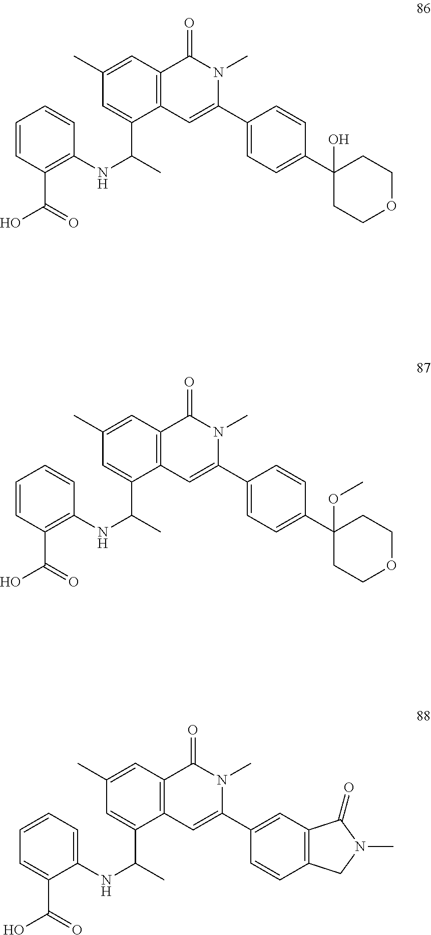 [SMILES: Cc1cc(C(C)Nc2ccccc2C(=O)O)c2cc(-c3ccc(C4(O)CCOCC4)cc3)n(C)c(=O)c2c1 ; COC1(c2ccc(-c3cc4c(C(C)Nc5ccccc5C(=O)O)cc(C)cc4c(=O)n3C)cc2)CCOCC1 ; Cc1cc(C(C)Nc2ccccc2C(=O)O)c2cc(-c3ccc4c(c3)C(=O)N(C)C4)n(C)c(=O)c2c1]

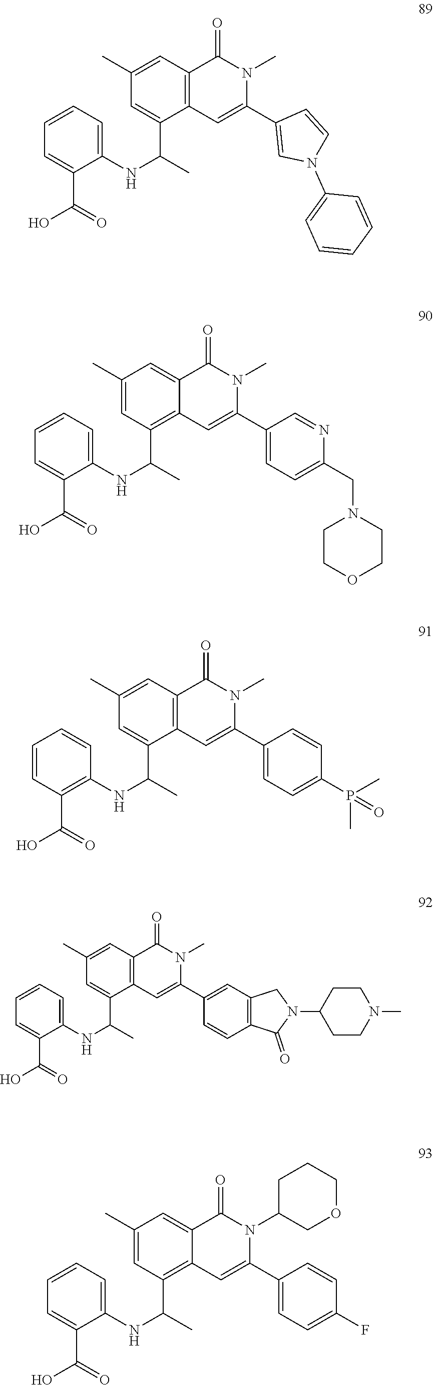 [SMILES: Cc1cc(C(C)Nc2ccccc2C(=O)O)c2cc(-c3ccn(-c4ccccc4)c3)n(C)c(=O)c2c1 ; Cc1cc(C(C)Nc2ccccc2C(=O)O)c2cc(-c3ccc(CN4CCOCC4)nc3)n(C)c(=O)c2c1 ; Cc1cc(C(C)Nc2ccccc2C(=O)O)c2cc(-c3ccc(P(C)(C)=O)cc3)n(C)c(=O)c2c1 ; Cc1cc(C(C)Nc2ccccc2C(=O)O)c2cc(-c3ccc4c(c3)CN(C3CCN(C)CC3)C4=O)n(C)c(=O)c2c1 ; Cc1cc(C(C)Nc2ccccc2C(=O)O)c2cc(-c3ccc(F)cc3)n(C3CCCOC3)c(=O)c2c1]

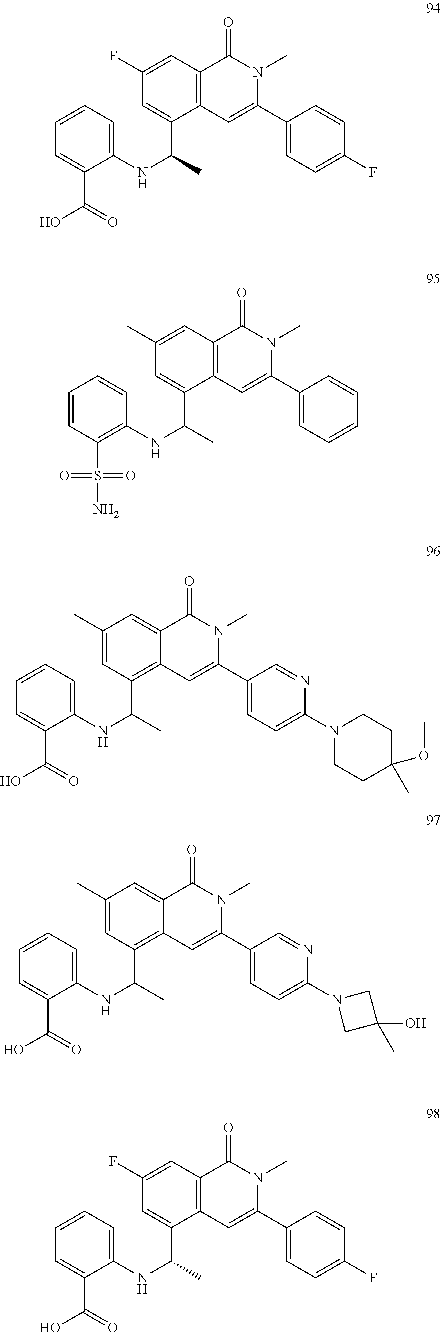 [SMILES: C[C@@H](Nc1ccccc1C(=O)O)c1cc(F)cc2c(=O)n(C)c(-c3ccc(F)cc3)cc12 ; Cc1cc(C(C)Nc2ccccc2S(N)(=O)=O)c2cc(-c3ccccc3)n(C)c(=O)c2c1 ; COC1(C)CCN(c2ccc(-c3cc4c(C(C)Nc5ccccc5C(=O)O)cc(C)cc4c(=O)n3C)cn2)CC1 ; Cc1cc(C(C)Nc2ccccc2C(=O)O)c2cc(-c3ccc(N4CC(C)(O)C4)nc3)n(C)c(=O)c2c1 ; C[C@H](Nc1ccccc1C(=O)O)c1cc(F)cc2c(=O)n(C)c(-c3ccc(F)cc3)cc12]

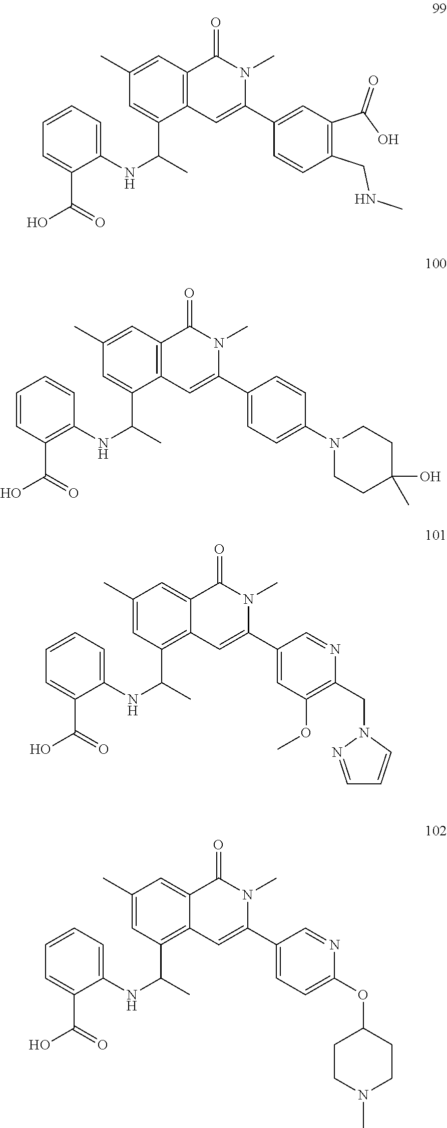 [SMILES: CNCc1ccc(-c2cc3c(C(C)Nc4ccccc4C(=O)O)cc(C)cc3c(=O)n2C)cc1C(=O)O ; Cc1cc(C(C)Nc2ccccc2C(=O)O)c2cc(-c3ccc(N4CCC(C)(O)CC4)cc3)n(C)c(=O)c2c1 ; COc1cc(-c2cc3c(C(C)Nc4ccccc4C(=O)O)cc(C)cc3c(=O)n2C)cnc1Cn1cccn1 ; Cc1cc(C(C)Nc2ccccc2C(=O)O)c2cc(-c3ccc(OC4CCN(C)CC4)nc3)n(C)c(=O)c2c1]

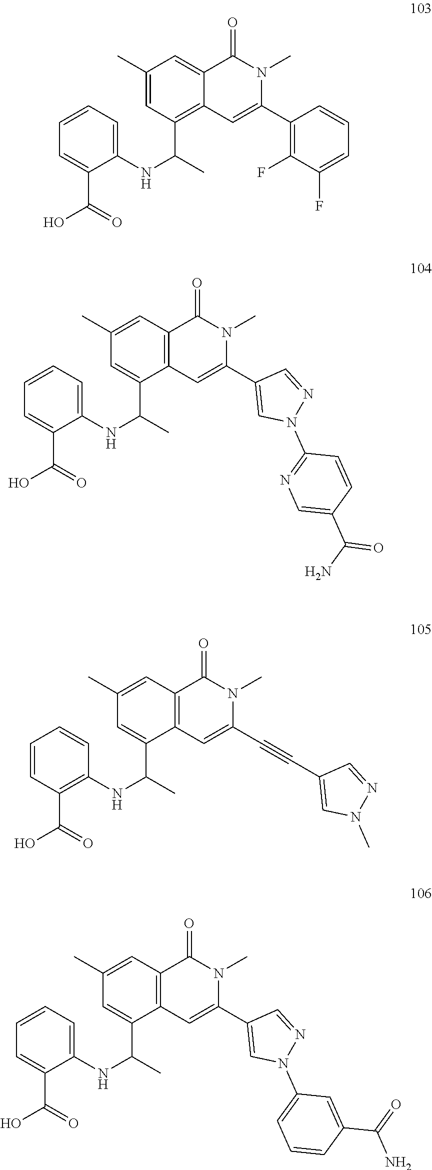 [SMILES: Cc1cc(C(C)Nc2ccccc2C(=O)O)c2cc(-c3cccc(F)c3F)n(C)c(=O)c2c1 ; Cc1cc(C(C)Nc2ccccc2C(=O)O)c2cc(-c3cnn(-c4ccc(C(N)=O)cn4)c3)n(C)c(=O)c2c1 ; Cc1cc(C(C)Nc2ccccc2C(=O)O)c2cc(C#Cc3cnn(C)c3)n(C)c(=O)c2c1 ; Cc1cc(C(C)Nc2ccccc2C(=O)O)c2cc(-c3cnn(-c4cccc(C(N)=O)c4)c3)n(C)c(=O)c2c1]

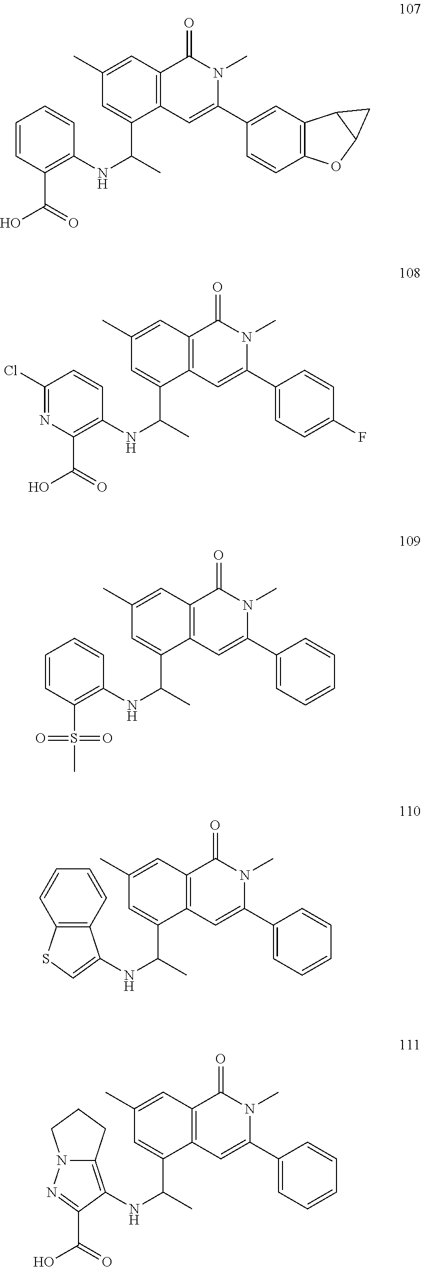 [SMILES: Cc1cc(C(C)Nc2ccccc2C(=O)O)c2cc(-c3ccc4c(c3)C3CC3O4)n(C)c(=O)c2c1 ; Cc1cc(C(C)Nc2ccc(Cl)nc2C(=O)O)c2cc(-c3ccc(F)cc3)n(C)c(=O)c2c1 ; Cc1cc(C(C)Nc2ccccc2S(C)(=O)=O)c2cc(-c3ccccc3)n(C)c(=O)c2c1 ; Cc1cc(C(C)Nc2csc3ccccc23)c2cc(-c3ccccc3)n(C)c(=O)c2c1 ; Cc1cc(C(C)Nc2c(C(=O)O)nn3c2CCC3)c2cc(-c3ccccc3)n(C)c(=O)c2c1]

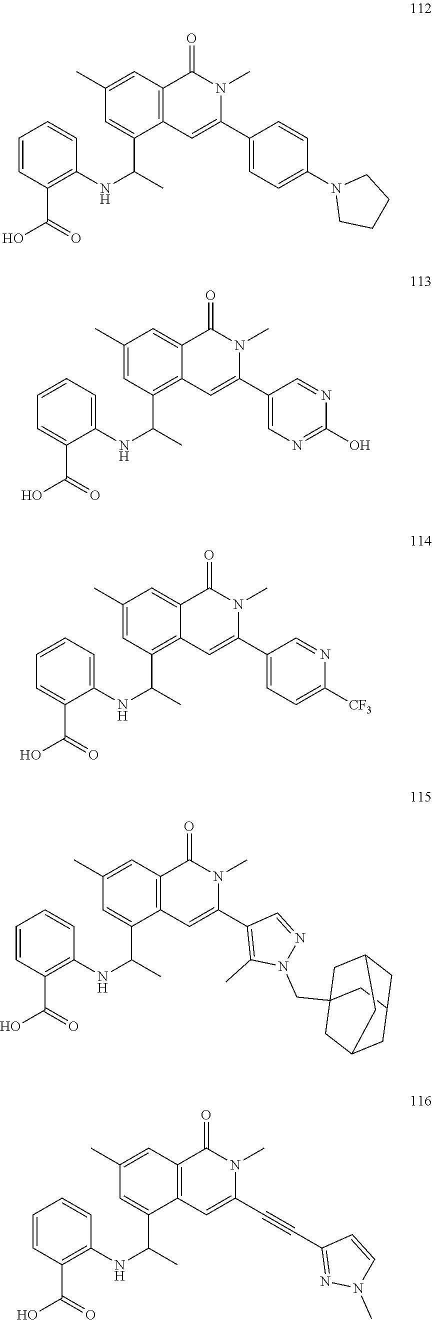 [SMILES: Cc1cc(C(C)Nc2ccccc2C(=O)O)c2cc(-c3ccc(N4CCCC4)cc3)n(C)c(=O)c2c1 ; Cc1cc(C(C)Nc2ccccc2C(=O)O)c2cc(-c3cnc(O)nc3)n(C)c(=O)c2c1 ; Cc1cc(C(C)Nc2ccccc2C(=O)O)c2cc(-c3ccc(C(F)(F)F)nc3)n(C)c(=O)c2c1 ; Cc1cc(C(C)Nc2ccccc2C(=O)O)c2cc(-c3cnn(CC45CC6CC(CC(C6)C4)C5)c3C)n(C)c(=O)c2c1 ; Cc1cc(C(C)Nc2ccccc2C(=O)O)c2cc(C#Cc3ccn(C)n3)n(C)c(=O)c2c1]

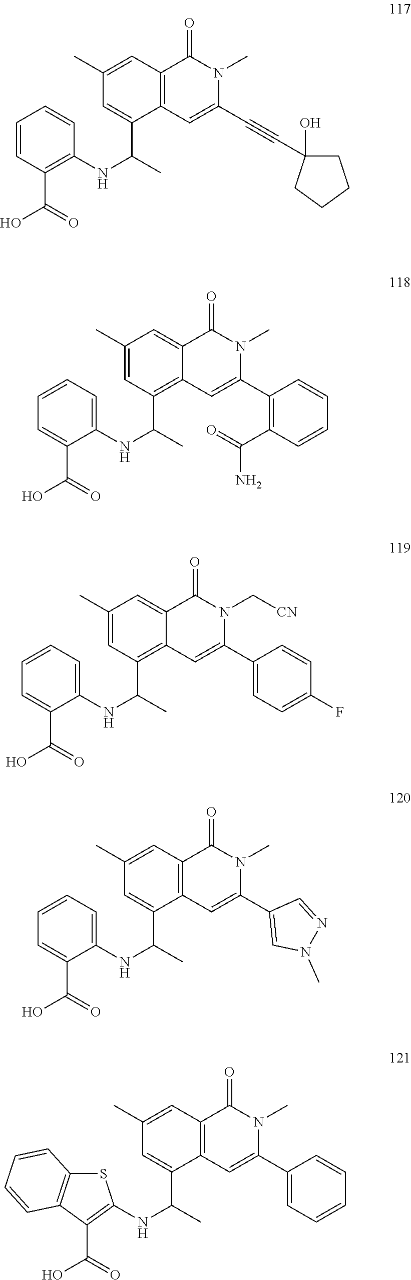 [SMILES: Cc1cc(C(C)Nc2ccccc2C(=O)O)c2cc(C#CC3(O)CCCC3)n(C)c(=O)c2c1 ; Cc1cc(C(C)Nc2ccccc2C(=O)O)c2cc(-c3ccccc3C(N)=O)n(C)c(=O)c2c1 ; Cc1cc(C(C)Nc2ccccc2C(=O)O)c2cc(-c3ccc(F)cc3)n(CC#N)c(=O)c2c1 ; Cc1cc(C(C)Nc2ccccc2C(=O)O)c2cc(-c3cnn(C)c3)n(C)c(=O)c2c1 ; Cc1cc(C(C)Nc2sc3ccccc3c2C(=O)O)c2cc(-c3ccccc3)n(C)c(=O)c2c1]

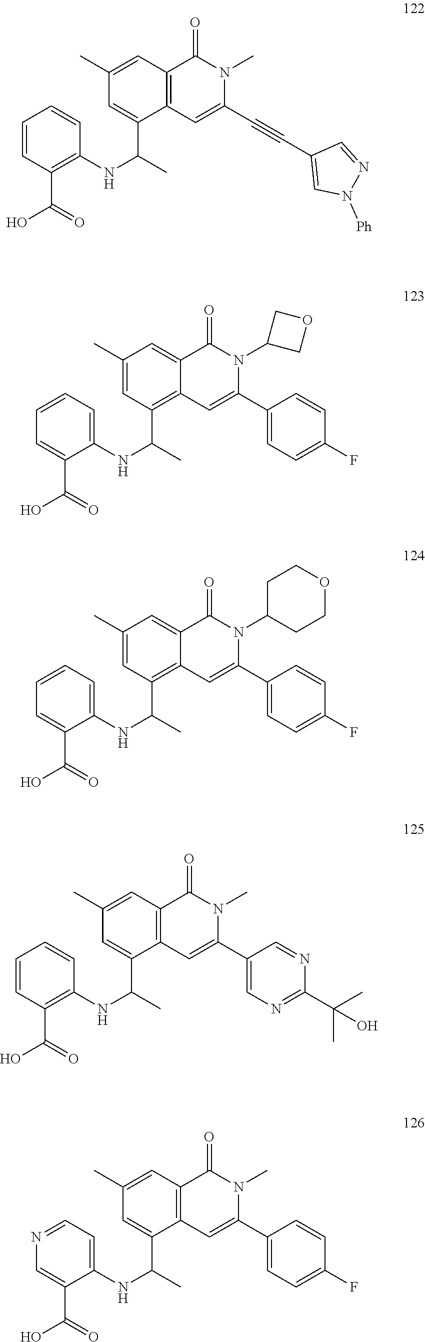 [SMILES: Cc1cc(C(C)Nc2ccccc2C(=O)O)c2cc(C#Cc3cnn(-c4ccccc4)c3)n(C)c(=O)c2c1 ; Cc1cc(C(C)Nc2ccccc2C(=O)O)c2cc(-c3ccc(F)cc3)n(C3COC3)c(=O)c2c1 ; Cc1cc(C(C)Nc2ccccc2C(=O)O)c2cc(-c3ccc(F)cc3)n(C3CCOCC3)c(=O)c2c1 ; Cc1cc(C(C)Nc2ccccc2C(=O)O)c2cc(-c3cnc(C(C)(C)O)nc3)n(C)c(=O)c2c1 ; Cc1cc(C(C)Nc2ccncc2C(=O)O)c2cc(-c3ccc(F)cc3)n(C)c(=O)c2c1]

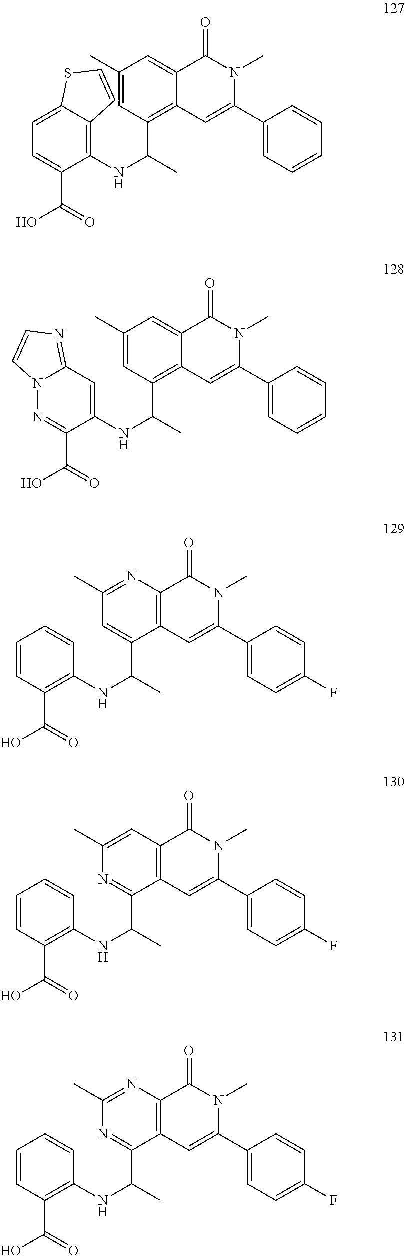 [SMILES: Cc1cc(C(C)Nc2c(C(=O)O)ccc3sccc23)c2cc(-c3ccccc3)n(C)c(=O)c2c1 ; Cc1cc(C(C)Nc2cc3nccn3nc2C(=O)O)c2cc(-c3ccccc3)n(C)c(=O)c2c1 ; Cc1cc(C(C)Nc2ccccc2C(=O)O)c2cc(-c3ccc(F)cc3)n(C)c(=O)c2n1 ; Cc1cc2c(=O)n(C)c(-c3ccc(F)cc3)cc2c(C(C)Nc2ccccc2C(=O)O)n1 ; Cc1nc(C(C)Nc2ccccc2C(=O)O)c2cc(-c3ccc(F)cc3)n(C)c(=O)c2n1]

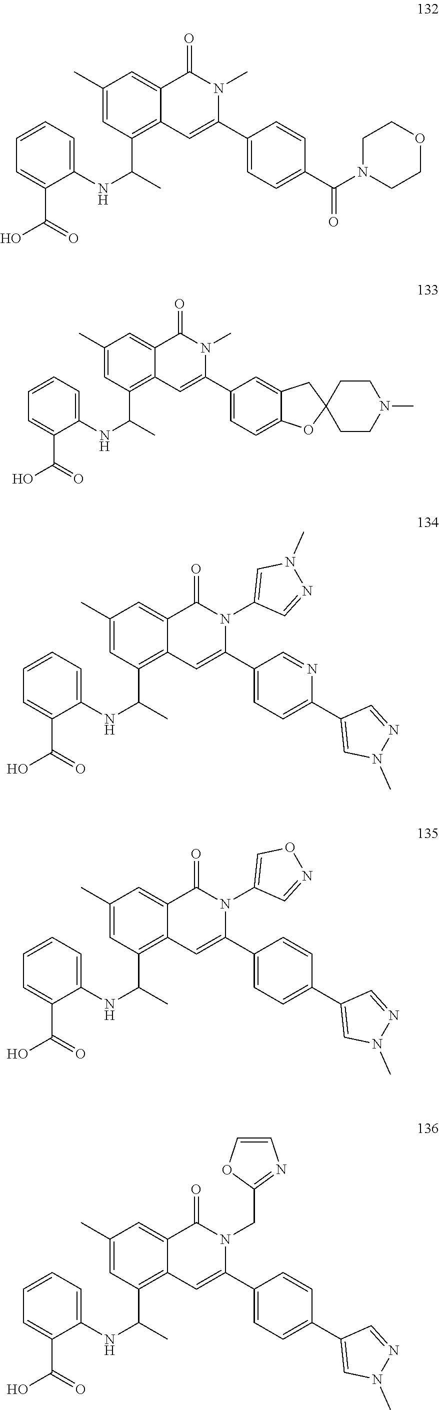 [SMILES: Cc1cc(C(C)Nc2ccccc2C(=O)O)c2cc(-c3ccc(C(=O)N4CCOCC4)cc3)n(C)c(=O)c2c1 ; Cc1cc(C(C)Nc2ccccc2C(=O)O)c2cc(-c3ccc4c(c3)CC3(CCN(C)CC3)O4)n(C)c(=O)c2c1 ; Cc1cc(C(C)Nc2ccccc2C(=O)O)c2cc(-c3ccc(-c4cnn(C)c4)nc3)n(-c3cnn(C)c3)c(=O)c2c1 ; Cc1cc(C(C)Nc2ccccc2C(=O)O)c2cc(-c3ccc(-c4cnn(C)c4)cc3)n(-c3cnoc3)c(=O)c2c1 ; Cc1cc(C(C)Nc2ccccc2C(=O)O)c2cc(-c3ccc(-c4cnn(C)c4)cc3)n(Cc3ncco3)c(=O)c2c1]

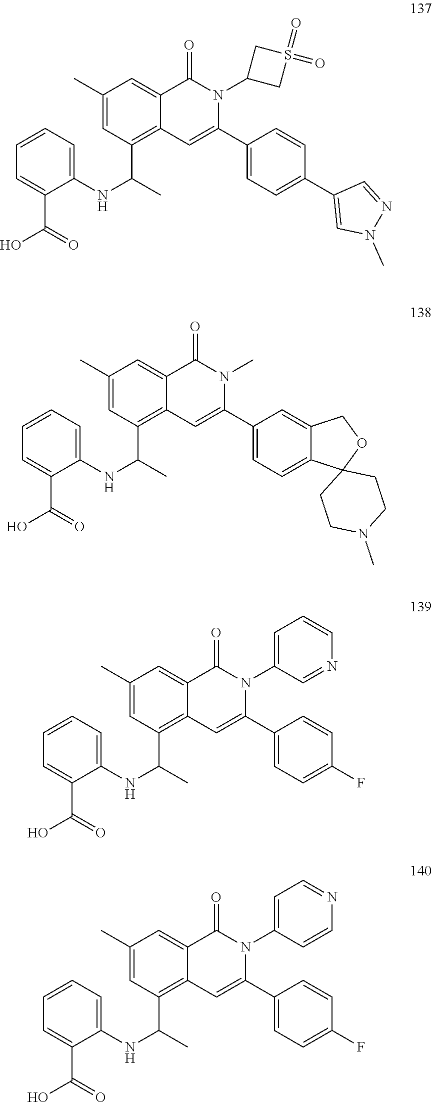 [SMILES: Cc1cc(C(C)Nc2ccccc2C(=O)O)c2cc(-c3ccc(-c4cnn(C)c4)cc3)n(C3CS(=O)(=O)C3)c(=O)c2c1 ; Cc1cc(C(C)Nc2ccccc2C(=O)O)c2cc(-c3ccc4c(c3)COC43CCN(C)CC3)n(C)c(=O)c2c1 ; Cc1cc(C(C)Nc2ccccc2C(=O)O)c2cc(-c3ccc(F)cc3)n(-c3cccnc3)c(=O)c2c1 ; Cc1cc(C(C)Nc2ccccc2C(=O)O)c2cc(-c3ccc(F)cc3)n(-c3ccncc3)c(=O)c2c1]

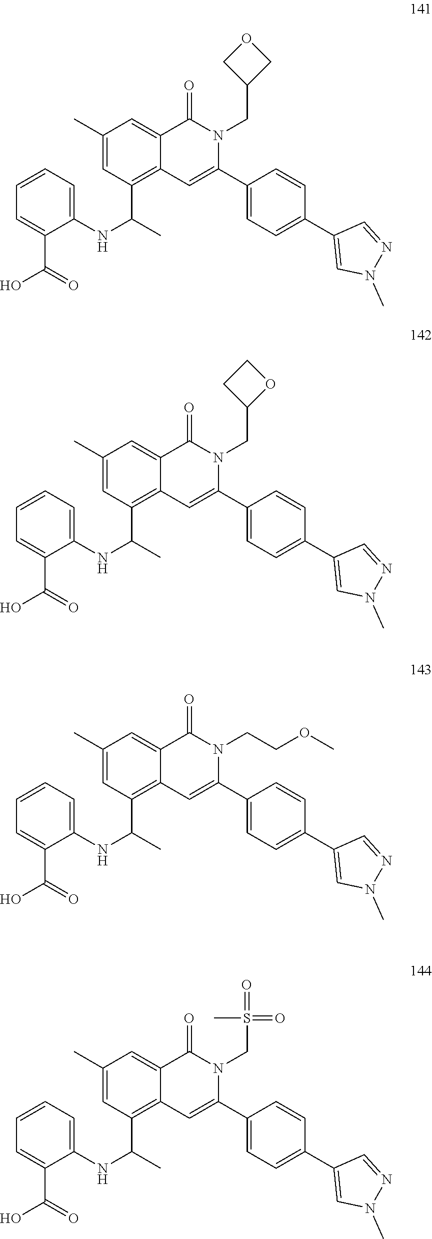 [SMILES: Cc1cc(C(C)Nc2ccccc2C(=O)O)c2cc(-c3ccc(-c4cnn(C)c4)cc3)n(CC3COC3)c(=O)c2c1 ; Cc1cc(C(C)Nc2ccccc2C(=O)O)c2cc(-c3ccc(-c4cnn(C)c4)cc3)n(CC3CCO3)c(=O)c2c1 ; COCCn1c(-c2ccc(-c3cnn(C)c3)cc2)cc2c(C(C)Nc3ccccc3C(=O)O)cc(C)cc2c1=O ; Cc1cc(C(C)Nc2ccccc2C(=O)O)c2cc(-c3ccc(-c4cnn(C)c4)cc3)n(CS(C)(=O)=O)c(=O)c2c1]

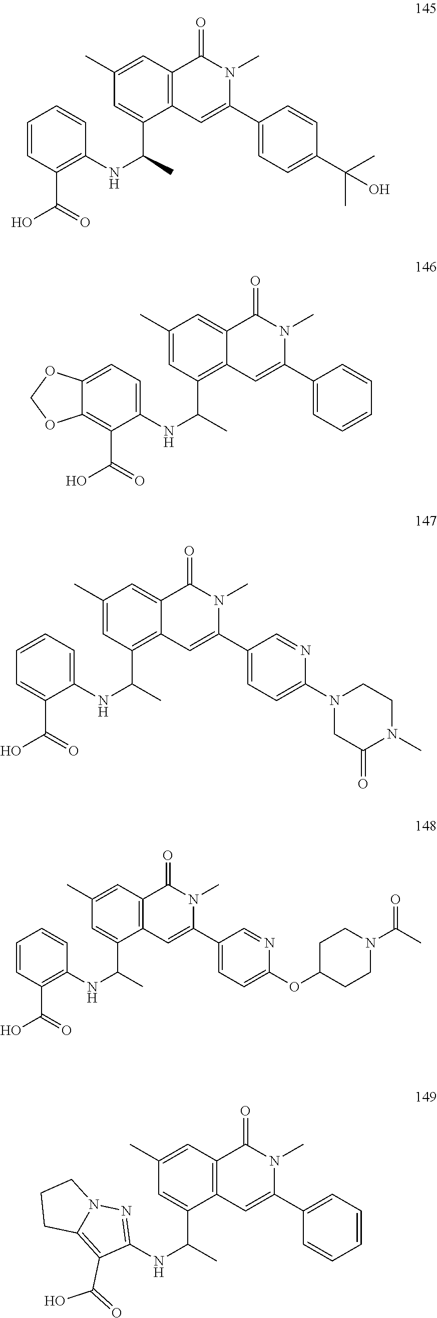 [SMILES: Cc1cc([C@@H](C)Nc2ccccc2C(=O)O)c2cc(-c3ccc(C(C)(C)O)cc3)n(C)c(=O)c2c1 ; Cc1cc(C(C)Nc2ccc3c(c2C(=O)O)OCO3)c2cc(-c3ccccc3)n(C)c(=O)c2c1 ; Cc1cc(C(C)Nc2ccccc2C(=O)O)c2cc(-c3ccc(N4CCN(C)C(=O)C4)nc3)n(C)c(=O)c2c1 ; CC(=O)N1CCC(Oc2ccc(-c3cc4c(C(C)Nc5ccccc5C(=O)O)cc(C)cc4c(=O)n3C)cn2)CC1 ; Cc1cc(C(C)Nc2nn3c(c2C(=O)O)CCC3)c2cc(-c3ccccc3)n(C)c(=O)c2c1]

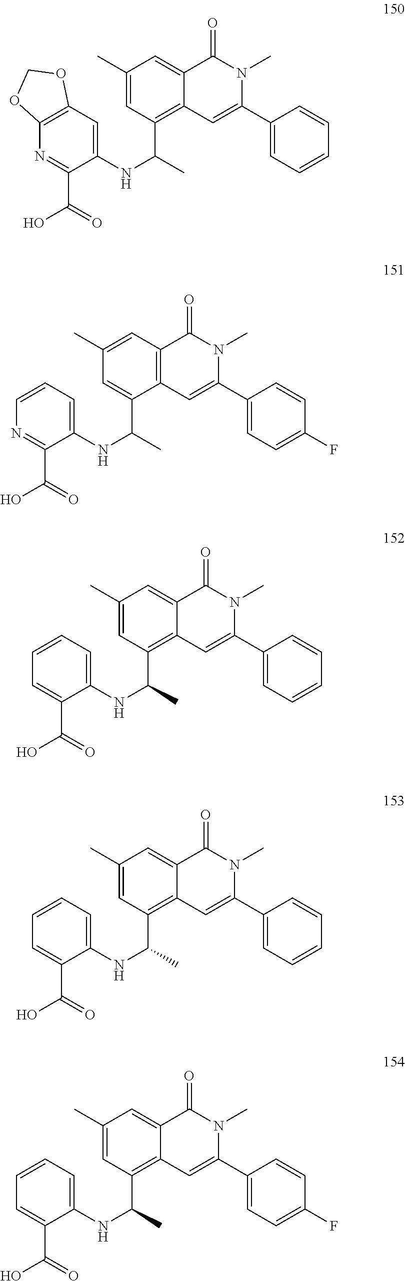 [SMILES: Cc1cc(C(C)Nc2cc3c(nc2C(=O)O)OCO3)c2cc(-c3ccccc3)n(C)c(=O)c2c1 ; Cc1cc(C(C)Nc2cccnc2C(=O)O)c2cc(-c3ccc(F)cc3)n(C)c(=O)c2c1 ; Cc1cc([C@@H](C)Nc2ccccc2C(=O)O)c2cc(-c3ccccc3)n(C)c(=O)c2c1 ; Cc1cc([C@H](C)Nc2ccccc2C(=O)O)c2cc(-c3ccccc3)n(C)c(=O)c2c1 ; Cc1cc([C@@H](C)Nc2ccccc2C(=O)O)c2cc(-c3ccc(F)cc3)n(C)c(=O)c2c1]

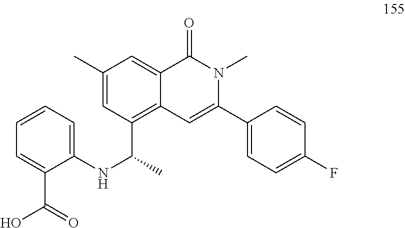 [SMILES: Cc1cc([C@H](C)Nc2ccccc2C(=O)O)c2cc(-c3ccc(F)cc3)n(C)c(=O)c2c1]

In embodiments of Aspect 41, the compound is selected from:

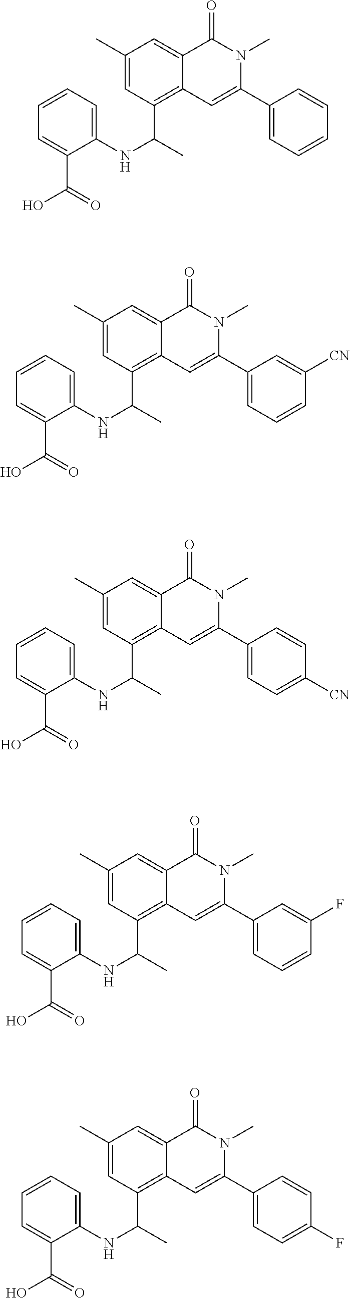 [SMILES: Cc1cc(C(C)Nc2ccccc2C(=O)O)c2cc(-c3ccccc3)n(C)c(=O)c2c1 ; Cc1cc(C(C)Nc2ccccc2C(=O)O)c2cc(-c3cccc(C#N)c3)n(C)c(=O)c2c1 ; Cc1cc(C(C)Nc2ccccc2C(=O)O)c2cc(-c3ccc(C#N)cc3)n(C)c(=O)c2c1 ; Cc1cc(C(C)Nc2ccccc2C(=O)O)c2cc(-c3cccc(F)c3)n(C)c(=O)c2c1 ; Cc1cc(C(C)Nc2ccccc2C(=O)O)c2cc(-c3ccc(F)cc3)n(C)c(=O)c2c1]

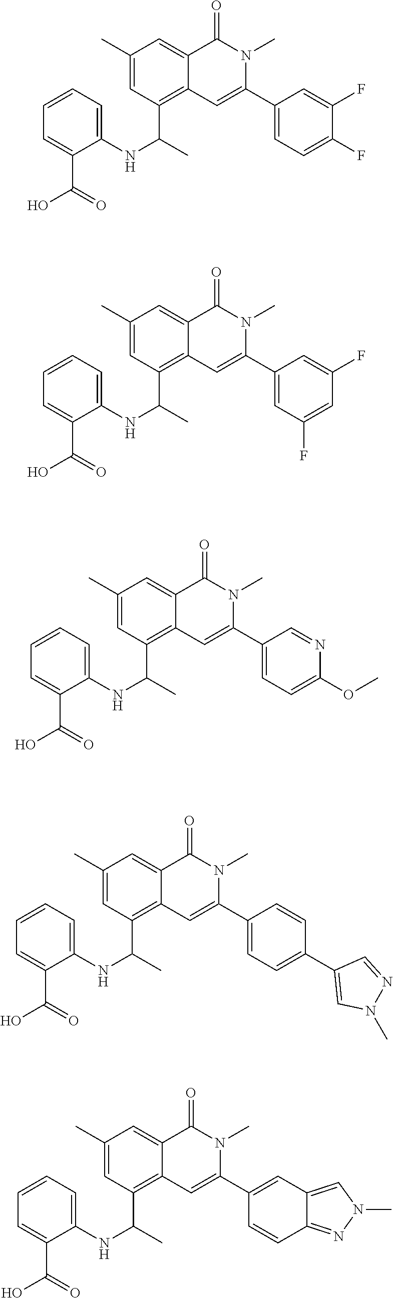 [SMILES: Cc1cc(C(C)Nc2ccccc2C(=O)O)c2cc(-c3ccc(F)c(F)c3)n(C)c(=O)c2c1 ; Cc1cc(C(C)Nc2ccccc2C(=O)O)c2cc(-c3cc(F)cc(F)c3)n(C)c(=O)c2c1 ; COc1ccc(-c2cc3c(C(C)Nc4ccccc4C(=O)O)cc(C)cc3c(=O)n2C)cn1 ; Cc1cc(C(C)Nc2ccccc2C(=O)O)c2cc(-c3ccc(-c4cnn(C)c4)cc3)n(C)c(=O)c2c1 ; Cc1cc(C(C)Nc2ccccc2C(=O)O)c2cc(-c3ccc4nn(C)cc4c3)n(C)c(=O)c2c1]

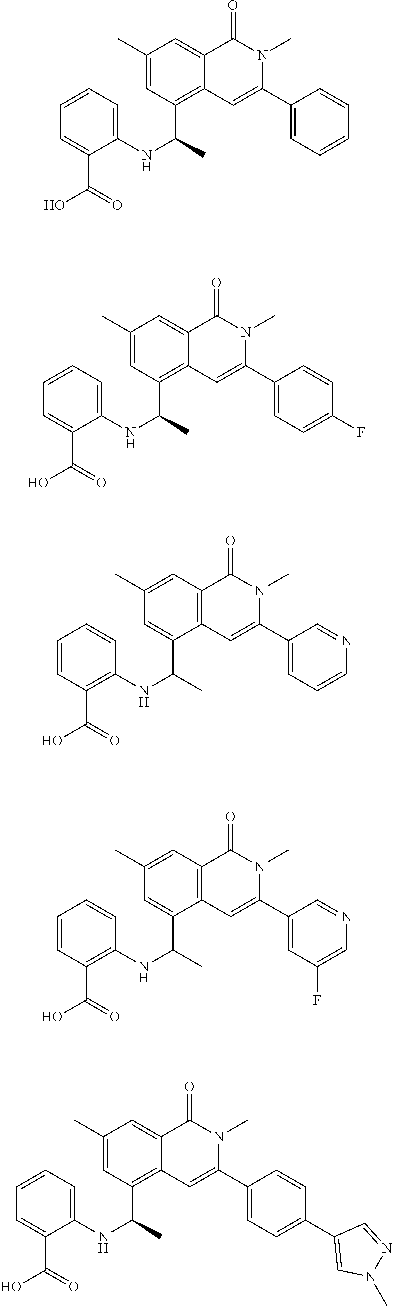 [SMILES: Cc1cc([C@@H](C)Nc2ccccc2C(=O)O)c2cc(-c3ccccc3)n(C)c(=O)c2c1 ; Cc1cc([C@@H](C)Nc2ccccc2C(=O)O)c2cc(-c3ccc(F)cc3)n(C)c(=O)c2c1 ; Cc1cc(C(C)Nc2ccccc2C(=O)O)c2cc(-c3cccnc3)n(C)c(=O)c2c1 ; Cc1cc(C(C)Nc2ccccc2C(=O)O)c2cc(-c3cncc(F)c3)n(C)c(=O)c2c1 ; Cc1cc([C@@H](C)Nc2ccccc2C(=O)O)c2cc(-c3ccc(-c4cnn(C)c4)cc3)n(C)c(=O)c2c1]

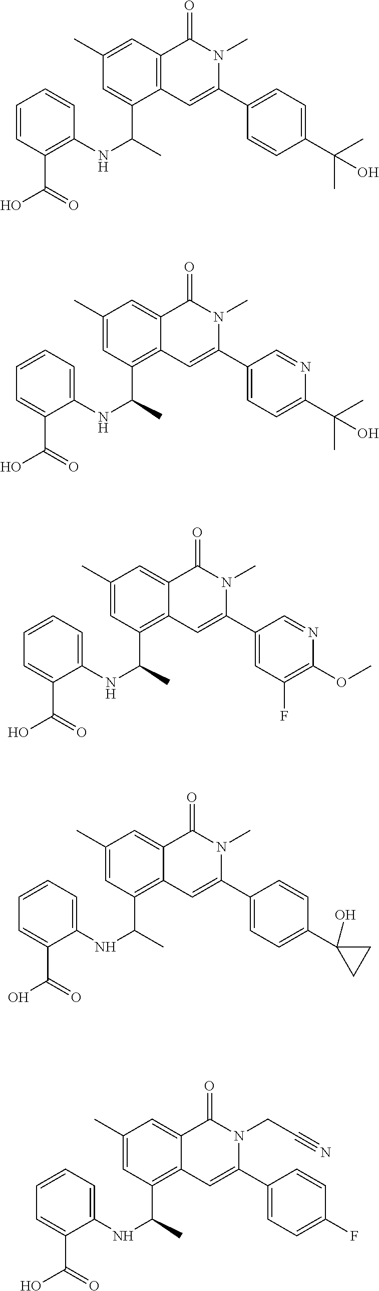 [SMILES: Cc1cc(C(C)Nc2ccccc2C(=O)O)c2cc(-c3ccc(C(C)(C)O)cc3)n(C)c(=O)c2c1 ; Cc1cc([C@@H](C)Nc2ccccc2C(=O)O)c2cc(-c3ccc(C(C)(C)O)nc3)n(C)c(=O)c2c1 ; COc1ncc(-c2cc3c([C@@H](C)Nc4ccccc4C(=O)O)cc(C)cc3c(=O)n2C)cc1F ; Cc1cc(C(C)Nc2ccccc2C(=O)O)c2cc(-c3ccc(C4(O)CC4)cc3)n(C)c(=O)c2c1 ; Cc1cc([C@@H](C)Nc2ccccc2C(=O)O)c2cc(-c3ccc(F)cc3)n(CC#N)c(=O)c2c1]

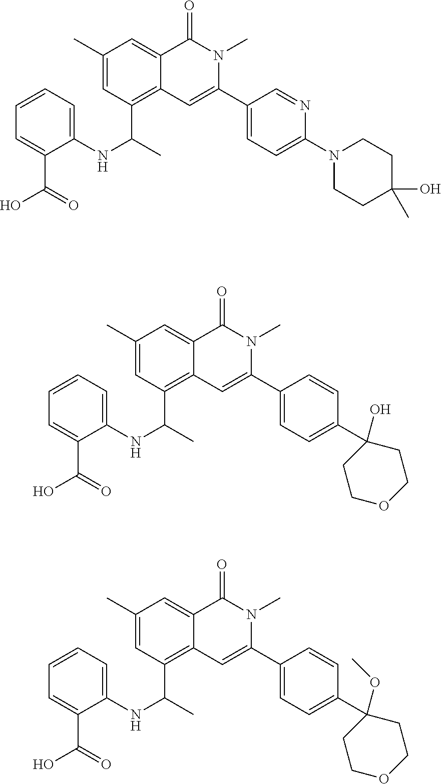 [SMILES: Cc1cc(C(C)Nc2ccccc2C(=O)O)c2cc(-c3ccc(N4CCC(C)(O)CC4)nc3)n(C)c(=O)c2c1 ; Cc1cc(C(C)Nc2ccccc2C(=O)O)c2cc(-c3ccc(C4(O)CCOCC4)cc3)n(C)c(=O)c2c1 ; COC1(c2ccc(-c3cc4c(C(C)Nc5ccccc5C(=O)O)cc(C)cc4c(=O)n3C)cc2)CCOCC1]

or a stereoisomer, tautomer, or pharmaceutically acceptable salt thereof.

Aspect 42. A compound of the following formula:

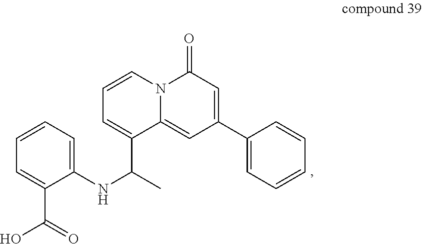 [SMILES: CC(Nc1ccccc1C(=O)O)c1cccn2c(=O)cc(-c3ccccc3)cc12]

or a stereoisomer, tautomer, or pharmaceutically acceptable salt thereof.

Aspect 43. A pharmaceutical composition comprising a therapeutically effective amount of a compound disclosed herein (e.g., a compound of any one of Aspects 1-42), or a stereoisomer, tautomer, or pharmaceutically acceptable salt thereof, and a pharmaceutically acceptable excipient.

Aspect 44. A method for treating or preventing a disorder or a disease responsive to the inhibition of PI3Kα activity in a subject (e.g., a subject in need thereof), comprising administering to the subject a compound disclosed herein (e.g., a compound of any one of Aspects 1-42, or a stereoisomer, tautomer, or pharmaceutically acceptable salt thereof, e.g., a therapeutically effective amount of the compound disclosed herein, or a stereoisomer, tautomer, or pharmaceutically acceptable salt thereof.

In an embodiment of Aspect 44, the subject has PI3Kα containing at least one of the following mutations: H1047R, E542K, and E545K.

In an embodiment of Aspect 44, the disorder or disease is an inflammatory disorder, an autoimmune disease, or a cancer.

In an embodiment of Aspect 44, the disorder or disease is a cancer.

In an embodiment of Aspect 44, the disorder or disease is selected from the group consisting of: endometrial cancer, gastric cancer, leukemia, lymphoma, sarcoma, colorectal cancer, lung cancer, ovarian cancer, skin cancer, head and neck cancer, breast cancer, brain cancer, prostate cancer, cervical cancer, glioblastoma, pancreatic cancer, bladder cancer, CLOVES syndrome (Congenital Lipomatous Overgrowth, Vascular Malformations, Epidermal Nevis, Spinal/Skeletal Anomalies/Scoliosis) and PROS syndrome (PIK3CA-related overgrowth syndrome).

### DETAILED DESCRIPTION OF THE INVENTION

The following terms have the indicated meaning throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “hydrogen” includes protium, tritium and deuterium. Any hydrogen can be replaced by tritium or deuterium. In preferred embodiments, hydrogen is protium.

The term “alkyl” refers to a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[1.1.1]pentane bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 12 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, or fused cycloalkenyl, preferably cyclohexenyl.

The term “fused cycloalkenyl” refers to a bicyclic cycloalkyl group as defined herein which contain at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond. For example, the cycloalkynyl is a fused cycloalkynyl.

The term “fused cycloalkynyl” refers to a bicyclic cycloalkyl group as defined herein which contains at least one triple bond and is formed by two or more rings sharing two adjacent atoms.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄ 6 cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused ring, which refer to cyclic structures containing 8 to 9 ring atoms within the above examples.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. The group can be attached to the remainder of the molecule through either ring. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring or bicyclic fused aryl. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene or

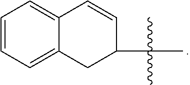 [SMILES: *C1C=Cc2ccccc2C1]

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

For example, the heteroaryl is a bicyclic fused heteroaryl, or a benzo fused heteroaryl.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

Representative examples of bicyclic fused heteroaryl include, but not limited to, the following groups benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeably throughout the disclosure herein. The group can be attached to the remainder of the molecule through either ring. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Heterocyclyl”, “heterocycle” or “heterocyclic” are interchangeable and refer to a non-aromatic heterocyclyl group comprising one or more heteroatoms selected from nitrogen, oxygen, silicon or optionally oxidized sulfur as ring members, with the remaining ring members being carbon, including monocyclic, fused ring, i.e., containing monocyclic heterocyclyl, and fused heterocyclic groups, bridged heterocyclic groups or spiro heterocyclic groups.

The term “unsaturated heterocyclic ring” referred to “heterocyclyl”, “heterocycle” or “heterocyclic” comprising at least one carbon-carbon double bond. For example, heterocyclyl includes

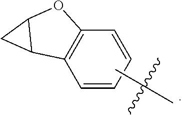

The term “optionally oxidized sulfur” used herein refers to S, SO or SO₂.

The term “monocyclic heterocyclyl” refers to monocyclic groups in which at least one ring member (e.g., 1-3 heteroatoms, 1 or 2 heteroatom(s)) is a heteroatom selected from nitrogen, oxygen, silicon or optionally oxidized sulfur. A heterocycle may be saturated or partially saturated (i.e., not forming a completely conjugated pi-electron system).

Exemplary monocyclic 4 to 9-membered heterocyclyl groups include, but not limited to, pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, 1,1-dioxo-thiomorpholinyl, oxazolidinyl, or oxazolidin-4-yl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl is divided into bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include, but not limited to, the following groups octahydrocyclopenta[c]pyrrole, octahydropyrrolo[3,4-c]pyrrolyl, octahydroisoindolyl, isoindolinyl, octahydro-benzo[b][1,4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, or tetrahydropyrazolopyrimidinyl (e.g., 4,5,6,7-tetrahydropyrazolo[1,5-a]pyrimidin-3-yl).

The term a “benzo fused heterocyclyl” is a polycyclic (e.g., bicyclic) fused heterocyclyl in which a monocyclic or polycyclic (e.g., bicyclic) 4- to 14-membered heterocyclyl as defined herein (e.g., 4 to 9-membered heterocyclyl, preferably 5- or 6-membered heterocyclyl) is fused to a benzene ring.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. Specifically, the bridge including the two bridgeheads contains 1-6 atoms selected from carbon, oxygen, nitrogen and sulfur with no two heteroatoms (oxygen, nitrogen and sulfur) being connected to each other. One or more rings of a bridged heterocyclyl group may contain one or more double bonds, but none of the rings has a completely conjugated pi-electron system. Preferably, a bridged heterocyclyl is 6- to 14-membered, or 7- to 12-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

“Spiro heterocyclyl” refers to a 5- to 20-membered polycyclic heterocyclyl with rings connected through one common carbon atom (called a spiro atom), wherein said rings have one or more heteroatoms selected from the group consisting of N, O, S, SO or SO₂ heteroatoms as ring atoms, with the remaining ring atoms being C. Preferably a spiro heterocyclyl is 6- to 14-membered, and more preferably 7- to 10-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 4-membered/5-membered, 4-membered/6-membered, 5-membered/5-membered, or 5-membered/6-membered mono-spiro heterocyclyl. Representative examples of spiro heterocyclyls include, but are not limited to the following groups: 1,7-dioxaspiro[4.5]decyl, 2-oxa-7-aza-spiro[4.4]nonyl, 7-oxa spiro[3.5]nonyl, 5-oxa-spiro[2.4]heptyl, and 2-oxa-6-azaspiro[3.3]heptyl.

“N-linked heterocyclyl” disclosed herein refers to a heterocyclyl group which is connected to the other part of the molecule by a bond from a nitrogen atom of the heterocyclyl ring. “N-linked heterocyclyl comprising 0, 1 or 2 additional heteroatoms selected from nitrogen, oxygen or optionally oxidized sulfur as ring member(s)” refers to a heterocyclyl group which is connected to the other part of the molecule by a bond from a nitrogen atom of the heterocyclyl ring, and which comprises 0, 1 or 2 additional heteroatoms in addition to the nitrogen atom linked to the other part of the molecule.

“C-linked heterocyclyl” disclosed herein refers to a heterocyclyl group which is connected to the other part of the molecule by a bond from a carbon atom of the heterocyclyl ring. “Si-linked heterocyclyl” disclosed herein refers to a heterocyclyl group which is connected to the other part of the molecule by a bond from a silicon atom of the heterocyclyl ring.

The term “at least one substituent” disclosed herein includes, for example, from 1 to 4, such as from 1 to 3, further as 1 or 2, substituents, provided that the theory of valence is met. For example, “at least one substituent R^{d}” disclosed herein includes from 1 to 4, such as from 1 to 3, further as 1 or 2, substituents selected from the list of R^{d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

Compounds disclosed herein also comprise deuterated compounds. The term “deuterated compound” refers to a compound wherein one or more carbon-bound hydrogen(s) are replaced by one or more deuterium(s). Similarly, the term “deuterated” is be used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium. “Deuterated” may be mono-substituted, bi-substituted, multi-substituted or completely-substituted.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

The configuration of diastereomeric or enantiomeric isomers could be assigned by technologies including but not limited to: 1D- or 2D-NMR spectroscopy of compounds or their derivatives (e.g. Mosher ester); optical rotatory dispersion; circular dichroism spectroscopy; X-ray diffractometry; in silico calculation (e.g. QM or MMGBSA).

Some of the compounds disclosed herein may exist with different points of attachment of hydrogen, referred to tautomers. For example, compounds including carbonyl —CH₂C(O)— groups (keto forms) may undergo tautomerism to form hydroxyl —CH═C(OH) (enol forms). Both keto and enol forms, individually as well as mixtures thereof, are also intended to be included where applicable. For example

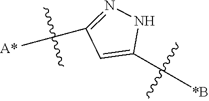 [SMILES: *c1cc(*)[nH]n1]

*B may undergo tautomerism to form

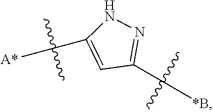

wherein A* and B* refer to the position substituents connect to pyrazole.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administered via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ratio used.

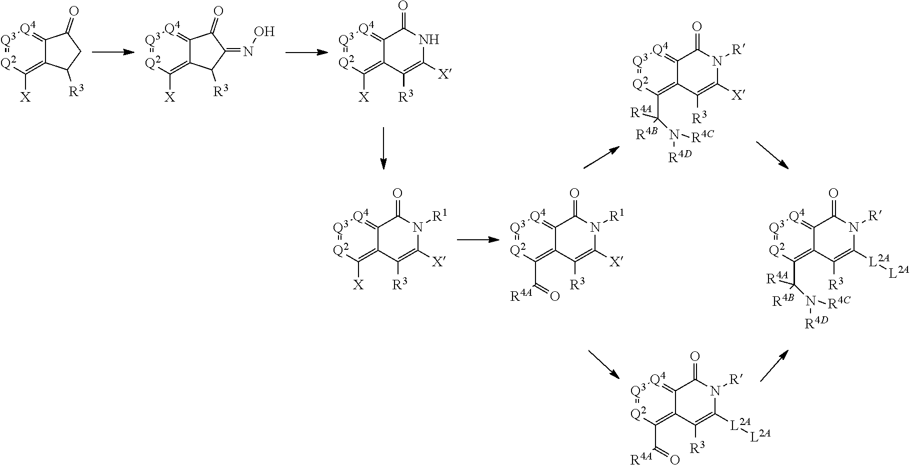

Compounds of Formula (I) can be prepared as shown in Scheme I, wherein R¹, L², R², R³, R^{4A}, R^{4B}, R^{4C}, R^{4D}, R⁵, R⁶, and R⁷ are as described herein (e.g., in Formula (I), or any aspect thereof, such as in Formula (IV)); X and X′ are each independently —F, —Cl, —Br, —I, or —OTf.

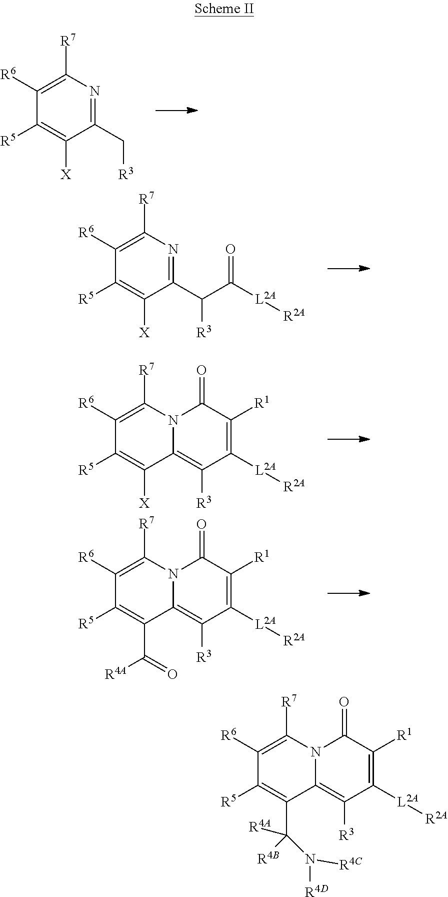

Compounds of Formula (VI) can be prepared as shown in Scheme II, wherein R¹, L^{2A}, R^{2A}, R³, R^{4A}, R^{4B}, R^{4C}, R^{4D}, R⁵, R⁶, and R⁷ are as described herein (e.g., in Formula (VI)); X is —F, —Cl, —Br, —I, or —OTf.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). Compound names except the reagents were generated by ChemDraw version 12.0. Chiral purity of compounds are determined by chiral HPLC or chiral SFC.

### Abbreviations

| | |
|---|---|
| AcOH | Acetic acid |
| ACN | Acetonitrile |
| AIBN | 2,2′-Azobis(2-methylpropionitrile) |
| Aq | Aqueous |
| Brine | Saturated aqueous sodium chloride solution |
| Bn | Benzyl |
| BnBr | Benzyl Bromide |
| Boc₂O | Di-tert-butyl dicarbonate |
| CMBP | Cyanomethylenetributylphosphorane |
| dba | Dibenzylideneacetone |
| DMF | N,N-Dimethylformamide |
| Dppf | 1,1″-bis(diphenylphosphino)ferrocene |
| DBU | 1,8-diazabicyclo[5.4.0]undec-7-ene |
| DEA | Diethylamine |
| DIEA or DIPEA | N-ethyl-N-isopropylpropan-2-amine |
| DMAP | 4-N,N-dimethylaminopyridine |
| DMF | N,N-dimethylformamide |
| DMSO | Dimethyl sulfoxide |
| EtOAc or EA | Ethyl acetate |
| EtOH | Ethanol |
| Et₂O or ether | Diethyl ether |
| Et₃N or TEA | Triethyl amine |
| HATU | O-(7-Azabenzotriazol-1-y1)-N,N,N′,N′-tetramethyluronium |
| | hexafluorophosphate |
| HPLC | High-performance liquid chromatography |
| IPA or i-PrOH | 2-propanol or Isopropyl alcohol |
| LiHMDS | Lithium Hexamethylenedisilazane |
| MeOH | Methanol |
| MOM-Br | Bromomethyl methyl ether |
| ms or MS | Mass spectrum |
| NaHMDS | Sodium Hexamethylenedisilazane |
| NBS | N-Bromosuccinimide |
| n-BuLi | n-Butyl lithium |
| n-BuOH | n-Butanol |
| n-Bu₃SnH | tri-n-Butyltinhydride |
| NIS | N-Iodosuccinimide |
| PE | petroleum ether |
| PPA | Polyphosphoric acid |
| p-TSA or TsOH | p-Tolunesulfonic acid |
| Rt | Retention time |
| Rt or rt | Room temperature |
| SEM-Cl | 2-chloromethyl 2-(trimethylsilyl)ethyl ether |
| TBAF | Tetra-butyl ammonium fluoride |
| TBSCl | tert-Butyldimethylsilyl chloride |
| TFA | Trifluoroacetic acid |
| THF | tetrahydrofuran |
| TLC | thin layer chromatography |
| Trt | trityl |
| Tf | trifluoromethanesulfonyl |

### Preparation of intermediate 5-acetyl-3-chloro-2,7-dimethylisoquinolin-1(2H)-one

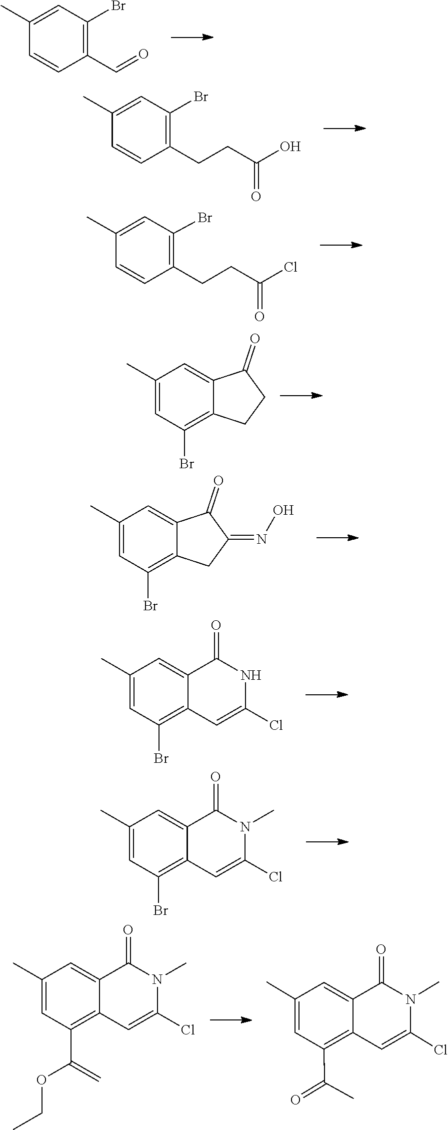

### Step 1: 3-(2-bromo-4-methylphenyl)propanoic acid

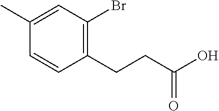

A mixture of 2-bromo-4-methylbenzaldehyde (100.0 g, 502.40 mmol), 2,2-dimethyl-1,3-dioxane-4,6-dione (87.0 g, 602.88 mmol) was added TEA (100 mL) at 0° C. After stirring for 10 min, formic acid (100 mL) was added dropwise at 0° C. Then the mixture was heated to 100° C. and stirred for 2 h. LCMS showed the reaction was completed. Water (50 mL) was added slowly and yellow solid was crashed out. The solid was collected by filtration, slurried with EA (200 mL), and filtered to give the desired product (90.0 g, 74%). MS (ESI) m/e [M+1]⁺=243, 245.

### Step 2: 3-(2-bromo-4-methylphenyl)propanoyl chloride

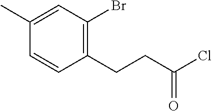

To a solution of 3-(2-bromo-4-methylphenyl)propanoic acid (90.0 g, 370.22 mmol) in DCM (900 mL) was added (COCl)₂ (94.0 g, 740.45 mmol) at 0° C. slowly. Then DMF (2.85 mL, 37.02 mmol) was added and the mixture was stirred at 20° C. for 2 h. TLC indicated the reaction was completed. The reaction mixture was concentrated under reduced pressure to give the desired product (95.0 g, crude), which was used directly in next step.

### Step 3: 4-bromo-6-methyl-2,3-dihydro-1H-inden-1-one

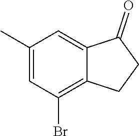

To a solution of 3-(2-bromo-4-methylphenyl)propanoyl chloride (95.0 g, crude) in DCM (1000 mL) was added AlCl₃ (96.9 g, 726.46 mmol) in portions at 0° C. The mixture was stirred at 20° C. for 2 h.

LCMS showed the reaction was completed. The mixture was poured into ice-water (1000 mL) slowly and stirred for 30 min. The aqueous phase was extracted with DCM (1000 mL×3). The combined organic phase was washed with brine (300 mL), dried with anhydrous Na₂SO₄, filtered and concentrated in vacuum. The crude product was slurried with EtOAc (150 mL) and filtered to give the desired product (75.0 g, 92%). MS (ESI) m/e [M+1]⁺=225, 227.

### Step 4: 4-bromo-2-(hydroxyimino)-6-methyl-2,3-dihydro-1H-inden-1-one

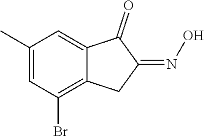

To a solution of 4-bromo-6-methyl-2,3-dihydro-1H-inden-1-one (30.0 g, 133.29 mmol) in 2-isopropoxypropane (300 mL) and HCl (150 mL) was added isoamyl nitrite (17.2 g, 146.61 mmol). The mixture was stirred at 20° C. for 3 h. LCMS showed the reaction was completed. The mixture was diluted with PE and the solid collected by filtration. The filter cake was dried under reduced pressure to give the desired product (30.0 g, 89%). MS (ESI) m/e [M+1]⁺=254, 256.

### Step 5: 5-bromo-3-chloro-7-methylisoquinolin-1(2H)-one

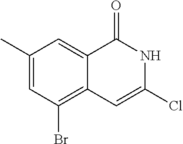

To a solution of 4-bromo-2-(hydroxyimino)-6-methyl-2,3-dihydro-1H-inden-1-one (10.0 g, 39.36 mmol) in CHCl₃ (200 mL) was added PCl₅ (13.1 g, 62.97 mmol) in portions. The mixture was stirred at 20° C. for 10 h. The reaction mixture was concentrated in vacuo and re-dissolved in HCl/EtOAc (100 mL). The resulting mixture was stirred at 20° C. for 2 h. LCMS showed the reaction was completed. The mixture was concentrated under reduced pressure. The crude product was slurried with EtOAc (50 mL) and filtered to give the desired product (6.0 g, 56%). MS (ESI) m/e [M+1]⁺=272.

### Step 6: 5-bromo-3-chloro-2,7-dimethylisoquinolin-1(2H)-one

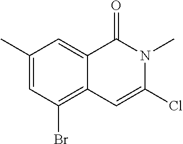

A mixture of 5-bromo-3-chloro-7-methylisoquinolin-1(2H)-one (10.0 g, 36.69 mmol), CH₃I (10.4 g, 73.39 mmol), and K₂CO₃ (10.1 g, 73.39 mmol) in DMF (100 mL) was stirred at 20° C. for 2 h. LCMS showed the reaction was completed. Water (50 mL) was added and the aqueous phase was extracted with ethyl acetate (100 mL×3). The combined organic phase was washed with brine (50 mL), dried with anhydrous Na₂SO₄, filtered and concentrated in vacuum. The residue was slurried with EtOAc (50 mL) and filtered to give the desired product (6.5 g, 62%). MS (ESI) m/e [M+1]⁺=286.

### Step 7: 3-chloro-5-(1-ethoxyvinyl)-2,7-dimethylisoquinolin-1(2H)-one

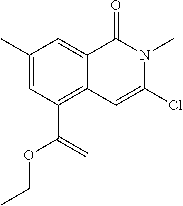

To a solution of 5-bromo-3-chloro-2,7-dimethylisoquinolin-1(2H)-one (6.5 g, 22.68 mmol) in dioxane (70 mL) was added tributyl(1-ethoxyvinyl)stannane (9.0 g, 24.95 mmol) and dichloropalladium; triphenylphosphane (1.7 g, 2.50 mmol) under N₂ atmosphere. The mixture was stirred at 60° C. for 16 h. LCMS showed the reaction was completed. The reaction was quenched by aq. KF (50 mL) and stirred at 20° C. for 0.5 h. The mixture was filtered and the filtrate was extracted with EtOAc (100 mL×3). The combined organic phase was washed with brine (100 mL), dried over anhydrous Na₂SO₄, filtered and concentrated in vacuo to give the desired product (6.0 g, 95%), which was used directly in next step. MS (ESI) m/e [M+1]⁺=278.

### Step 8: 5-acetyl-3-chloro-2,7-dimethylisoquinolin-1(2H)-one

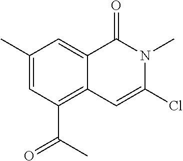

A mixture of 3-chloro-5-(1-ethoxyvinyl)-2,7-dimethylisoquinolin-1(2H)-one (6.0 g, 21.60 mmol) in dioxane (60 mL) and HCl (3M, 20 mL) was stirred at 20° C. for 0.5 h. LCMS showed the reaction was completed. The resulting mixture was extracted with DCM (100 mL×3). The combined organic phase was washed with brine (100 mL), dried over anhydrous Na₂SO₄, filtered and concentrated in vacuo. The crude product was purified by re-crystallization with MTBE (30 mL) to give the desired product (2.5 g, 46%). ¹H NMR (400 MHz, CDCl₃) δ ppm 8.40 (s, 1H), 7.93 (d, J=1.59 Hz, 1H), 7.74 (s, 1H), 3.76 (s, 3H), 2.69 (s, 3H), 2.53 (s, 3H). MS (ESI) m/e [M+1]⁺=250.

### Example 1: 2-((1-(2,7-dimethyl-1-oxo-3-phenyl-1,2-dihydroisoquinolin-5-yl)ethyl)amino)benzoic acid

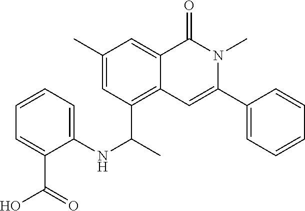 [SMILES: Cc1cc(C(C)Nc2ccccc2C(=O)O)c2cc(-c3ccccc3)n(C)c(=O)c2c1]

### Step 1: 5-acetyl-2,7-dimethyl-3-phenylisoquinolin-1(2H)-one

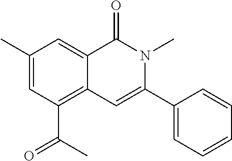 [SMILES: CC(=O)c1cc(C)cc2c(=O)n(C)c(-c3ccccc3)cc12]

A mixture of 5-acetyl-3-chloro-2,7-dimethylisoquinolin-1(2H)-one (130 mg, 0.52 mmol), phenylboronic acid (95 mg, 0.78 mmol), Pd(PPh₃)₄ (30 mg, 0.03 mmol), and K₃PO₄ (331 mg, 1.56 mmol) in dioxane (10 mL) and H₂O (1 mL) was stirred at 100° C. for 6 h under N₂. The mixture was poured into H₂O and extracted with EtOAc. The organic layer was dried over Na₂SO₄, filtered and concentrated in vacuum. The residue was purified by flash chromatography on silica gel eluted with 40%-45% EtOAc in petroleum ether to give the desired product (120 mg, 79%). MS (ESI) m/e [M+1]⁺=292.

### Step 2: 5-(1-aminoethyl)-2,7-dimethyl-3-phenylisoquinolin-1(2H)-one

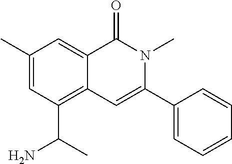 [SMILES: Cc1cc(C(C)N)c2cc(-c3ccccc3)n(C)c(=O)c2c1]

To a solution of 5-acetyl-2,7-dimethyl-3-phenylisoquinolin-1(2H)-one (50 mg, 0.17 mmol) and ammonium acetate (66 mg, 0.86 mmol) in 5 mL of ethanol was added NaBH₃CN (14 mg, 0.22 mmol). The resulting solution was stirred overnight at 90° C. After cooled to room temperature, the solution was concentrated in vacuum and the residue was purified by CombiFlash on silica gel eluted with 8%-10% MeOH in DCM to give the desired product (45 mg, 90%). MS (ESI) m/e [M+1]⁺=293.

### Step 3: 2-((1-(2,7-dimethyl-1-oxo-3-phenyl-1,2-dihydroisoquinolin-5-yl)ethyl)amino)benzoic acid

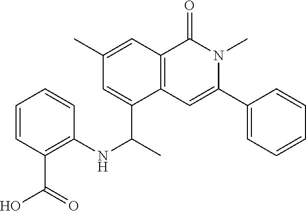 [SMILES: Cc1cc(C(C)Nc2ccccc2C(=O)O)c2cc(-c3ccccc3)n(C)c(=O)c2c1]

To a solution of 5-(1-aminoethyl)-2,7-dimethyl-3-phenylisoquinolin-1(2H)-one (45 mg, 0.15 mmol), 2-iodobenzoic acid (57 mg, 0.23 mmol) and K₂CO₃ (64 mg, 0.46 mmol) in 3 mL of DMSO was added CuI (3 mg, 0.02 mmol) and L-proline (4 mg, 0.03 mmol). The resulting solution was stirred overnight at 100° C. under N₂. After cooled to room temperature, the solution was concentrated in vacuum. The residue was redissolved in H₂O and acidized to pH 5˜6 with HCl (2 M in water). The mixture was filtered and the filter cake was purified by Prep-HPLC to give the desired product (12 mg, 19%). ¹H NMR (400 MHz, DMSO-d₆) δ ppm 12.75 (s, 1H), 8.37 (s, 1H), 8.00 (s, 1H), 7.80 (d, J=7.4 Hz, 1H), 7.62-7.45 (m, 6H), 7.21-7.13 (m, 1H), 6.83 (s, 1H), 6.56-6.47 (m, 1H), 6.34 (d, J=8.1 Hz, 1H), 5.20 (s, 1H), 3.33 (s, 3H), 2.37 (s, 3H), 1.51 (d, J=3.7 Hz, 7H). MS (ESI) m/e [M+1]⁺=413.

### Example 2: 2-((1-(2,7-dimethyl-1-oxo-3-(o-tolyl)-1,2-dihydroisoquinolin-5-yl)ethyl)amino)benzoic acid

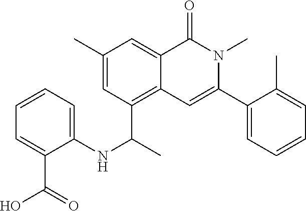 [SMILES: Cc1cc(C(C)Nc2ccccc2C(=O)O)c2cc(-c3ccccc3C)n(C)c(=O)c2c1]

### Step 1: 5-acetyl-2,7-dimethyl-3-(o-tolyl)isoquinolin-1(2H)-one

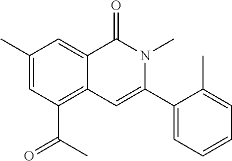 [SMILES: CC(=O)c1cc(C)cc2c(=O)n(C)c(-c3ccccc3C)cc12]

A mixture of 5-acetyl-3-chloro-2,7-dimethylisoquinolin-1(2H)-one (120 mg, 0.48 mmol), o-tolylboronic acid (98 mg, 0.72 mmol), Pd(PPh₃)₄ (28 mg, 0.02 mmol), and K₃PO₄ (306 mg, 1.44 mmol) in dioxane (10 mL) and H₂O (1 mL) was stirred at 100° C. for 15 h under N₂. The mixture was poured into H₂O and extracted with EtOAc. The organic layer was dried over anhydrous Na₂SO₄, filtered and concentrated in vacuum. The residue was purified by flash chromatography on silica gel to give the desired product (141 mg, 95%). MS (ESI) m/e [M+1]⁺=306.

### Step 2: 5-(1-hydroxyethyl)-2,7-dimethyl-3-(o-tolyl)isoquinolin-1(2H)-one

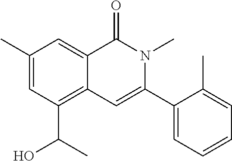 [SMILES: Cc1cc(C(C)O)c2cc(-c3ccccc3C)n(C)c(=O)c2c1]

A solution of 5-acetyl-2,7-dimethyl-3-(o-tolyl)isoquinolin-1(2H)-one (141 mg, 0.46 mmol) in DCM (10 mL) and MeOH (10 mL) was treated with NaBH₄ (35 mg, 0.92 mmol) in portions at 0° C. The resulting solution was stirred at RT for 1 h. The mixture was diluted with H₂O (10 mL) and extracted with DCM (30 mL×2). The combined organic phase were washed with brine (20 mL), dried over anhydrous Na₂SO₄, filtered, and concentrated in vacuum. The residue was purified by flash chromatography on silica gel to give the desired product (140 mg, 99%). MS (ESI) m/e [M+1]⁺=308.

### Step 3: 5-(1-bromoethyl)-2,7-dimethyl-3-(o-tolyl)isoquinolin-1(2H)-one

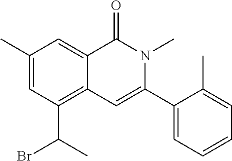 [SMILES: Cc1cc(C(C)Br)c2cc(-c3ccccc3C)n(C)c(=O)c2c1]

To a solution of 5-(1-hydroxyethyl)-2,7-dimethyl-3-(o-tolyl)isoquinolin-1(2H)-one (140 mg, 0.45 mmol) in DCM (5 ml) was added PBr₃ (366 mg, 1.35 mmol) dropwise at 0° C. The resulting solution was stirred for 2 hat RT. The reaction was quenched with water (20 ml) at 0° C. and the pH was adjusted to 8 with saturated aqueous NaHCO₃. The mixture was extracted with DCM (10 mL×2). The combined organic phase were washed with brine, dried over anhydrous Na₂SO₄, filtered, and concentrated to give the desired product (80 mg, 48%). MS (ESI) m/e [M+1]⁺=370.

### Step 4: methyl 2-((1-(2,7-dimethyl-1-oxo-3-(o-tolyl)-1,2-dihydroisoquinolin-5-yl)ethyl)amino)benzoate

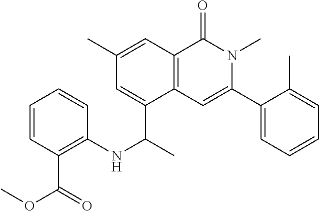 [SMILES: COC(=O)c1ccccc1NC(C)c1cc(C)cc2c(=O)n(C)c(-c3ccccc3C)cc12]

To a solution of 5-(1-bromoethyl)-2,7-dimethyl-3-(o-tolyl)isoquinolin-1(2H)-one (80 mg, 0.22 mmol) and methyl 2-aminobenzoate (48 mg, 0.32 mmol) in DMF (3 ml) was added DIEA (84 mg, 0.65 mmol). The resulting solution was stirred for 15 h at 90° C. After cooled to room temperature. The reaction was diluted with water (30 ml) and extracted with EtOAc (30 mL×2). The combined organic phase was washed with brine (20 mL), dried over anhydrous Na₂SO₄, filtered, and concentrated in vacuum. The residue was purified on flash chromatography on silica gel to give the desired product (35 mg, 37%). MS (ESI) m/e [M+1]⁺=441.

### Step 5: 2-((1-(2,7-dimethyl-1-oxo-3-(o-tolyl)-1,2-dihydroisoquinolin-5-yl)ethyl)amino)benzoic acid

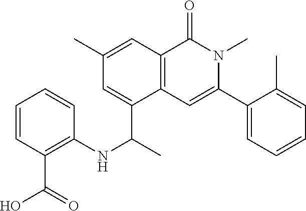 [SMILES: Cc1cc(C(C)Nc2ccccc2C(=O)O)c2cc(-c3ccccc3C)n(C)c(=O)c2c1]

To a solution of methyl 2-((1-(2,7-dimethyl-1-oxo-3-(o-tolyl)-1,2-dihydroisoquinolin-5-yl)ethyl)amino)benzoate (35 mg, 0.07 mmol) in methanol (4 mL), H₂O (2 ml) and THF (4 ml) was added lithium hydroxide monohydrate (18 mg, 0.37 mmol). The resulting solution was stirred for 4 h at 50° C. The solution was concentrated in vacuum. The residue was redissolved in H₂O and acidized to pH 5-6 with HCl (2 M in water). The mixture was filtered, and the filter cake was purified by Prep-HPLC to give the desired product (13 mg, 38%). ¹H NMR (399 MHz, DMSO-d₆) δ ppm 12.72 (brs, 1H), 8.38 (brs, 1H), 8.00 (s, 1H), 7.84-7.75 (m, 1H), 7.53-7.29 (m, 5H), 7.21-7.09 (m, 1H), 6.83-6.73 (m, 1H), 6.55-6.46 (m, 1H), 6.40-6.27 (m, 1H), 5.24-5.10 (m, 1H), 3.16 (s, 3H), 2.55-2.46 (m, 3H), 2.40-2.31 (m, 3H), 2.24-2.12 (m, 3H), 1.53-1.44 (m, 3H). MS (ESI) m/e [M+1]⁺=427.

### Example 3: 2-((1-(3-(3-cyanophenyl)-2,7-dimethyl-1-oxo-1,2-dihydroisoquinolin-5-yl)ethyl)amino)benzoic acid

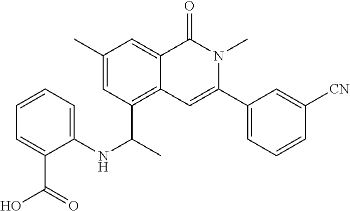 [SMILES: Cc1cc(C(C)Nc2ccccc2C(=O)O)c2cc(-c3cccc(C#N)c3)n(C)c(=O)c2c1]

The desired product (7.7 mg) was obtained following the similar procedure as Example 1. ¹H NMR (399 MHz, DMSO-d₆) δ ppm 12.73 (brs, 1H), 8.36 (s, 1H), 8.13 (s, 1H), 8.04-7.89 (m, 3H), 7.84-7.61 (m, 2H), 7.46 (s, 1H), 7.21-7.08 (m, 1H), 6.91 (s, 1H), 6.55-6.40 (m, 1H), 6.31 (d, J=8.2 Hz, 1H), 5.23 (s, 1H), 3.30 (s, 3H), 2.33 (s, 3H), 1.49 (brs, 3H). MS (ESI) m/e [M+1]⁺=437.

### Example 4: 2-((1-(3-(4-cyanophenyl)-2,7-dimethyl-1-oxo-1,2-dihydroisoquinolin-5-yl)ethyl)amino)benzoic acid

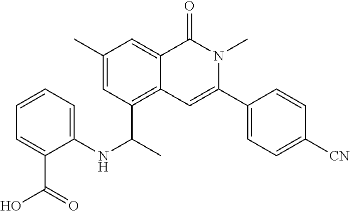 [SMILES: Cc1cc(C(C)Nc2ccccc2C(=O)O)c2cc(-c3ccc(C#N)cc3)n(C)c(=O)c2c1]

The desired product (10 mg) was obtained following the similar procedure as Example 1. ¹H NMR (399 MHz, DMSO-d₆) δ ppm 12.72 (brs, 1H), 8.36 (s, 1H), 8.04-7.92 (m, 3H), 7.85-7.71 (m, 3H), 7.47 (s, 1H), 7.22-7.08 (m, 1H), 6.90 (s, 1H), 6.57-6.45 (m, 1H), 6.31 (d, J=8.4 Hz, 1H), 5.21 (s, 1H), 3.30 (s, 3H), 2.33 (s, 3H), 1.48 (brs, 3H). MS (ESI) m/e [M+1]⁺=437.

### Example 5: 2-((1-(3-(3-(2-cyanopropan-2-yl)phenyl)-2,7-dimethyl-1-oxo-1,2-dihydroisoquinolin-5-yl)ethyl)amino)benzoic acid

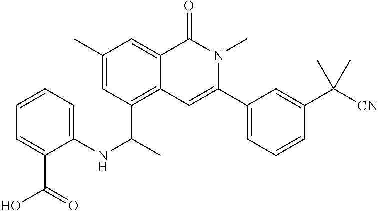 [SMILES: Cc1cc(C(C)Nc2ccccc2C(=O)O)c2cc(-c3cccc(C(C)(C)C#N)c3)n(C)c(=O)c2c1]

The desired product (10.5 mg) was obtained following the similar procedure as Example 1. ¹H NMR (399 MHz, DMSO-d₆) δ ppm 12.69 (brs, 1H), 8.33 (s, 1H), 7.98 (s, 1H), 7.78 (d, J=7.6 Hz, 1H), 7.74-7.63 (m, 2H), 7.60-7.51 (m, 2H), 7.47 (s, 1H), 7.23-7.09 (m, 1H), 6.85 (s, 1H), 6.56-6.42 (m, 1H), 6.34 (d, J=8.4 Hz, 1H), 5.22 (s, 1H), 3.30 (s, 3H), 2.36 (s, 3H), 1.72 (s, 6H), 1.49 (d, J=4.1 Hz, 3H). MS (ESI) m/e [M+1]⁺=480.

### Example 6: 2-((1-(3-(2-fluorophenyl)-2,7-dimethyl-1-oxo-1,2-dihydroisoquinolin-5-yl)ethyl)amino)benzoic acid

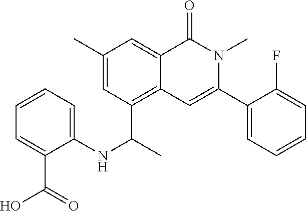 [SMILES: Cc1cc(C(C)Nc2ccccc2C(=O)O)c2cc(-c3ccccc3F)n(C)c(=O)c2c1]

### Step 1: 1-(1-(3-chloro-2,7-dimethyl-1-oxo-1,2-dihydroisoquinolin-5-yl)ethyl)-2H-benzo[d][1,3]oxazine-2,4(1H)-dione

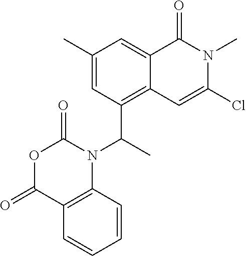 [SMILES: Cc1cc(C(C)n2c(=O)oc(=O)c3ccccc32)c2cc(Cl)n(C)c(=O)c2c1]

A solution of 3-chloro-5-(1-hydroxyethyl)-2,7-dimethylisoquinolin-1(2H)-one (50 mg, 0.20 mmol), PPh₃ (79 mg, 0.30 mmol) and 2H-benzo[d][1,3]oxazine-2,4(1H)-dione (49 mg, 0.30 mmol) in THF (1 mL) was added DIAD (60 mg, 0.30 mmol) at 0° C. The mixture was stirred at 0° C. for 1 h. The mixture was concentrated in vacuum and the residue was purified by prep-TLC to give the desired product (28 mg, 35%). MS (ESI) m/e [M+Na]⁺=419.

### Step 2: 2-((1-(3-(2-fluorophenyl)-2,7-dimethyl-1-oxo-1,2-dihydroisoquinolin-5-yl)ethyl)amino)benzoic acid

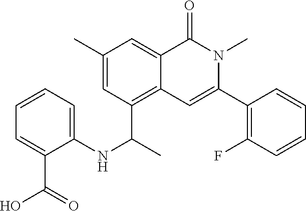 [SMILES: Cc1cc(C(C)Nc2ccccc2C(=O)O)c2cc(-c3ccccc3F)n(C)c(=O)c2c1]

To a solution of methyl 2-((1-(3-chloro-2,7-dimethyl-1-oxo-1,2-dihydroisoquinolin-5-yl)ethyl)amino)benzoate (28 mg, 0.07 mmol), (3-fluorophenyl)boronic acid (13 mg, 0.10 mmol) and Na₂CO₃ (22 mg, 0.20 mmol) in 3 mL of dioxane and 1 mL of H₂O was added Pd(PPh₃)₄ (12 mg, 0.01 mmol) under nitrogen atmosphere. The resulting solution was stirred overnight at 100° C. After cooled to room temperature, the mixture was poured into H₂O and extracted with EtOAc. The organic layer was dried over Na₂SO₄, filtered, and concentrated in vacuum. The residue was purified by prep-HPLC to give the desired product (12 mg, 39%). ¹H NMR (400 MHz, DMSO-d₆) δ ppm 12.74 (brs, 1H), 8.37 (s, 1H), 8.01 (s, 1H), 7.78 (d, J=6.7 Hz, 1H), 7.68-7.56 (m, 2H), 7.50 (s, 1H), 7.47-7.36 (m, 2H), 7.25-7.11 (m, 1H), 6.96 (d, J=10.5 Hz, 1H), 6.58-6.45 (m, 1H), 6.42-6.26 (dd, J=10.5, 6.7 Hz, 1H), 5.30-5.13 (m, 1H), 3.33 (s, 3H), 2.38 (s, 3H), 1.60-1.41 (m, 3H). MS (ESI) m/e [M+Na]⁺=453.

### Example 7: 2-((1-(3-(3-fluorophenyl)-2,7-dimethyl-1-oxo-1,2-dihydroisoquinolin-5-yl)ethyl)amino)benzoic acid

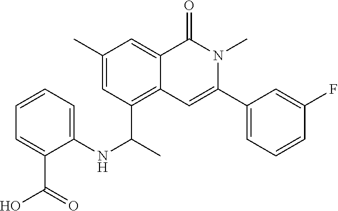 [SMILES: Cc1cc(C(C)Nc2ccccc2C(=O)O)c2cc(-c3cccc(F)c3)n(C)c(=O)c2c1]

The desired product (24 mg) was obtained following the similar procedure as Example 2. ¹H NMR (400 MHz, DMSO-d₆) δ ppm 12.74 (s, 1H), 8.37 (d, J=4.0 Hz, 1H), 7.99 (s, 1H), 7.80 (d, J=7.1 Hz, 1H), 7.62-7.42 (m, 4H), 7.36 (t, J=8.4 Hz, 1H), 7.17 (t, J=4.8 Hz, 1H), 6.88 (s, 1H), 6.52 (t, J=7.2 Hz, 1H), 6.34 (d, J=8.4 Hz, 1H), 5.27-5.18 (m, 1H), 3.33 (s, 3H), 2.35 (s, 3H), 1.51 (d, J=4.8 Hz, 3H). MS (ESI) m/e [M+1]⁺=431

### Example 8: 2-((1-(3-(4-fluorophenyl)-2,7-dimethyl-1-oxo-1,2-dihydroisoquinolin-5-yl)ethyl)amino)benzoic acid

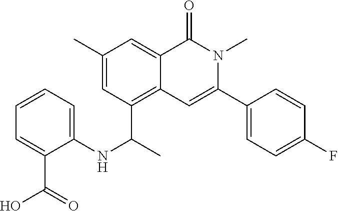 [SMILES: Cc1cc(C(C)Nc2ccccc2C(=O)O)c2cc(-c3ccc(F)cc3)n(C)c(=O)c2c1]

The desired product (21 mg) was obtained following the similar procedure as Example 2. ¹H NMR (400 MHz, DMSO-d₆) δ ppm 12.74 (brs, 1H), 8.37 (d, J=4.8 Hz, 1H), 7.99 (s, 1H), 7.79 (d, J=7.8 Hz, 1H), 7.68-7.62 (m, 2H), 7.47 (s, 1H), 7.36 (t, J=7.8 Hz, 2H), 7.17 (t, J=7.2 Hz, 1H), 6.83 (s, 1H), 6.51 (t, J=6.4 Hz, 1H), 6.33 (d, J=8.4 Hz, 1H), 5.24-5.15 (m, 1H), 3.32 (s, 3H), 2.36 (s, 3H), 1.50 (d, J=4.8 Hz, 3H). MS (ESI) m/e [M+1]⁺=431

### Example 9: 2-((1-(3-(3,4-difluorophenyl)-2,7-dimethyl-1-oxo-1,2-dihydroisoquinolin-5-yl)ethyl)amino)benzoic acid

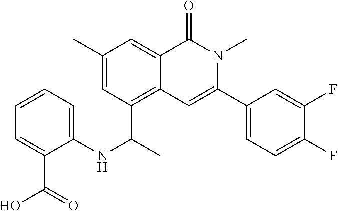 [SMILES: Cc1cc(C(C)Nc2ccccc2C(=O)O)c2cc(-c3ccc(F)c(F)c3)n(C)c(=O)c2c1]

The desired product (27 mg) was obtained following the similar procedure as Example 2. ¹H NMR (400 MHz, DMSO-d₆) δ ppm 12.75 (s, 1H), 8.65-8.27 (m, 1H), 7.99 (s, 1H), 7.86-7.70 (m, 2H), 7.64-7.57 (m, 1H), 7.51-7.44 (m, 2H), 7.15 (t, J=4.2 Hz, 1H), 6.89 (s, 1H), 6.51 (t, J=6.8 Hz, 1H), 6.32 (d, J=8.0 Hz, 1H), 5.25-5.15 (m, 1H), 3.34 (s, 3H), 2.37 (s, 3H), 1.50 (d, J=4.8 Hz, 3H). MS (ESI) m/e [M+1]⁺=439

### Example 10: 2-((1-(3-(3,5-difluorophenyl)-2,7-dimethyl-1-oxo-1,2-dihydroisoquinolin-5-yl)ethyl)amino)benzoic acid

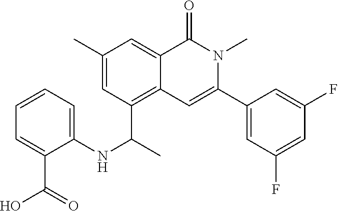 [SMILES: Cc1cc(C(C)Nc2ccccc2C(=O)O)c2cc(-c3cc(F)cc(F)c3)n(C)c(=O)c2c1]

### Step 1: 3-chloro-5-(1-hydroxyethyl)-2,7-dimethylisoquinolin-1(2H)-one

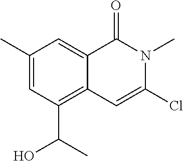

A solution of 5-acetyl-3-chloro-2,7-dimethylisoquinolin-1(2H)-one (200 mg, 0.80 mmol) in THF (10 mL) was added NaBH₄ (76 mg, 2.00 mmol) at 0° C. The mixture was stirred at room temperature for 2 h. The mixture was poured into H₂O and extracted with EtOAc. The organic layer was dried over Na₂SO₄, filtered, and concentrated in vacuum. The residue was purified by flash chromatography on silica gel to give the desired product (120 mg, 60%). MS (ESI) m/e [M+1]⁺=252.

### Step 2: 5-(1-bromoethyl)-3-chloro-2,7-dimethylisoquinolin-1(2H)-one

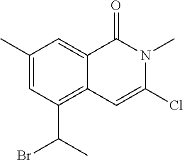

A solution of 3-chloro-5-(1-hydroxyethyl)-2,7-dimethylisoquinolin-1(2H)-one (120 mg, 0.48 mmol) in DCM (10 mL) was added PBr₃ (540 mg, 2.00 mmol) at 0° C. The mixture was stirred at room temperature for further 12 h. The mixture was poured into H₂O and extracted with DCM. The organic layer was dried over Na₂SO₄, filtered, and concentrated in vacuum to give the desired product (100 mg, 67%). MS (ESI) m/e [M+1]⁺=314.

### Step 3: methyl 2-((1-(3-chloro-2,7-dimethyl-1-oxo-1,2-dihydroisoquinolin-5-yl)ethyl)amino)benzoate

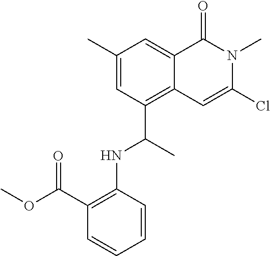

To a solution of 5-(1-bromoethyl)-3-chloro-2,7-dimethylisoquinolin-1(2H)-one (100 mg, 0.32 mmol), methyl 2-aminobenzoate (150 mg, 1.0 mmol) and DIEA (258 mg, 2.0 mmol) in DMSO (160 mL) was stirred at 90° C. for 12 h. The mixture was poured into H₂O and extracted with EtOAc. The organic layer was dried over Na₂SO₄, filtered, and concentrated under vacuum. The residue was purified by flash chromatography on silica gel to give the desired product (30 mg, 25%). MS (ESI) m/e [M+Na]⁺=407.

### Step 4: methyl 2-((1-(3-(3,5-difluorophenyl)-2,7-dimethyl-1-oxo-1,2-dihydroisoquinolin-5-yl)ethyl)amino)benzoate

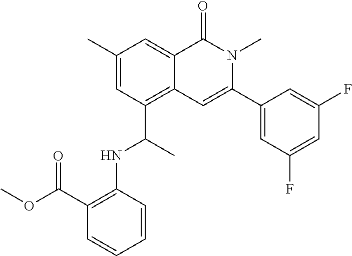 [SMILES: COC(=O)c1ccccc1NC(C)c1cc(C)cc2c(=O)n(C)c(-c3cc(F)cc(F)c3)cc12]

To a solution of methyl 2-((1-(3-chloro-2,7-dimethyl-1-oxo-1,2-dihydroisoquinolin-5-yl)ethyl)amino)benzoate (30 mg, 0.08 mmol), (3,5-difluorophenyl)boronic acid (15 mg, 0.10 mmol) and Na₂CO₃ (22 mg, 0.20 mmol) in 3 mL of dioxane and 1 mL of H₂O was added Pd(PPh₃)₄ (12 mg, 0.01 mmol) under nitrogen atmosphere. The resulting solution was stirred overnight at 100° C. After cooled to room temperature, the solution was concentrated in vacuum and the residue was purified by prep-TLC to give the desired product (15 mg, 42%). MS (ESI) m/e [M+Na]⁺=485.

### Step 5: 2-((1-(3-(3,5-difluorophenyl)-2,7-dimethyl-1-oxo-1,2-dihydroisoquinolin-5-yl)ethyl)amino)benzoic acid

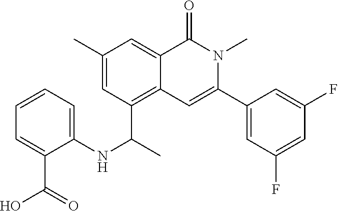 [SMILES: Cc1cc(C(C)Nc2ccccc2C(=O)O)c2cc(-c3cc(F)cc(F)c3)n(C)c(=O)c2c1]

To a solution of methyl 2-((1-(3-(3,5-difluorophenyl)-2,7-dimethyl-1-oxo-1,2-dihydroisoquinolin-5-yl)ethyl)amino)benzoate (15 mg, 0.03 mmol) in methanol (0.5 mL) and THF (0.5 mL) was added 1N NaOH aqueous solution (1 mL). The resulting solution was stirred for 12 h at 25° C. The solution was concentrated in vacuum. The residue was redissolved in H₂O and acidized to pH 5˜6 with HCl (2 M in water). The resulting mixture was extracted with EtOAc (10 mL×2). The combined organic layer was washed with brine (10 mL), dried over Na₂SO₄, filtered and concentrated in vacuo. The residue was purified by Prep-HPLC to give the desired product (6 mg, 41%). ¹H NMR (400 MHz, DMSO-d₆) δ ppm 12.78 (s, 1H), 8.42 (s, 1H), 8.00 (s, 1H), 7.81 (d, J=7.8 Hz, 1H), 7.55-7.36 (m, 4H), 7.22-7.10 (m, 1H), 6.94 (s, 1H), 6.58-6.46 (m, 1H), 6.34 (d, J=7.8 Hz, 1H), 5.24 (m, 1H), 3.30 (s, 3H), 2.37 (s, 3H), 1.56-1.44 (m, 3H). MS (ESI) m/e [M+Na]⁺=471

### Example 11: 2-((1-(3-(2,4-difluorophenyl)-2,7-dimethyl-1-oxo-1,2-dihydroisoquinolin-5-yl)ethyl)amino)benzoic acid

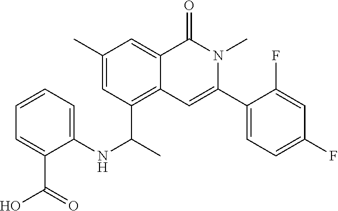 [SMILES: Cc1cc(C(C)Nc2ccccc2C(=O)O)c2cc(-c3ccc(F)cc3F)n(C)c(=O)c2c1]

The desired product (23 mg) was obtained following the similar procedure as Example 1. ¹H NMR (400 MHz, DMSO-d₆) δ ppm 12.74 (s, 1H), 8.65-8.27 (m, 1H), 8.00 (s, 1H), 7.79 (d, J=7.2 Hz, 1H), 7.74-7.63 (m, 1H), 7.59-7.45 (m, 2H), 7.31 (t, J=7.2 Hz, 1H), 7.22-7.16 (m, 1H), 6.97 (d, J=7.6 Hz, 1H), 6.52 (s, 1H), 6.39-6.27 (m, 1H), 5.26-5.14 (m, 1H), 3.28 (s, 3H), 2.38 (s, 3H), 1.49 (d, J=6.8 Hz, 3H). MS (ESI) m/e [M+1]⁺=439

### Example 12: 2-((1-(3-(2,3-difluorophenyl)-2,7-dimethyl-1-oxo-1,2-dihydroisoquinolin-5-yl)ethyl)amino)benzoic acid

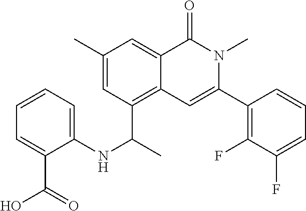 [SMILES: Cc1cc(C(C)Nc2ccccc2C(=O)O)c2cc(-c3cccc(F)c3F)n(C)c(=O)c2c1]

The desired product (6 mg) was obtained following the similar procedure as Example 11. ¹H NMR (400 MHz, DMSO-d₆) δ ppm 12.71 (s, 1H), 8.35 (s, 1H), 8.00 (s, 1H), 7.83-7.70 (m, 1H), 7.71-7.54 (m, 1H), 7.49 (s, 1H), 7.46-7.33 (m, 2H), 7.21-7.09 (m, 1H), 7.01 (s, 1H), 6.57-6.43 (m, 1H), 6.38-6.25 (m, 1H), 5.27-5.11 (m, 1H), 3.27 (s, 3H), 2.36 (s, 3H), 1.56-1.41 (m, 3H). MS (ESI) m/e [M+Na]⁺=471

### Example 13: 2-((1-(3-(3-methoxyphenyl)-2,7-dimethyl-1-oxo-1,2-dihydroisoquinolin-5-yl)ethyl)amino)benzoic acid

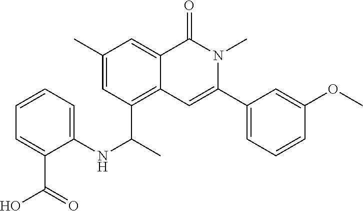 [SMILES: COc1cccc(-c2cc3c(C(C)Nc4ccccc4C(=O)O)cc(C)cc3c(=O)n2C)c1]

### Step 1: 5-acetyl-3-(3-methoxyphenyl)-2,7-dimethylisoquinolin-1(2H)-one

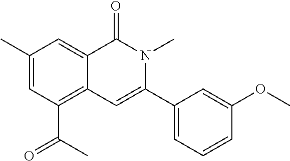

A mixture of 5-acetyl-3-chloro-2,7-dimethylisoquinolin-1(2H)-one (100 mg, 0.40 mmol), phenylboronic acid (91 mg, 0.60 mmol), Pd(PPh₃)₄ (23 mg, 0.02 mmol), and K₃PO₄ (254 mg, 1.20 mmol) in dioxane (10 mL) and H₂O (1 mL) was stirred at 100° C. for 15 h under N₂. The mixture was poured into H₂O and extracted with EtOAc. The organic layer was dried over anhydrous Na₂SO₄, filtered and concentrated in vacuum. The residue was purified by flash chromatography on silica gel to give the desired product (110 mg, 86%). MS (ESI) m/e [M+1]⁺=322.

### Step 2: 5-(1-aminoethyl)-3-(3-methoxyphenyl)-2,7-dimethylisoquinolin-1(2H)-one

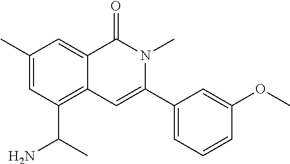

To a solution of 5-acetyl-3-(3-methoxyphenyl)-2,7-dimethylisoquinolin-1(2H)-one (100 mg, 0.31 mmol), formic acid (29 mg, 0.62 mmol) and ammonium formate (59 mg, 0.93 mmol) in 5 mL of methanol was added Cp*Ir [N-[4-(Trifluoromethyl)phenyl]-2-pyridinecarboxamidato]-Cl (10 mg, 0.02 mmol) under N₂. The resulting solution was stirred for 8 h at 60° C. After cooled to room temperature, the solution was concentrated in vacuum and the residue was purified by CombiFlash on silica gel to give the desired product (95 mg, 95%). MS (ESI) m/e [M+1]⁺=323.

### Step 3: 2-((1-(3-(3-methoxyphenyl)-2,7-dimethyl-1-oxo-1,2-dihydroisoquinolin-5-yl)ethyl)amino)benzoic acid

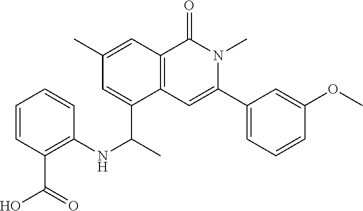 [SMILES: COc1cccc(-c2cc3c(C(C)Nc4ccccc4C(=O)O)cc(C)cc3c(=O)n2C)c1]

To a solution of 5-(1-aminoethyl)-3-(3-methoxyphenyl)-2,7-dimethylisoquinolin-1(2H)-one (95 mg, 0.29 mmol), 2-iodobenzoic acid (146 mg, 0.59 mmol) and K₂CO₃ (122 mg, 0.88 mmol) in 5 mL of DMSO was added CuI (6 mg, 0.03 mmol) and L-proline (7 mg, 0.06 mmol). The resulted solution was stirred overnight at 100° C. under N₂. After cooled to room temperature. The solution was concentrated in vacuum. The residue was redissolved in H₂O and acidized to pH 5˜6 with HCl (2 M in water). The mixture was filtered, and the filter cake was purified by Prep-HPLC to give the desired product (29 mg, 22%). ¹H NMR (400 MHz, DMSO-d₆) δ ppm 12.73 (brs, 1H), 8.34 (s, 1H), 7.97 (s, 1H), 7.78 (d, J=7.6 Hz, 1H), 7.43 (m, 2H), 7.14 (m, 3H), 7.05 (d, J=7.7 Hz, 1H), 6.81 (s, 1H), 6.50 (t, J=7.1 Hz, 1H), 6.33 (d, J=8.3 Hz, 1H), 5.18 (m, 1H), 3.80 (s, 3H), 3.32 (s, 3H), 2.35 (s, 3H), 1.49 (d, J=5.2 Hz, 3H). MS (ESI) m/e [M+1]⁺=443.

### Example 14: 2-((1-(3-(4-methoxyphenyl)-2,7-dimethyl-1-oxo-1,2-dihydroisoquinolin-5-yl)ethyl)amino)benzoic acid

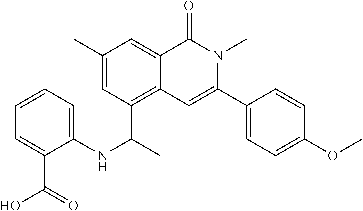 [SMILES: COc1ccc(-c2cc3c(C(C)Nc4ccccc4C(=O)O)cc(C)cc3c(=O)n2C)cc1]

The desired product (28 mg) was obtained following the similar procedure as Example 1. ¹H NMR (400 MHz, DMSO-d₆) δ ppm 12.76 (brs, 1H), 8.36 (s, 1H), 8.02-7.89 (m, 1H), 7.79 (s, 1H), 7.62-7.32 (m, 3H), 7.21-6.96 (m, 2H), 6.82-6.68 (m, 1H), 6.56-6.40 (m, 1H), 6.32 (s, 1H), 5.16 (s, 1H), 3.81 (s, 3H), 3.31 (s, 3H), 2.35 (s, 3H), 1.49 (brs, 3H). MS (ESI) m/e [M+1]⁺=443.

### Example 15: 2-((1-(3-(6-methoxypyridin-3-yl)-2,7-dimethyl-1-oxo-1,2-dihydroisoquinolin-5-yl)ethyl)amino)benzoic acid

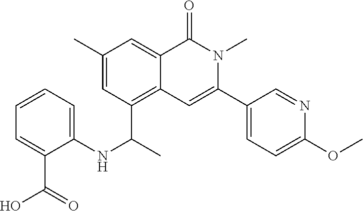 [SMILES: COc1ccc(-c2cc3c(C(C)Nc4ccccc4C(=O)O)cc(C)cc3c(=O)n2C)cn1]

The desired product (21 mg) was obtained following the similar procedure as Example 1.1H NMR (399 MHz, DMSO-d₆) δ ppm 12.73 (brs, 1H), 8.45-8.29 (m, 2H), 8.02-7.90 (m, 2H), 7.78 (d, J=7.6 Hz, 1H), 7.46 (s, 1H), 7.21-7.09 (m, 1H), 7.01-6.82 (m, 2H), 6.56-6.41 (m, 1H), 6.32 (d, J=7.9 Hz, 1H), 5.21 (s, 1H), 3.91 (s, 3H), 3.33 (s, 3H), 2.35 (s, 3H), 1.48 (s, 3H). MS (ESI) m/e [M+1]⁺=444.

### Example 16: 2-((1-(3-(1-(5-carbamoylpyridin-2-yl)-1H-pyrazol-4-yl)-2,7-dimethyl-1-oxo-1,2-dihydroisoquinolin-5-yl)ethyl)amino)benzoic acid

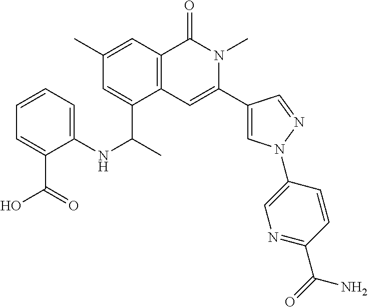 [SMILES: Cc1cc(C(C)Nc2ccccc2C(=O)O)c2cc(-c3cnn(-c4ccc(C(N)=O)nc4)c3)n(C)c(=O)c2c1]

### Step 1: methyl 2-((1-(3-(1-(5-cyanopyridin-2-yl)-1H-pyrazol-4-yl)-2,7-dimethyl-1-oxo-1,2-dihydroisoquinolin-5-yl)ethyl)amino)benzoate

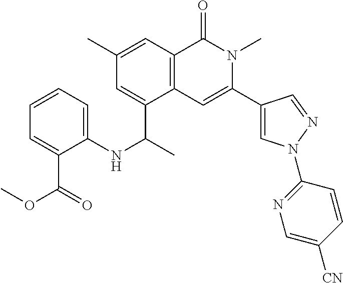 [SMILES: COC(=O)c1ccccc1NC(C)c1cc(C)cc2c(=O)n(C)c(-c3cnn(-c4ccc(C#N)cn4)c3)cc12]

To a solution of methyl 2-((1-(3-chloro-2,7-dimethyl-1-oxo-1,2-dihydroisoquinolin-5-yl)ethyl)amino)benzoate (50 mg, 0.13 mmol), 6-(4-(4,4,5,5-tetramethyl-1,3,2-dioxaborolan-2-yl)-1H-pyrazol-1-yl)nicotinonitrile (77 mg, 0.26 mmol) and K₃PO₄ (83 mg, 0.39 mmol) in dioxane (8 ml) and H₂O (1 ml) was added Pd(PPh₃)₄ (15 mg, 0.01 mmol) under N₂. The resulting solution was stirred at 100° C. for 15 h. The mixture was poured into H₂O and extracted with EtOAc. The combined organic extracts were washed with brine (20 mL), dried over anhydrous Na₂SO₄, filtered and concentrated in vacuum. The residue was purified by flash chromatography on silica gel to give the desired product (50 mg, 75%). MS (ESI) m/e [M+1]⁺=519.

### Step 2: 2-((1-(3-(1-(5-carbamoylpyridin-2-yl)-1H-pyrazol-4-yl)-2,7-dimethyl-1-oxo-1,2-dihydroisoquinolin-5-yl)ethyl)amino)benzoic acid

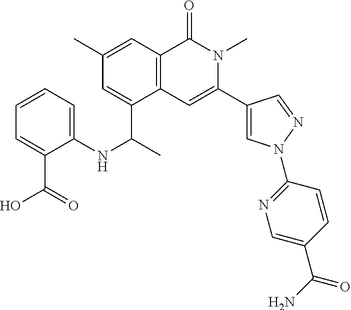 [SMILES: Cc1cc(C(C)Nc2ccccc2C(=O)O)c2cc(-c3cnn(-c4ccc(C(N)=O)cn4)c3)n(C)c(=O)c2c1]

To a solution of methyl 2-((1-(3-(1-(5-cyanopyridin-2-yl)-1H-pyrazol-4-yl)-2,7-dimethyl-1-oxo-1,2-dihydroisoquinolin-5-yl)ethyl)amino)benzoate (50 mg, 0.10 mmol) in methanol (4 mL), H₂O (2 ml) and THF (4 ml) was added lithium hydroxide monohydrate (20 mg, 0.48 mmol). The resulting solution was stirred for 6 h at 45° C. The solution was concentrated in vacuum. The residue was redissolved in H₂O and acidized to pH 5˜6 with HCl (2 M in water). The mixture was filtered, and the filter cake was purified by Prep-HPLC to give the desired product (8 mg, 16%). ¹H NMR (399 MHz, DMSO-d₆) δ ppm 9.10 (s, 1H), 8.94 (d, J=1.9 Hz, 1H), 8.58 (brs, 1H), 8.42 (dd, J=8.6, 2.1 Hz, 1H), 8.27 (s, 1H), 8.21 (s, 1H), 8.04 (d, J=8.6 Hz, 1H), 7.93 (s, 1H), 7.78 (d, J=7.7 Hz, 1H), 7.64 (s, 1H), 7.44 (s, 1H), 7.15-7.03 (m, 2H), 6.46 (t, J=7.5 Hz, 1H), 6.30 (d, J=8.4 Hz, 1H), 5.25 (d, J=5.7 Hz, 1H), 3.56 (s, 3H), 2.32 (s, 3H), 1.48 (d, J=6.4 Hz, 3H). MS (ESI) m/e [M+1]⁺=523.

### Example 17: 2-((1-(3-(benzo[d]thiazol-2-yl)-2,7-dimethyl-1-oxo-1,2-dihydroisoquinolin-5-yl)ethyl)amino)benzoic acid

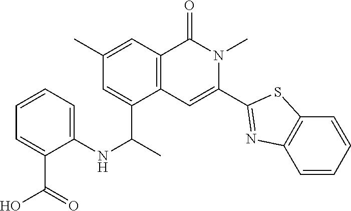 [SMILES: Cc1cc(C(C)Nc2ccccc2C(=O)O)c2cc(-c3nc4ccccc4s3)n(C)c(=O)c2c1]

The desired product (18 mg) was obtained following the similar procedure as Example 2. ¹H NMR (400 MHz, DMSO-d₆) δ ppm 12.85 (brzs, 1H), 8.65-8.27 (m, 1H), 8.24 (d, J=7.2 Hz, 1H), 8.18 (d, J=6.8 Hz, 1H), 8.05 (s, 1H), 7.85-7.78 (m, 1H), 7.68-7.54 (m, 3H), 7.45 (s, 1H), 7.22-7.10 (m, 1H), 6.55-6.47 (m, 1H), 6.37 (d, J=4.4 Hz, 1H), 5.34-5.22 (m, 1H), 3.68 (s, 3H), 2.41 (s, 3H), 1.53 (brs, 3H). MS (ESI) m/e [M+1]⁺=470

### Example 18: 2-((1-(2,7-dimethyl-3-(2-methylbenzo[d]thiazol-6-yl)-1-oxo-1,2-dihydroisoquinolin-5-yl)ethyl)amino)benzoic acid

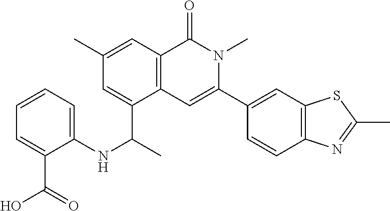 [SMILES: Cc1cc(C(C)Nc2ccccc2C(=O)O)c2cc(-c3ccc4nc(C)sc4c3)n(C)c(=O)c2c1]

### Step 1: 2-methyl-6-(4,4,5,5-tetramethyl-1,3,2-dioxaborolan-2-yl)benzo[d]thiazole

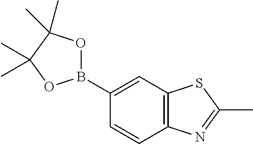

A mixture of 6-bromo-2-methylbenzo[d]thiazole (2.0 g, 8.80 mmol), bis(pinacolato)diboron (4.4 g, 17.50 mmol), Pd(dppf)Cl₂ (290 mg, 0.40 mmol), and potassium acetate (1.7 g, 17.50 mmol) in dioxane (30 mL) was stirred at 110° C. for 12 h under N₂. The mixture was poured into H₂O and extracted with EtOAc. The organic layer was dried over Na₂SO₄, filtered, and concentrated in vacuum. The residue was purified by CombiFlash chromatography on silica gel to give the desired product (1.8 g, 75%). MS (ESI) m/e [M+1]⁺=276

### Step 2: methyl 2-((1-(2,7-dimethyl-3-(2-methylbenzo[d]thiazol-6-yl)-1-oxo-1,2-dihydroisoquinolin-5-yl)ethyl)amino)benzoate

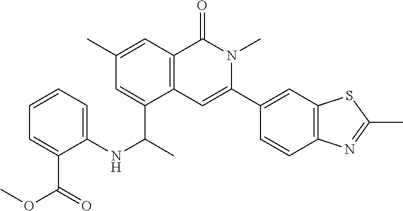

A mixture of methyl 2-((1-(3-chloro-2,7-dimethyl-1-oxo-1,2-dihydroisoquinolin-5-yl)ethyl)amino)benzoate (50 mg, 0.13 mmol), 2-methyl-6-(4,4,5,5-tetramethyl-1,3,2-dioxaborolan-2-yl)benzo[d]thiazole (54 mg, 0.2 mmol), Pd(PPh₃)₄ (12 mg, 0.01 mmol), and K₃PO₄ (83 mg, 0.39 mmol) in dioxane (5 mL) and H₂O (0.5 mL) was stirred at 100° C. for 5 h under N₂. The mixture was poured into H₂O and extracted with EtOAc. The organic layer was dried over Na₂SO₄, filtered, and concentrated in vacuum. The residue was purified by Prep-TLC to give the desired product (55 mg, 85%). MS (ESI) m/e [M+1]⁺=498

### Step 3: 2-((1-(2,7-dimethyl-3-(2-methylbenzo[d]thiazol-6-yl)-1-oxo-1,2-dihydroisoquinolin-5-yl)ethyl)amino)benzoic acid

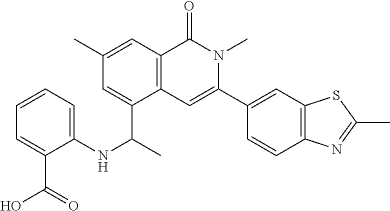 [SMILES: Cc1cc(C(C)Nc2ccccc2C(=O)O)c2cc(-c3ccc4nc(C)sc4c3)n(C)c(=O)c2c1]

To a solution of methyl 2-((1-(2,7-dimethyl-3-(2-methylbenzo[d]thiazol-6-yl)-1-oxo-1,2-dihydroisoquinolin-5-yl)ethyl)amino)benzoate (55 mg, 0.11 mmol) in methanol (3 mL) was added NaOH (3M in water, 1 mL). The resulting solution was stirred for 15 h at 60° C. The solution was concentrated in vacuum. The residue was redissolved in H₂O and acidized to pH 5˜6 with HCl (2 M in water). The mixture was filtered and the solid was purified by Prep-HPLC to give the desired product (25 mg, 47%). ¹H NMR (399 MHz, DMSO-d₆) δ ppm 12.76 (brs, 1H), 8.40 (s, 1H), 8.26 (s, 1H), 8.08-7.95 (m, 2H), 7.78 (s, 1H), 7.67 (s, 1H), 7.47 (s, 1H), 7.14 (s, 1H), 6.90 (s, 1H), 6.49 (s, 1H), 6.34 (d, J=6.1 Hz, 1H), 5.20 (s, 1H), 3.33 (s, 3H), 2.83 (s, 3H), 2.35 (s, 3H), 1.49 (brs, 3H). MS (ESI) m/e [M+1]⁺=484.

### Example 19: 2-((1-(2,7-dimethyl-3-(2-methyl-2H-indazol-5-yl)-1-oxo-1,2-dihydroisoquinolin-5-yl)ethyl)amino)benzoic acid

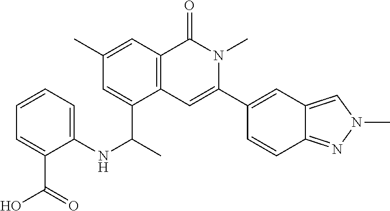 [SMILES: Cc1cc(C(C)Nc2ccccc2C(=O)O)c2cc(-c3ccc4nn(C)cc4c3)n(C)c(=O)c2c1]

### Step 1: methyl 2-((1-(2,7-dimethyl-3-(2-methyl-2H-indazol-5-yl)-1-oxo-1,2-dihydroisoquinolin-5-yl)ethyl)amino)benzoate

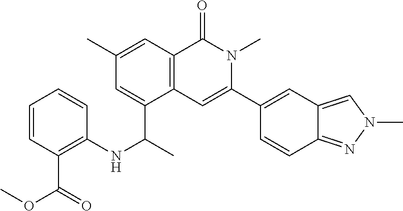

A mixture of methyl 2-((1-(3-chloro-2,7-dimethyl-1-oxo-1,2-dihydroisoquinolin-5-yl)ethyl)amino)benzoate (25 mg, 0.065 mmol), (2-methyl-2H-indazol-5-yl)boronic acid (14 mg, 0.078 mmol), Pd(PPh₃)₄ (7.5 mg, 0.0065 mmol) and K₃PO₄ (41 mg, 0.195 mmol) in dioxane and H₂O (5:1) (2 mL) was stirred at 90° C. for 16 h. The mixture was concentrated in vacuum. The residue was purified by prep-TLC to give the desired product (30 mg, 96%). MS (ESI) m/e [M+1]⁺=481.

### Step 2: 2-((1-(2,7-dimethyl-3-(2-methyl-2H-indazol-5-yl)-1-oxo-1,2-dihydroisoquinolin-5-yl)ethyl)amino)benzoic acid

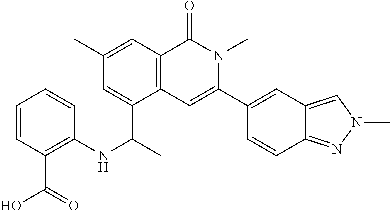 [SMILES: Cc1cc(C(C)Nc2ccccc2C(=O)O)c2cc(-c3ccc4nn(C)cc4c3)n(C)c(=O)c2c1]

To a solution of methyl 2-((1-(2,7-dimethyl-3-(2-methyl-2H-indazol-5-yl)-1-oxo-1,2-dihydroisoquinolin-5-yl)ethyl)amino)benzoate in MeOH (1 mL) and THF (0.5 mL) was added 4N NaOH (0.2 mL). The reaction mixture was stirred at 50° C. for 16 h. The mixture was adjusted to pH 5 with 1N HCl and concentrated in vacuum. The residue was purified by prep-HPLC to give the desired product (17 mg, 59%). ¹H NMR (500 MHz, DMSO-d₆) δ ppm 12.73 (brs, 1H), 8.46 (s, 1H), 8.40 (s, 1H), 8.00 (s, 1H), 7.91 (s, 1H), 7.81 (d, J=6.5 Hz, 1H), 7.70 (d, J=9.0 Hz, 1H), 7.49 (s, 1H), 7.39 (dd, J=9.0, 1.5 Hz, 1H), 7.17 (t, J=8.0 Hz, 1H), 6.87 (s, 1H), 6.52 (t, J=7.5 Hz, 1H), 6.35 (d, J=8.5 Hz, 1H), 5.20 (s, 1H), 4.22 (s, 3H), 3.36 (s, 3H), 2.38 (s, 3H), 1.52 (d, J=6.5 Hz, 3H). MS (ESI) m/e [M+1]⁺=467.

### Example 20: 2-((1-(2-methyl-1-oxo-3-phenyl-1,2-dihydroisoquinolin-5-yl)ethyl)amino)benzoic acid

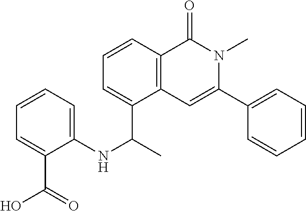 [SMILES: CC(Nc1ccccc1C(=O)O)c1cccc2c(=O)n(C)c(-c3ccccc3)cc12]

### Step 1: 4-bromo-2-(hydroxyamino)-2,3-dihydro-1H-inden-1-one

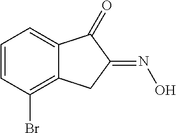 [SMILES: O=C1/C(=N/O)Cc2c(Br)cccc21]

To a solution of 4-bromo-1-indanone (10.5 g, 50.00 mmol) in diethyl ether (100 mL) in concentrated HCl (32%, 50 mL) was added isopentyl nitrite (7.7 g, 75.00 mmol) dropwise in 1 h. The resulting solution was vigorously stirred at room temperature for 3 h. The precipitated solid was collected by filtration and washed with ether (20 mL×2) to give the desired product (8.0 g, 67%). MS (ESI) m/e [M+1]⁺=240.

### Step 2: 5-bromo-3-chloroisoquinolin-1(2H)-one

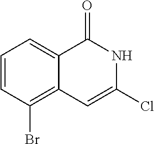 [SMILES: O=c1[nH]c(Cl)cc2c(Br)cccc12]

To a solution of 4-bromo-2-(hydroxyimino)-2,3-dihydro-1H-inden-1-one (2.5 g, 10.42 mmol) in CCl₄ (50 ml) was added PCl₅ (3.2 g, 15.63 mmol) and stirred at RT for 16 h. The reaction mixture was concentrated in vacuo and the residue was dissolved in 4M HCl in dioxane (100 ml). The resulting mixture was stirred at RT for 16 h. The reaction mixture was concentrated in vacuo and redissolved with water. The precipitated solid was collected by filtration and triturated with Petroleum ether/EtOAc (10:1) to give the desired product (1.6 g, 59%). MS (ESI) m/e [M+1]⁺=258.

### Step 3: 5-bromo-3-chloro-2-methylisoquinolin-1(2H)-one

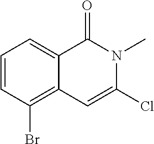

To a solution of 5-bromo-3-chloroisoquinolin-1(2H)-one (1.4 g, 5.28 mmol) and K₂CO₃ (2.2 g, 15.84 mmol) in DMF (8 mL) was added CH₃I (750 mg, 5.28 mmol) at 0° C. The reaction mixture was stirred for 1 h at RT. The mixture was poured into H₂O (30 mL) and extracted with EA (20 mL×3). The combined organic phase was washed with brine, dried over anhydrous Na₂SO₄, filtered, ad concentrated in vacuum. The residue was purified by column chromatography on silica gel to give the desired product (1.2 g, 83%). MS (ESI) m/e [M+1]⁺=272.

### Step 4: 5-acetyl-3-chloro-2-methylisoquinolin-1(2H)-one

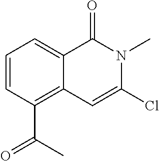 [SMILES: CC(=O)c1cccc2c(=O)n(C)c(Cl)cc12]

To a solution of 5-bromo-3-chloro-2-methylisoquinolin-1(2H)-one (900 mg, 3.30 mmol) and tributyl(1-ethoxyvinyl)stannane (1.2 g, 3.30 mmol) in dioxane (30 mL) was added bis(triphenylphosphine)palladium(II) chloride (232 mg, 0.33 mmol) under nitrogen atmosphere. The resulting mixture was stirred for 15 h at 100° C. under N₂. After cooled to room temperature, the reaction solution was added 1M aqueous HCl (10 mL) and stirred for 30 min. The mixture was quenched with saturated aqueous KF (50 mL), stirred for 30 min, and filtered. The filtration was extracted with 10% MeOH in DCM (20 ml×3). The combined extracts were dried over anhydrous Na₂SO₄, filtered, and concentrated. The residue was purified by column chromatography on silica gel to give the desired product (447 mg, 57%). MS (ESI) m/e [M+1]⁺=236.

### Step 5: 5-acetyl-2-methyl-3-phenylisoquinolin-1(2H)-one

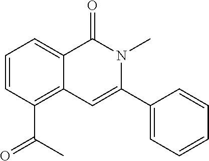 [SMILES: CC(=O)c1cccc2c(=O)n(C)c(-c3ccccc3)cc12]

A mixture of 5-acetyl-3-chloro-2-methylisoquinolin-1(2H)-one (447 mg, 1.89 mmol), phenylboronic acid (347 mg, 2.84 mmol), Pd(PPh₃)₄ (219 mg, 0.19 mmol), and K₃PO₄ (1.20 g, 5.67 mmol) in dioxane (20 mL) and H₂O (2 mL) was stirred at 100° C. for 15 h under N₂. The mixture was poured into H₂O and extracted with EtOAc. The organic layer was dried over anhydrous Na₂SO₄, filtered and concentrated in vacuum. The residue was purified by flash chromatography on silica gel to give the desired product (454 mg, 86%). MS (ESI) m/e [M+1]⁺=278.

### Step 6: 5-(1-hydroxyethyl)-2-methyl-3-phenylisoquinolin-1(2H)-one

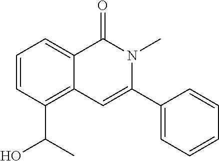 [SMILES: CC(O)c1cccc2c(=O)n(C)c(-c3ccccc3)cc12]

A solution of 5-acetyl-2-methyl-3-phenylisoquinolin-1(2H)-one (454 mg, 1.64 mmol) in DCM (10 mL) and MeOH (10 mL) was added NaBH₄ (93 mg, 2.46 mmol) in portions at 0° C. The resulting solution was stirred at RT for 1 h. The mixture was diluted with H₂O (10 mL) and extracted with DCM (30 mL×2). The combined organic extracts were washed with brine (20 mL), dried over anhydrous Na₂SO₄, filtered and concentrated in vacuum. The residue was purified by flash chromatography on silica gel to give the desired product (357 mg, 99%). MS (ESI) m/e [M+1]⁺=280.

### Step 7: 5-(1-bromoethyl)-2-methyl-3-phenylisoquinolin-1(2H)-one

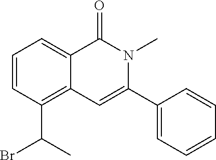 [SMILES: CC(Br)c1cccc2c(=O)n(C)c(-c3ccccc3)cc12]

To a solution of 5-(1-hydroxyethyl)-2-methyl-3-phenylisoquinolin-1(2H)-one (100 mg, 0.36 mmol) in DCM (5 mL) was added PBr₃ (291 mg, 1.08 mmol) dropwise at 0° C. The resulting solution was stirred for 2 h at RT. The reaction was quenched with water (20 mL) at 0° C. and the pH was adjusted to 8 with saturated aqueous NaHCO₃. The mixture was extracted with DCM (10 mL×2). The combined organic extracts were washed with brine, dried over anhydrous Na₂SO₄, filtered, and concentrated to give the desired product (100 mg, 90%). MS (ESI) m/e [M+1]⁺=342.

### Step 8: methyl 2-((1-(2-methyl-1-oxo-3-phenyl-1,2-dihydroisoquinolin-5-yl)ethyl)amino)benzoate

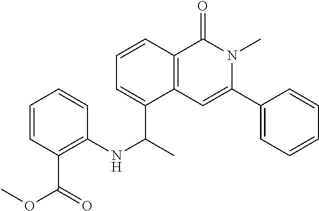 [SMILES: COC(=O)c1ccccc1NC(C)c1cccc2c(=O)n(C)c(-c3ccccc3)cc12]

To a solution of 5-(1-bromoethyl)-2-methyl-3-phenylisoquinolin-1(2H)-one (90 mg, 0.26 mmol) and methyl 2-aminobenzoate (48 mg, 0.32 mmol) in DMF (3 mL) was added DIEA (100 mg, 0.78 mmol). The resulted solution was stirred for 4 h at 90° C. After cooled to room temperature, the reaction was diluted with water (30 mL) and extracted with EtOAc (30 mL×2). The combined organic extracts were washed with brine (20 mL), dried over anhydrous Na₂SO₄, filtered, and concentrated in vacuum. The residue was purified by flash chromatography on silica gel to give the desired product (50 mg, 46%). MS (ESI) m/e [M+1]⁺=413.

### Step 9: 2-((1-(2-methyl-1-oxo-3-phenyl-1,2-dihydroisoquinolin-5-yl)ethyl)amino)benzoic acid

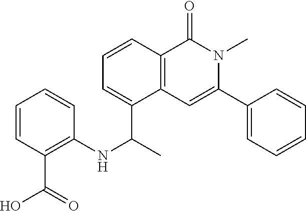 [SMILES: CC(Nc1ccccc1C(=O)O)c1cccc2c(=O)n(C)c(-c3ccccc3)cc12]

To a solution of methyl 2-((1-(2-methyl-1-oxo-3-phenyl-1,2-dihydroisoquinolin-5-yl)ethyl)amino)benzoate (50 mg, 0.12 mmol) in methanol (4 mL), H₂O (2 mL) and THF (4 mL) was added lithium hydroxide monohydrate (51 mg, 1.21 mmol). The resulting solution was stirred for 4 h at 50° C. The solution was concentrated in vacuum. The residue was redissolved in H₂O and acidized to pH 5-6 with HCl (2 M in water). The mixture was filtered, and the filter cake was purified by Prep-HPLC to give the desired product (27 mg, 56%). ¹H NMR (400 MHz, DMSO-d₆) δ ppm 12.76 (brs, 1H), 8.38 (d, J=3.7 Hz, 1H), 8.15 (d, J=7.8 Hz, 1H), 7.78 (d, J=7.8 Hz, 1H), 7.64-7.56 (m, 3H), 7.52-7.48 (m, 3H), 7.41 (t, J=7.6 Hz, 1H), 7.13 (t, J=7.6 Hz, 1H), 6.84 (s, 1H), 6.49 (t, J=7.4 Hz, 1H), 6.30 (d, J=8.5 Hz, 1H), 5.23 (m, 1H), 3.31 (s, 3H), 1.50 (d, J=6.2 Hz, 3H). MS (ESI) m/e [M+1]⁺=399.

### Example 21: 2-((1-(2-methyl-1-oxo-3-(phenylethynyl)-1,2-dihydroisoquinolin-5-yl)ethyl)amino)benzoic acid

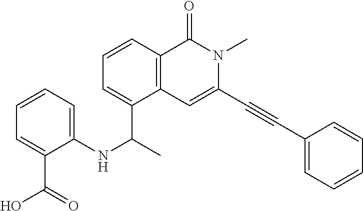 [SMILES: CC(Nc1ccccc1C(=O)O)c1cccc2c(=O)n(C)c(C#Cc3ccccc3)cc12]

### Step 1: 5-(1-aminoethyl)-3-chloro-2-methylisoquinolin-1(2H)-one

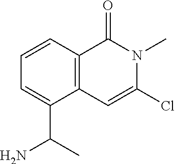 [SMILES: CC(N)c1cccc2c(=O)n(C)c(Cl)cc12]

A mixture of 5-acetyl-3-chloro-2-methylisoquinolin-1(2H)-one (200 mg, 0.85 mmol), HCOOH (78.00 mg, 1.70 mmol), HCOONH₄ (160 mg, 2.54 mmol) and Cp*Ir [N-[4-(Trifluoromethyl)phenyl]-2-pyridinecarboxamidato]-Cl in MeOH (5 mL) was stirred at 60° C. for 16 h under N₂ atmosphere. The reaction mixture was concentrated in vacuum. The residue was purified by combiflash to give the desired product (80 mg, 40%). MS (ESI) m/e [M+1]⁺=237.

### Step 2: 5-(1-aminoethyl)-2-methyl-3-(phenylethynyl)isoquinolin-1(2H)-one

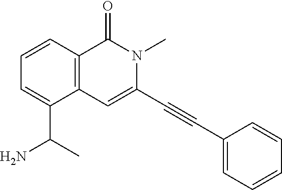 [SMILES: CC(N)c1cccc2c(=O)n(C)c(C#Cc3ccccc3)cc12]

A mixture of 5-(1-aminoethyl)-3-chloro-2-methylisoquinolin-1(2H)-one (80 mg, 0.34 mmol), ethynylbenzene (41 mg, 0.40 mmol), Pd(PPh₃)₂Cl₂ (12 mg, 0.017 mmol) and CuI (6.4 mg, 0.034 mmol) in Et₃N (1 mL) and DMF (1 mL) was stirred at 80° C. for 16 h under N₂ atmosphere. The reaction was diluted with water and extracted with DCM (50 mL). The organic phase was concentrated in vacuum and the residue was purified by prep-TLC to give the desired product (90 mg, 88%).

### Step 3: 2-((1-(2-methyl-1-oxo-3-(phenylethynyl)-1,2-dihydroisoquinolin-5-yl)ethyl)amino)benzoic acid

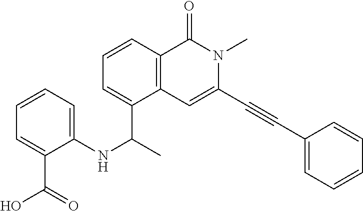 [SMILES: CC(Nc1ccccc1C(=O)O)c1cccc2c(=O)n(C)c(C#Cc3ccccc3)cc12]

A mixture of 5-(1-aminoethyl)-2-methyl-3-(phenylethynyl)isoquinolin-1(2H)-one (90 mg, 0.30 mmol), 2-iodobenzoic acid (74 mg, 0.30 mmol), CuI (5.7 mg, 0.030 mmol), L-Proline (6.9 mg, 0.060 mmol) and K₂CO₃ (82 mg, 0.60 mmol) in DMSO (2 mL) was stirred at 100° C. for 16 h under N₂ atmosphere. The reaction was extracted with DCM (30 mL×3). The combined organic phase was concentrated in vacuum and the residue was purified by prep-HPLC to give the desired product (7.4 mg, 6%). 11H NMR (400 MHz, DMSO-d₆) δ ppm 12.72 (brs, 1H), 8.51 (s, 1H), 8.13 (d, J=6.4 Hz, 1H), 7.80 (d, J=7.6 Hz, 1H), 7.73-7.60 (m, 3H), 7.59-7.37 (m, 5H), 7.12 (s, 1H), 6.49 (s, 1H), 6.28 (d, J=8.0 Hz, 1H), 5.30 (s, 1H), 3.72 (s, 3H), 1.52 (brs, 3H). MS (ESI) m/e [M+1]⁺=423.

### Example 22: 2-((1-(2,7-dimethyl-3-(2-methyl-2H-indazol-7-yl)-1-oxo-1,2-dihydroisoquinolin-5-yl)ethyl)amino)benzoic acid

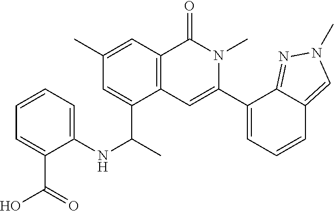 [SMILES: Cc1cc(C(C)Nc2ccccc2C(=O)O)c2cc(-c3cccc4cn(C)nc34)n(C)c(=O)c2c1]

### Step 1: 5-acetyl-2,7-dimethyl-3-(2-methyl-2H-indazol-7-yl)isoquinolin-1(2H)-one

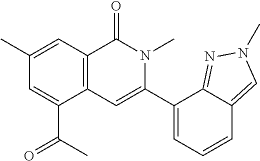

A mixture of 5-acetyl-3-chloro-2,7-dimethylisoquinolin-1(2H)-one (100 mg, 4.00 mmol), 2-methyl-7-(4,4,5,5-tetramethyl-1,3,2-dioxaborolan-2-yl)-2H-indazole (155 mg, 6.00 mmol), Pd(PPh₃)₄ (23 mg, 0.20 mmol), and K₃PO₄ (254 mg, 12.00 mmol) in dioxane (5 mL) and H₂O (0.5 mL) was stirred at 100° C. for 12 h under N₂. The mixture was poured into H₂O and extracted with EtOAc. The organic layer was dried over Na₂SO₄, filtered, and concentrated in vacuum. The residue was purified by CombiFlash chromatography on silica gel to give the desired product (135 mg, 98%). MS (ESI) m/e [M+1]⁺=346.

### Step 2: 5-(1-aminoethyl)-2,7-dimethyl-3-(2-methyl-2H-indazol-7-yl)isoquinolin-1(2H)-one

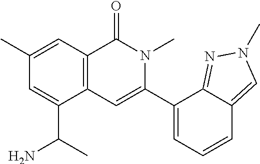

To a solution of 5-acetyl-2,7-dimethyl-3-(2-methyl-2H-indazol-7-yl)isoquinolin-1(2H)-one (135 mg, 0.4 mmol) in ethanol (5 mL) was added ammonium acetate (154 mg, 2.00 mmol), and NaBH₃CN (30 mg, 0.48 mmol). The resulting solution was stirred for 15 h at 90° C. The solution was concentrated in vacuum. The residue was purified by CombiFlash chromatography on silica gel to give the desired product (78 mg, 56%). MS (ESI) m/e [M+1]⁺=347.

### Step 3: 2-((1-(2,7-dimethyl-3-(2-methyl-2H-indazol-7-yl)-1-oxo-1,2-dihydroisoquinolin-5-yl)ethyl)amino)benzoic acid

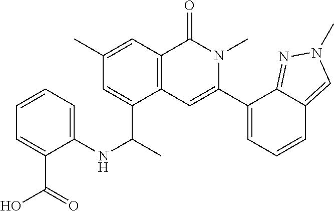 [SMILES: Cc1cc(C(C)Nc2ccccc2C(=O)O)c2cc(-c3cccc4cn(C)nc34)n(C)c(=O)c2c1]

To a solution of 5-(1-aminoethyl)-2,7-dimethyl-3-(2-methyl-2H-indazol-7-yl)isoquinolin-1(2H)-one (78 mg, 0.22 mmol), 2-aminobenzoic acid (84 mg, 0.34 mmol), L-proline (4 mg, 0.02 mmol) and K₂CO₃ (82 mg, 0.60 mmol) in 2 mL of DMSO was added CuI (6 mg, 0.02 mmol). The resulted solution was stirred overnight at 100° C. under N₂. After cooled to room temperature, the solution was concentrated in vacuum and the residue was dissolved in water. The pH value was adjusted to 4-5 and the resulting mixture was filtered. The filter cake was purified by Prep-HPLC to give the desired product (21 mg, 20%). ¹H NMR (400 MHz, DMSO-d₆) δ ppm 12.73 (brs, 1H), 8.47 (s, 1H), 8.35 (s, 1H), 8.02 (s, 1H), 7.86 (d, J=8.3 Hz, 1H), 7.78 (d, J=7.7 Hz, 1H), 7.47 (s, 1H), 7.39 (d, J=6.4 Hz, 1H), 7.24-6.95 (m, 2H), 6.91 (s, 1H), 6.49 (t, J=7.0 Hz, 1H), 6.31 (d, J=8.1 Hz, 1H), 5.16 (s, 1H), 4.15 (s, 3H), 3.27 (s, 3H), 2.37 (s, 3H), 1.49 (d, J=5.3 Hz, 3H). MS (ESI) m/e [M+1]⁺=467.

### Example 23: 2-((1-(2,7-dimethyl-3-(4-(1-methyl-1H-pyrazol-4-yl)phenyl)-1-oxo-1,2-dihydroisoquinolin-5-yl)ethyl)amino)benzoic acid

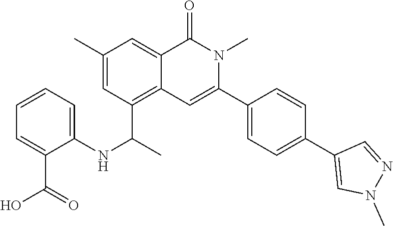 [SMILES: Cc1cc(C(C)Nc2ccccc2C(=O)O)c2cc(-c3ccc(-c4cnn(C)c4)cc3)n(C)c(=O)c2c1]

### Step 1: 5-acetyl-2,7-dimethyl-3-(4-(1-methyl-1H-pyrazol-4-yl)phenyl)isoquinolin-1(2H)-one

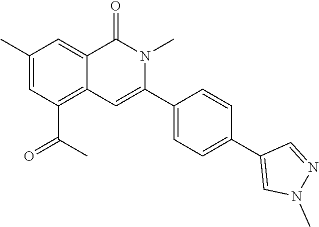 [SMILES: CC(=O)c1cc(C)cc2c(=O)n(C)c(-c3ccc(-c4cnn(C)c4)cc3)cc12]

A mixture of 5-acetyl-3-chloro-2,7-dimethylisoquinolin-1(2H)-one (100 mg, 4.00 mmol), 1-methyl-4-(4-(4,4,5,5-tetramethyl-1,3,2-dioxaborolan-2-yl)phenyl)-1H-pyrazole (170 mg, 6.00 mmol), Pd(PPh₃)₄ (23 mg, 0.20 mmol), and K₃PO₄ (254 mg, 12.00 mmol) in dioxane (5 mL) and H₂O (0.5 mL) was stirred at 100° C. for 12 h under N₂. The mixture was poured into H₂O and extracted with EtOAc. The organic layer was dried over Na₂SO₄, filtered, and concentrated in vacuum. The residue was purified by CombiFlash chromatography on silica gel to give the desired product (85 mg, 57%). MS (ESI) m/e [M+1]⁺=372

### Step 2: 5-(1-aminoethyl)-2,7-dimethyl-3-(4-(1-methyl-1H-pyrazol-4-yl)phenyl)isoquinolin-1(2H)-one

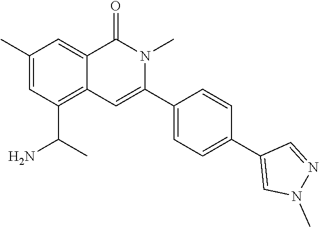 [SMILES: Cc1cc(C(C)N)c2cc(-c3ccc(-c4cnn(C)c4)cc3)n(C)c(=O)c2c1]

To a solution of 5-acetyl-2,7-dimethyl-3-(4-(1-methyl-1H-pyrazol-4-yl)phenyl)isoquinolin-1(2H)-one (85 mg, 0.2 mmol) in ethanol (4 ml) was added Ammonium acetate (77 mg, 1.00 mmol) and NaCNBH₃ (15 mg, 0.24 mmol). The resulting solution was stirred for 15 h at 90° C. The solution was concentrated in vacuum. The residue was purified by CombiFlash chromatography on silica gel to give the desired product (38 mg, 45%). MS (ESI) m/e [M+1]⁺=373.

### Step 3: 2-((1-(2,7-dimethyl-3-(4-(1-methyl-1H-pyrazol-4-yl)phenyl)-1-oxo-1,2-dihydroisoquinolin-5-yl)ethyl)amino)benzoic acid

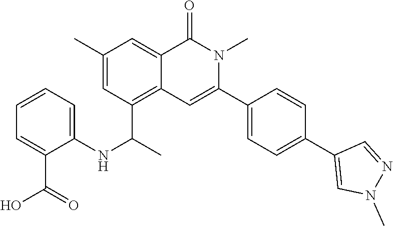 [SMILES: Cc1cc(C(C)Nc2ccccc2C(=O)O)c2cc(-c3ccc(-c4cnn(C)c4)cc3)n(C)c(=O)c2c1]

To a solution of 5-(1-aminoethyl)-2,7-dimethyl-3-(4-(1-methyl-1H-pyrazol-4-yl)phenyl)isoquinolin-1(2H)-one (38 mg, 0.10 mmol), 2-aminobenzoic acid (37 mg, 0.15 mmol), L-proline (2 mg, 0.01 mmol) and K₂CO₃ (41 mg, 0.30 mmol) in 2 mL of DMSO was added CuI (3 mg, 0.01 mmol). The resulted solution was stirred overnight at 100° C. under N₂. After cooled to room temperature, the solution was concentrated in vacuum and the residue was dissolved in water. The pH value was adjusted to 4-5 and the resulting mixture was filtered. The filter cake was purified by Prep-HPLC to give the desired product (10 mg, 20%). ¹H NMR (400 MHz, DMSO-d₆) δ ppm 12.75 (brs, 1H), 8.39 (s, 1H), 8.22 (s, 1H), 8.03-7.89 (m, 2H), 7.84-7.73 (m, 1H), 7.73-7.62 (m, 2H), 7.61-7.51 (m, 2H), 7.46 (s, 1H), 7.15 (t, J=7.2 Hz, 1H), 6.83 (s, 1H), 6.50 (t, J=6.9 Hz, 1H), 6.32 (d, J=8.1 Hz, 1H), 5.19 (s, 1H), 3.87 (s, 3H), 3.34 (s, 3H), 2.35 (s, 3H), 1.50 (d, J=4.5 Hz, 3H). MS (ESI) m/e [M+1]⁺=493.

### Example 24: 2-((1-(3-benzamido-2,7-dimethyl-1-oxo-1,2-dihydroisoquinolin-5-yl)ethyl)amino)benzoic acid

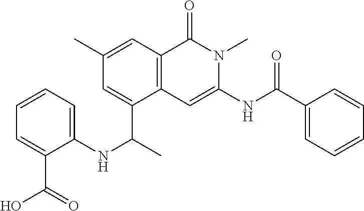 [SMILES: Cc1cc(C(C)Nc2ccccc2C(=O)O)c2cc(NC(=O)c3ccccc3)n(C)c(=O)c2c1]

### Step 1: methyl 2-((1-(3-benzamido-2,7-dimethyl-1-oxo-1,2-dihydroisoquinolin-5-yl)ethyl)amino)benzoate

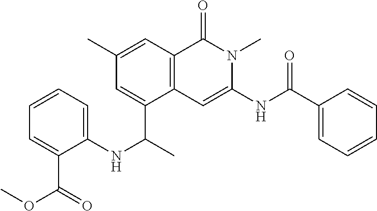 [SMILES: COC(=O)c1ccccc1NC(C)c1cc(C)cc2c(=O)n(C)c(NC(=O)c3ccccc3)cc12]

To a solution of methyl 2-((1-(3-chloro-2,7-dimethyl-1-oxo-1,2-dihydroisoquinolin-5-yl)ethyl)amino)benzoate (60 mg, 0.16 mmol), benzamide (38 mg, 0.31 mmol), xantPhos (18 mg, 0.03 mmol) and Cs₂CO₃ (152 mg, 0.47 mmol) in 10 mL of dioxane was added Pd₂(dba)₃·CHCl₃ (16 mg, 0.02 mmol). The resulted solution was stirred overnight at 130° C. under N₂. After cooled to room temperature, the solution was concentrated in vacuum and the residue was purified by CombiFlash to give the desired product (60 mg, 82%). MS (ESI) m/e [M+1]⁺=470.

### Step 2: 2-((1-(3-benzamido-2,7-dimethyl-1-oxo-1,2-dihydroisoquinolin-5-yl)ethyl)amino)benzoic acid

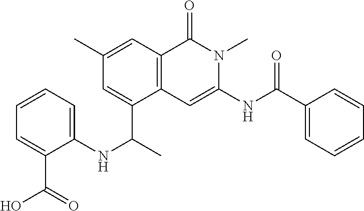 [SMILES: Cc1cc(C(C)Nc2ccccc2C(=O)O)c2cc(NC(=O)c3ccccc3)n(C)c(=O)c2c1]

To a solution of methyl 2-((1-(3-benzamido-2,7-dimethyl-1-oxo-1,2-dihydroisoquinolin-5-yl)ethyl)amino)benzoate (60 mg, 0.13 mmol) in methanol (4 mL), H₂O (2 ml) and THF (4 ml) was added lithium hydroxide monohydrate (54 mg, 1.28 mmol). The resulting solution was stirred for 6 h at 50° C. The solution was concentrated in vacuum. The residue was redissolved in H₂O and acidized to pH 5-6 with HCl (2 M in water). The mixture was filtered, and the filter cake was purified by Prep-HPLC to give the desired product. MS (ESI) m/e [M+1]⁺=456.

### Example 25: 2-((1-(4-oxo-2-phenyl-4H-quinolizin-9-yl)ethyl)amino)benzoic acid

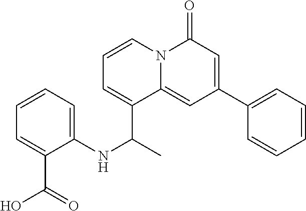 [SMILES: CC(Nc1ccccc1C(=O)O)c1cccn2c(=O)cc(-c3ccccc3)cc12]

### Step 1: 2-(3-bromopyridin-2-yl)-1-phenylethan-1-one

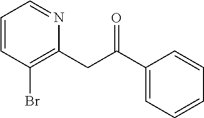 [SMILES: O=C(Cc1ncccc1Br)c1ccccc1]

To a solution of 3-bromo-2-methylpyridine (5.0 g, 29.07 mmol) in THF (50 ml) was added LDA (2.0 M in THF, 31 mL, 61.05 mmol) dropwise at −65° C. The reaction mixture was stirred at −65° C. for 0.5 h, then N-methoxy-N-methylbenzamide (5.3 g in 20 mL of THF, 32.00 mmol) was added dropwise at −65° C. The reaction mixture was allowed to warm up to 0° C. and stirred for 2 h. The resulting mixture was quenched with aq. NH₄Cl (50 ml) and extracted with EA (50 mL×3). The combined organic phase was washed with brine (50 mL), dried over Na₂SO₄, filtered and concentrated in vacuum. The crude product was purified by CombiFlash on silica gel to give the desired product (2.7 g, 34%). MS (ESI) m/e [M+1]⁺=276

### Step 2: 9-bromo-2-phenyl-4H-quinolizin-4-one

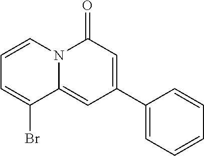 [SMILES: O=c1cc(-c2ccccc2)cc2c(Br)cccn12]

To a solution of 2-(3-bromopyridin-2-yl)-1-phenylethan-1-one (2.3 g, 8.3 mmol) in toluene (30 mL) was added NaH (60%, 667 mg, 16.6 mmol) in batches, and the reaction mixture was stirred at 25° C. for 1 h. Then ethyl 2-(diethoxyphosphoryl)acetate (3.7 g, 16.6 mmol) was added. The reaction mixture was stirred at 25° C. for 1 h, then at 120° C. for 15 h. The resulting solution was quenched with aq. NH₄Cl (20 mL) and extracted with EA (30 mL×3). The combined organic phase was washed with brine (20 mL), dried over Na₂SO₄, filtered and concentrated in vacuum. The crude product was purified by CombiFlash on silica gel to give the desired product (1.1 g, 44%). MS (ESI) m/e [M+1]⁺=300

### Step 3: 9-acetyl-2-phenyl-4H-quinolizin-4-one

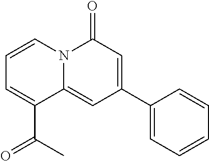 [SMILES: CC(=O)c1cccn2c(=O)cc(-c3ccccc3)cc12]

A mixture of 9-bromo-2-phenyl-4H-quinolizin-4-one (1.1 g, 3.70 mmol), tributyl(1-ethoxyvinyl)stannane (1.6 g, 4.40 mmol) and Pd(PPh₃)₂Cl₂ (260 mg, 0.37 mmol) in dioxane (30 mL) was stirred at 100° C. for 15 h under N₂. After cooled to room temperature, HCl (2M in water, 5 mL) was added and the reaction mixture was stirred for 1 h at room temperature. The resulting solution was diluted with EtOAc and washed with water & brine. The organic layer was dried over Na₂SO₄, filtered, and concentrated in vacuum. The residue was purified by CombiFlash on silica gel to give the desired product (750 mg, 77%). MS (ESI) m/e [M+1]⁺=264

### Step 4: 9-(1-aminoethyl)-2-phenyl-4H-quinolizin-4-one

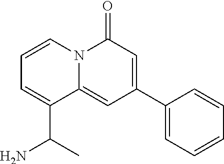 [SMILES: CC(N)c1cccn2c(=O)cc(-c3ccccc3)cc12]

To a solution of 9-acetyl-2-phenyl-4H-quinolizin-4-one (500 mg, 1.90 mmol) in ethanol (15 mL) was added ammonium acetate (732 mg, 9.50 mmol), followed by NaBH₃CN (144 mg, 2.30 mmol). The resulting solution was stirred for 15 h at 90° C. The reaction solution was concentrated in vacuum and the residue was purified by CombiFlash on silica gel to give the desired product (310 mg, 62%). MS (ESI) m/e [M+1]⁺=265.

### Step 5: 2-((1-(4-oxo-2-phenyl-4H-quinolizin-9-yl)ethyl)amino)benzoic acid

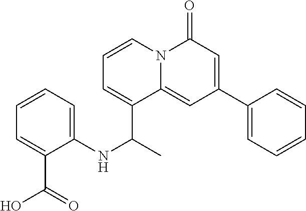 [SMILES: CC(Nc1ccccc1C(=O)O)c1cccn2c(=O)cc(-c3ccccc3)cc12]

To a solution of 9-(1-aminoethyl)-2-phenyl-4H-quinolizin-4-one (100 mg, 0.38 mmol), 2-aminobenzoic acid (112 mg, 0.46 mmol), L-proline (4 mg, 0.04 mmol) and K₂CO₃ (157 mg, 1.14 mmol) in 3 mL of DMSO was added CuI (7 mg, 0.04 mmol). The resulted solution was stirred overnight at 100° C. under N₂. After cooled to room temperature, the reaction solution was concentrated in vacuum. The residue was dissolved in water and the pH value was adjusted to 4-5. The resulting mixture was filtered and the solid was purified by Prep-HPLC to give the desired product (18 mg, 12%). ¹H NMR (400 MHz, DMSO-d₆) δ ppm 12.82 (s, 1H), 8.94 (d, J=7.2 Hz, 1H), 8.55-8.29 (m, 1H), 7.89 (d, J=7.7 Hz, 2H), 7.82 (d, J=7.9 Hz, 1H), 7.60-7.39 (m, 4H), 7.32 (s, 1H), 7.20 (t, J=7.8 Hz, 1H), 7.12 (t, J=7.1 Hz, 1H), 6.78 (s, 1H), 6.55 (t, J=7.5 Hz, 1H), 6.36 (d, J=8.5 Hz, 1H), 5.54-5.17 (m, 1H), 1.60 (d, J=6.3 Hz, 3H). MS (ESI) m/e [M+1]⁺=385.

### Example 26: 2-((1-(7-methyl-1-oxo-3-phenyl-1,2-dihydroisoquinolin-5-yl)ethyl)amino)benzoic acid

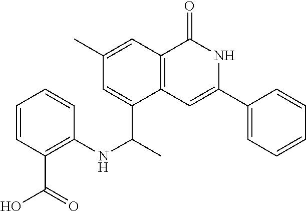 [SMILES: Cc1cc(C(C)Nc2ccccc2C(=O)O)c2cc(-c3ccccc3)[nH]c(=O)c2c1]

### Step 1: 5-acetyl-3-chloro-7-methylisoquinolin-1(2H)-one

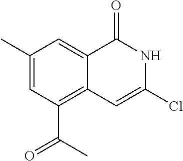 [SMILES: CC(=O)c1cc(C)cc2c(=O)[nH]c(Cl)cc12]

To a solution of 5-bromo-3-chloro-7-methylisoquinolin-1(2H)-one (200 mg, 0.73 mmol) and tributyl(1-ethoxyvinyl)stannane (278 mg, 0.77 mmol) in dioxane (20 mL) was added bis(triphenylphosphine)palladium(II) chloride (51 mg, 0.07 mmol) under nitrogen atmosphere. The resulting mixture was stirred for 15 h at 100° C. After cooled to room temperature, treated the reaction with 1M aqueous HCl (5 mL) and stirred for 30 min. The mixture was quenched with saturated aqueous KF (10 mL), stirred for 30 min, and filtered. The filter cake was washed with 10% MeOH in DCM (20 ml×3). The combined extracts were dried over anhydrous Na₂SO₄, filtered, and concentrated to give a residue which was purified by column chromatography on silica gel eluted with 50%-55% EtOAc in petroleum ether to give the product (140 mg, 81%). MS (ESI) m/e [M+1]⁺=236.

### Step 2: 5-acetyl-7-methyl-3-phenylisoquinolin-1(2H)-one

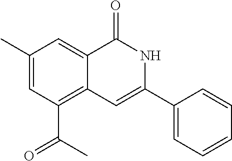 [SMILES: CC(=O)c1cc(C)cc2c(=O)[nH]c(-c3ccccc3)cc12]

A mixture of 5-acetyl-3-chloro-7-methylisoquinolin-1(2H)-one (140 mg, 0.59 mmol), phenylboronic acid (109 mg, 0.89 mmol), Pd(PPh₃)₄ (34 mg, 0.03 mmol), and K₃PO₄ (377 mg, 1.78 mmol) in dioxane (10 ml) and H₂O (1 ml) was stirred at 100° C. for 15 h under N₂. The mixture was poured into H₂O and extracted with EtOAc. The organic layer was dried over anhydrous Na₂SO₄, filtered and concentrated under vacuum. The residue was purified by flash chromatography on silica gel to give the product (120 mg, 73%). MS (ESI) m/e [M+1]⁺=278.

### Step 3: 5-(1-aminoethyl)-7-methyl-3-phenylisoquinolin-1(2H)-one

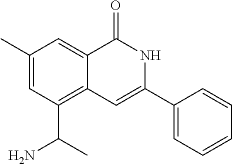 [SMILES: Cc1cc(C(C)N)c2cc(-c3ccccc3)[nH]c(=O)c2c1]

To a solution of 5-acetyl-3-chloro-7-methylisoquinolin-1(2H)-one (100 mg, 0.26 mmol) and Ammonium acetate (102 mg, 1.32 mmol) in 5 mL of ethanol was added NaBH₃CN (22 mg, 0.34 mmol). The resulting solution was stirred overnight at 90° C. After cooled to room temperature, the solution was concentrated under vacuum and the residue was purified by CombiFlash on silica gel eluted with 20%-30% MeOH in DCM to give the desired product (80 mg, 80%). MS (ESI) m/e [M+1]⁺=279.

### Step 4: 2-((1-(7-methyl-1-oxo-3-phenyl-1,2-dihydroisoquinolin-5-yl)ethyl)amino)benzonitrile

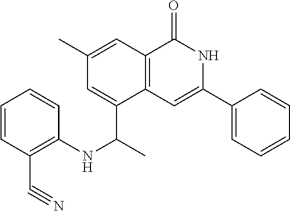 [SMILES: Cc1cc(C(C)Nc2ccccc2C#N)c2cc(-c3ccccc3)[nH]c(=O)c2c1]

To a solution of 5-(1-aminoethyl)-7-methyl-3-phenylisoquinolin-1(2H)-one (80 mg, 0.29 mmol) and 2-fluorobenzonitrile (52 mg, 0.43 mmol) in DMSO (3 ml) was added DIEA (148 mg, 1.15 mmol). The resulting solution was stirred for 48 h at 130° C. The reaction was quenched with water (10 ml) and extracted with EtOAc (10 mL×2). The combined organic extracts were washed with brine, dried over anhydrous Na₂SO₄, filtered, and concentrated to give the desired product (30 mg, 27%). MS (ESI) m/e [M+1]⁺=380.

### Step 5: 2-((1-(7-methyl-1-oxo-3-phenyl-1,2-dihydroisoquinolin-5-yl)ethyl)amino)benzoic acid

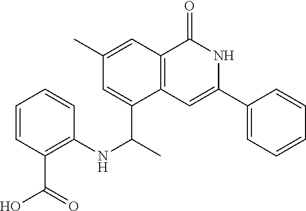 [SMILES: Cc1cc(C(C)Nc2ccccc2C(=O)O)c2cc(-c3ccccc3)[nH]c(=O)c2c1]

To a solution of 2-((1-(7-methyl-1-oxo-3-phenyl-1,2-dihydroisoquinolin-5-yl)ethyl)amino)benzonitrile (30 mg, 0.08 mmol) in ethanol (4 ml) was added NaOH (6 M in water, 1 mL). The resulting solution was stirred for 16 h at 100° C. The solution was concentrated under vacuum. The residue was redissolved in H₂O and acidized to pH 5˜6 with HCl (2 M in water). The mixture was filtered, and the filter cake was purified by Prep-HPLC to give the desired product (1 mg, 3%). ¹H NMR (400 MHz, DMSO-d₆) δ ppm 12.79 (brs, 1H), 11.55 (s, 1H), 8.53 (s, 1H), 7.93 (s, 1H), 7.86-7.74 (m, 3H), 7.53-7.37 (m, 4H), 7.21-7.05 (m, 2H), 6.49 (t, J=7.5 Hz, 1H), 6.37 (d, J=8.5 Hz, 1H), 5.38-5.25 (m, 1H), 2.34 (s, 3H), 1.53 (d, J=6.5 Hz, 3H). MS (ESI) m/e [M+1]⁺=399.

### Example 27: 2-((1-(2,7-dimethyl-1-oxo-3-(prop-1-yn-1-yl)-1,2-dihydroisoquinolin-5-yl)ethyl)amino)benzoic acid

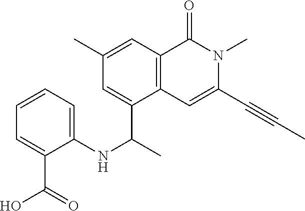 [SMILES: CC#Cc1cc2c(C(C)Nc3ccccc3C(=O)O)cc(C)cc2c(=O)n1C]

### Step 1: methyl 2-((1-(2,7-dimethyl-1-oxo-3-(prop-1-yn-1-yl)-1,2-dihydroisoquinolin-5-yl)ethyl)amino)benzoate

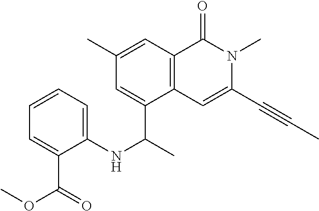 [SMILES: CC#Cc1cc2c(C(C)Nc3ccccc3C(=O)OC)cc(C)cc2c(=O)n1C]

To a solution of methyl 2-((1-(3-chloro-2,7-dimethyl-1-oxo-1,2-dihydroisoquinolin-5-yl)ethyl)amino)benzoate (60 mg, 0.16 mmol) and propyne (1 M in THF, 0.30 mL, 0.31 mmol) in DMF (4 ml) and TEA (2 ml) was added Pd(PPh₃)₄ (9 mg, 0.01 mmol) and CuI (3 mg, 0.02 mmol) under N₂. The resulting solution was stirred at 70° C. for 15 h. The mixture was poured into H₂O and extracted with EtOAc. The combined organic extracts were washed with brine (20 mL), dried over anhydrous Na₂SO₄, filtered, and concentrated under vacuum. The residue was purified by flash chromatography on silica gel eluted with 25%-30% EtOAc in petroleum ether to give the product (41 mg, 66%) as a yellow oil. MS (ESI) m/e [M+1]⁺=389.

### Step 2: 2-((1-(3-(1-(3-cyanophenyl)-1H-pyrazol-4-yl)-2,7-dimethyl-1-oxo-1,2-dihydroisoquinolin-5-yl)ethyl)amino)benzoic acid

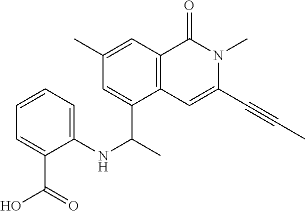 [SMILES: CC#Cc1cc2c(C(C)Nc3ccccc3C(=O)O)cc(C)cc2c(=O)n1C]

To a solution of methyl 2-((1-(2,7-dimethyl-1-oxo-3-(prop-1-yn-1-yl)-1,2-dihydroisoquinolin-5-yl)ethyl)amino)benzoate (43 mg, 0.11 mmol) in methanol (4 mL), H₂O (2 ml) and THF (4 ml) was added Lithium hydroxide monohydrate (23 mg, 0.55 mmol). The resulting solution was stirred for 7 h at 50° C. The solution was concentrated under vacuum. The residue was redissolved in H₂O and acidized to pH 5˜6 with HCl (2 M in water). The mixture was filtered, and the filter cake was purified by Prep-HPLC to give the desired product (14 mg, 34%). ¹H NMR (400 MHz, DMSO-d₆). δ ppm 12.80 (brs, 1H), 8.39 (s, 1H), 7.91 (s, 1H), 7.78 (d, J=7.3 Hz, 1H), 7.44 (s, 1H), 7.23 (s, 1H), 7.15 (t, J=7.5 Hz, 1H), 6.50 (t, J=7.5 Hz, 1H), 6.27 (d, J=8.5 Hz, 1H), 5.24-5.13 (m, 1H), 3.60 (s, 3H), 2.32 (s, 3H), 2.17 (s, 3H), 1.47 (d, J=6.5 Hz, 3H). MS (ESI) m/e [M+1]⁺=375.

### Example 28: 2-((1-(3-(cyclohexylethynyl)-2,7-dimethyl-1-oxo-1,2-dihydroisoquinolin-5-yl)ethyl)amino)benzoic acid

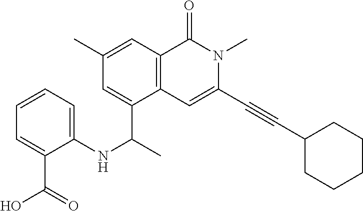 [SMILES: Cc1cc(C(C)Nc2ccccc2C(=O)O)c2cc(C#CC3CCCCC3)n(C)c(=O)c2c1]

### Step 1: methyl 2-((1-(3-(cyclohexylethynyl)-2,7-dimethyl-1-oxo-1,2-dihydroisoquinolin-5-yl)ethyl)amino)benzoate

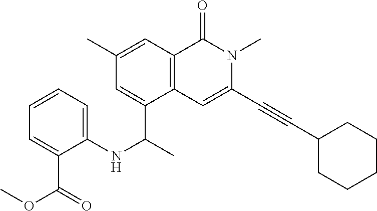 [SMILES: COC(=O)c1ccccc1NC(C)c1cc(C)cc2c(=O)n(C)c(C#CC3CCCCC3)cc12]

To a solution of methyl 2-((1-(3-chloro-2,7-dimethyl-1-oxo-1,2-dihydroisoquinolin-5-yl)ethyl)amino)benzoate (60 mg, 0.16 mmol) and ethynylcyclohexane (34 mg, 0.31 mmol) in DMF (4 ml) and TEA (2 ml) was added Pd(PPh₃)₄ (9 mg, 0.01 mmol) and CuI (3 mg, 0.02 mmol) under N₂. The resulting solution was stirred at 70° C. for 15 h. The mixture was poured into H₂O and extracted with EtOAc. The combined organic extracts were washed with brine (20 mL), dried over anhydrous Na₂SO₄, filtered, and concentrated under vacuum. The residue was purified by flash chromatography on silica gel eluted with 20%-25% EtOAc in petroleum ether to give the product (55 mg, 75%) as a yellow oil. MS (ESI) m/e [M+1]⁺=457.

### Step 2: 2-((1-(3-(cyclohexylethynyl)-2,7-dimethyl-1-oxo-1,2-dihydroisoquinolin-5-yl)ethyl)amino)benzoic acid

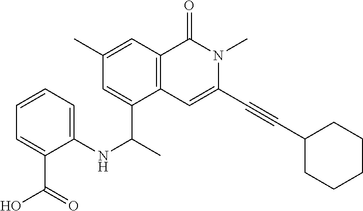 [SMILES: Cc1cc(C(C)Nc2ccccc2C(=O)O)c2cc(C#CC3CCCCC3)n(C)c(=O)c2c1]

To a solution of methyl 2-((1-(3-(cyclohexylethynyl)-2,7-dimethyl-1-oxo-1,2-dihydroisoquinolin-5-yl)ethyl)amino)benzoate (55 mg, 0.12 mmol) in methanol (4 mL), H₂O (2 ml) and THF (4 ml) was added Lithium hydroxide monohydrate (25 mg, 0.60 mmol). The resulting solution was stirred for 7 h at 50° C. The solution was concentrated under vacuum. The residue was redissolved in H₂O and acidized to pH 5˜6 with HCl (2 M in water). The mixture was filtered, and the filter cake was purified by Prep-HPLC to give the desired product (21 mg, 40%). ¹H NMR (400 MHz, DMSO-d₆). δ ppm 12.77 (brs, 1H), 8.36 (s, 1H), 7.91 (s, 1H), 7.78 (d, J=7.9 Hz, 1H), 7.44 (s, 1H), 7.20 (s, 1H), 7.15 (t, J=7.5 Hz, 1H), 6.50 (t, J=7.5 Hz, 1H), 6.27 (d, J=8.5 Hz, 1H), 5.28-5.10 (m, 1H), 3.61 (s, 3H), 2.84-2.72 (m, 1H), 2.32 (s, 3H), 1.91-1.77 (m, 2H), 1.73-1.61 (m, 2H), 1.60-1.43 (m, 6H), 1.43-1.27 (m, 3H). MS (ESI) m/e [M+1]⁺=443.

### Example 29: 2-((1-(3-(1-(3-cyanophenyl)-1H-pyrazol-4-yl)-2,7-dimethyl-1-oxo-1,2-dihydroisoquinolin-5-yl)ethyl)amino)benzoic acid

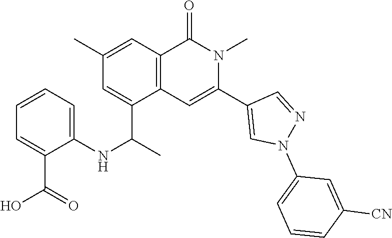 [SMILES: Cc1cc(C(C)Nc2ccccc2C(=O)O)c2cc(-c3cnn(-c4cccc(C#N)c4)c3)n(C)c(=O)c2c1]

### Step 1: methyl 2-((1-(3-(1-(3-cyanophenyl)-1H-pyrazol-4-yl)-2,7-dimethyl-1-oxo-1,2-dihydroisoquinolin-5-yl)ethyl)amino)benzoate

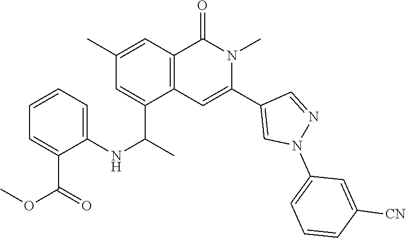 [SMILES: COC(=O)c1ccccc1NC(C)c1cc(C)cc2c(=O)n(C)c(-c3cnn(-c4cccc(C#N)c4)c3)cc12]

To a solution of methyl 2-((1-(3-chloro-2,7-dimethyl-1-oxo-1,2-dihydroisoquinolin-5-yl)ethyl)amino)benzoate (50 mg, 0.13 mmol), 3-(4-(4,4,5,5-tetramethyl-1,3,2-dioxaborolan-2-yl)-1H-pyrazol-1-yl)benzonitrile (77 mg, 0.26 mmol) and K₃PO₄ (83 mg, 0.39 mmol) in dioxane (8 ml) and H₂O (1 ml) was added Pd(PPh₃)₄ (15 mg, 0.01 mmol) under N₂. The resulting solution was stirred at 100° C. for 15 h. The mixture was poured into H₂O and extracted with EtOAc. The combined organic extracts were washed with brine (20 mL), dried over anhydrous Na₂SO₄, filtered and concentrated under vacuum. The residue was purified by flash chromatography on silica gel eluted with 55%-60% EtOAc in petroleum ether to give the product (62 mg, 93%). MS (ESI) m/e [M+1]⁺=518.

### Step 2: 2-((1-(3-(1-(3-cyanophenyl)-1H-pyrazol-4-yl)-2,7-dimethyl-1-oxo-1,2-dihydroisoquinolin-5-yl)ethyl)amino)benzoic acid

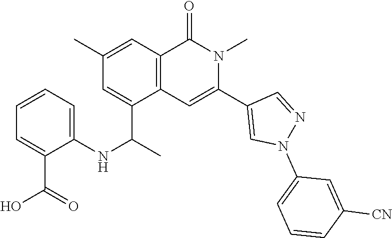 [SMILES: Cc1cc(C(C)Nc2ccccc2C(=O)O)c2cc(-c3cnn(-c4cccc(C#N)c4)c3)n(C)c(=O)c2c1]

To a solution of methyl 2-((1-(3-(1-(3-cyanophenyl)-1H-pyrazol-4-yl)-2,7-dimethyl-1-oxo-1,2-dihydroisoquinolin-5-yl)ethyl)amino)benzoate (62 mg, 0.12 mmol) in methanol (4 mL), H₂O (2 ml) and THF (4 ml) was added Lithium hydroxide monohydrate (25 mg, 0.60 mmol). The resulting solution was stirred for 13 h at 45° C. The solution was concentrated under vacuum. The residue was redissolved in H₂O and acidized to pH 5˜6 with HCl (2 M in water). The mixture was filtered, and the filter cake was purified by Prep-HPLC to the desired product (2 mg, 3%). ¹H NMR (400 MHz, DMSO-d₆). δ ppm 9.05 (s, 1H), 8.59 (brs, 1H), 8.41 (s, 1H), 8.33-8.27 (m, 1H), 8.25 (s, 1H), 7.95 (s, 1H), 7.88-7.70 (m, 3H), 7.46 (s, 1H), 7.12 (t, J=7.5 Hz, 1H), 7.06 (s, 1H), 6.48 (t, J=7.4 Hz, 1H), 6.29 (d, J=8.5 Hz, 1H), 5.27-5.15 (m, 1H), 3.60 (s, 3H), 2.34 (s, 3H), 1.51 (d, J=6.5 Hz, 3H). MS (ESI) m/e [M+1]⁺=504.

### Example 30: (R)-2-((1-(2,7-dimethyl-1-oxo-3-phenyl-1,2-dihydroisoquinolin-5-yl)ethyl)amino)benzoic acid

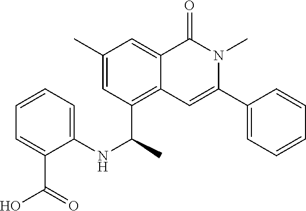 [SMILES: Cc1cc([C@@H](C)Nc2ccccc2C(=O)O)c2cc(-c3ccccc3)n(C)c(=O)c2c1]

Chiral separation of racemic 2-((1-(2,7-dimethyl-1-oxo-3-phenyl-1,2-dihydroisoquinolin-5-yl)ethyl)amino)benzoic acid (100 mg) by SFC (column: DAICEL CHIRALCEL OD (250 mm*30 mm, 10 um); mobile phase: [CO₂-MeOH (0.1% NH3H2O)]; B %: 32%, isocratic elution mode) to give (R)-2-((1-(2,7-dimethyl-1-oxo-3-phenyl-1,2-dihydroisoquinolin-5-yl)ethyl)amino)benzoic acid (30 mg), Rt: 0.989 min. 11H NMR (400 MHz, DMSO-d₆) δ ppm 12.75 (s, 1H), 8.37 (s, 1H), 8.00 (s, 1H), 7.80 (d, J=7.4 Hz, 1H), 7.62-7.45 (m, 6H), 7.21-7.13 (m, 1H), 6.83 (s, 1H), 6.56-6.47 (m, 1H), 6.34 (d, J=8.1 Hz, 1H), 5.20 (s, 1H), 3.33 (s, 3H), 2.37 (s, 3H), 1.51 (d, J=3.7 Hz, 7H). MS (ESI) m/e [M+1]⁺=413.

### Example 31: (S)-2-((1-(2,7-dimethyl-1-oxo-3-phenyl-1,2-dihydroisoquinolin-5-yl)ethyl)amino)benzoic acid

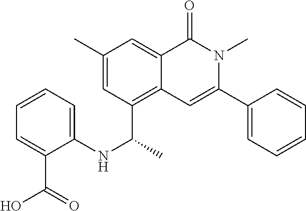 [SMILES: Cc1cc([C@H](C)Nc2ccccc2C(=O)O)c2cc(-c3ccccc3)n(C)c(=O)c2c1]

Chiral separation of racemic 2-((1-(2,7-dimethyl-1-oxo-3-phenyl-1,2-dihydroisoquinolin-5-yl)ethyl)amino)benzoic acid (100 mg) by SFC (column: DAICEL CHIRALCEL OD (250 mm*30 mm, 10 μm); mobile phase: [CO2-MeOH (0.1% NH3H2O)]; B %: 32%, isocratic elution mode) to give (S)-2-((1-(2,7-dimethyl-1-oxo-3-phenyl-1,2-dihydroisoquinolin-5-yl)ethyl)amino)benzoic acid (29.1 mg), Rt: 1.502 min. 11H NMR (400 MHz, DMSO-d₆) δ ppm 12.75 (s, 1H), 8.37 (s, 1H), 8.00 (s, 1H), 7.80 (d, J=7.4 Hz, 1H), 7.62-7.45 (m, 6H), 7.21-7.13 (m, 1H), 6.83 (s, 1H), 6.56-6.47 (m, 1H), 6.34 (d, J=8.1 Hz, 1H), 5.20 (s, 1H), 3.33 (s, 3H), 2.37 (s, 3H), 1.51 (d, J=3.7 Hz, 7H). MS (ESI) m/e [M+1]⁺=413.

### Example 32: 2-((1-(3-(2-cyanophenyl)-2,7-dimethyl-1-oxo-1,2-dihydroisoquinolin-5-yl)ethyl)amino)benzoic acid

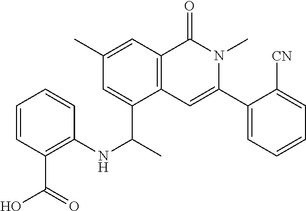 [SMILES: Cc1cc(C(C)Nc2ccccc2C(=O)O)c2cc(-c3ccccc3C#N)n(C)c(=O)c2c1]

The desired product (2.8 mg) was obtained following the similar procedure as Example 10. ¹H NMR (400 MHz, DMSO-d₆) δ ppm 8.46 (s, 1H), 8.10-7.99 (m, 2H), 7.92-7.70 (m, 4H), 7.51 (m, 1H), 7.11 (m, 2H), 6.50 (m, 1H), 6.32 (m, 1H), 5.23 (m, 1H), 3.27 (d, J=2.4 Hz, 3H), 2.38 (d, J=7.2 Hz, 3H), 1.55-1.44 (m, 3H). MS (ESI) m/e [M+1]⁺=438.

### Example 33: 3-((1-(3-(4-fluorophenyl)-2,7-dimethyl-1-oxo-1,2-dihydroisoquinolin-5-yl)ethyl)amino)picolinic acid

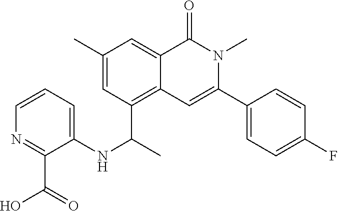 [SMILES: Cc1cc(C(C)Nc2cccnc2C(=O)O)c2cc(-c3ccc(F)cc3)n(C)c(=O)c2c1]

### Step 1: methyl 3-((1-(3-(4-fluorophenyl)-2,7-dimethyl-1-oxo-12-dihydroisoquinolin-5-yl)ethyl)amino)picolinate

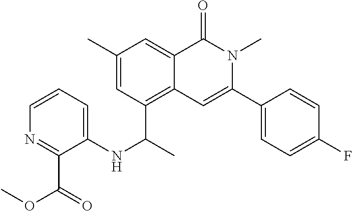 [SMILES: COC(=O)c1ncccc1NC(C)c1cc(C)cc2c(=O)n(C)c(-c3ccc(F)cc3)cc12]

A mixture of 5-(1-bromoethyl)-3-(4-fluorophenyl)-2,7-dimethylisoquinolin-1(2H)-one (60 mg, 0.16 mmol), methyl 3-aminopicolinate (37 mg, 0.24 mmol), DIEA (62 mg, 0.48 mmol) in DMF (5 ml) was stirred at 90° C. for 1 h. The mixture was concentrated under vacuum. The residue was purified by Prep-TLC to give the desired product (30 mg, 42%). MS (ESI) m/e [M+1]⁺=446.

### Step 2: 3-((1-(3-(4-fluorophenyl)-2,7-dimethyl-1-oxo-1,2-dihydroisoquinolin-5-yl)ethyl)amino)picolinic acid

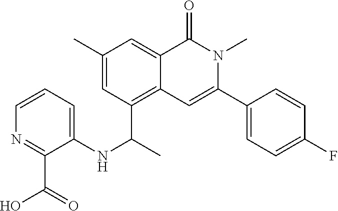 [SMILES: Cc1cc(C(C)Nc2cccnc2C(=O)O)c2cc(-c3ccc(F)cc3)n(C)c(=O)c2c1]

To a solution of methyl 3-((1-(3-(4-fluorophenyl)-2,7-dimethyl-1-oxo-1,2-dihydroisoquinolin-5-yl)ethyl)amino)picolinate (30 mg, 0.07 mmol) in MeOH (1 ml) and THF (0.5 ml) was added 4N NaOH (0.2 mL). The reaction mixture was stirred at rt for 1 h. The mixture was adjusted to pH=5 by 1N HCl and concentrated under vacuum. The residue was purified by prep-HPLC to give the desired product (6.0 mg, 21%). ¹H NMR (400 MHz, DMSO-d₆) δ ppm 8.95-8.50 (m, 1H), 7.99 (s, 1H), 7.84-7.81 (m, 1H), 7.67-7.64 (m, 2H), 7.48 (s, 1H), 7.39-7.34 (m, 2H), 7.33-7.23 (m, 1H), 6.88 (d, J=7.2 Hz, 1H), 6.83 (s, 1H), 5.35-5.20 (m, 1H), 3.53 (s, 3H), 2.36 (s, 3H), 1.52 (d, J=6.8 Hz, 3H). MS (ESI) m/e [M+1]⁺=432.

### Example 34: (R)-2-((1-(3-(4-fluorophenyl)-2,7-dimethyl-1-oxo-1,2-dihydroisoquinolin-5-yl)ethyl)amino)benzoic acid

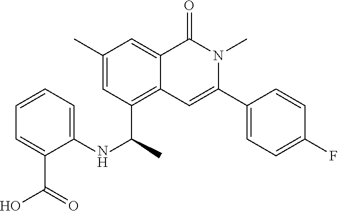 [SMILES: Cc1cc([C@@H](C)Nc2ccccc2C(=O)O)c2cc(-c3ccc(F)cc3)n(C)c(=O)c2c1]

Chiral separation of racemic 2-((1-(3-(4-fluorophenyl)-2,7-dimethyl-1-oxo-1,2-dihydroisoquinolin-5-yl)ethyl)amino)benzoic acid (110 mg) by SFC (column: DAICEL CHIRALPAK OD (250 mm*50 mm, 10 um); mobile phase::[CO₂-MeOH (0.1% NH₃H₂O)]; B %: 35%, isocratic elution mode) to give (R)-2-((1-(3-(4-fluorophenyl)-2,7-dimethyl-1-oxo-1,2-dihydroisoquinolin-5-yl)ethyl)amino)benzoic acid (43 mg), Rt: 4.033 min. 1′H NMR (400 MHz, DMSO-d₆) δ ppm 12.74 ppm (brs, 1H), 8.37 (d, J=4.8 Hz, 1H), 7.99 (s, 1H), 7.79 (d, J=7.8 Hz, 1H), 7.68-7.62 (m, 2H), 7.47 (s, 1H), 7.36 (t, J=7.8 Hz, 2H), 7.17 (t, J=7.2 Hz, 1H), 6.83 (s, 1H), 6.51 (t, J=6.4 Hz, 1H), 6.33 (d, J=8.4 Hz, 1H), 5.24-5.15 (m, 1H), 3.32 (s, 3H), 2.36 (s, 3H), 1.50 (d, J=4.8 Hz, 3H). MS (ESI) m/e [M+1]⁺=431.

### Example 35: (S)-2-((1-(3-(4-fluorophenyl)-2,7-dimethyl-1-oxo-1,2-dihydroisoquinolin-5-yl)ethyl)amino)benzoic acid

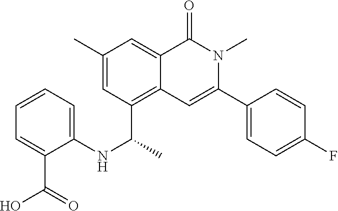 [SMILES: Cc1cc([C@H](C)Nc2ccccc2C(=O)O)c2cc(-c3ccc(F)cc3)n(C)c(=O)c2c1]

Chiral separation of racemic 2-((1-(3-(4-fluorophenyl)-2,7-dimethyl-1-oxo-1,2-dihydroisoquinolin-5-yl)ethyl)amino)benzoic acid (110 mg) by SFC (column: DAICEL CHIRALPAK OD (250 mm*50 mm, 10 um); mobile phase::[CO₂-MeOH (0.1% NH₃H₂O)]; B %: 35%, isocratic elution mode) to give (S)-2-((1-(3-(4-fluorophenyl)-2,7-dimethyl-1-oxo-1,2-dihydroisoquinolin-5-yl)ethyl)amino)benzoic acid (18 mg), Rt: 3.630 min. ¹H NMR (400 MHz, DMSO-d₆) δ ppm 12.74 (brs, 1H), 8.37 (d, J=4.8 Hz, 1H), 7.99 (s, 1H), 7.79 (d, J=7.8 Hz, 1H), 7.68-7.62 (m, 2H), 7.47 (s, 1H), 7.36 (t, J=7.8 Hz, 2H), 7.17 (t, J=7.2 Hz, 1H), 6.83 (s, 1H), 6.51 (t, J=6.4 Hz, 1H), 6.33 (d, J=8.4 Hz, 1H), 5.24-5.15 (m, 1H), 3.32 (s, 3H), 2.36 (s, 3H), 1.50 (d, J=4.8 Hz, 3H). MS (ESI) m/e [M+1]⁺=431.

### Example 36: 2-((1-(2,7-dimethyl-1-oxo-3-(pyridin-2-yl)-1,2-dihydroisoquinolin-5-yl)ethyl)amino)benzoic acid

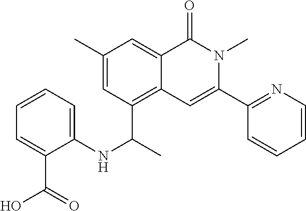 [SMILES: Cc1cc(C(C)Nc2ccccc2C(=O)O)c2cc(-c3ccccn3)n(C)c(=O)c2c1]

The desired product (13 mg) was obtained following the similar procedure as Example 10. ¹H NMR (399 MHz, DMSO-d₆) δ ppm 12.76 (s, 1H), 8.74 (d, J=4.1 Hz, 1H), 8.39 (d, J=5.4 Hz, 1H), 8.08-7.96 (m, 2H), 7.80 (dd, J=13.2, 4.9 Hz, 2H), 7.58-7.47 (m, 2H), 7.18 (t, J=7.1 Hz, 1H), 7.02 (s, 1H), 6.52 (m, 1H), 6.35 (d, J=8.5 Hz, 1H), 5.44-5.07 (m, 1H), 3.40 (s, 3H), 2.39 (s, 3H), 1.52 (d, J=6.5 Hz, 3H). MS (ESI) m/e [M+1]⁺=414.

### Example 37: 2-((1-(2,7-dimethyl-1-oxo-3-(pyridin-3-yl)-1,2-dihydroisoquinolin-5-yl)ethyl)amino)benzoic acid

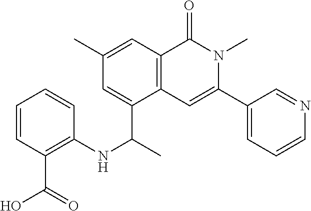 [SMILES: Cc1cc(C(C)Nc2ccccc2C(=O)O)c2cc(-c3cccnc3)n(C)c(=O)c2c1]

The desired product (23.56 mg) was obtained following the similar procedure as Example 10. ¹H NMR (400 MHz, DMSO-d₆) δ ppm 12.78 (s, 1H), 8.82 (d, J=1.6 Hz, 1H), 8.74-8.67 (m, 1H), 8.46 (s, 1H), 8.07 (d, J=8.0 Hz, 1H), 8.00 (s, 1H), 7.79 (d, J=6.8 Hz, 1H), 7.57 (m, 1H), 7.49 (s, 1H), 7.15 (t, J=7.2 Hz, 1H), 6.94 (s, 1H), 6.50 (m, 1H), 6.33 (d, J=8.4 Hz, 1H), 5.24 (m, 1H), 3.34 (s, 3H), 2.37 (s, 3H), 1.50 (d, J=6.4 Hz, 3H). MS (ESI) m/e [M+1]⁺=414.

### Example 38: 2-((1-(3-(5-fluoropyridin-3-yl)-2,7-dimethyl-1-oxo-1,2-dihydroisoquinolin-5-yl)ethyl)amino)benzoic acid

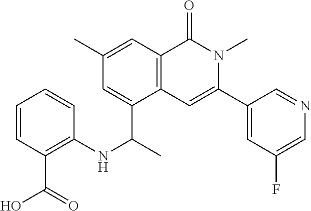 [SMILES: Cc1cc(C(C)Nc2ccccc2C(=O)O)c2cc(-c3cncc(F)c3)n(C)c(=O)c2c1]

The desired product (14.5 mg) was obtained following the similar procedure as Example 10. ¹H NMR (400 MHz, DMSO-d₆) δ ppm 12.76 (brs, 1H), 8.84-8.65 (m, 2H), 8.42 (s, 1H), 8.12 (d, J=10.0 Hz, 1H), 8.00 (s, 1H), 7.79 (d, J=6.4 Hz, 1H), 7.49 (s, 1H), 7.15 (m, 1H), 7.01 (s, 1H), 6.51 (m, 1H), 6.33 (d, J=8.8 Hz, 1H), 5.25 (m, 1H), 3.36 (s, 3H), 2.37 (s, 3H), 1.50 (d, J=6.8 Hz, 3H). MS (ESI) m/e [M+1]⁺=432.

### Example 39: 2-((1-(2,7-dimethyl-3-(2-methyl-2H-pyrazolo[4,3-b]pyridin-5-yl)-1-oxo-1,2-dihydroisoquinolin-5-yl)ethyl)amino)benzoic acid

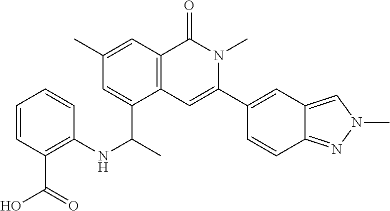 [SMILES: Cc1cc(C(C)Nc2ccccc2C(=O)O)c2cc(-c3ccc4nn(C)cc4c3)n(C)c(=O)c2c1]

The desired product (49.3 mg) was obtained following the similar procedure as Example 10. ¹H NMR (400 MHz, DMSO-d₆) δ ppm 8.77 (s, 2H), 8.25 (d, J=8.8 Hz, 1H), 8.01 (s, 1H), 7.79 (m, 1H), 7.63 (d, J=8.8 Hz, 1H), 7.53 (s, 1H), 7.18-6.95 (m, 2H), 6.47 (m, 1H), 6.29 (d, J=8.4 Hz, 1H), 5.19 (m, 1H), 4.27 (s, 3H), 3.42 (s, 3H), 2.38 (s, 3H), 1.50 (d, J=6.8 Hz, 3H). MS (ESI) m/e [M+1]⁺=468.

### Example 40: (R)-2-((1-(2,7-dimethyl-3-(4-(1-methyl-1H-pyrazol-4-yl)phenyl)-1-oxo-1,2-dihydroisoquinolin-5-yl)ethyl)amino)benzoic acid

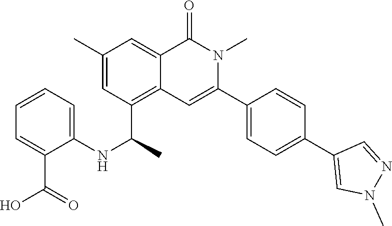 [SMILES: Cc1cc([C@@H](C)Nc2ccccc2C(=O)O)c2cc(-c3ccc(-c4cnn(C)c4)cc3)n(C)c(=O)c2c1]

Chiral separation of racemic 2-((1-(2,7-dimethyl-3-(4-(1-methyl-1H-pyrazol-4-yl)phenyl)-1-oxo-1,2-dihydroisoquinolin-5-yl)ethyl)amino)benzoic acid (110 mg) by SFC (Column Name: CHIRALPAK AD-H 20*250 mm, 5 m; Co-Solvent: 35% MeOH (0.1% DEA); Temperature (° C.): 35; Flow (ml/min): 40 mL/min; Back Pressure: 100 bar) to give (R)-2-((1-(2,7-dimethyl-3-(4-(1-methyl-1H-pyrazol-4-yl)phenyl)-1-oxo-1,2-dihydroisoquinolin-5-yl)ethyl)amino)benzoic acid (52 mg), Rt: 6.45 min. ¹H NMR (400 MHz, DMSO-d₆) δ ppm 12.75 (brs, 1H), 8.39 (s, 1H), 8.22 (s, 1H), 8.03-7.89 (m, 2H), 7.84-7.73 (m, 1H), 7.73-7.62 (m, 2H), 7.61-7.51 (m, 2H), 7.46 (s, 1H), 7.15 (t, J=7.2 Hz, 1H), 6.83 (s, 1H), 6.50 (t, J=6.9 Hz, 1H), 6.32 (d, J=8.1 Hz, 1H), 5.19 (s, 1H), 3.87 (s, 3H), 3.34 (s, 3H), 2.35 (s, 3H), 1.50 (d, J=4.5 Hz, 3H). MS (ESI) m/e [M+1]⁺=493.

### Example 41: (S)-2-((1-(2,7-dimethyl-3-(4-(1-methyl-1H-pyrazol-4-yl)phenyl)-1-oxo-1,2-dihydroisoquinolin-5-yl)ethyl)amino)benzoic acid

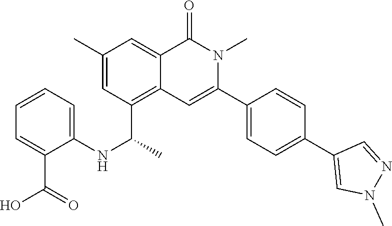 [SMILES: Cc1cc([C@H](C)Nc2ccccc2C(=O)O)c2cc(-c3ccc(-c4cnn(C)c4)cc3)n(C)c(=O)c2c1]

Chiral separation of racemic 2-((1-(2,7-dimethyl-3-(4-(1-methyl-1H-pyrazol-4-yl)phenyl)-1-oxo-1,2-dihydroisoquinolin-5-yl)ethyl)amino)benzoic acid (110 mg) by SFC (Column Name: CHIRALPAK AD-H 20*250 mm, 5 m; Co-Solvent: 35% MeOH (0.1% DEA); Temperature (° C.): 35; Flow (ml/min): 40 mL/min; Back Pressure: 100 bar) to give (S)-2-((1-(2,7-dimethyl-3-(4-(1-methyl-1H-pyrazol-4-yl)phenyl)-1-oxo-1,2-dihydroisoquinolin-5-yl)ethyl)amino)benzoic acid (49.6 mg), Rt: 10.05 min. ¹H NMR (400 MHz, DMSO-d₆) δ ppm 12.75 (brs, 1H), 8.39 (s, 1H), 8.22 (s, 1H), 8.03-7.89 (m, 2H), 7.84-7.73 (m, 1H), 7.73-7.62 (m, 2H), 7.61-7.51 (m, 2H), 7.46 (s, 1H), 7.15 (t, J=7.2 Hz, 1H), 6.83 (s, 1H), 6.50 (t, J=6.9 Hz, 1H), 6.32 (d, J=8.1 Hz, 1H), 5.19 (s, 1H), 3.87 (s, 3H), 3.34 (s, 3H), 2.35 (s, 3H), 1.50 (d, J=4.5 Hz, 3H). MS (ESI) m/e [M+1]⁺=493.

### Example 42: 2-((1-(2,7-dimethyl-1-oxo-3-(phenylcarbamoyl)-1,2-dihydroisoquinolin-5-yl)ethyl)amino)benzoic acid

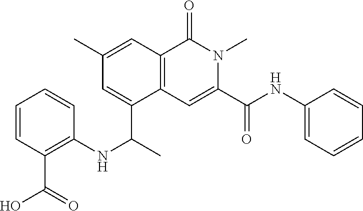 [SMILES: Cc1cc(C(C)Nc2ccccc2C(=O)O)c2cc(C(=O)Nc3ccccc3)n(C)c(=O)c2c1]

### Step 1: methyl 5-acetyl-2,7-dimethyl-1-oxo-1,2-dihydroisoquinoline-3-carboxylate

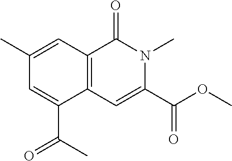

A mixture of 5-acetyl-3-chloro-2,7-dimethylisoquinolin-1(2H)-one (500 mg, 2.0 mmol), Pd(dppf)Cl₂ (163 mg, 0.2 mmol), and TEA (606 mg, 6.0 mmol) in MeOH (15 ml) was stirred at 100° C. for 15 h under CO atmosphere (25 atm). The mixture was concentrated under vacuum. The residue was purified by CombiFlash chromatography on silica gel (PE/EA=1/1) to give the desired product (320 mg, 58%). MS (ESI) m/e [M+1]⁺=274.

### Step 2: 5-acetyl-2,7-dimethyl-1-oxo-1,2-dihydroisoquinoline-3-carboxylic acid

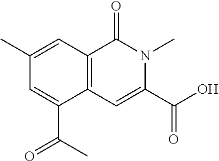 [SMILES: CC(=O)c1cc(C)cc2c(=O)n(C)c(C(=O)O)cc12]

To a solution of methyl 5-acetyl-2,7-dimethyl-1-oxo-1,2-dihydroisoquinoline-3-carboxylate (320 mg, 1.2 mmol) in methanol (5 ml) was added NaOH (3M, 1 mL). The resulting solution was stirred for 2 h at 25° C. The solution was concentrated under vacuum. The residue was redissolved in H₂O and acidized to pH 5˜6 with HCl (2 M in water). The mixture was filtered and the solid was collected to afford the desired product (280 mg, crude). MS (ESI) m/e [M+1]⁺=260.

### Step 3: 5-acetyl-2,7-dimethyl-1-oxo-N-phenyl-1,2-dihydroisoquinoline-3-carboxamide

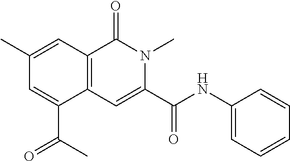 [SMILES: CC(=O)c1cc(C)cc2c(=O)n(C)c(C(=O)Nc3ccccc3)cc12]

A mixture of 5-acetyl-2,7-dimethyl-1-oxo-1,2-dihydroisoquinoline-3-carboxylic acid (280 mg, 1.1 mmol), aniline (113 mg, 1.2 mmol), HATU (627 mg, 1.6 mmol) and DIEA (426 mg, 3.3 mmol) in DMF (5 ml) was stirred at 25° C. for 15 h under N₂. The mixture was concentrated under vacuum. The residue was purified by CombiFlash chromatography on silica gel (PE/EA=2/3) to give the desired product (230 mg, 62%). MS (ESI) m/e [M+1]⁺=335.

### Step 4: 5-(1-aminoethyl)-2,7-dimethyl-1-oxo-N-phenyl-1,2-dihydroisoquinoline-3-carboxamide

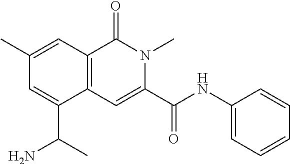 [SMILES: Cc1cc(C(C)N)c2cc(C(=O)Nc3ccccc3)n(C)c(=O)c2c1]

To a solution of 5-acetyl-2,7-dimethyl-1-oxo-N-phenyl-1,2-dihydroisoquinoline-3-carboxamide (230 mg, 0.7 mmol) in ethanol (10 ml) was added ammonium acetate (539 mg, 7 mmol), followed by the addition of NaBH₃CN (70 mg, 1.1 mmol). The resulting solution was stirred for 15 h at 90° C. The solution was concentrated under vacuum. The residue was purified by CombiFlash chromatography on silica gel (DCM/MeOH with 1% of ammonium hydroxide) to give the desired product (130 mg, 55%). MS (ESI) m/e [M+1]⁺=336.

### Step 5: 2-((1-(2,7-dimethyl-1-oxo-3-(phenylcarbamoyl)-1,2-dihydroisoquinolin-5-yl)ethyl)amino)benzoic acid

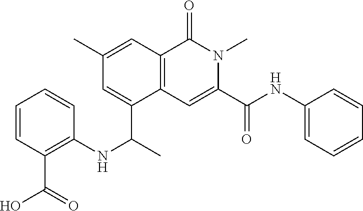 [SMILES: Cc1cc(C(C)Nc2ccccc2C(=O)O)c2cc(C(=O)Nc3ccccc3)n(C)c(=O)c2c1]

To a solution of 5-(1-aminoethyl)-2,7-dimethyl-1-oxo-N-phenyl-1,2-dihydroisoquinoline-3-carboxamide (130 mg, 0.39 mmol), 2-aminobenzoic acid (69 mg, 0.50 mmol), L-proline (4 mg, 0.04 mmol) and K₂CO₃ (166 mg, 1.2 mmol) in 3 mL of DMSO was added CuI (7 mg, 0.04 mmol). The resulted solution was stirred overnight at 110° C. under N₂. After cooled to room temperature, the solution was concentrated under vacuum and the residue was dissolved in water. The pH value of the solution was adjusted to 4-5, and the solid was collected by filtration. The crude product was purified by Prep-HPLC (mobile phase, water (10 mmol/L FA) and ACN (25% Phase B up to 80% in 8 min); Detector, UV 254 nm) to give 2-((1-(2,7-dimethyl-1-oxo-3-(phenylcarbamoyl)-1,2-dihydroisoquinolin-5-yl)ethyl)amino)benzoic acid (19 mg, 11%). ¹H NMR (400 MHz, DMSO-d₆) δ ppm 12.78 (brs, 1H), 10.87 (s, 1H), 8.41 (s, 1H), 7.99 (s, 1H), 7.86-7.64 (m, 3H), 7.56-7.05 (m, 6H), 6.51 (m, 1H), 6.31 (d, J=8.4 Hz, 1H), 5.36-5.17 (m, 1H), 3.52 (s, 3H), 2.36 (s, 3H), 1.53 (d, J=6.1 Hz, 3H). MS (ESI) m/e [M+1]⁺=456.

### Example 43: 2-((1-(2,7-dimethyl-1-oxo-3-(phenylethynyl)-1,2-dihydroisoquinolin-5-yl)ethyl)amino)benzoic acid

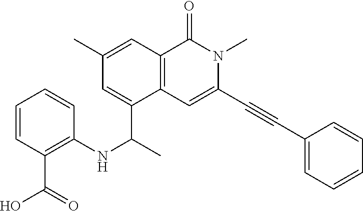 [SMILES: Cc1cc(C(C)Nc2ccccc2C(=O)O)c2cc(C#Cc3ccccc3)n(C)c(=O)c2c1]

### Step 1: methyl 2-((1-(2,7-dimethyl-1-oxo-3-(phenylethynyl)-1,2-dihydroisoquinolin-5-yl)ethyl)amino)benzoate

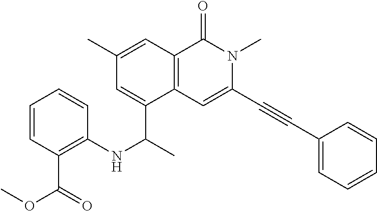 [SMILES: COC(=O)c1ccccc1NC(C)c1cc(C)cc2c(=O)n(C)c(C#Cc3ccccc3)cc12]

A mixture of methyl 2-((1-(3-chloro-2,7-dimethyl-1-oxo-1,2-dihydroisoquinolin-5-yl)ethyl)amino)benzoate (80 mg, 0.2 mmol), ethynylbenzene (31 mg, 0.3 mmol), Pd(PPh₃)₂Cl₂ (14 mg, 0.02 mmol) in TEA (1 ml) and DMF (2 ml) was stirred at 100° C. for 15 h under N₂. The mixture was poured into H₂O and extracted with EtOAc. The organic layer was dried over Na₂SO₄, filtered, and concentrated under vacuum. The residue was purified by Prep-TLC (PE/EA=2:1) to give the desired product (50 mg, 55%). MS (ESI) m/e [M+1]⁺=451.

### Step 2: 2-((1-(2,7-dimethyl-1-oxo-3-(phenylethynyl)-1,2-dihydroisoquinolin-5-yl)ethyl) amino)benzoic acid

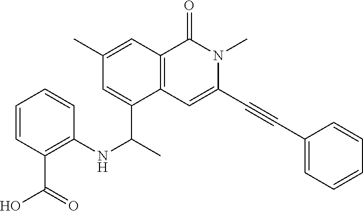 [SMILES: Cc1cc(C(C)Nc2ccccc2C(=O)O)c2cc(C#Cc3ccccc3)n(C)c(=O)c2c1]

To a solution of methyl 2-((1-(2,7-dimethyl-1-oxo-3-(phenylethynyl)-1,2-dihydroisoquinolin-5-yl)ethyl)amino)benzoate (25 mg, 0.06 mmol) in methanol (3 ml) was added NaOH (3M, 1 mL). The resulting solution was stirred for 5 h at 50° C. The solution was concentrated under vacuum. The residue was redissolved in H₂O and acidized to pH 5˜6 with HCl (2 M in water). The mixture was filtered and the solid was collected, purified by Prep-HPLC (mobile phase, water (10 mmol/L FA) and ACN (25% Phase B up to 80% in 8 min); Detector, UV 254 nm) to give the desired product (15 mg, 46%). ¹H NMR (400 MHz, DMSO-d₆) δ ppm 12.79 (s, 1H), 8.42 (s, 1H), 7.96 (s, 1H), 7.83-7.73 (m, 1H), 7.72-7.61 (m, 2H), 7.56-7.41 (m, 5H), 7.16 (m, 1H), 6.51 (m, 1H), 6.30 (d, J=8.5 Hz, 1H), 5.33-5.17 (m, 1H), 3.71 (s, 3H), 2.35 (s, 3H), 1.51 (d, J=6.4 Hz, 3H). MS (ESI) m/e [M+1]⁺=437.

### Example 44: (E)-2-((1-(2,7-dimethyl-1-oxo-3-styryl-1,2-dihydroisoquinolin-5-yl)ethyl)amino)benzoic acid

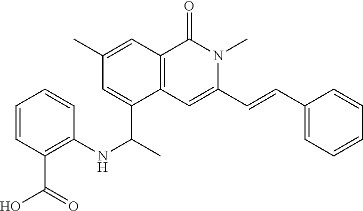 [SMILES: Cc1cc(C(C)Nc2ccccc2C(=O)O)c2cc(/C=C/c3ccccc3)n(C)c(=O)c2c1]

### Step 1: methyl (E)-2-((1-(2,7-dimethyl-1-oxo-3-styryl-1,2-dihydroisoquinolin-5-yl)ethyl)amino)benzoate

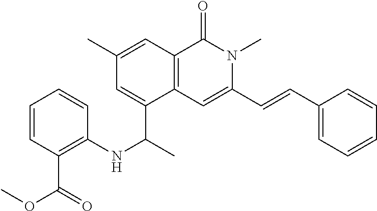 [SMILES: COC(=O)c1ccccc1NC(C)c1cc(C)cc2c(=O)n(C)c(/C=C/c3ccccc3)cc12]

To a solution of methyl 2-((1-(2,7-dimethyl-1-oxo-3-(phenylethynyl)-1,2-dihydroisoquinolin-5-yl)ethyl)amino)benzoate (25 mg, 0.06 mmol) in MeOH (3 mL) was added NaBH₄ (7 mg, 0.18 mmol). The resulting solution was stirred at 0-10° C. for 1 h. The mixture was quenched with water, concentrated under vacuum. The residue was purified by Prep-TLC (PE/EA=2:1) to give the desired product (20 mg, 74%). MS (ESI) m/e [M+1]⁺=453.

### Step 2: (E)-2-((1-(2,7-dimethyl-1-oxo-3-styryl-1,2-dihydroisoquinolin-5-yl)ethyl)amino) benzoic acid

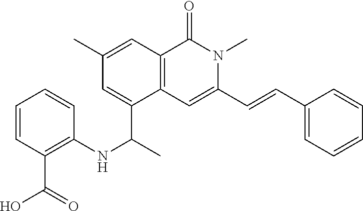 [SMILES: Cc1cc(C(C)Nc2ccccc2C(=O)O)c2cc(/C=C/c3ccccc3)n(C)c(=O)c2c1]

To a solution of methyl (E)-2-((1-(2,7-dimethyl-1-oxo-3-styryl-1,2-dihydroisoquinolin-5-yl)ethyl)amino)benzoate (20 mg, 0.04 mmol) in methanol (3 mL) was added NaOH (3M, 1 mL). The resulting solution was stirred for 5 h at 50° C. The solution was concentrated under vacuum. The residue was redissolved in H₂O and acidized to pH 5˜6 with HCl (2 M in water). The mixture was filtered and the solid was collected, purified by Prep-HPLC (mobile phase, water (10 mmol/L FA) and ACN (25% Phase B up to 80% in 8 min); Detector, UV 254 nm) to give (E)-2-((1-(2,7-dimethyl-1-oxo-3-styryl-1,2-dihydroisoquinolin-5-yl)ethyl)amino)benzoic acid (10 mg, 57%). ¹H NMR (400 MHz, DMSO-d₆) δ ppm 12.76 (brs, 1H), 8.27 (s, 1H), 7.92 (s, 1H), 7.78-7.72 (m, 1H), 7.33 (s, 1H), 7.30-7.13 (m, 5H), 7.12-7.04 (m, 1H), 6.96-6.89 (m, 1H), 6.71-6.58 (m, 2H), 6.48 (m, 1H), 5.96 (d, J=8.5 Hz, 1H), 4.76-4.62 (m, 1H), 3.56 (s, 3H), 2.30 (s, 3H), 1.16 (t, J=5.2 Hz, 3H). MS (ESI) m/e [M+1]⁺=439.

### Example 45: 2-((1-(3-(3,4-dihydronaphthalen-2-yl)-2,7-dimethyl-1-oxo-1,2-dihydroisoquinolin-5-yl)ethyl)amino)benzoic acid

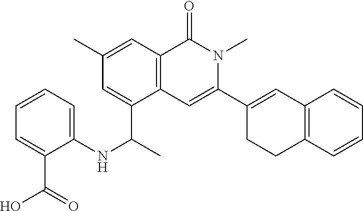 [SMILES: Cc1cc(C(C)Nc2ccccc2C(=O)O)c2cc(C3=Cc4ccccc4CC3)n(C)c(=O)c2c1]

The desired product (13.6 mg) was obtained following the similar procedure as Example 10. ¹H NMR (399 MHz, DMSO-d₆) δ ppm 12.76 (brs, 1H), 8.40 (s, 1H), 7.94 (s, 1H), 7.79 (dd, J=7.9, 1.4 Hz, 1H), 7.45 (s, 1H), 7.29-7.06 (m, 5H), 6.87 (s, 1H), 6.79 (s, 1H), 6.50 (m, 1H), 6.33 (d, J=8.5 Hz, 1H), 5.23-5.09 (m, 1H), 3.50 (s, 3H), 2.93 (t, J=8.0 Hz, 2H), 2.56 (t, J=7.9 Hz, 2H), 2.34 (s, 3H), 1.52 (d, J=6.5 Hz, 3H). MS (ESI) m/e [M+1]⁺=465.3. MS (ESI) m/e [M+1]⁺=465.

### Example 46: 2-((1-(2,7-dimethyl-1-oxo-3-(1-phenyl-1,2,3,6-tetrahydropyridin-4-yl)-1,2-dihydroisoquinolin-5-yl)ethyl)amino)benzoic acid

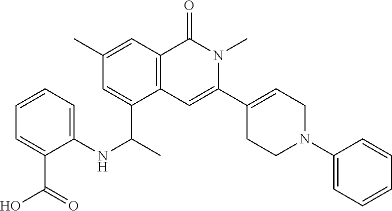 [SMILES: Cc1cc(C(C)Nc2ccccc2C(=O)O)c2cc(C3=CCN(c4ccccc4)CC3)n(C)c(=O)c2c1]

The desired product (10 mg) was obtained following the similar procedure as Example 10. ¹H NMR (399 MHz, DMSO-d₆) δ ppm 12.78 (brs, 1H), 8.47 (s, 1H), 7.92 (s, 1H), 7.81-7.72 (m, 1H), 7.44 (s, 1H), 7.29-7.19 (m, 2H), 7.14 (t, J=7.5 Hz, 1H), 6.98 (d, J=8.1 Hz, 2H), 6.82-6.68 (m, 2H), 6.49 (m, 1H), 6.29 (d, J=8.4 Hz, 1H), 6.11 (s, 1H), 5.15 (s, 1H), 3.85 (s, 2H), 3.49 (t, J=5.4 Hz, 2H), 3.42 (s, 3H), 2.54-2.50 (m, 2H), 2.32 (s, J=8.8 Hz, 3H), 1.50 (d, J=6.5 Hz, 3H). MS (ESI) m/e [M+1]⁺=494.

### Example 47: 5-((1-(2,7-dimethyl-1-oxo-3-phenyl-1,2-dihydroisoquinolin-5-yl)ethyl)amino)benzo[b]thiophene-6-carboxylic acid

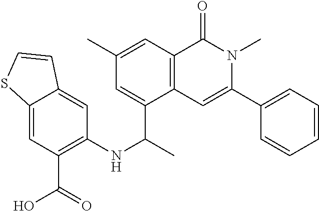 [SMILES: Cc1cc(C(C)Nc2cc3ccsc3cc2C(=O)O)c2cc(-c3ccccc3)n(C)c(=O)c2c1]

### Step 1: methyl 6-bromo-5-nitrobenzo[b]thiophene-2-carboxylate

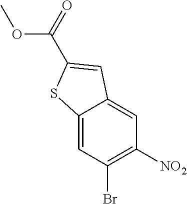 [SMILES: COC(=O)c1cc2cc([N+](=O)[O-])c(Br)cc2s1]

A mixture of 4-bromo-2-fluoro-5-nitrobenzaldehyde (1.0 g, 4.03 mmol), methyl 2-mercaptoacetate (427 mg, 4.03 mmol), K₂CO₃ (1.1 g, 8.06 mmol) in DMF (10 ml) was stirred at 80° C. for 2 h. The mixture was allowed to cool down to room temperature, diluted with water (50 ml) and extracted with ethyl acetate. The combined organic layers were washed with brine, dried over anhydrous Na₂SO₄, filtrated and concentrated under reduced pressure. The residue was purified by flash chromatography on silica gel to give the desired product (1.0 g, 79%). MS (ESI) m/e [M+1]⁺=316.

### Step 2: 6-bromo-5-nitrobenzo[b]thiophene-2-carboxylic acid

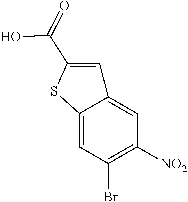 [SMILES: O=C(O)c1cc2cc([N+](=O)[O-])c(Br)cc2s1]

A mixture of methyl 6-bromo-5-nitrobenzo[b]thiophene-2-carboxylate (1.0 g, 3.17 mmol) and LiOH (762 mg, 31.74 mmol) in MeOH (3 mL), THF (3 ml) and H₂O (3 ml) was stirred at rt for 5 h. The mixture was adjusted to pH=5 by 1 N HCl and concentrated under vacuum. The mixture was filtered, and the filter cake was washed with water to give the desired product (600 mg, 63%). MS (ESI) m/e [M+1]⁺=302.

### Step 3: 6-bromo-5-nitrobenzo[b]thiophene

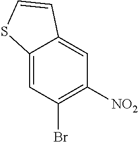 [SMILES: O=[N+]([O-])c1cc2ccsc2cc1Br]

A mixture of 6-bromo-5-nitrobenzo[b]thiophene-2-carboxylic acid (500 mg, 1.66 mmol) and LiCl (349 mg, 8.31 mmol) in DMSO (10 ml) was stirred at 190° C. for 2 h. The mixture was allowed to cool down to room temperature, diluted with water (50 ml) and extracted with ethyl acetate. The combined organic layers were washed with brine, dried over anhydrous Na₂SO₄, filtrated and concentrated under reduced pressure. The residue was purified by flash chromatography on silica gel to give the desired product (300 mg, 70%). MS (ESI) m/e [M+1]⁺=258.

### Step 4: 6-bromobenzo[b]thiophen-5-amine

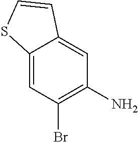 [SMILES: Nc1cc2ccsc2cc1Br]

A mixture of 6-bromo-5-nitrobenzo[b]thiophene (300 mg, 1.17 mmol) and Fe (654 mg, 11.67 mmol) in MeOH (5 ml) and saturated NH₄Cl (aq, 5 ml) was stirred at 60° C. for 30 min. The mixture was allowed to cool down to room temperature, diluted with water and extracted with ethyl acetate. The combined organic layers were washed with brine, dried over anhydrous Na₂SO₄, filtrated and concentrated under reduced pressure. The residue was purified by flash chromatography on silica gel to give the desired product (220 mg, 83%). MS (ESI) m/e [M+1]⁺=228.

### Step 5: methyl 5-aminobenzo[b]thiophene-6-carboxylate

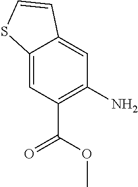

A mixture of 6-bromobenzo[b]thiophen-5-amine (300 mg, 1.17 mmol), Pd(dppf)Cl₂ (654 mg, 11.67 mmol) and TEA (654 mg, 11.67 mmol) in MeOH (10 mL) was stirred at 120° C. overnight under CO₂ atmosphere (20 atm). The mixture was allowed to cool down to room temperature, diluted with water and extracted with ethyl acetate. The combined organic layers were washed with brine, dried over anhydrous Na₂SO₄, filtrated and concentrated under reduced pressure. The residue was purified by flash chromatography on silica gel to give the desired product (100 mg, 55%). MS (ESI) m/e [M+1]⁺=208.

### Step 6: methyl 5-((1-(2,7-dimethyl-1-oxo-3-phenyl-1,2-dihydroisoquinolin-5-yl)ethyl)amino)benzo[b]thiophene-6-carboxylate

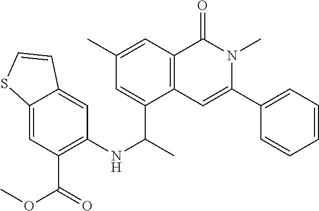 [SMILES: COC(=O)c1cc2sccc2cc1NC(C)c1cc(C)cc2c(=O)n(C)c(-c3ccccc3)cc12]

A mixture of 5-(1-bromoethyl)-2,7-dimethyl-3-phenylisoquinolin-1(2H)-one (50 mg, 0.14 mmol), methyl 5-aminobenzo[b]thiophene-6-carboxylate (29 mg, 0.14 mmol) and DIEA (54 mg, 0.42 mmol) in DMF (2 mL) was stirred at 90° C. for 1 h. The mixture was concentrated under vacuum. The residue was purified by Prep-TLC to give the desired product (30 mg, 44%). MS (ESI) m/e [M+1]⁺=483.

### Step 7: 5-((1-(2,7-dimethyl-1-oxo-3-phenyl-1,2-dihydroisoquinolin-5-yl)ethyl)amino)benzo[b]thiophene-6-carboxylic acid

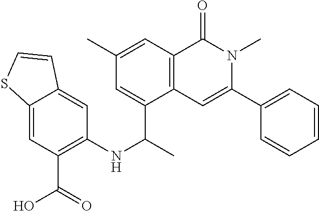 [SMILES: Cc1cc(C(C)Nc2cc3ccsc3cc2C(=O)O)c2cc(-c3ccccc3)n(C)c(=O)c2c1]

A mixture of methyl 5-((1-(2,7-dimethyl-1-oxo-3-phenyl-1,2-dihydroisoquinolin-5-yl)ethyl)amino)benzo[b]thiophene-6-carboxylate (30 mg, 0.06 mmol) in NaOH (1 mL, 4N), MeOH (1 mL) and THF (1 mL) was stirred at 50° C. for 3 h. The mixture was adjusted to PH 5 by 1 N HCl and concentrated under vacuum. The residue was purified by prep-HPLC to give the desired product (6.99 mg, 24%). ¹H NMR (400 MHz, DMSO-d₆) δ ppm 12.94 (brs, 1H), 8.44 (s, 1H), 8.31 (brs, 1H), 7.99 (s, 1H), 7.75 (d, J=8.0 Hz, 1H), 7.60-7.52 (m, 6H), 7.06 (d, J=5.4 Hz, 1H), 6.86 (s, 1H), 6.72 (s, 1H), 5.21-5.20 (m, 1H), 3.33 (s, 3H), 2.36 (s, 3H), 1.54 (d, J=8.0 Hz, 3H). MS (ESI) m/e [M+1]⁺=469.

### Example 48: 3-((1-(2,7-dimethyl-1-oxo-3-phenyl-1,2-dihydroisoquinolin-5-yl)ethyl)amino)benzo[b]thiophene-2-carboxylic acid

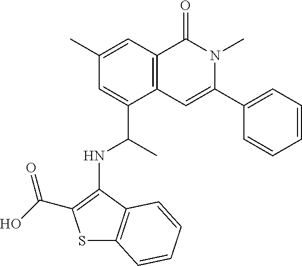 [SMILES: Cc1cc(C(C)Nc2c(C(=O)O)sc3ccccc23)c2cc(-c3ccccc3)n(C)c(=O)c2c1]

### Step 1: methyl 3-((1-(2,7-dimethyl-1-oxo-3-phenyl-1,2-dihydroisoquinolin-5-yl)ethyl)amino)benzo[b]thiophene-2-carboxylate

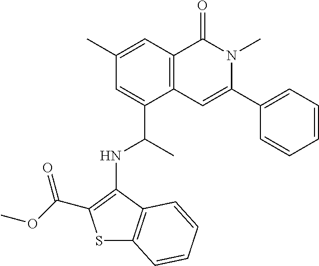 [SMILES: COC(=O)c1sc2ccccc2c1NC(C)c1cc(C)cc2c(=O)n(C)c(-c3ccccc3)cc12]

A solution of 5-(1-bromoethyl)-2,7-dimethyl-3-phenylisoquinolin-1(2H)-one (70 mg, 0.2 mmol), methyl 3-aminobenzo[b]thiophene-2-carboxylate (83 mg, 0.4 mmol) and TEA (101 mg, 1 mmol) in DMF (4 mL) was stirred at 90° C. for 2 h. The mixture was cooled to rt. The solvent was removed, and the crude residue was purified by Prep-TLC to give the desired product (45 mg, 47%). MS (ESI) m/e [M+H]⁺=483.

### Step 2: 3-((1-(2,7-dimethyl-1-oxo-3-phenyl-1,2-dihydroisoquinolin-5-yl)ethyl)amino)benzo[b]thiophene-2-carboxylic acid

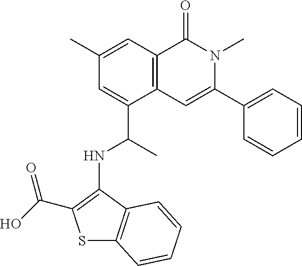 [SMILES: Cc1cc(C(C)Nc2c(C(=O)O)sc3ccccc23)c2cc(-c3ccccc3)n(C)c(=O)c2c1]

A solution of methyl 3-((1-(2,7-dimethyl-1-oxo-3-phenyl-1,2-dihydroisoquinolin-5-yl)ethyl)amino)benzo[b]thiophene-2-carboxylate (45 mg, 0.093 mmol) in NaOH (1N, 10 mL) and MeOH (10 mL) was stirred at 70° C. for 4 h. The solvent was removed, the residue was redissolved in H₂O (2 mL). A white solid was collected and the solid was purified on Prep-HPLC to give the desired product (21 mg, 48%). ¹H NMR (400 MHz, DMSO-d₆) δ ppm 8.41-8.15 (m, 1H), 7.91 (s, 1H), 7.82 (d, J=8.4 Hz, 1H), 7.76 (s, 1H), 7.68 (d, J=7.9 Hz, 1H), 7.51 (s, 5H), 7.23 (s, 1H), 7.10 (m, 1H), 6.79 (s, 1H), 5.74 (s, 1H), 3.28 (s, 3H), 2.37 (s, 3H), 1.49 (d, J=6.2 Hz, 3H). MS (ESI) m/e [M+H]⁺=469.

### Example 49: 2-((1-(2,7-dimethyl-1-oxo-3-(4-(trifluoromethyl)phenyl)-1,2-dihydroisoquinolin-5-yl)ethyl)amino)benzoic acid

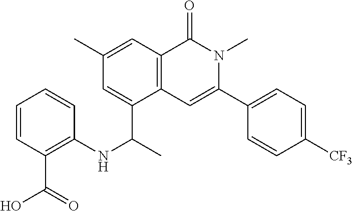 [SMILES: Cc1cc(C(C)Nc2ccccc2C(=O)O)c2cc(-c3ccc(C(F)(F)F)cc3)n(C)c(=O)c2c1]

The desired product (46 mg) was obtained following the similar procedure as Example 10. ¹H NMR (399 MHz, DMSO-d₆) δ ppm 12.75 (brs, 1H), 8.74 (s, 1H), 7.99 (s, 1H), 7.93-7.72 (m, 5H), 7.51 (s, 1H), 7.08 (s, 1H), 6.93 (s, 1H), 6.50-6.40 (m, 1H), 6.27 (d, J=8.4 Hz, 1H), 5.18 (s, 1H), 3.34 (s, 3H), 2.37 (s, 3H), 1.48 (d, J=6.5 Hz, 3H). MS (ESI) m/e [M+1]⁺=481.

### Example 50: 2-((1-(2,7-dimethyl-1-oxo-3-(3-(trifluoromethyl)phenyl)-1,2-dihydroisoquinolin-5-yl)ethyl)amino)benzoic acid

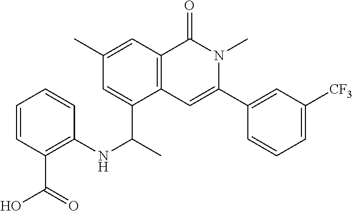 [SMILES: Cc1cc(C(C)Nc2ccccc2C(=O)O)c2cc(-c3cccc(C(F)(F)F)c3)n(C)c(=O)c2c1]

The desired product (42 mg) was obtained following the similar procedure as Example 10. ¹H NMR (399 MHz, DMSO-d₆) δ ppm 9.83 (s, 1H), 8.06-7.94 (m, 2H), 7.94-7.84 (m, 2H), 7.81-7.72 (m, 2H), 7.66-7.49 (m, 2H), 6.96-6.83 (m, 2H), 6.35 (m, 1H), 6.12 (d, J=8.1 Hz, 1H), 5.09 (s, 1H), 3.31 (s, 3H), 2.36 (s, 3H), 1.44 (d, J=6.5 Hz, 3H). MS (ESI) m/e [M+1]⁺=481.

### Example 51: 2-((1-(3-(4-(2-hydroxypropan-2-yl)phenyl)-2,7-dimethyl-1-oxo-1,2-dihydroisoquinolin-5-yl)ethyl)amino)benzoic acid

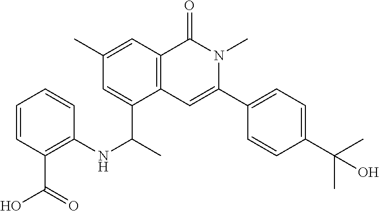 [SMILES: Cc1cc(C(C)Nc2ccccc2C(=O)O)c2cc(-c3ccc(C(C)(C)O)cc3)n(C)c(=O)c2c1]

### Step 1: methyl 2-((1-(3-(4-(2-hydroxypropan-2-yl)phenyl)-2,7-dimethyl-1-oxo-1,2-dihydroisoquinolin-5-yl)ethyl)amino)benzoate

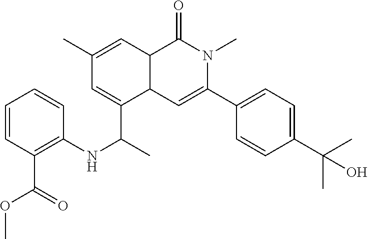 [SMILES: COC(=O)c1ccccc1NC(C)C1=CC(C)=CC2C(=O)N(C)C(c3ccc(C(C)(C)O)cc3)=CC12]

A mixture of methyl 2-((1-(3-chloro-2,7-dimethyl-1-oxo-1,2-dihydroisoquinolin-5-yl)ethyl)amino)benzoate (50 mg, 0.13 mmol), (4-(2-hydroxypropan-2-yl)phenyl)boronic acid (46.8 mg, 0.26 mmol), Pd(dppf)Cl₂ (9.5 mg, 0.013 mmol) and K₃PO₄ (55 mg, 0.26 mmol) in dioxane (8 ml) and H₂O (2 ml) was stirred at 90° C. under N₂ for 5 h. The solvent was removed, and the residue was purified by silica gel flash column to give the desired crude product (100 mg, crude). MS (ESI) m/e [M+H]⁺=485.

### Step 2: 2-((1-(3-(4-(2-hydroxypropan-2-yl)phenyl)-2,7-dimethyl-1-oxo-1,2-dihydroisoquinolin-5-yl)ethyl)amino)benzoic acid

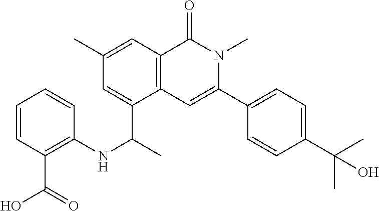 [SMILES: Cc1cc(C(C)Nc2ccccc2C(=O)O)c2cc(-c3ccc(C(C)(C)O)cc3)n(C)c(=O)c2c1]

A solution of methyl 2-((1-(3-(4-(2-hydroxypropan-2-yl)phenyl)-2,7-dimethyl-1-oxo-1,2-dihydroisoquinolin-5-yl)ethyl)amino)benzoate (100 mg, crude) in NaOH (1N, 5 mL) and MeOH (5 ml) was stirred at 60° C. for 2 h. The solvent was removed, the aqueous layer was acidified by HCl (2 N), extracted with EA. The organic layer was dried over Na₂SO₄, filtered and concentrated under reduced pressure. The residue was purified on Prep-HPLC to give the desired product (48 mg, 78% for two steps). ¹H NMR (400 MHz, DMSO-d₆) δ ppm 8.08 (s, 1H), 7.90 (d, J=7.9 Hz, 1H), 7.66 (d, J=8.3 Hz, 2H), 7.58 (s, 1H), 7.48 (d, J=8.4 Hz, 2H), 7.11 (dd, J=11.3, 4.3 Hz, 1H), 6.90 (s, 1H), 6.52 (t, J=7.5 Hz, 1H), 6.29 (d, J=8.5 Hz, 1H), 5.12 (m, 1H), 3.45 (s, 3H), 2.41 (s, 3H), 1.61-1.56 (m, 9H). MS (ESI) m/e [M−H]⁻=469.

### Example 52: 2-((1-(2,7-dimethyl-1-oxo-3-(4-(prop-1-yn-1-yl)phenyl)-1,2-dihydroisoquinolin-5-yl)ethyl)amino)benzoic acid

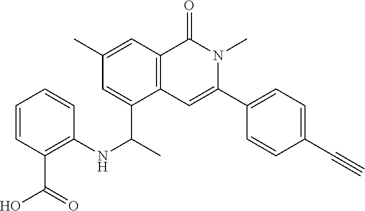 [SMILES: C#Cc1ccc(-c2cc3c(C(C)Nc4ccccc4C(=O)O)cc(C)cc3c(=O)n2C)cc1]

The desired product (13.5 mg) was obtained following the similar procedure as Example 10. ¹H NMR (400 MHz, DMSO-d₆) δ ppm 12.96 (brs, 1H), 8.52 (s, 1H), 7.98 (s, 1H), 7.80 (d, J=7.7 Hz, 1H), 7.61-7.44 (m, 5H), 7.18-7.08 (m, 1H), 6.84 (s, 1H), 6.59-6.39 (m, 1H), 6.31 (d, J=8.4 Hz, 1H), 5.27-5.13 (m, 1H), 3.32 (s, 3H), 2.36 (s, 3H), 2.08 (s, 3H), 1.49 (d, J=6.4 Hz, 3H). MS (ESI) m/e [M+1]⁺=451.

### Example 53: 2-((1-(3-(1a,6b-dihydro-1H-cyclopropa[b]benzofuran-5-yl)-2,7-dimethyl-1-oxo-1,2-dihydroisoquinolin-5-yl)ethyl)amino)benzoic acid

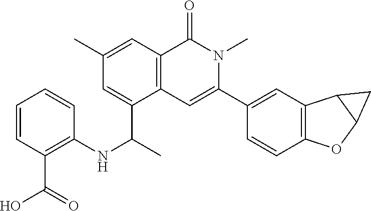 [SMILES: Cc1cc(C(C)Nc2ccccc2C(=O)O)c2cc(-c3ccc4c(c3)C3CC3O4)n(C)c(=O)c2c1]

The desired product (12.6 mg) was obtained following the similar procedure as Example 10. ¹H NMR (400 MHz, DMSO-d₆) δ ppm 12.68 (brs, 1H), 8.32-8.23 (m, 1H), 7.88 (s, 1H), 7.73 (d, J=8.0 Hz, 1H), 7.51 (s, 1H), 7.39 (s, 1H), 7.20 (d, J=8.0 Hz, 1H), 7.15-7.05 (m, 1H), 6.88 (d, J=8.0 Hz, 1H), 6.71 (s, 1H), 6.44 (t, J=7.2 Hz, 1H), 6.26 (d, J=8.0 Hz, 1H), 5.18-5.05 (m, 1H), 5.00-4.89 (m, 1H), 3.22 (s, 3H), 2.73-2.63 (m, 1H), 2.47-2.38 (m, 1H), 2.29 (s, 3H), 1.43 (d, J=6.8 Hz, 3H), 1.11-1.01 (m, 1H). MS (ESI) m/e [M+1]⁺=467.

### Example 54: 3-((1-(3-(4-fluorophenyl)-2,7-dimethyl-1-oxo-1,2-dihydroisoquinolin-5-yl)ethyl)amino)isonicotinic acid

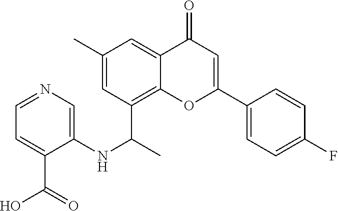 [SMILES: Cc1cc(C(C)Nc2cnccc2C(=O)O)c2oc(-c3ccc(F)cc3)cc(=O)c2c1]

### Step 1: methyl 3-((1-(3-(4-fluorophenyl)-2,7-dimethyl-1-oxo-1,2-dihydroisoquinolin-5-yl)ethyl)amino)isonicotinate

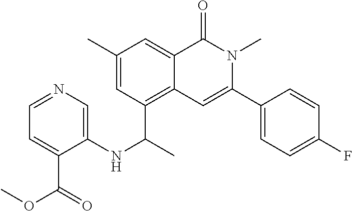 [SMILES: COC(=O)c1ccncc1NC(C)c1cc(C)cc2c(=O)n(C)c(-c3ccc(F)cc3)cc12]

A mixture of 5-(1-bromoethyl)-3-(4-fluorophenyl)-2,7-dimethylisoquinolin-1(2H)-one (60 mg, 0.16 mmol), methyl 3-aminoisonicotinate (37 mg, 0.24 mmol), DIEA (62 mg, 0.48 mmol) in DMF (5 ml) was stirred at 90° C. for 1 h. The mixture was concentrated under vacuum. The residue was purified by Prep-TLC to give the desired product (20 mg, 29%). MS (ESI) m/e [M+1]⁺=446.

### Step 2: 3-((1-(3-(4-fluorophenyl)-2,7-dimethyl-1-oxo-1,2-dihydroisoquinolin-5-yl)ethyl)amino)isonicotinic acid

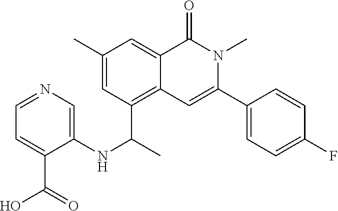 [SMILES: Cc1cc(C(C)Nc2cnccc2C(=O)O)c2cc(-c3ccc(F)cc3)n(C)c(=O)c2c1]

To a solution of methyl 3-((1-(3-(4-fluorophenyl)-2,7-dimethyl-1-oxo-1,2-dihydroisoquinolin-5-yl)ethyl)amino)isonicotinate_(20 mg, 0.04 mmol) in MeOH (1 ml) and THF (0.5 ml) was added 4 N NaOH (0.2 mL). The reaction mixture was stirred at rt for 1 h. The mixture was adjusted to PH 5 by 1 N HCl and concentrated under vacuum. The residue was purified by prep-HPLC to give the desired product (2.9 mg, 15%). ¹H NMR (400 MHz, DMSO-d₆) δ ppm 8.43-8.23 (m, 1H), 8.00 (s, 1H), 7.75-7.72 (m, 2H), 7.68-7.65 (m, 2H), 7.57 (d, J=5.0 Hz, 1H), 7.51 (s, 1H), 7.39-7.34 (m, 2H), 6.87 (s, 1H), 5.40-5.30 (m, 1H), 3.56 (s, 3H), 2.37 (s, 3H), 1.51 (d, J=8.0 Hz, 3H). MS (ESI) m/e [M+1]⁺=432.

### Example 55: 2-((1-(3-(6-(2-hydroxypropan-2-yl)pyridin-3-yl)-2,7-dimethyl-1-oxo-1,2-dihydroisoquinolin-5-yl)ethyl)amino)benzoic acid

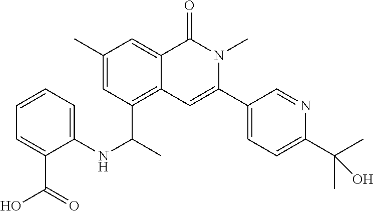 [SMILES: Cc1cc(C(C)Nc2ccccc2C(=O)O)c2cc(-c3ccc(C(C)(C)O)nc3)n(C)c(=O)c2c1]

The desired product (70 mg) was obtained following the similar procedure as Example 10. ¹H NMR (400 MHz, DMSO-d₆) δ ppm 8.77-8.67 (m, 1H), 8.53 (s, 1H), 8.10-8.01 (m, 1H), 7.99 (s, 1H), 7.84-7.74 (m, 2H), 7.49 (d, J=1.3 Hz, 1H), 7.19-7.09 (m, 1H), 6.95 (s, 1H), 6.49 (m, 1H), 6.32 (d, J=8.5 Hz, 1H), 5.43-5.30 (m, 1H), 5.29-5.16 (m, 1H), 3.35 (s, 3H), 2.37 (s, 3H), 1.50 (m, 9H). MS (ESI) m/e [M+1]⁺=472.

### Example 56: (R)-2-((1-(3-(6-(2-hydroxypropan-2-yl)pyridin-3-yl)-2,7-dimethyl-1-oxo-1,2-dihydroisoquinolin-5-yl)ethyl)amino)benzoic acid

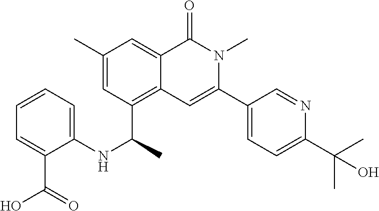 [SMILES: Cc1cc([C@@H](C)Nc2ccccc2C(=O)O)c2cc(-c3ccc(C(C)(C)O)nc3)n(C)c(=O)c2c1]

Chiral separation of racemic 2-((1-(3-(6-(2-hydroxypropan-2-yl)pyridin-3-yl)-2,7-dimethyl-1-oxo-1,2-dihydroisoquinolin-5-yl)ethyl)amino)benzoic acid (68 mg) by SFC (Column Name: YMC Cellulose-C 20*250 mm, 5 m; Co-Solvent: 38% MeOH; Temperature (° C.): 35; Flow (mL/min): 40 mL/min; Back Pressure: 100 bar) to give (R)-2-((1-(3-(6-(2-hydroxypropan-2-yl)pyridin-3-yl)-2,7-dimethyl-1-oxo-1,2-dihydroisoquinolin-5-yl)ethyl)amino)benzoic acid (23.7 mg), Rt: 7.15 min. ¹H NMR (400 MHz, DMSO-d₆) δ ppm 8.77-8.67 (m, 1H), 8.53 (s, 1H), 8.10-8.01 (m, 1H), 7.99 (s, 1H), 7.84-7.74 (m, 2H), 7.49 (d, J=1.3 Hz, 1H), 7.19-7.09 (m, 1H), 6.95 (s, 1H), 6.49 (m, 1H), 6.32 (d, J=8.5 Hz, 1H), 5.43-5.30 (m, 1H), 5.29-5.16 (m, 1H), 3.35 (s, 3H), 2.37 (s, 3H), 1.50 (m, 9H). MS (ESI) m/e [M+1]⁺=472.

### Example 57: (S)-2-((1-(3-(6-(2-hydroxypropan-2-yl)pyridin-3-yl)-2,7-dimethyl-1-oxo-1,2-dihydroisoquinolin-5-yl)ethyl)amino)benzoic acid

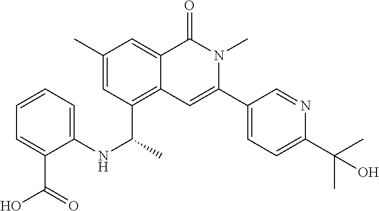 [SMILES: Cc1cc([C@H](C)Nc2ccccc2C(=O)O)c2cc(-c3ccc(C(C)(C)O)nc3)n(C)c(=O)c2c1]

Chiral separation of racemic 2-((1-(3-(6-(2-hydroxypropan-2-yl)pyridin-3-yl)-2,7-dimethyl-1-oxo-1,2-dihydroisoquinolin-5-yl)ethyl)amino)benzoic acid (68 mg) by SFC (Column Name: YMC Cellulose-C 20*250 mm, 5 m; Co-Solvent: 38% MeOH; Temperature (° C.): 35; Flow (mL/min): 40 mL/min; Back Pressure: 100 bar) to give (S)-2-((1-(3-(6-(2-hydroxypropan-2-yl)pyridin-3-yl)-2,7-dimethyl-1-oxo-1,2-dihydroisoquinolin-5-yl)ethyl)amino)benzoic acid (21.4 mg), Rt: 5.12 min. 11H NMR (400 MHz, DMSO-d₆) δ ppm 8.77-8.67 (m, 1H), 8.53 (s, 1H), 8.10-8.01 (m, 1H), 7.99 (s, 1H), 7.84-7.74 (m, 2H), 7.49 (d, J=1.3 Hz, 1H), 7.19-7.09 (m, 1H), 6.95 (s, 1H), 6.49 (m, 1H), 6.32 (d, J=8.5 Hz, 1H), 5.43-5.30 (m, 1H), 5.29-5.16 (m, 1H), 3.35 (s, 3H), 2.37 (s, 3H), 1.50 (m, 9H). MS (ESI) m/e [M+1]⁺=472.

### Example 58: 2-((1-(3-(5-fluoro-6-methoxypyridin-3-yl)-2,7-dimethyl-1-oxo-1,2-dihydroisoquinolin-5-yl)ethyl)amino)benzoic acid

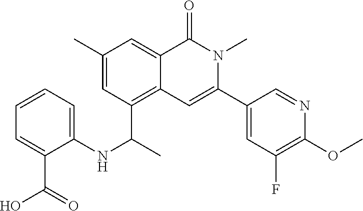 [SMILES: COc1ncc(-c2cc3c(C(C)Nc4ccccc4C(=O)O)cc(C)cc3c(=O)n2C)cc1F]

The desired product (49 mg) was obtained following the similar procedure as Example 10. ¹H NMR (400 MHz, DMSO-d₆) δ ppm 12.76 (brs, 1H), 8.41 (s, 1H), 8.25 (d, J=1.7 Hz, 1H), 8.08 (dd, J=11.2, 1.7 Hz, 1H), 7.99 (s, 1H), 7.86-7.73 (m, 1H), 7.48 (s, 1H), 7.21-7.10 (m, 1H), 6.95 (s, 1H), 6.55-6.45 (m, 1H), 6.33 (d, J=8.5 Hz, 1H), 5.28-5.18 (m, 1H), 4.03 (s, 3H), 3.37 (s, 3H), 2.37 (s, 3H), 1.50 (d, J=6.5 Hz, 3H). MS (ESI) m/e [M+1]⁺=462.

### Example 59: (R)-2-((1-(3-(5-fluoro-6-methoxypyridin-3-yl)-2,7-dimethyl-1-oxo-1,2-dihydroisoquinolin-5-yl)ethyl)amino)benzoic acid

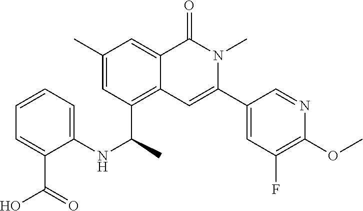 [SMILES: COc1ncc(-c2cc3c([C@@H](C)Nc4ccccc4C(=O)O)cc(C)cc3c(=O)n2C)cc1F]

Chiral separation of racemic 2-((1-(3-(5-fluoro-6-methoxypyridin-3-yl)-2,7-dimethyl-1-oxo-1,2-dihydroisoquinolin-5-yl)ethyl)amino)benzoic acid (47 mg) by SFC (Column Name: YMC Cellulose-C 20*250 mm, 5 m; Co-Solvent: 35% MeOH; Temperature (° C.): 35; Flow (mL/min): 40 ml/min; Back Pressure: 100 bar) to give (R)-2-((1-(3-(5-fluoro-6-methoxypyridin-3-yl)-2,7-dimethyl-1-oxo-1,2-dihydroisoquinolin-5-yl)ethyl)amino)benzoic acid (18.1 mg), Rt: 7.55 min. ¹H NMR (400 MHz, DMSO-d₆) δ ppm 12.76 (s, 1H), 8.41 (s, 1H), 8.25 (d, J=1.7 Hz, 1H), 8.08 (dd, J=11.2, 1.7 Hz, 1H), 7.99 (s, 1H), 7.86-7.73 (m, 1H), 7.48 (s, 1H), 7.21-7.10 (m, 1H), 6.95 (s, 1H), 6.51 (m, 1H), 6.33 (d, J=8.5 Hz, 1H), 5.28-5.18 (m, 1H), 4.03 (s, 3H), 3.37 (s, 3H), 2.37 (s, 3H), 1.50 (d, J=6.5 Hz, 3H). MS (ESI) m/e [M+1]⁺=462.

### Example 60: (S)-2-((1-(3-(5-fluoro-6-methoxypyridin-3-yl)-2,7-dimethyl-1-oxo-1,2-dihydroisoquinolin-5-yl)ethyl)amino)benzoic acid

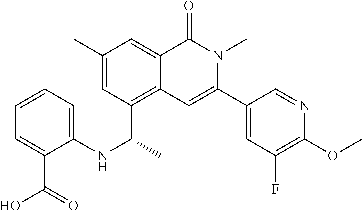 [SMILES: COc1ncc(-c2cc3c([C@H](C)Nc4ccccc4C(=O)O)cc(C)cc3c(=O)n2C)cc1F]

Chiral separation of racemic 2-((1-(3-(5-fluoro-6-methoxypyridin-3-yl)-2,7-dimethyl-1-oxo-1,2-dihydroisoquinolin-5-yl)ethyl)amino)benzoic acid (47 mg) by SFC (Column Name: YMC Cellulose-C 20*250 mm, 5 m; Co-Solvent: 35% MeOH; Temperature (° C.): 35; Flow (mL/min): 40 ml/min; Back Pressure: 100 bar) to give (S)-2-((1-(3-(5-fluoro-6-methoxypyridin-3-yl)-2,7-dimethyl-1-oxo-1,2-dihydroisoquinolin-5-yl)ethyl)amino)benzoic acid (16.8 mg), Rt: 5.73 min. ¹H NMR (400 MHz, DMSO-d₆) δ ppm 12.76 (s, 1H), 8.41 (s, 1H), 8.25 (d, J=1.7 Hz, 1H), 8.08 (dd, J=11.2, 1.7 Hz, 1H), 7.99 (s, 1H), 7.86-7.73 (m, 1H), 7.48 (s, 1H), 7.21-7.10 (m, 1H), 6.95 (s, 1H), 6.51 (m, 1H), 6.33 (d, J=8.5 Hz, 1H), 5.28-5.18 (m, 1H), 4.03 (s, 3H), 3.37 (s, 3H), 2.37 (s, 3H), 1.50 (d, J=6.5 Hz, 3H). MS (ESI) m/e [M+1]⁺=462.

### Example 61: 2-((1-(2,7-dimethyl-1-oxo-3-(pyrimidin-5-yl)-1,2-dihydroisoquinolin-5-yl)ethyl)amino)benzoic acid

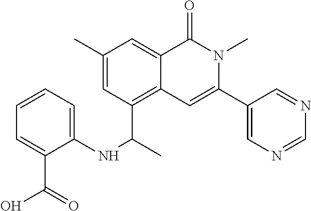 [SMILES: Cc1cc(C(C)Nc2ccccc2C(=O)O)c2cc(-c3cncnc3)n(C)c(=O)c2c1]

The desired product (35 mg) was obtained following the similar procedure as Example 10. ¹H NMR (400 MHz, DMSO-d₆) δ ppm 12.70 (brs, 1H), 9.32 (s, 1H), 9.12 (s, 2H), 8.47-8.34 (m, 1H), 8.01 (s, 1H), 7.79 (dd, J=7.9, 1.4 Hz, 1H), 7.53-7.47 (m, 1H), 7.21-7.12 (m, 1H), 7.08 (s, 1H), 6.51 (m, 1H), 6.34 (d, J=8.5 Hz, 1H), 5.32-5.20 (m, 1H), 3.39 (s, 3H), 2.38 (s, 3H), 1.50 (d, J=6.5 Hz, 3H). MS (ESI) m/e [M+1]⁺=415.

### Example 62: 2-((1-(3-(4-(1-hydroxycyclopropyl)phenyl)-2,7-dimethyl-1-oxo-1,2-dihydroisoquinolin-5-yl)ethyl)amino)benzoic acid

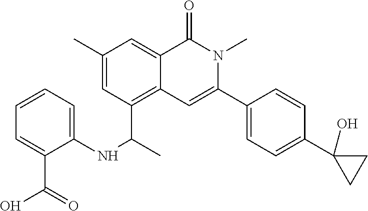 [SMILES: Cc1cc(C(C)Nc2ccccc2C(=O)O)c2cc(-c3ccc(C4(O)CC4)cc3)n(C)c(=O)c2c1]

The desired product (15 mg) was obtained following the similar procedure as Example 10. ¹H NMR (400 MHz, DMSO-d₆) δ ppm 12.81 (brs, 1H), 8.44 (s, 1H), 8.13-8.05 (m, 2H), 8.00 (s, 1H), 7.84-7.70 (m, 3H), 7.50 (s, 1H), 7.15 (m, 1H), 6.89 (s, 1H), 6.51 (t, J=7.5 Hz, 1H), 6.33 (d, J=8.4 Hz, 1H), 5.28-5.13 (m, 1H), 3.33 (s, 3H), 3.18-3.05 (m, 2H), 2.37 (s, 3H), 1.50 (d, J=6.5 Hz, 3H), 1.12 (t, J=7.1 Hz, 2H). MS (ESI) m/e [M+1]⁺=469.

### Example 63: 2-((1-(2-(cyanomethyl)-3-(4-fluorophenyl)-7-methyl-1-oxo-1,2-dihydroisoquinolin-5-yl)ethyl)amino)benzoic acid

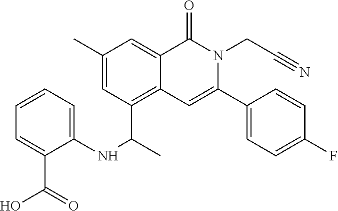 [SMILES: Cc1cc(C(C)Nc2ccccc2C(=O)O)c2cc(-c3ccc(F)cc3)n(CC#N)c(=O)c2c1]

### Step 1: 2-(5-acetyl-3-chloro-7-methyl-1-oxoisoquinolin-2(1H)-yl)acetonitrile

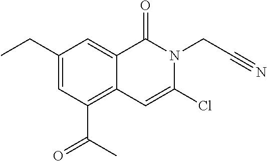 [SMILES: CCc1cc(C(C)=O)c2cc(Cl)n(CC#N)c(=O)c2c1]

To a solution of 5-acetyl-3-chloro-7-methylisoquinolin-1(2H)-one (350 mg, 1.49 mmol) and K₂CO₃ (616 mg, 4.46 mmol) in DMF (15 ml) was added 2-bromoacetonitrile (267 mg, 2.23 mmol) at 0° C. The reaction mixture was stirred for 1 h at room temperature. The mixture was poured into H₂O and extracted with EA. The combined organic solution was washed with brine, dried over anhydrous Na₂SO₄, filtered, and concentrated under vacuum. The residue was purified by column chromatography on silica gel to give the product (150 mg, 37%). MS (ESI) m/e [M+1]⁺=275.

### Step 2: 2-(5-acetyl-3-(4-fluorophenyl)-7-methyl-1-oxoisoquinolin-2(1H)-yl)acetonitrile

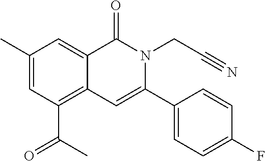 [SMILES: CC(=O)c1cc(C)cc2c(=O)n(CC#N)c(-c3ccc(F)cc3)cc12]

To a solution of 2-(5-acetyl-3-chloro-7-methyl-1-oxoisoquinolin-2(1H)-yl)acetonitrile (150 mg, 0.55 mmol), (4-fluorophenyl)boronic acid (115 mg, 0.82 mmol) and K₃PO₄ (350 mg, 1.65 mmol) in dioxane (10 ml) and H₂O (1 ml) was add Pd(PPh₃)₄ (31 mg, 0.03 mmol) under N₂. The resulting solution was stirred overnight at 100° C. The mixture was poured into H₂O and extracted with EtOAc. The organic layer was dried over Na₂SO₄, filtered and concentrated under vacuum. The residue was purified by flash chromatography on silica gel to give the product (175 mg, 93%). MS (ESI) m/e [M+1]⁺=335.

### Step 3: 2-(3-(4-fluorophenyl)-5-(1-hydroxyethyl)-7-methyl-1-oxoisoquinolin-2(1H)-yl)acetonitrile

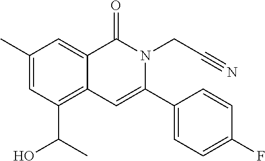 [SMILES: Cc1cc(C(C)O)c2cc(-c3ccc(F)cc3)n(CC#N)c(=O)c2c1]

A solution of 2-(5-acetyl-3-(4-fluorophenyl)-7-methyl-1-oxoisoquinolin-2(1H)-yl)acetonitrile (100 mg, 0.30 mmol) in DCM (10 ml) and MeOH (10 ml) was treated with NaBH₄ (12 mg, 0.33 mmol) in portions at 0° C. The resulting solution was stirred at room temperature for 1 h. The mixture was diluted with H₂O (10 ml) and extracted with DCM (20 mL×2). The combined organic phase were washed with brine (20 mL), dried over anhydrous Na₂SO₄, filtered, and concentrated in vacuum. The residue was purified by flash chromatography on silica gel to give the product (99 mg, 95%). MS (ESI) m/e [M+1]⁺=337.

### Step 4: 2-((1-(2-(cyanomethyl)-3-(4-fluorophenyl)-7-methyl-1-oxo-1,2-dihydroisoquinolin-5-yl)ethyl)amino)benzoic acid

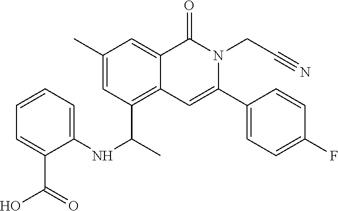 [SMILES: Cc1cc(C(C)Nc2ccccc2C(=O)O)c2cc(-c3ccc(F)cc3)n(CC#N)c(=O)c2c1]

A solution of 2-(3-(4-fluorophenyl)-5-(1-hydroxyethyl)-7-methyl-1-oxoisoquinolin-2(1H)-yl)acetonitrile (99 mg, 0.30 mmol), PPh₃ (117 mg, 0.45 mmol) and 2H-benzo[d][1,3]oxazine-2,4(1H)-dione (63 mg, 0.39 mmol) in THF (10 ml) was added DIAD (91 mg, 0.45 mmol) at 0° C. After stirring for 1 h at 0° C., 1 mL of NaOH (1 M in water) was added to the above mixture. The resulting solution was stirred for 1 h at room temperature, then concentrated under reduced pressure. The residue was re-dissolved in H₂O and acidized to pH 5˜6 with HCl (2 M in water). The mixture was filtered, and the filter cake was purified by Prep-HPLC to give desired product (17 mg, 13%). ¹H NMR (400 MHz, DMSO-d₆) δ ppm 12.78 (brs, 1H), 8.47-8.28 (m, 1H), 8.01 (s, 1H), 7.80 (dd, J=7.9, 1.5 Hz, 1H), 7.72-7.59 (m, 2H), 7.55 (d, J=1.3 Hz, 1H), 7.50-7.40 (m, 2H), 7.22-7.09 (m, 1H), 6.97 (s, 1H), 6.55-6.45 (m, 1H), 6.33 (d, J=8.5 Hz, 1H), 5.30-5.18 (m, 1H), 4.79 (s, 2H), 2.38 (s, 3H), 1.50 (d, J=6.5 Hz, 3H). MS (ESI) m/e [M+1]⁺=456.

### Example 64: (R)-2-((1-(2-(cyanomethyl)-3-(4-fluorophenyl)-7-methyl-1-oxo-1,2-dihydroisoquinolin-5-yl)ethyl)amino)benzoic acid

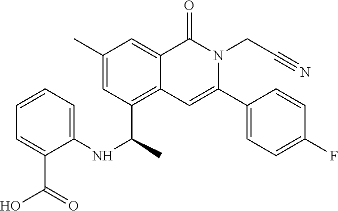 [SMILES: Cc1cc([C@@H](C)Nc2ccccc2C(=O)O)c2cc(-c3ccc(F)cc3)n(CC#N)c(=O)c2c1]

Chiral separation of racemic 2-((1-(2-(cyanomethyl)-3-(4-fluorophenyl)-7-methyl-1-oxo-1,2-dihydroisoquinolin-5-yl)ethyl)amino)benzoic acid (20 mg) by SFC (Column Name: YMC Cellulose-C 20*250 mm, 5 μm; Co-Solvent: 32% MeOH; Temperature (° C.): 35; Flow (mL/min): 40 mL/min; Back Pressure: 100 bar) to give (R)-2-((1-(2-(cyanomethyl)-3-(4-fluorophenyl)-7-methyl-1-oxo-1,2-dihydroisoquinolin-5-yl)ethyl)amino)benzoic acid (8.8 mg), Rt: 7.23 min. 11H NMR (400 MHz, DMSO-d₆) δ ppm 12.78 (s, 1H), 8.47-8.28 (m, 1H), 8.01 (s, 1H), 7.80 (dd, J=7.9, 1.5 Hz, 1H), 7.72-7.59 (m, 2H), 7.55 (d, J=1.3 Hz, 1H), 7.44 (m, 2H), 7.22-7.09 (m, 1H), 6.97 (s, 1H), 6.51 (m, 1H), 6.33 (d, J=8.5 Hz, 1H), 5.30-5.18 (m, 1H), 4.79 (s, 2H), 2.38 (s, 3H), 1.50 (d, J=6.5 Hz, 3H). MS (ESI) m/e [M+1]⁺=456.

### Example 65: (S)-2-((1-(2-(cyanomethyl)-3-(4-fluorophenyl)-7-methyl-1-oxo-1,2-dihydroisoquinolin-5-yl)ethyl)amino)benzoic acid

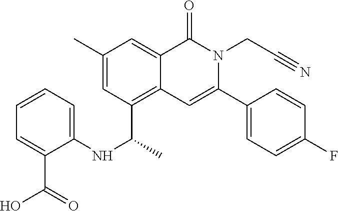 [SMILES: Cc1cc([C@H](C)Nc2ccccc2C(=O)O)c2cc(-c3ccc(F)cc3)n(CC#N)c(=O)c2c1]

Chiral separation of racemic 2-((1-(2-(cyanomethyl)-3-(4-fluorophenyl)-7-methyl-1-oxo-1,2-dihydroisoquinolin-5-yl)ethyl)amino)benzoic acid (20 mg) by SFC (Column Name: YMC Cellulose-C 20*250 mm, 5 μm; Co-Solvent: 32% MeOH; Temperature (° C.): 35; Flow (mL/min): 40 mL/min; Back Pressure: 100 bar) to give (S)-2-((1-(2-(cyanomethyl)-3-(4-fluorophenyl)-7-methyl-1-oxo-1,2-dihydroisoquinolin-5-yl)ethyl)amino)benzoic acid (8.1 mg), Rt: 5.43 min. ¹H NMR (400 MHz, DMSO-d₆) δ ppm 12.78 (s, 1H), 8.47-8.28 (m, 1H), 8.01 (s, 1H), 7.80 (dd, J=7.9, 1.5 Hz, 1H), 7.72-7.59 (m, 2H), 7.55 (d, J=1.3 Hz, 1H), 7.44 (m, 2H), 7.22-7.09 (m, 1H), 6.97 (s, 1H), 6.51 (m, 1H), 6.33 (d, J=8.5 Hz, 1H), 5.30-5.18 (m, 1H), 4.79 (s, 2H), 2.38 (s, 3H), 1.50 (d, J=6.5 Hz, 3H). MS (ESI) m/e [M+1]⁺=456.

### Example 66: 2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid

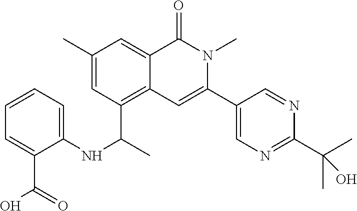 [SMILES: Cc1cc(C(C)Nc2ccccc2C(=O)O)c2cc(-c3cnc(C(C)(C)O)nc3)n(C)c(=O)c2c1]

### Step 1: 2-(5-(4,4,5,5-tetramethyl-1,3,2-dioxaborolan-2-yl)pyrimidin-2-yl)propan-2-ol

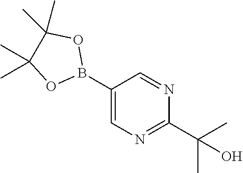 [SMILES: CC(C)(O)c1ncc(B2OC(C)(C)C(C)(C)O2)cn1]

To a solution of 2-(5-bromopyrimidin-2-yl)propan-2-ol (100 mg, 0.46 mmol), bis(pinacolato)diboron (129 mg, 0.51 mmol) and KOAc (135 mg, 1.38 mmol) in dioxane (30 ml) was added Pd(dppf)Cl2·DCM (290 mg, 0.40 mmol) under N₂. The resulting solution was stirred at 90° C. for 12 h. The solution was filtered, and concentrated under vacuum. The residue was purified by flash chromatography on silica gel eluted to give the product (95 mg, 78%). MS (ESI) m/e [M+1]⁺=265.

### Step 2: methyl 2-((1-(3-(2-(2-hydroxypropan-2-yl)pyrimidin-5-yl)-2,7-dimethyl-1-oxo-1,2-dihydroisoquinolin-5-yl)ethyl)amino)benzoate

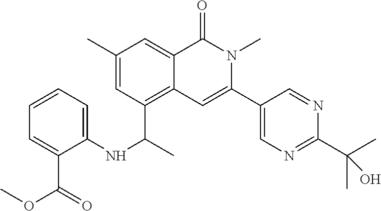 [SMILES: COC(=O)c1ccccc1NC(C)c1cc(C)cc2c(=O)n(C)c(-c3cnc(C(C)(C)O)nc3)cc12]

To a solution of methyl 2-((1-(3-(2-(2-hydroxypropan-2-yl)pyrimidin-5-yl)-2,7-dimethyl-1-oxo-1,2-dihydroisoquinolin-5-yl)ethyl)amino)benzoate (70 mg, 0.18 mmol) and 2-(5-(4,4,5,5-tetramethyl-1,3,2-dioxaborolan-2-yl)pyrimidin-2-yl)propan-2-ol (95 mg, 0.36 mmol) and K₃PO₄ (116 mg, 0.55 mmol) in dioxane (8 ml) and H₂O (1 ml) was added Pd(PPh₃)₄ (21 mg, 0.02 mmol) under N₂. The resulting solution was stirred at 100° C. for 15 h. The mixture was poured into H₂O and extracted with EtOAc. The combined organic extracts were washed with brine, dried over anhydrous Na₂SO₄, filtered, and concentrated under vacuum. The residue was purified by flash chromatography on silica gel to give the product (80 mg, 89%). MS (ESI) m/e [M+1]⁺=487.

### Step 3: 2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid

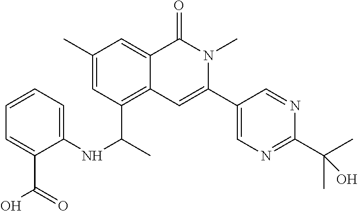 [SMILES: Cc1cc(C(C)Nc2ccccc2C(=O)O)c2cc(-c3cnc(C(C)(C)O)nc3)n(C)c(=O)c2c1]

To a solution of methyl 2-((1-(3-(2-(2-hydroxypropan-2-yl)pyrimidin-5-yl)-2,7-dimethyl-1-oxo-1,2-dihydroisoquinolin-5-yl)ethyl)amino)benzoate (80 mg, 0.16 mmol) in methanol (4 mL), H₂O (2 mL) and THF (4 ml) was added 1N NaOH (0.5 mL). The resulting solution was stirred for 7 h at 50° C. The solution was concentrated under vacuum. The residue was redissolved in H₂O and acidized to pH 5˜6 with HCl (2 M in water). The mixture was filtered, and the filter cake was purified by Prep-HPLC to give desired product (24 mg, 25%). ¹H NMR (400 MHz, DMSO-d₆) δ ppm 12.80 (brs, 1H), 9.07 (s, 2H), 8.51-8.36 (m, 1H), 8.01 (s, 1H), 7.84-7.75 (m, 1H), 7.49 (s, 1H), 7.22-7.11 (m, 1H), 7.09 (s, 1H), 6.55-6.45 (m, 1H), 6.33 (d, J=8.5 Hz, 1H), 5.34-5.25 (m, 1H), 5.20 (s, 1H), 3.39 (s, 3H), 2.37 (s, 3H), 1.56 (s, 6H), 1.50 (d, J=6.5 Hz, 3H). MS (ESI) m/e [M+1]⁺=473.

### Example 67: (R)-2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid

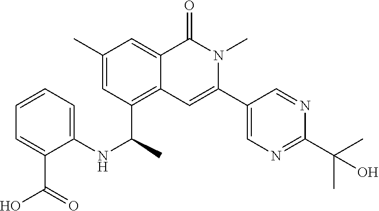 [SMILES: Cc1cc([C@@H](C)Nc2ccccc2C(=O)O)c2cc(-c3cnc(C(C)(C)O)nc3)n(C)c(=O)c2c1]

Chiral separation of racemic 2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid (45 mg) by SFC (Column Name: YMC Cellulose-C 20*250 mm, 5 m; Co-Solvent: 40% MeOH; Temperature (° C.): 35; Flow (ml/min): 40 mL/min; Back Pressure: 100 bar) to give (R)-2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid (20.3 mg), Rt: 9.62 min. ¹H NMR (400 MHz, DMSO-d₆) δ ppm 12.80 (s, 1H), 9.07 (s, 2H), 8.51-8.36 (m, 1H), 8.01 (s, 1H), 7.84-7.75 (m, 1H), 7.49 (s, 1H), 7.22-7.11 (m, 1H), 7.09 (s, 1H), 6.51 (m, 1H), 6.33 (d, J=8.5 Hz, 1H), 5.34-5.25 (m, 1H), 5.20 (s, 1H), 3.39 (s, 3H), 2.37 (s, 3H), 1.56 (s, 6H), 1.50 (d, J=6.5 Hz, 3H). MS (ESI) m/e [M+1]⁺=473.

### Example 68: (S)-2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid

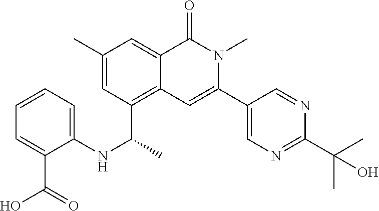 [SMILES: Cc1cc([C@H](C)Nc2ccccc2C(=O)O)c2cc(-c3cnc(C(C)(C)O)nc3)n(C)c(=O)c2c1]

Chiral separation of racemic 2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid (45 mg) by SFC (Column Name: YMC Cellulose-C 20*250 mm, 5 m; Co-Solvent: 40% MeOH; Temperature (° C.): 35; Flow (ml/min): 40 mL/min; Back Pressure: 100 bar) to give (S)-2-((1-(3-(2-(2-hydroxypropan-2-yl)pyrimidin-5-yl)-2,7-dimethyl-1-oxo-1,2-dihydroisoquinolin-5-yl)ethyl)amino)benzoic acid (21.0 mg), Rt: 5.97 min. 11H NMR (400 MHz, DMSO-d₆) δ ppm 12.80 (s, 1H), 9.07 (s, 2H), 8.51-8.36 (m, 1H), 8.01 (s, 1H), 7.84-7.75 (m, 1H), 7.49 (s, 1H), 7.22-7.11 (m, 1H), 7.09 (s, 1H), 6.51 (m, 1H), 6.33 (d, J=8.5 Hz, 1H), 5.34-5.25 (m, 1H), 5.20 (s, 1H), 3.39 (s, 3H), 2.37 (s, 3H), 1.56 (s, 6H), 1.50 (d, J=6.5 Hz, 3H). MS (ESI) m/e [M+1]⁺=473.

### Example 69: 2-((1-(3-(4-fluorophenyl)-7-methyl-1-oxo-2-(tetrahydrofuran-3-yl)-1,2-dihydroisoquinolin-5-yl)ethyl)amino)benzoic acid

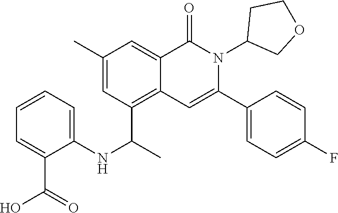 [SMILES: Cc1cc(C(C)Nc2ccccc2C(=O)O)c2cc(-c3ccc(F)cc3)n(C3CCOC3)c(=O)c2c1]

The desired product (65 mg) was obtained following the similar procedure as Example 63. ¹H NMR (400 MHz, DMSO-d₆) δ ppm 12.82 (s, 1H), 8.53 (s, 1H), 8.36-8.27 (m, 2H), 8.22 (s, 1H), 7.88 (s, 1H), 7.81 (dd, J=7.9, 1.5 Hz, 1H), 7.60-7.50 (m, 1H), 7.40-7.27 (m, 2H), 7.21-7.08 (m, 1H), 6.51 (m, 1H), 6.39 (d, J=8.5 Hz, 1H), 5.92-5.80 (m, 1H), 5.66-5.48 (m, 1H), 4.20-4.06 (m, 1H), 4.01-3.91 (m, 2H), 3.90-3.77 (m, 1H), 2.41 (s, 3H), 2.40-2.32 (m, 1H), 2.26-2.16 (m, 1H), 1.59 (d, J=6.5 Hz, 3H). MS (ESI) m/e [M+1]⁺=487.

### Example 70: 2-((1-(2,7-dimethyl-3-(2-methyl-1-oxoisoindolin-5-yl)-1-oxo-1,2-dihydroisoquinolin-5-yl)ethyl)amino)benzoic acid

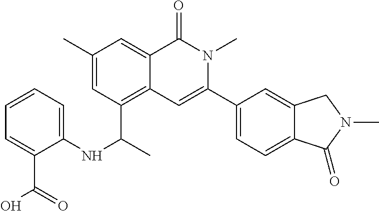 [SMILES: Cc1cc(C(C)Nc2ccccc2C(=O)O)c2cc(-c3ccc4c(c3)CN(C)C4=O)n(C)c(=O)c2c1]

The desired product (55 mg) was obtained following the similar procedure as Example 10. ¹H NMR (400 MHz, DMSO-d₆) δ ppm 8.90 (s, 1H), 7.99 (s, 1H), 7.84-7.74 (m, 3H), 7.72-7.65 (m, 1H), 7.55-7.46 (m, 1H), 7.06 (m, 1H), 6.88 (s, 1H), 6.45 (m, 1H), 6.26 (d, J=8.4 Hz, 1H), 5.21-5.06 (m, 1H), 4.67-4.41 (m, 2H), 3.32 (s, 3H), 3.09 (s, J=17.2 Hz, 3H), 2.37 (s, 3H), 1.49 (d, J=6.6 Hz, 3H). MS (ESI) m/e [M+1]⁺=482.

### Example 71: 2-((1-(3-(4-fluorophenyl)-7-methyl-1-oxo-2-(thiazol-2-yl)-1,2-dihydroisoquinolin-5-yl)ethyl)amino)benzoic acid

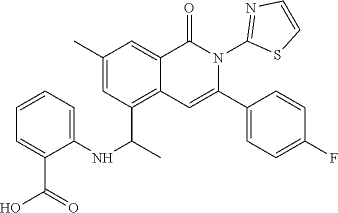 [SMILES: Cc1cc(C(C)Nc2ccccc2C(=O)O)c2cc(-c3ccc(F)cc3)n(-c3nccs3)c(=O)c2c1]

### Step 1: 5-acetyl-3-(4-fluorophenyl)-7-methylisoquinolin-1(2H)-one

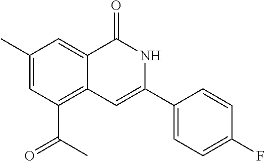 [SMILES: CC(=O)c1cc(C)cc2c(=O)[nH]c(-c3ccc(F)cc3)cc12]

To a solution of 5-acetyl-3-chloro-7-methylisoquinolin-1(2H)-one (737 mg, 3.13 mmol), (4-fluorophenyl)boronic acid (657 mg, 4.69 mmol) and K₃PO₄ (2.00 g, 9.38 mmol) in dioxane (20 ml) and H₂O (3 ml) was add Pd(PPh₃)₄ (180 mg, 0.16 mmol) under N₂. The resulting solution was stirred overnight at 100° C. The mixture was poured into H₂O and extracted with EtOAc. The organic layer was dried over Na₂SO₄, filtered and concentrated under vacuum. The residue was purified by flash chromatography on silica gel to give the product (370 mg, 93%). MS (ESI) m/e [M+1]⁺=296.

### Step 2: 5-acetyl-3-(4-fluorophenyl)-7-methyl-2-(thiazol-2-ylisoquinolin-1(2H)-one

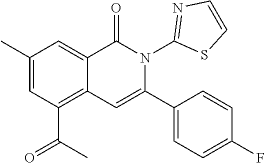 [SMILES: CC(=O)c1cc(C)cc2c(=O)n(-c3nccs3)c(-c3ccc(F)cc3)cc12]

To a solution of 5-acetyl-3-(4-fluorophenyl)-7-methylisoquinolin-1(2H)-one (180 mg, 0.61 mmol), 2-bromothiazole (200 mg, 1.22 mmol) and K₂CO₃ (253 mg, 1.83 mmol) in DMAc (10 ml) was add CuI (12 mg, 0.06 mmol) and 4,7-dimethoxy-1,10-phenanthroline (29 mg, 0.12 mmol) under N₂. The resulting solution was radiated by mw at 150° C. for 3 h. Cooled to room temperature, concentrated under vacuum. The residue was purified by flash chromatography on silica gel to give the isomers (105 mg, 46%). MS (ESI) m/e [M+1]⁺=379.

### Step 3: 3-(4-fluorophenyl)-5-(1-hydroxyethyl)-7-methyl-2-(thiazol-2-yl)isoquinolin-1(2H)-one

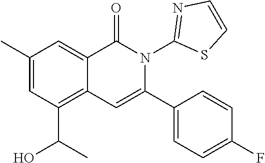 [SMILES: Cc1cc(C(C)O)c2cc(-c3ccc(F)cc3)n(-c3nccs3)c(=O)c2c1]

A solution of 5-acetyl-3-(4-fluorophenyl)-7-methyl-2-(thiazol-2-yl)isoquinolin-1(2H)-one (105 mg, 0.26 mmol) in DCM (10 ml) and MeOH (10 ml) was treated with NaBH₄ (20 mg, 0.53 mmol) in portions at 0° C. The resulting solution was stirred at room temperature for 1 h. The mixture was diluted with H₂O (10 ml) and extracted with DCM (20 mL×2). The combined organic phase was washed with brine (20 mL), dried over anhydrous Na₂SO₄, filtered, and concentrated in vacuum. The residue was purified by flash chromatography on silica to give the product (35 mg, 33%). MS (ESI) m/e [M+1]⁺=381.

### Step 4: 5-(1-bromoethyl)-3-(4-fluorophenyl)-7-methyl-2-(thiazol-2-yl)isoquinolin-1(2H)-one

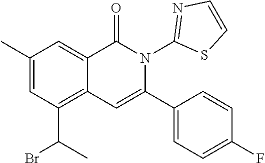 [SMILES: Cc1cc(C(C)Br)c2cc(-c3ccc(F)cc3)n(-c3nccs3)c(=O)c2c1]

To a solution of 3-(4-fluorophenyl)-5-(1-hydroxyethyl)-7-methyl-2-(thiazol-2-yl)isoquinolin-1(2H)-one (35 mg, 0.09 mmol) in DCM (5 ml) was added dropwise PBr₃ (75 mg, 0.27 mmol) at 0° C. The resulting solution was stirred for 2 h at RT. The reaction was quenched with water (5 ml) at 0° C. and the pH adjusted to 8 with saturated aqueous NaHCO₃. The mixture was extracted with DCM (10 mL×2). The combined organic extracts were washed with brine, dried over anhydrous Na₂SO₄, filtered, and concentrated to give the desired product (30 mg, 78%). MS (ESI) m/e [M+1]⁺=443.

### Step 5: methyl 2-((1-(3-(4-fluorophenyl)-7-methyl-1-oxo-2-(thiazol-2-yl)-1,2-dihydroisoquinolin-5-yl)ethyl)amino)benzoate

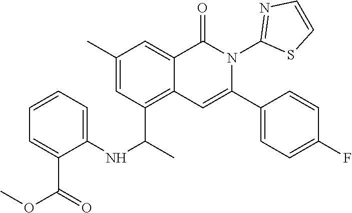 [SMILES: COC(=O)c1ccccc1NC(C)c1cc(C)cc2c(=O)n(-c3nccs3)c(-c3ccc(F)cc3)cc12]

To a solution of 5-(1-bromoethyl)-3-(4-fluorophenyl)-7-methyl-2-(thiazol-2-yl)isoquinolin-1(2H)-one (30 mg, 0.07 mmol) and methyl 2-aminobenzoate (30 mg, 0.20 mmol) in DMF (3 ml) was added DIEA (44 mg, 0.34 mmol). The resulted solution was stirred for 4 h at 90° C. After cooled to room temperature. The reaction was diluted with water (30 ml) and extracted with EtOAc (30 mL×2). The combined organic extracts were washed with brine (20 mL), dried over anhydrous Na₂SO₄, filtered, and concentrated under vacuum. The residue was purified on a silica gel column to give the desired product (20 mg, 57%). MS (ESI) m/e [M+1]⁺=514.

### Step 6: 2-((1-(3-(4-fluorophenyl)-7-methyl-1-oxo-2-(thiazol-2-yl)-1,2-dihydroisoquinolin-5-yl)ethyl)amino)benzoic acid

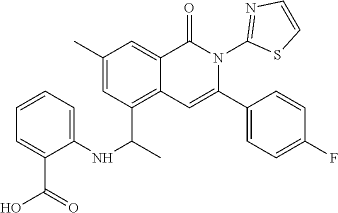 [SMILES: Cc1cc(C(C)Nc2ccccc2C(=O)O)c2cc(-c3ccc(F)cc3)n(-c3nccs3)c(=O)c2c1]

To a solution of methyl 2-((1-(3-(4-fluorophenyl)-7-methyl-1-oxo-2-(thiazol-2-yl)-1,2-dihydroisoquinolin-5-yl)ethyl)amino)benzoate (20 mg, 0.04 mmol) in methanol (4 mL), H₂O (2 ml) and THF (4 ml) was added Lithium hydroxide monohydrate (16 mg, 0.39 mmol). The resulting solution was stirred for 4 h at 50° C. The solution was concentrated under vacuum. The residue was redissolved in H₂O and acidized to pH 5˜6 with HCl (2 M in water). The mixture was filtered, and the filter cake was purified by Prep-HPLC to desired product (6 mg, 32%). ¹H NMR (400 MHz, DMSO-d₆) δ ppm 8.55-8.30 (m, 1H), 7.99 (s, 1H), 7.86-7.72 (m, 2H), 7.69-7.61 (m, 1H), 7.61-7.55 (m, 1H), 7.48 (s, 1H), 7.45-7.32 (m, 2H), 7.26-7.05 (m, 3H), 6.84 (s, 1H), 6.59-6.47 (m, 1H), 6.43-6.28 (m, 1H), 5.38-5.12 (m, 1H), 2.38 (s, 3H), 1.63-1.45 (m, 3H). MS (ESI) m/e [M+1]⁺=500.

### Example 72: 2-((1-(3-(6-(4-hydroxy-4-methylpiperidin-1-yl)pyridin-3-yl)-2,7-dimethyl-1-oxo-1,2-dihydroisoquinolin-5-yl)ethyl)amino)benzoic acid

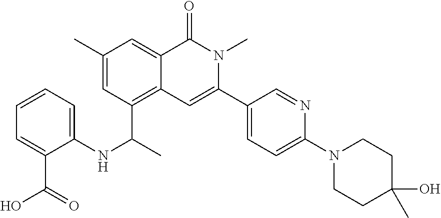 [SMILES: Cc1cc(C(C)Nc2ccccc2C(=O)O)c2cc(-c3ccc(N4CCC(C)(O)CC4)nc3)n(C)c(=O)c2c1]

### Step 1: methyl 2-((1-(3-(6-fluoropyridin-3-yl)-2,7-dimethyl-1-oxo-1,2-dihydroisoquinolin-5-yl)ethyl)amino)benzoate

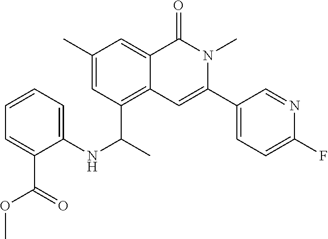 [SMILES: COC(=O)c1ccccc1NC(C)c1cc(C)cc2c(=O)n(C)c(-c3ccc(F)nc3)cc12]

A mixture of methyl 2-((1-(3-chloro-2,7-dimethyl-1-oxo-1,2-dihydroisoquinolin-5-yl)ethyl)amino)benzoate (150 mg, 0.39 mmol), 2-fluoro-5-(4,4,5,5-tetramethyl-1,3,2-dioxaborolan-2-yl)pyridine (131 mg, 0.59 mmol), Pd(PPh₃)₄ (45 mg, 0.04 mmol) and K₃PO₄ (248 mg, 1.17 mmol) in dioxane and H₂O (4:1) (10 ml) was stirred at 100° C. for 1 h. The mixture was concentrated under vacuum. The residue was purified by flash chromatography on silica gel to give the desired product (120 mg, 69%). MS (ESI) m/e [M+1]⁺=446.

### Step 2: methyl 2-((1-(3-(6-(4-hydroxy-4-methylpiperidin-1-yl)pyridin-3-yl)-2,7-dimethyl-1-oxo-1,2-dihydroisoquinolin-5-yl)ethyl)amino)benzoate

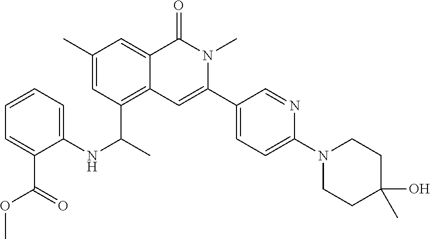 [SMILES: COC(=O)c1ccccc1NC(C)c1cc(C)cc2c(=O)n(C)c(-c3ccc(N4CCC(C)(O)CC4)nc3)cc12]

A mixture of methyl 2-((1-(3-(6-fluoropyridin-3-yl)-2,7-dimethyl-1-oxo-1,2-dihydroisoquinolin-5-yl)ethyl)amino)benzoate_(30 mg, 0.07 mmol), 4-methylpiperidin-4-ol (16 mg, 0.13 mmol) and Cs₂CO₃ (66 mg, 0.20 mmol) in DMSO (3 mL) was stirred at 120° C. for 1 h. The mixture was concentrated under vacuum. The residue was purified by prep-TLC to give the desired product (20 mg, 55%). MS (ESI) m/e [M+1]⁺=541.

### Step 3: 2-((1-(3-(6-(4-hydroxy-4-methylpiperidin-1-yl)pyridin-3-yl)-2,7-dimethyl-1-oxo-1,2-dihydroisoquinolin-5-yl)ethyl)amino)benzoic acid

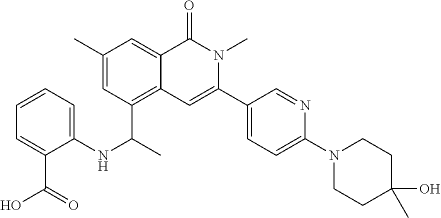 [SMILES: Cc1cc(C(C)Nc2ccccc2C(=O)O)c2cc(-c3ccc(N4CCC(C)(O)CC4)nc3)n(C)c(=O)c2c1]

To a solution of methyl 2-((1-(3-(6-(4-hydroxy-4-methylpiperidin-1-yl)pyridin-3-yl)-2,7-dimethyl-1-oxo-1,2-dihydroisoquinolin-5-yl)ethyl)amino)benzoate_(20 mg, 0.04 mmol) in MeOH (1 mL) and THF (0.5 mL) was added 4N NaOH (0.2 mL). The reaction mixture was stirred at 50° C. for 16 h. The mixture was adjusted to pH 5 by 1N HCl and concentrated under vacuum. The residue was purified by prep-HPLC to give the desired product (10.3 mg, 53%). ¹H NMR (400 MHz, DMSO-d₆) δ ppm 8.65 (brs, 1H), 8.29 (s, 1H), 7.97 (s, 1H), 7.81 (d, J=8.0 Hz, 1H), 7.73 (d, J=8.0 Hz, 1H), 7.48 (s, 1H), 7.14-7.10 (m, 1H), 6.94 (d, J=8.9 Hz, 1H), 6.84 (s, 1H), 6.51-6.47 (m, 1H), 6.30 (d, J=8.5 Hz, 1H), 5.25-5.10 (m, 1H), 4.39 (brs, 1H), 3.96-3.92 (m, 2H), 3.43 (s, 3H), 3.23-3.05 (m, 2H), 2.36 (s, 3H), 1.61-1.34 (m, 7H), 1.17 (s, 3H). MS (ESI) m/e [M+1]⁺=527.

### Example 73: 2-((1-(3-(4-(4-hydroxytetrahydro-2H-pyran-4-yl)phenyl)-2,7-dimethyl-1-oxo-1,2-dihydroisoquinolin-5-yl)ethyl)amino)benzoic acid

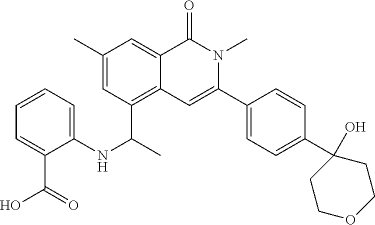 [SMILES: Cc1cc(C(C)Nc2ccccc2C(=O)O)c2cc(-c3ccc(C4(O)CCOCC4)cc3)n(C)c(=O)c2c1]

### Step 1: 4-(4-(4,4,5,5-tetramethyl-1,3,2-dioxaborolan-2-yl)phenyl)tetrahydro-2H-pyran-4-ol

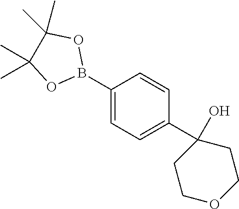 [SMILES: CC1(C)OB(c2ccc(C3(O)CCOCC3)cc2)OC1(C)C]

A mixture of 4-(4-bromophenyl)tetrahydro-2H-pyran-4-ol (300 mg, 1.2 mmol), Bis(pinacolato)diboron (366 mg, 1.4 mmol), Pd(dppf)Cl₂ (82 mg, 0.1 mmol), and Potassium acetate (352 mg, 3.6 mmol) in dioxane (10 mL) was stirred at 90° C. for 12 h under N₂. The mixture was poured into H₂O and extracted with EtOAc. The organic layer was dried over Na₂SO₄, filtered, and concentrated under vacuum. The residue was purified by CombiFlash chromatography on silica gel to give the desired product (310 mg, 85%). MS (ESI) m/e [M+1]⁺=305.

### Step 2: methyl 2-((1-(3-(4-(4-hydroxytetrahydro-2H-pyran-4-yl)phenyl)-2,7-dimethyl-1-oxo-1,2-dihydroisoquinolin-5-yl)ethyl)amino)benzoate

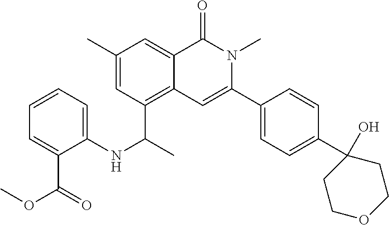 [SMILES: COC(=O)c1ccccc1NC(C)c1cc(C)cc2c(=O)n(C)c(-c3ccc(C4(O)CCOCC4)cc3)cc12]

A mixture of methyl 2-((1-(3-chloro-2,7-dimethyl-1-oxo-1,2-dihydroisoquinolin-5-yl)ethyl)amino)benzoate (80 mg, 0.2 mmol), 4-(4-(4,4,5,5-tetramethyl-1,3,2-dioxaborolan-2-yl)phenyl)tetrahydro-2H-pyran-4-ol (92 mg, 0.3 mmol), Pd(PPh₃)₄ (23 mg, 0.02 mmol), and K₃PO₄ (127 mg, 0.6 mmol) in dioxane (5 mL) and H₂O (0.5 mL) was stirred at 110° C. for 15 h under N₂. The mixture was poured into H₂O and extracted with EtOAc. The organic layer was dried over Na₂SO₄, filtered, and concentrated under vacuum. The residue was purified by Prep-TLC to give the desired product (45 mg, 43%). MS (ESI) m/e [M+1]⁺=527.

### Step 3: 2-((1-(3-(4-(4-hydroxytetrahydro-2H-pyran-4-yl)phenyl)-2,7-dimethyl-1-oxo-1,2-dihydroisoquinolin-5-yl)ethyl)amino)benzoic acid

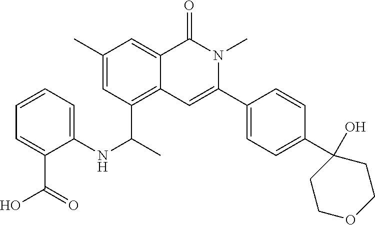 [SMILES: Cc1cc(C(C)Nc2ccccc2C(=O)O)c2cc(-c3ccc(C4(O)CCOCC4)cc3)n(C)c(=O)c2c1]

To a solution of methyl 2-((1-(3-(4-(4-hydroxytetrahydro-2H-pyran-4-yl)phenyl)-2,7-dimethyl-1-oxo-1,2-dihydroisoquinolin-5-yl)ethyl)amino)benzoate (45 mg, 0.09 mmol) in methanol (3 mL) was added NaOH (3M, 1 mL). The resulting solution was stirred for 5 h at 60° C. The solution was concentrated under vacuum. The residue was redissolved in H₂O and acidized to pH 5˜6 with HCl (2 M in water). The mixture was filtered and the solid was collected, purified by Prep-HPLC to give the desired product (21 mg, 46%). ¹H NMR (400 MHz, DMSO-d₆) δ ppm 12.79 (brs, 1H), 8.44 (s, 1H), 7.99 (s, 1H), 7.84-7.76 (m, 1H), 7.69-7.51 (m, 4H), 7.47 (s, 1H), 7.15 (m, 1H), 6.83 (s, 1H), 6.50 (m, 1H), 6.32 (d, J=8.5 Hz, 1H), 5.28-5.10 (m, 2H), 3.89-3.65 (m, 4H), 3.33 (s, 3H), 2.36 (s, 3H), 2.11-1.95 (m, 2H), 1.65-1.53 (m, 2H), 1.50 (d, J=6.5 Hz, 3H). MS (ESI) m/e [M+1]⁺=513.

### Example 74: 2-((1-(3-(4-(4-methoxytetrahydro-2H-pyran-4-yl)phenyl)-2,7-dimethyl-1-oxo-1,2-dihydroisoquinolin-5-yl)ethyl)amino)benzoic acid

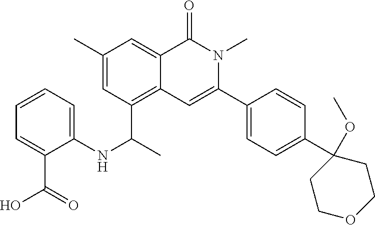 [SMILES: COC1(c2ccc(-c3cc4c(C(C)Nc5ccccc5C(=O)O)cc(C)cc4c(=O)n3C)cc2)CCOCC1]

### Step 1: 4-(4-bromophenyl)-4-methoxytetrahydro-2H-pyran

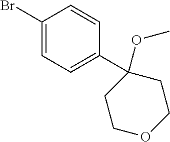

To a solution of 4-(4-bromophenyl)tetrahydro-2H-pyran-4-ol (668 mg, 2.6 mmol) in DMF (10 ml) was added NaH (60% w/w in mineral oil, 125 mg, 3.1 mmol) at 0-10° C. After stirring at 0-10° C. for 1 h, CH₃I (443 mg in DMF, 3.1 mmol) was added dropwise for 5 min. The resulting solution was stirred at 25° C. for 12 h under N₂. The mixture was poured into H₂O and extracted with EtOAc. The organic layer was dried over Na₂SO₄, filtered, and concentrated under vacuum. The residue was purified by CombiFlash chromatography on silica gel to give the desired product (636 mg, 90%). MS (ESI) m/e [M+1]⁺=271.

### Step 2: 2-(4-(4-methoxytetrahydro-2H-pyran-4-yl)phenyl)-4,4,5,5-tetramethyl-1,3,2-dioxaborolane

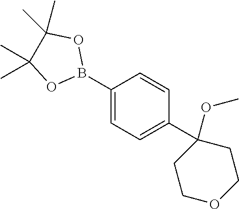

A mixture of 4-(4-bromophenyl)-4-methoxytetrahydro-2H-pyran (636 mg, 2.3 mmol), Bis(pinacolato)diboron (894 mg, 3.5 mmol), Pd(dppf)Cl₂ (163 mg, 0.2 mmol), and Potassium acetate (460 mg, 4.6 mmol) in dioxane (10 ml) was stirred at 90° C. for 12 h under N₂. The mixture was poured into H₂O and extracted with EtOAc. The organic layer was dried over Na₂SO₄, filtered, and concentrated under vacuum. The residue was purified by CombiFlash chromatography to give the desired product (650 mg, 88%). MS (ESI) m/e [M+1]⁺=319.

### Step 3: methyl 2-((1-(3-(4-(4-methoxytetrahydro-2H-pyran-4-yl)phenyl)-2,7-dimethyl-1-oxo-1,2-dihydroisoquinolin-5-yl)ethyl)amino)benzoate

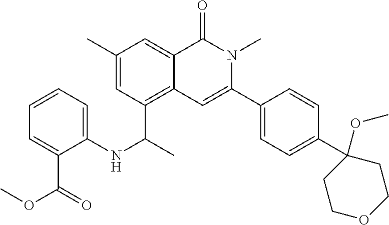

A mixture of methyl 2-((1-(3-chloro-2,7-dimethyl-1-oxo-1,2-dihydroisoquinolin-5-yl)ethyl)amino)benzoate (80 mg, 0.2 mmol), 2-(4-(4-methoxytetrahydro-2H-pyran-4-yl)phenyl)-4,4,5,5-tetramethyl-1,3,2-dioxaborolane (96 mg, 0.3 mmol), Pd(PPh₃)₄ (23 mg, 0.02 mmol), and K₃PO₄ (127 mg, 0.6 mmol) in dioxane (5 mL) and H₂O (0.5 mL) was stirred at 110° C. for 15 h under N₂. The mixture was poured into H₂O and extracted with EtOAc. The organic layer was dried over Na₂SO₄, filtered, and concentrated under vacuum. The residue was purified by Prep-TLC (PE/EA=2:1) to give the desired product (52 mg, 48%). MS (ESI) m/e [M+1]⁺=541.

### Step 4: 2-((1-(3-(4-(4-methoxytetrahydro-2H-pyran-4-yl)phenyl)-2,7-dimethyl-1-oxo-1,2-dihydroisoquinolin-5-yl)ethyl)amino)benzoic acid

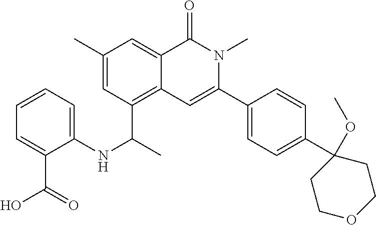 [SMILES: COC1(c2ccc(-c3cc4c(C(C)Nc5ccccc5C(=O)O)cc(C)cc4c(=O)n3C)cc2)CCOCC1]

To a solution of methyl 2-((1-(3-(4-(4-methoxytetrahydro-2H-pyran-4-yl)phenyl)-2,7-dimethyl-1-oxo-1,2-dihydroisoquinolin-5-yl)ethyl)amino)benzoate (52 mg, 0.1 mmol) in methanol (3 mL) was added NaOH (3M, 1 mL). The resulting solution was stirred for 5 h at 60° C. The solution was concentrated under vacuum. The residue was redissolved in H₂O and acidized to pH 5˜6 with HCl (2 M in water). The solid was collected by filtration and purified by Prep-HPLC (mobile phase, water (10 mmol/L FA) and ACN (25% Phase B up to 80% in 8 min); Detector, UV 254 nm) to give the desired product (28 mg, 53%). ¹H NMR (400 MHz, DMSO-d₆) δ ppm 8.44-8.31 (m, 1H), 7.99 (s, 1H), 7.84-7.76 (m, 1H), 7.64-7.50 (m, 4H), 7.47 (d, J=1.3 Hz, 1H), 7.21-7.10 (m, 1H), 6.85 (s, 1H), 6.55-6.45 (m, 1H), 6.33 (d, J=8.4 Hz, 1H), 5.29-5.13 (m, 1H), 3.79-3.67 (m, 4H), 3.35 (s, 3H), 2.95 (s, 3H), 2.36 (s, 3H), 2.01-1.90 (m, 4H), 1.50 (d, J=6.5 Hz, 3H). MS (ESI) m/e [M+1]⁺=527.

### Example 75: 2-((1-(2,7-dimethyl-3-(2-methyl-3-oxoisoindolin-5-yl)-1-oxo-1,2-dihydroisoquinolin-5-yl)ethyl)amino)benzoic acid

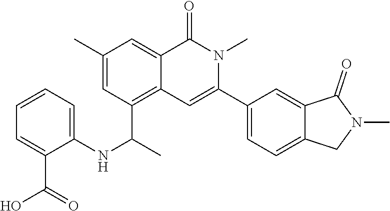 [SMILES: Cc1cc(C(C)Nc2ccccc2C(=O)O)c2cc(-c3ccc4c(c3)C(=O)N(C)C4)n(C)c(=O)c2c1]

The desired product (11.9 mg) was obtained following the similar procedure as Example 10. ¹H NMR (400 MHz, DMSO-d₆) δ ppm 12.77 (brs, 1H), 8.46-8.31 (m, 1H), 8.00 (s, 1H), 7.88-7.69 (m, 4H), 7.51-7.44 (m, 1H), 7.20-7.12 (m, 1H), 6.87 (s, 1H), 6.51 (m, 1H), 6.33 (d, J=8.5 Hz, 1H), 5.31-5.12 (m, 1H), 4.55 (s, 2H), 3.31 (s, 3H), 3.11 (s, 3H), 2.37 (s, 3H), 1.50 (d, J=6.5 Hz, 3H). MS (ESI) m/e [M+1]⁺=482.

### Example 76: 2-((1-(2,7-dimethyl-1-oxo-3-(1-phenyl-2,5-dihydro-1H-pyrrol-3-yl)-1,2-dihydroisoquinolin-5-yl)ethyl)amino)benzoic acid

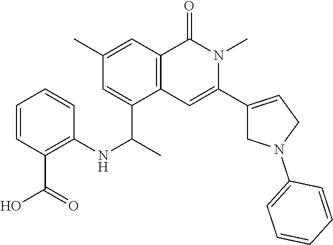 [SMILES: Cc1cc(C(C)Nc2ccccc2C(=O)O)c2cc(C3=CCN(c4ccccc4)C3)n(C)c(=O)c2c1]

### Step 1: tert-butyl 3-(5-(1-((2-(methoxycarbonyl)phenyl)amino)ethyl)-2,7-dimethyl-1-oxo-1,2-dihydroisoquinolin-3-yl)-2,5-dihydro-11H-pyrrole-1-carboxylate

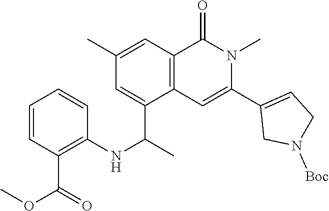 [SMILES: COC(=O)c1ccccc1NC(C)c1cc(C)cc2c(=O)n(C)c(C3=CCN(C(=O)OC(C)(C)C)C3)cc12]

A mixture of methyl 2-((1-(3-chloro-2,7-dimethyl-1-oxo-1,2-dihydroisoquinolin-5-yl)ethyl)amino)benzoate (50.00 mg, 0.13 mmol), tert-butyl 3-(4,4,5,5-tetramethyl-1,3,2-dioxaborolan-2-yl)-2,5-dihydro-1H-pyrrole-1-carboxylate (46.00 mg, 0.156 mmol), Pd(PPh₃)₄ (15.00 mg, 0.013 mmol) and K₃PO₄ (82.8 mg, 0.39 mmol) in dioxane and H₂O (5:1) (1 mL) was stirred at 90° C. for 16 h. The mixture was concentrated under vacuum. The residue was purified by prep-TLC to give the desired product (56 mg, 83.2%).

### Step 2: methyl 2-((1-(3-(2,5-dihydro-1H-pyrrol-3-yl)-2,7-dimethyl-1-oxo-1,2-dihydroisoquinolin-5-yl)ethyl)amino)benzoate

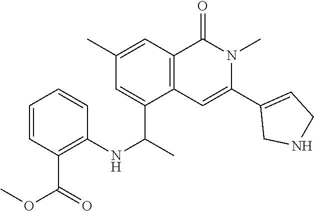 [SMILES: COC(=O)c1ccccc1NC(C)c1cc(C)cc2c(=O)n(C)c(C3=CCNC3)cc12]

To a solution of tert-butyl 3-(5-(1-((2-(methoxycarbonyl)phenyl)amino)ethyl)-2,7-dimethyl-1-oxo-1,2-dihydroisoquinolin-3-yl)-2,5-dihydro-1H-pyrrole-1-carboxylate (56 mg, 0.108 mmol) in DCM (2 mL) was added TFA (1 mL). The reaction mixture was stirred at room temperature for 1 h. The mixture was concentrated and neutralized with aqueous saturated NaHCO₃. Then extract with DCM and dry over Na₂SO₄. The organic layer was filtered and concentrated to afford the desired product (50 mg, crude).

### Step 3: methyl 2-((1-(2,7-dimethyl-1-oxo-3-(1-phenyl-2,5-dihydro-1H-pyrrol-3-yl)-1,2-dihydroisoquinolin-5-yl)ethyl)amino)benzoate

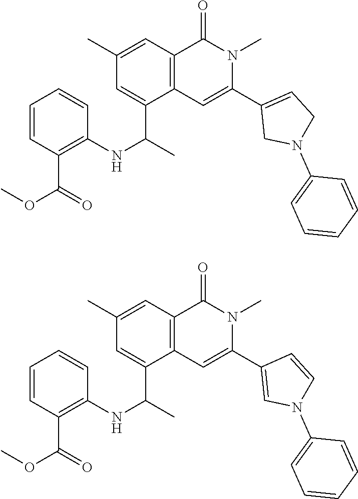 [SMILES: COC(=O)c1ccccc1NC(C)c1cc(C)cc2c(=O)n(C)c(C3=CCN(c4ccccc4)C3)cc12 ; COC(=O)c1ccccc1NC(C)c1cc(C)cc2c(=O)n(C)c(-c3ccn(-c4ccccc4)c3)cc12]

A mixture of methyl 2-((1-(3-(2,5-dihydro-1H-pyrrol-3-yl)-2,7-dimethyl-1-oxo-1,2-dihydroisoquinolin-5-yl)ethyl)amino)benzoate (30 mg, 0.07 mmol), bromobenzene (13.5 mg, 0.086 mmol), XantPhos (8.1 mg, 0.014 mmol), Pd₂(dba)₃ (6.4 mg, 0.007 mmol) and cesium carbonate (68.4 mg, 0.21 mmol) in dioxane (2 mL) under a nitrogen balloon was stirred at 100° C. for 2 h. The reaction was diluted with water and extracted with DCM (50 mL×2). The organic phase was concentrated in vacuum, and the crude product was purified by prep-TLC (PE/EA=3/1) to afford a mixture of methyl 2-((1-(2,7-dimethyl-1-oxo-3-(1-phenyl-2,5-dihydro-1H-pyrrol-3-yl)-1,2-dihydroisoquinolin-5-yl)ethyl)amino)benzoate and methyl 2-((1-(2,7-dimethyl-1-oxo-3-(1-phenyl-1H-pyrrol-3-yl)-1,2-dihydroisoquinolin-5-yl)ethyl)amino)benzoate as product (35 mg, 98% yield).

### Step 4: 2-((1-(2,7-dimethyl-1-oxo-3-(1-phenyl-2,5-dihydro-1H-pyrrol-3-yl)-1,2-dihydroisoquinolin-5-yl)ethyl)amino)benzoic acid

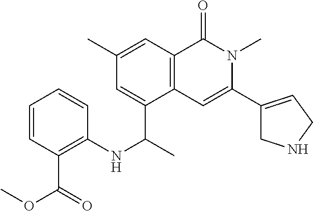 [SMILES: COC(=O)c1ccccc1NC(C)c1cc(C)cc2c(=O)n(C)c(C3=CCNC3)cc12]

To a solution of the mixture material (35 mg, 0.07 mmol) in MeOH (1 mL) and THF (1 mL) was added 4 N NaOH (0.5 mL). The reaction mixture was stirred at 50° C. for 1 h. The mixture was adjusted to pH 5 by 1N HCl and concentrated under vacuum. The residue was purified by prep-HPLC to give the desired product 2-((1-(2,7-dimethyl-1-oxo-3-(1-phenyl-2,5-dihydro-1H-pyrrol-3-yl)-1,2-dihydroisoquinolin-5-yl)ethyl)amino)benzoic acid (1.33 mg, 4% yield). 11H NMR (400 MHz, DMSO-d₆) δ ppm 12.75 (brs, 1H), 8.54 (s, 1H), 7.96 (s, 1H), 7.81 (d, J=7.2 Hz, 1H), 7.50 (s, 1H), 7.25-7.15 (m, 3H), 7.01 (s, 1H), 6.70-6.57 (m, 3H), 6.55-6.45 (m, 2H), 6.34 (d, J=8.0 Hz, 1H), 5.30-5.20 (m, 1H), 4.44 (s, 2H), 4.30 (s, 2H), 3.57 (s, 3H), 2.36 (s, 3H), 1.52 (d, J=6.4 Hz, 3H). MS (ESI) m/e [M+1]⁺=480.

### Example 77: 2-((1-(2,7-dimethyl-1-oxo-3-(1-phenyl-1H-pyrrol-3-yl)-1,2-dihydroisoquinolin-5-yl)ethyl)amino)benzoic acid

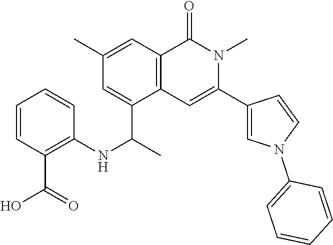 [SMILES: Cc1cc(C(C)Nc2ccccc2C(=O)O)c2cc(-c3ccn(-c4ccccc4)c3)n(C)c(=O)c2c1]

The desired product (19.71 mg) was obtained as a side product in the synthesis of Example 76. ¹H NMR (400 MHz, DMSO-d₆) δ ppm 12.69 (brs, 1H), 8.37 (d, J=6.0 Hz, 1H), 7.96 (s, 1H), 7.85-7.76 (m, 2H), 7.69 (d, J=8.0 Hz, 2H), 7.60-7.50 (m, 3H), 7.45 (s, 1H), 7.31 (t, J=7.6 Hz, 1H), 7.23-7.15 (m, 1H), 6.94 (s, 1H), 6.70-6.60 (m, 1H), 6.55-6.45 (m, 1H), 6.37 (d, J=8.8 Hz, 1H), 5.34-5.07 (m, 1H), 3.61 (s, 3H), 2.35 (s, 3H), 1.54 (d, J=6.4 Hz, 3H). MS (ESI) m/e [M+1]⁺=478.

### Example 78: 2-((1-(2,7-dimethyl-3-(6-(morpholinomethyl)pyridin-3-yl)-1-oxo-1,2-dihydroisoquinolin-5-yl)ethyl)amino)benzoic acid

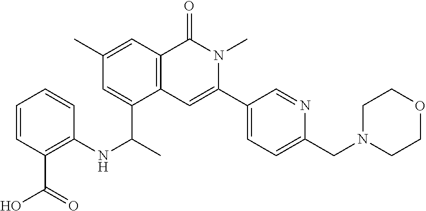 [SMILES: Cc1cc(C(C)Nc2ccccc2C(=O)O)c2cc(-c3ccc(CN4CCOCC4)nc3)n(C)c(=O)c2c1]

The desired product (18 mg) was obtained following the similar procedure as Example 10. ¹H NMR (400 MHz, DMSO-d₆) δ ppm 12.77 (brs, 1H), 8.73 (d, J=1.6 Hz, 1H), 8.45 (s, 1H), 8.08-7.96 (m, 2H), 7.80 (d, J=6.8 Hz, 1H), 7.60 (d, J=8.4 Hz, 1H), 7.49 (s, 1H), 7.20-7.10 (m, 1H), 6.94 (s, 1H), 6.55-6.45 (m, 1H), 6.33 (d, J=8.8 Hz, 1H), 5.30-5.20 (m, 1H), 3.68 (s, 2H), 3.65-3.57 (m, 4H), 3.34-3.23 (m, 3H), 2.50-2.40 (m, 4H), 2.37 (s, 3H), 1.50 (d, J=6.4 Hz, 3H). MS (ESI) m/e [M+1]⁺=513.

### Example 79: 2-((1-(3-(4-(dimethylphosphoryl)phenyl)-2,7-dimethyl-1-oxo-1,2-dihydroisoquinolin-5-yl)ethyl)amino)benzoic acid

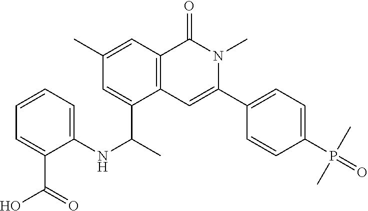 [SMILES: Cc1cc(C(C)Nc2ccccc2C(=O)O)c2cc(-c3ccc(P(C)(C)=O)cc3)n(C)c(=O)c2c1]

The desired product (49.24 mg) was obtained following the similar procedure as Example 10. ¹H NMR (400 MHz, DMSO-d₆) δ ppm 12.74 (brs, 1H), 8.38 (d, J=4.7 Hz, 1H), 8.00 (s, 1H), 7.92 (dd, J=11.0, 8.1 Hz, 2H), 7.80 (d, J=6.5 Hz, 1H), 7.78-7.72 (m, 2H), 7.49 (s, 1H), 7.17 (m, 1H), 6.89 (s, 1H), 6.52 (m, 1H), 6.34 (d, J=8.5 Hz, 1H), 5.33-5.14 (m, 1H), 3.34 (s, 3H), 2.38 (s, 3H), 1.71 (d, J=13.4 Hz, 6H), 1.51 (d, J=6.5 Hz, 3H). MS (ESI) m/e [M+1]⁺=489. MS (ESI) m/e [M+H]⁺=489.

### Example 80: 2-((1-(2,7-dimethyl-3-((1-methyl-1H-pyrazol-4-yl)ethynyl)-1-oxo-1,2-dihydroisoquinolin-5-yl)ethyl)amino)benzoic acid

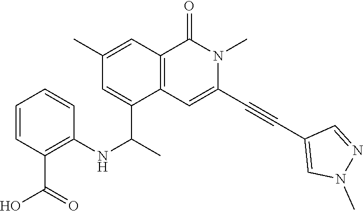 [SMILES: Cc1cc(C(C)Nc2ccccc2C(=O)O)c2cc(C#Cc3cnn(C)c3)n(C)c(=O)c2c1]

The desired product (10 mg) was obtained following the similar procedure as Example 43. ¹H NMR (400 MHz, DMSO-d₆) δ ppm 8.66 (s, 1H), 8.27-8.15 (m, 1H), 7.93 (s, 1H), 7.85-7.75 (m, 2H), 7.47 (s, 1H), 7.35 (s, 1H), 7.10 (m, 1H), 6.47 (m, 1H), 6.25 (d, J=8.3 Hz, 1H), 5.26-5.13 (m, 1H), 3.87 (s, 3H), 3.66 (s, 3H), 2.33 (s, 3H), 1.48 (d, J=6.4 Hz, 3H). MS (ESI) m/e [M+1]⁺=441.

### Example 81: 6-chloro-3-((1-(2-ethyl-3-(4-fluorophenyl)-7-methyl-1-oxo-1,2-dihydroisoquinolin-5-yl)ethyl)amino)picolinic acid

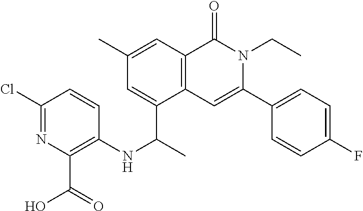 [SMILES: CCn1c(-c2ccc(F)cc2)cc2c(C(C)Nc3ccc(Cl)nc3C(=O)O)cc(C)cc2c1=O]

### Step 1: 5-bromo-3-chloro-2-ethyl-7-methylisoquinolin-1(2H)-one

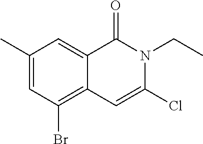 [SMILES: CCn1c(Cl)cc2c(Br)cc(C)cc2c1=O]

To a solution of 5-bromo-3-chloro-7-methylisoquinolin-1(2H)-one (300 mg, 1.10 mmol) and K₂CO₃ (455 mg, 3.30 mmol) in DMF (10 mL) was added C₂H5I (257 mg, 1.65 mmol) at 0° C. The reaction mixture was stirred for 1 h at 0° C. The mixture was poured into H₂O and extracted with EA. The combined organic solution was washed with brine. Dried over anhydrous Na₂SO₄, filtered, the filtrate was concentrated under vacuum and the residue was purified by column chromatography on silica to give the product (210 mg, 63%). MS (ESI) m/e [M+1]⁺=300.

### Step 2: 5-acetyl-3-chloro-2-ethyl-7-methylisoquinolin-1(2H)-one

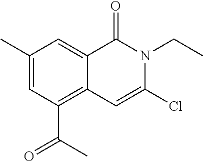 [SMILES: CCn1c(Cl)cc2c(C(C)=O)cc(C)cc2c1=O]

To a solution of 5-bromo-3-chloro-2-ethyl-7-methylisoquinolin-1(2H)-one (210 mg, 0.70 mmol) and tributyl(1-ethoxyvinyl)stannane (250 mg, 0.70 mmol) in dioxane (20 ml) was added bis(triphenylphosphine)palladium(II) chloride (49 mg, 0.07 mmol) under nitrogen atmosphere. The resulting mixture was stirred for 15 h at 90° C. After cooled to room temperature, treated the reaction with 1M aqueous HCl (5 ml) and stirred for 30 min. The mixture was quenched with saturated aqueous KF (20 mL), stirred for 30 min, and filtered. The filter cake was washed with 10% MeOH in DCM (20 ml×3). The combined extracts were dried over anhydrous Na₂SO₄, filtered, and concentrated to give a residue which was purified by column chromatography on silica gel to give the product (138 mg, 75%). MS (ESI) m/e [M+1]⁺=264.

### Step 3: 5-acetyl-2-ethyl-3-(4-fluorophenyl)-7-methylisoquinolin-1(2H)-one

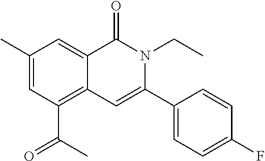 [SMILES: CCn1c(-c2ccc(F)cc2)cc2c(C(C)=O)cc(C)cc2c1=O]

A mixture of 5-acetyl-3-chloro-2-ethyl-7-methylisoquinolin-1(2H)-one (84 mg, 0.32 mmol), (4-fluorophenyl)boronic acid (67 mg, 0.48 mmol), Pd(PPh₃)₄ (37 mg, 0.03 mmol), and K₃PO₄ (204 mg, 0.96 mmol) in dioxane (8 ml) and H₂O (1 ml) was stirred at 100° C. for 12 h under N₂. The mixture was poured into H₂O and extracted with EtOAc. The organic layer was dried over Na₂SO₄, filtered and concentrated under vacuum. The residue was purified by flash chromatography on silica gel to give the product (65 mg, 63%). MS (ESI) m/e [M+1]⁺=324.

### Step 4: 5-(1-aminoethyl)-2-ethyl-3-(4-fluorophenyl)-7-methylisoquinolin-1(2H)-one

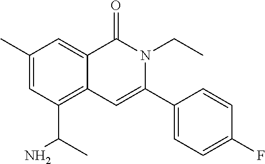 [SMILES: CCn1c(-c2ccc(F)cc2)cc2c(C(C)N)cc(C)cc2c1=O]

To a solution of 5-acetyl-2-ethyl-3-(4-fluorophenyl)-7-methylisoquinolin-1(2H)-one (65 mg, 0.20 mmol) and Ammonium acetate (154 mg, 2.00 mmol) in 5 mL of ethanol was added NaBH₃CN (15 mg, 0.24 mmol). The resulting solution was stirred overnight at 90° C. After cooled to room temperature, the solution was concentrated under vacuum and the residue was purified by CombiFlash on silica gel to give the desired product (38 mg, 59%). MS (ESI) m/e [M+1]⁺=325.

### Step 5: tert-butyl 6-chloro-3-((1-(2-ethyl-3-(4-fluorophenyl)-7-methyl-1-oxo-1,2-dihydroisoquinolin-5-yl)ethyl)amino)picolinate

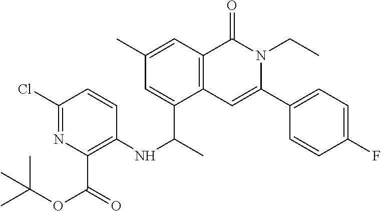 [SMILES: CCn1c(-c2ccc(F)cc2)cc2c(C(C)Nc3ccc(Cl)nc3C(=O)OC(C)(C)C)cc(C)cc2c1=O]

To a solution of 5-(1-aminoethyl)-2-ethyl-3-(4-fluorophenyl)-7-methylisoquinolin-1(2H)-one (38 mg, 0.12 mmol) and tert-butyl 6-chloro-3-fluoropicolinate (60 mg, 0.24 mmol) in DMF (3 ml) was added DIEA (67 mg, 0.52 mmol). The resulting solution was stirred for 15 h at 100° C. The reaction was quenched with water (10 ml) and extracted with EtOAc. The combined organic extracts were washed with brine, dried over anhydrous Na₂SO₄, filtered and concentrated under vacuum. The residue was purified by flash chromatography on silica gel eluted to give the desired product (21 mg, 34%). MS (ESI) m/e [M+1]⁺=536.

### Step 6: 6-chloro-3-((1-(2-ethyl-3-(4-fluorophenyl)-7-methyl-1-oxo-1,2-dihydroisoquinolin-5-yl)ethyl)amino)picolinic acid

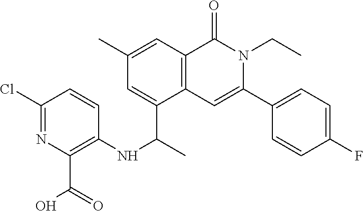 [SMILES: CCn1c(-c2ccc(F)cc2)cc2c(C(C)Nc3ccc(Cl)nc3C(=O)O)cc(C)cc2c1=O]

To a solution of tert-butyl 6-chloro-3-((1-(2-ethyl-3-(4-fluorophenyl)-7-methyl-1-oxo-1,2-dihydroisoquinolin-5-yl)ethyl)amino)picolinate (21 mg, 0.04 mmol) in 3 mL of DCM was added TFA (1 mL). The resulted solution was stirred for 4 h at room temperature. The solution was concentrated under vacuum. The residue was purified by Prep-HPLC to desired product (1.2 mg, 2%). ¹H NMR (400 MHz, DMSO-d₆) δ ppm 13.09 (s, 1H), 8.43-8.26 (m, 1H), 8.00 (s, 1H), 7.70-7.59 (m, 2H), 7.45 (s, 1H), 7.42-7.27 (m, 2H), 6.93-6.84 (m, 1H), 6.75 (s, 1H), 5.31-5.18 (m, 1H), 3.98-3.78 (m, 2H), 2.37 (s, 3H), 1.50 (d, J=6.3 Hz, 3H), 1.04 (t, J=6.8 Hz, 3H). MS (ESI) m/e [M+1]⁺=480.

### Example 82: 2,7-dimethyl-5-(1-((2-(methylsulfonyl)phenyl)amino)ethyl)-3-phenylisoquinolin-1(2H)-one

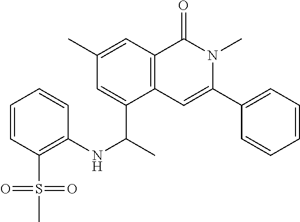 [SMILES: Cc1cc(C(C)Nc2ccccc2S(C)(=O)=O)c2cc(-c3ccccc3)n(C)c(=O)c2c1]

To a solution of 5-(1-bromoethyl)-2,7-dimethyl-3-phenylisoquinolin-1(2H)-one (90 mg, 0.25 mmol) and 2-(methylsulfonyl)aniline (86 mg, 0.51 mmol) in DMA (3 ml) was added DIEA (100 mg, 0.75 mmol). The resulting solution was stirred overnight at 110° C. After cooled to room temperature. The reaction was diluted with water and extracted with EtOAc. The combined organic extracts were washed with brine, dried over anhydrous Na₂SO₄, filtered, and concentrated under vacuum. The residue was purified by Prep-HPLC to give 2,7-dimethyl-5-(1-((2-(methylsulfonyl)phenyl)amino)ethyl)-3-phenylisoquinolin-1(2H)-one (27 mg, 56%). ¹H NMR (400 MHz, DMSO-d₆) δ ppm 8.01 (s, 1H), 7.65-7.48 (m, 7H), 7.35-7.25 (m, 1H), 6.83 (s, 1H), 6.75-6.65 (m, 1H), 6.59 (d, J=5.5 Hz, 1H), 6.46 (d, J=8.4 Hz, 1H), 5.28-5.17 (m, 1H), 3.33 (s, 3H), 3.22 (s, 3H), 2.38 (s, 3H), 1.52 (d, J=6.5 Hz, 3H). MS (ESI) m/e [M+1]⁺=447.

### Example 83: 3-((1-(2,7-dimethyl-1-oxo-3-phenyl-1,2-dihydroisoquinolin-5-yl)ethyl)amino)-5,6-dihydro-4H-pyrrolo[1,2-b]pyrazole-2-carboxylic acid

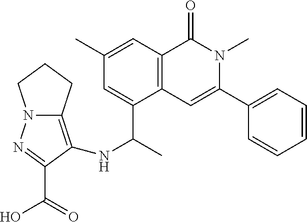 [SMILES: Cc1cc(C(C)Nc2c(C(=O)O)nn3c2CCC3)c2cc(-c3ccccc3)n(C)c(=O)c2c1]

### Step 1: 3-nitro-5,6-dihydro-4H-pyrrolo[1,2-b]pyrazole-2-carboxylic acid

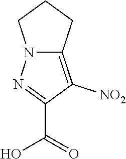 [SMILES: O=C(O)c1nn2c(c1[N+](=O)[O-])CCC2]

To a solution of 5,6-dihydro-4H-pyrrolo[1,2-b]pyrazole-2-carboxylic acid (1 g, 6.57 mmol) in con. H₂SO₄ (10 ml) was added HNO₃ (2 mL, 60%) dropwise at 0° C. The mixture was stirred at rt overnight. The mixture was poured into ice. The solid was collected by filter, washed with water, and dried in high vacuum to give the desired product (1.0 g, 79%). MS (ESI) m/e [M+H]⁺=198.

### Step 2: methyl 3-nitro-5,6-dihydro-4H-pyrrolo[1,2-b]pyrazole-2-carboxylate

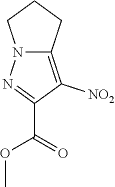

A solution of 3-nitro-5,6-dihydro-4H-pyrrolo[1,2-b]pyrazole-2-carboxylic acid (1 g, 5.1 mmol) in MeOH (15 ml) and HCl/dioxane (4N, 2 ml) was stirred at reflux for 2 h. The solvent was removed, and the crude residue was added NaHCO₃ (aq). The aqueous layer was extracted with EA. The organic layer was dried and concentrated to give the desired product as a white solid (870 mg, 81%). MS (ESI) m/e [M+H]⁺=212.

### Step 3: methyl 3-amino-5,6-dihydro-4H-pyrrolo[1,2-b]pyrazole-2-carboxylate

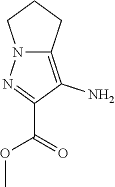

A solution of methyl 3-nitro-5,6-dihydro-4H-pyrrolo[1,2-b]pyrazole-2-carboxylate (870 mg, 4.12 mmol) and Pd/C (150 mg, 5%/wt. %) in MeOH (50 mL) was stirred at rt under H₂ (1 atm) for 5 h. The mixture was filtered, and the filtrate was concentrated to give the desired product (550 mg, 74%). MS (ESI) m/e [M+H]⁺=182.

### Step 4: methyl 3-((1-(2,7-dimethyl-1-oxo-3-phenyl-1,2-dihydroisoquinolin-5-yl)ethyl)amino)-5,6-dihydro-4H-pyrrolo[1,2-b]pyrazole-2-carboxylate

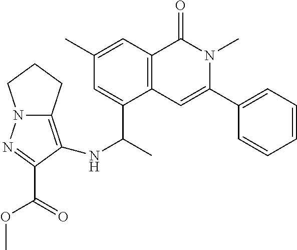 [SMILES: COC(=O)c1nn2c(c1NC(C)c1cc(C)cc3c(=O)n(C)c(-c4ccccc4)cc13)CCC2]

A solution of 5-(1-bromoethyl)-2,7-dimethyl-3-phenylisoquinolin-1(2H)-one (60 mg, 0.17 mmol), methyl 3-amino-5,6-dihydro-4H-pyrrolo[1,2-b]pyrazole-2-carboxylate (60.2 mg, 0.34 mmol) and DIEA (65.8 mg, 0.51 mmol) in DMF (5 mL) was stirred at 100° C. for 2 h. The mixture was cooled to R.T. The solvent was removed, and the crude residue was purified by Prep-TLC to give the desired product (50 mg, 64%). MS (ESI) m/e [M+H]⁺=457.

### Step 5: 3-((1-(2,7-dimethyl-1-oxo-3-phenyl-1,2-dihydroisoquinolin-5-yl)ethyl)amino)-5,6-dihydro-4H-pyrrolo[1,2-b]pyrazole-2-carboxylic acid

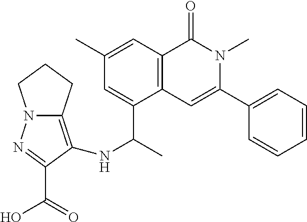 [SMILES: Cc1cc(C(C)Nc2c(C(=O)O)nn3c2CCC3)c2cc(-c3ccccc3)n(C)c(=O)c2c1]

A solution of methyl 3-((1-(2,7-dimethyl-1-oxo-3-phenyl-1,2-dihydroisoquinolin-5-yl)ethyl)amino)-5,6-dihydro-4H-pyrrolo[1,2-b]pyrazole-2-carboxylate (50 mg, 0.11 mmol) in NaOH (1N) (4 mL) and MeOH (4 mL) was stirred at 70° C. for 3 h. The solvent was removed, the aqueous layer was acidified by HCl (3 N), extracted with EA. The organic layer was dried and concentrated. The crude product was purified on Prep-HPLC to give the desired product (26 mg, 53%). ¹H NMR (400 MHz, DMSO-d₆) δ ppm 12.42 (brs, 1H), 7.97 (s, 1H), 7.62-7.45 (m, 6H), 6.67 (s, 1H), 5.42 (s, 1H), 4.94 (d, J=6.5 Hz, 1H), 4.02-3.73 (m, 2H), 3.31 (s, 3H), 2.79-2.61 (m, 1H), 2.38 (s, 3H), 2.29 (dd, J=12.9, 6.3 Hz, 1H), 2.25-2.13 (m, 1H), 2.01-1.88 (m, 1H), 1.40 (d, J=6.6 Hz, 3H). MS (ESI) m/e [M+H]⁺=443.

### Example 84: 2-((1-(2,7-dimethyl-1-oxo-3-(4-(pyrrolidin-1-yl)phenyl)-1,2-dihydroisoquinolin-5-yl)ethyl)amino)benzoic acid

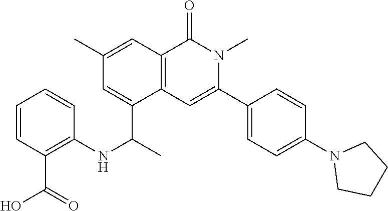 [SMILES: Cc1cc(C(C)Nc2ccccc2C(=O)O)c2cc(-c3ccc(N4CCCC4)cc3)n(C)c(=O)c2c1]

The desired product (16.8 mg) was obtained following the similar procedure as Example 10. ¹H NMR (400 MHz, DMSO-d₆) δ ppm 8.38 (s, 1H), 7.95 (s, 1H), 7.82-7.76 (m, 1H), 7.45 (s, 1H), 7.34 (d, J=8.5 Hz, 2H), 7.16 (m, 1H), 6.71 (s, 1H), 6.62 (d, J=8.6 Hz, 2H), 6.51 (m, 1H), 6.33 (d, J=8.5 Hz, 1H), 5.21-5.06 (m, 1H), 3.36 (s, 3H), 3.31-3.18 (m, 4H), 2.35 (s, 3H), 2.01-1.91 (m, 4H), 1.51 (d, J=6.5 Hz, 3H). MS (ESI) m/e [M+1]⁺=482.

### Example 85: 2-((1-(3-(2-hydroxypyrimidin-5-yl)-2,7-dimethyl-1-oxo-1,2-dihydroisoquinolin-5-yl)ethyl)amino)benzoic acid

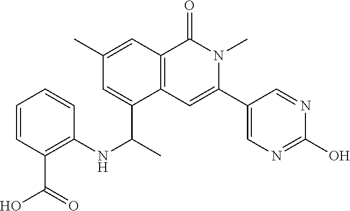 [SMILES: Cc1cc(C(C)Nc2ccccc2C(=O)O)c2cc(-c3cnc(O)nc3)n(C)c(=O)c2c1]

The desired product (32.66 mg) was obtained following the similar procedure as Example 10. ¹H NMR (400 MHz, DMSO-d₆) δ ppm 13.08-11.97 (m, 2H), 8.67-8.31 (m, 3H), 7.96 (s, 1H), 7.79 (d, J=8.0 Hz, 1H), 7.45 (s, 1H), 7.14 (m, 1H), 7.01 (s, 1H), 6.50 (m, 1H), 6.30 (d, J=8.4 Hz, 1H), 5.28-5.19 (m, 1H), 3.43 (s, 3H), 2.35 (s, 3H), 1.50 (d, J=6.8 Hz, 3H). MS (ESI) m/e [M+1]⁺=431

### Example 86: 2-((1-(2,7-dimethyl-1-oxo-3-(6-(trifluoromethyl)pyridin-3-yl)-1,2-dihydroisoquinolin-5-yl)ethyl)amino)benzoic acid

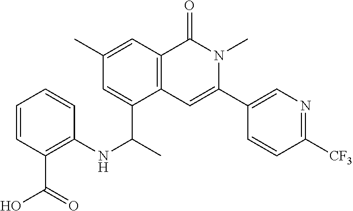 [SMILES: Cc1cc(C(C)Nc2ccccc2C(=O)O)c2cc(-c3ccc(C(F)(F)F)nc3)n(C)c(=O)c2c1]

The desired product (33.3 mg) was obtained following the similar procedure as Example 10. ¹H NMR (400 MHz, DMSO-d₆) δ ppm 12.75 (s, 1H), 9.05 (s, 1H), 8.50-8.32 (m, 2H), 8.09 (d, J=8.0 Hz, 1H), 8.01 (s, 1H), 7.81-7.78 (m, 1H), 7.50 (s, 1H), 7.16 (m, 1H), 7.08 (s, 1H), 6.51 (m, 1H), 6.33 (d, J=8.0 Hz, 1H), 5.35-5.14 (m, 1H), 3.36 (s, 3H), 2.38 (s, 3H), 1.50 (d, J=6.8 Hz, 3H). MS (ESI) m/e [M+1]⁺=482

### Example 87: 2-((1-(3-(1-(((3S,5S)-adamantan-1-yl)methyl)-5-methyl-1H-pyrazol-4-yl)-2,7-dimethyl-1-oxo-1,2-dihydroisoquinolin-5-yl)ethyl)amino)benzoic acid

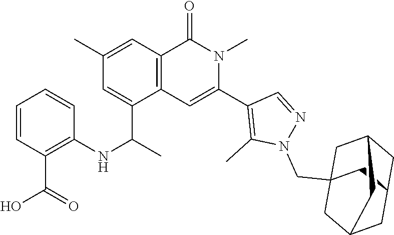 [SMILES: Cc1cc(C(C)Nc2ccccc2C(=O)O)c2cc(-c3cnn(CC45CC6C[C@H](C4)C[C@H](C6)C5)c3C)n(C)c(=O)c2c1]

The desired product (30 mg) was obtained following the similar procedure as Example 10. ¹H NMR (400 MHz, DMSO-d6) δ ppm 12.69 (s, 1H), 8.30 (d, J=6.0 Hz, 1H), 7.97 (s, 1H), 7.79 (dd, J=8.0, 1.6 Hz, 1H), 7.65 (s, 1H), 7.49 (s, 1H), 7.18 (m, 1H), 6.70 (s, 1H), 6.51 (m, 1H), 6.42 (d, J=8.8 Hz, 1H), 5.26-5.16 (m, 1H), 3.77 (s, 2H), 3.36 (s, 3H), 2.37 (s, 3H), 2.19 (s, 3H), 1.96 (s, 3H), 1.67 (m, 3H), 1.54 (m, 12H). MS (ESI) m/e [M+1]⁺=565.

### Example 88: 2-((1-(2,7-dimethyl-3-((1-methyl-1H-pyrazol-3-yl)ethynyl)-1-oxo-1,2-dihydroisoquinolin-5-yl)ethyl)amino)benzoic acid

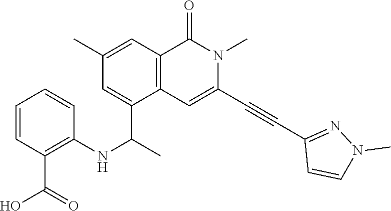 [SMILES: Cc1cc(C(C)Nc2ccccc2C(=O)O)c2cc(C#Cc3ccn(C)n3)n(C)c(=O)c2c1]

### Step 1: 1-methyl-3-((trimethylsilyl)ethynyl)-1H-pyrazole

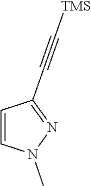

To a solution of 3-iodo-1-methyl-1H-pyrazole (1.00 g, 4.80 mmol) and ethynyltrimethylsilane (1.40 g, 14.42 mmol) in DMF (10 ml) and TEA (5 ml) was added Pd(PPh₃)₄ (278 mg, 0.24 mmol) and CuI (91 mg, 0.48 mmol) under N₂. The resulting solution was stirred at 70° C. for 15 h. The mixture was poured into H₂O and extracted with EtOAc. The combined organic extracts were washed with brine (20 mL), dried over anhydrous Na₂SO₄, filtered, and concentrated under vacuum. The residue was purified by flash chromatography on silica gel to give the product (750 mg, 88%). MS (ESI) m/e [M+1]⁺=179.

### Step 2: 3-ethynyl-1-methyl-1H-pyrazole

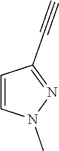

To a solution of 1-methyl-3-((trimethylsilyl)ethynyl)-1H-pyrazole (750 mg, 4.19 mmol) in THF (20 ml) and H₂O (4 ml) was added KOH (235 mg, 4.19 mmol). The reaction mixture was stirred for 1 h at RT. The mixture was poured into H₂O and extracted with EA. The combined organic phase was washed with brine, dried over anhydrous Na₂SO₄, filtered, ad concentrated in vacuum. The residue was used directly in the next step without further purification (350 mg, 79%). MS (ESI) m/e [M+1]⁺=107.

### Step 3: methyl 2-((1-(2,7-dimethyl-3-((1-methyl-1H-pyrazol-3-yl)ethynyl)-1-oxo-1,2-dihydroisoquinolin-5-yl)ethyl)amino)benzoate

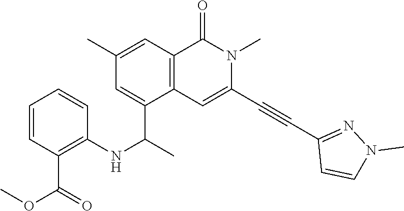

To a solution of methyl 2-((1-(3-chloro-2,7-dimethyl-1-oxo-1,2-dihydroisoquinolin-5-yl)ethyl)amino)benzoate (120 mg, 0.31 mmol) and 3-ethynyl-1-methyl-1H-pyrazole (67 mg, 0.62 mmol) in DMF (5 ml) and TEA (3 ml) was added Pd(PPh₃)₄ (18 mg, 0.02 mmol) and CuI (6 mg, 0.03 mmol) under N₂. The resulting solution was stirred at 70° C. for 15 h. The mixture was poured into H₂O and extracted with EtOAc. The combined organic extracts were washed with brine, dried over anhydrous Na₂SO₄, filtered, and concentrated under vacuum. The residue was purified by flash chromatography on silica gel to give the product (100 mg, 70%). MS (ESI) m/e [M+1]⁺=455.

### Step 4: methyl 2-((1-(2,7-dimethyl-3-((1-methyl-1H-pyrazol-3-yl)ethynyl)-1-oxo-1,2-dihydroisoquinolin-5-yl)ethyl)amino)benzoate

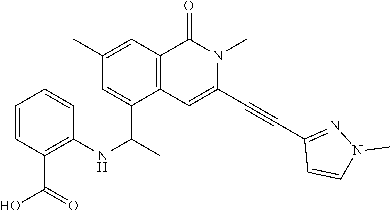 [SMILES: Cc1cc(C(C)Nc2ccccc2C(=O)O)c2cc(C#Cc3ccn(C)n3)n(C)c(=O)c2c1]

To a solution of methyl 2-((1-(2,7-dimethyl-3-((1-methyl-1H-pyrazol-3-yl)ethynyl)-1-oxo-1,2-dihydroisoquinolin-5-yl)ethyl)amino)benzoate (100 mg, 0.22 mmol) in methanol (4 mL), H₂O (2 ml) and THF (4 ml) was added 1N NaOH (0.5 mL). The resulting solution was stirred for 7 h at 50° C. The solution was concentrated under vacuum. The residue was redissolved in H₂O and acidized to pH 5˜6 with HCl (2 M in water). The mixture was filtered, and the filter cake was purified by Prep-HPLC to give the desired product (24 mg, 25%). ¹H NMR (400 MHz, DMSO-d₆) δ ppm 12.79 (s, 1H), 8.49-8.29 (m, 1H), 7.96 (s, 1H), 7.84 (m, 1H), 7.83-7.75 (m, 1H), 7.53-7.42 (m, 2H), 7.24-7.12 (m, 1H), 6.65 (d, J=2.2 Hz, 1H), 6.52 (m, 1H), 6.31 (d, J=8.5 Hz, 1H), 5.35-5.16 (m, 1H), 3.88 (s, 3H), 3.70 (s, 3H), 2.35 (s, 3H), 1.50 (d, J=6.5 Hz, 3H). MS (ESI) m/e [M+1]⁺=441.

### Example 89: 2-((1-(3-((1-hydroxycyclopentyl)ethynyl)-2,7-dimethyl-1-oxo-1,2-dihydroisoquinolin-5-yl)ethyl)amino)benzoic acid

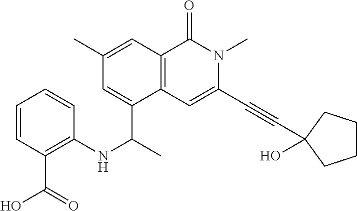 [SMILES: Cc1cc(C(C)Nc2ccccc2C(=O)O)c2cc(C#CC3(O)CCCC3)n(C)c(=O)c2c1]

The desired product (18 mg) was obtained following the similar procedure as Example 88. ¹H NMR (400 MHz, DMSO-d₆) δ ppm 12.79 (s, 1H), 8.45-8.28 (m, 1H), 7.94 (s, 1H), 7.80 (d, J=7.8 Hz, 1H), 7.47 (s, 1H), 7.25 (s, 1H), 7.22-7.09 (m, 1H), 6.52 (m, 1H), 6.29 (d, J=8.5 Hz, 1H), 5.58 (s, 1H), 5.30-5.13 (m, 1H), 3.62 (s, 3H), 2.35 (s, 3H), 1.99-1.87 (m, 4H), 1.84-1.63 (m, 4H), 1.49 (d, J=6.4 Hz, 3H). MS (ESI) m/e [M+1]⁺=445.

### Example 90: 2-((1-(3-(2-carbamoylphenyl)-2,7-dimethyl-1-oxo-1,2-dihydroisoquinolin-5-yl)ethyl)amino)benzoic acid

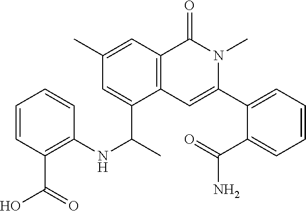 [SMILES: Cc1cc(C(C)Nc2ccccc2C(=O)O)c2cc(-c3ccccc3C(N)=O)n(C)c(=O)c2c1]

The desired product (9.86 mg) was obtained following the similar procedure as Example 10. ¹H NMR (400 MHz, DMSO-d₆) δ ppm 12.71 (s, 1H), 8.39 (s, 1H), 7.97 (s, 1H), 7.88 (d, J=7.2 Hz, 1H), 7.82-7.76 (m, 1H), 7.70-7.49 (m, 4H), 7.45-7.35 (m, 2H), 7.25-7.15 (m, 1H), 6.75-6.65 (m, 1H), 6.55-6.45 (m, 1H), 6.30-6.20 (m, 1H), 5.10-5.00 (m, 1H), 3.23 (d, J=7.2 Hz, 3H), 2.35 (d, J=5.2 Hz, 3H), 1.48 (d, J=6.4 Hz, 3H). MS (ESI) m/e [M+1]⁺=456.

### Example 91: 2-((1-(2,7-dimethyl-3-(1-methyl-1H-pyrazol-4-yl)-1-oxo-1,2-dihydroisoquinolin-5-yl)ethyl)amino)benzoic acid

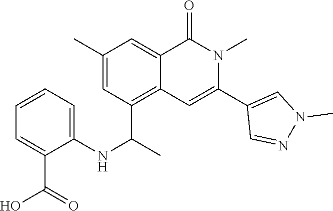 [SMILES: Cc1cc(C(C)Nc2ccccc2C(=O)O)c2cc(-c3cnn(C)c3)n(C)c(=O)c2c1]

The desired product (11.5 mg) was obtained following the similar procedure as Example 10. ¹H NMR (400 MHz, DMSO-d₆) δ ppm 12.79 (brs, 1H), 8.39 (s, 1H), 8.14 (s, 1H), 7.94 (s, 1H), 7.83-7.76 (m, 1H), 7.65-7.50 (m, 1H), 7.44 (s, 1H), 7.20-7.11 (m, 1H), 6.88 (s, 1H), 6.55-6.45 (m, 1H), 6.33 (d, J=8.5 Hz, 1H), 5.25-5.12 (m, 1H), 3.91 (s, 3H), 3.53 (s, 3H), 2.34 (s, 3H), 1.51 (d, J=6.6 Hz, 3H). MS (ESI) m/e [M+1]⁺=417.

### Example 92: 2-((1-(2,7-dimethyl-1-oxo-3-phenyl-1,2-dihydroisoquinolin-5-yl)ethyl)amino)benzo[b]thiophene-3-carboxylic acid

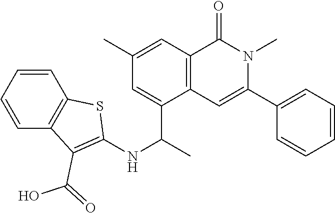 [SMILES: Cc1cc(C(C)Nc2sc3ccccc3c2C(=O)O)c2cc(-c3ccccc3)n(C)c(=O)c2c1]

### Step 1: ethyl 2-((1-(2,7-dimethyl-1-oxo-3-phenyl-1,2-dihydroisoquinolin-5-yl)ethyl)amino)benzo[b]thiophene-3-carboxylate

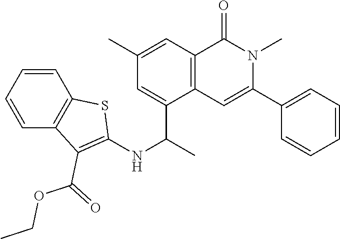 [SMILES: CCOC(=O)c1c(NC(C)c2cc(C)cc3c(=O)n(C)c(-c4ccccc4)cc23)sc2ccccc12]

A solution of 5-(1-bromoethyl)-2,7-dimethyl-3-phenylisoquinolin-1(2H)-one (120 mg, 0.34 mmol), ethyl 2-aminobenzo[b]thiophene-3-carboxylate (150.1 mg, 0.68 mmol) and TEA (172 mg, 1.7 mmol) in DMF (5 mL) was stirred at 80° C. for 2 h. The mixture was cooled to R.T. The solvent was removed, and the crude residue was purified by Prep-TLC to give the desired product (40 mg, 24%). MS (ESI) m/e [M+H]⁺=497.

### Step 2: 2-((1-(2,7-dimethyl-1-oxo-3-phenyl-1,2-dihydroisoquinolin-5-yl)ethyl)amino)benzo[b]thiophene-3-carboxylic acid

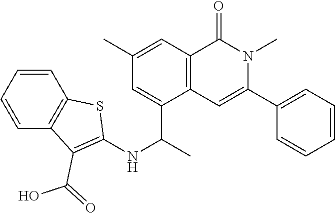 [SMILES: Cc1cc(C(C)Nc2sc3ccccc3c2C(=O)O)c2cc(-c3ccccc3)n(C)c(=O)c2c1]

A solution of ethyl 2-((1-(2,7-dimethyl-1-oxo-3-phenyl-1,2-dihydroisoquinolin-5-yl)ethyl)amino)benzo[b]thiophene-3-carboxylate (40 mg, 0.081 mmol) in NaOH (1 N, 5 mL) and MeOH (5 mL) was stirred at 70° C. for 4 h. The MeOH was removed, the aqueous layer was purified on Prep-HPLC to give the desired product (6.89 mg, 18%). 1′H NMR (400 MHz, DMSO-d₆) δ ppm 10.32 (s, 1H), 8.40 (s, 1H), 7.98 (s, 1H), 7.68-7.55 (m, 3H), 7.54-7.46 (m, 3H), 7.39 (d, J=7.9 Hz, 1H), 7.10-7.00 (m, 1H), 6.90-6.70 (m, 1H), 6.77 (s, 1H), 5.04 (s, 1H), 3.33 (s, 3H), 2.38 (s, 3H), 1.53 (d, J=6.4 Hz, 3H). MS (ESI) m/e [M+H]⁺=469.

### Example 93: 5-(1-(benzo[b]thiophen-3-ylamino)ethyl)-2,7-dimethyl-3-phenylisoquinolin-1(2H)-one

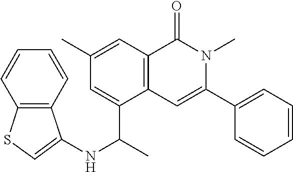 [SMILES: Cc1cc(C(C)Nc2csc3ccccc23)c2cc(-c3ccccc3)n(C)c(=O)c2c1]

A decarboxylation byproduct 5-(1-(benzo[b]thiophen-3-ylamino)ethyl)-2,7-dimethyl-3-phenylisoquinolin-1(2H)-one (19.62 mg) was obtained during the purification of 2-((1-(2,7-dimethyl-1-oxo-3-phenyl-1,2-dihydroisoquinolin-5-yl)ethyl)amino)benzo[b]thiophene-3-carboxylic acid (Example 92). ¹H NMR (400 MHz, DMSO-d₆) δ ppm 7.98 (s, 1H), 7.83 (s, 1H), 7.63 (d, J=7.1 Hz, 1H), 7.54 (s, 1H), 7.43 (s, 2H), 7.38 (s, 3H), 7.22 (d, J=8.2 Hz, 2H), 6.74 (s, 1H), 6.32 (s, 1H), 5.44 (s, 1H), 4.91 (s, 1H), 2.22 (s, 3H), 1.40 (d, J=6.6 Hz, 3H). MS (ESI) m/e [M+H]⁺=425.

### Example 94: 2-((1-(2,7-dimethyl-1-oxo-3-((1-phenyl-1H-pyrazol-4-yl)ethynyl)-1,2-dihydroisoquinolin-5-yl)ethyl)amino)benzoic acid

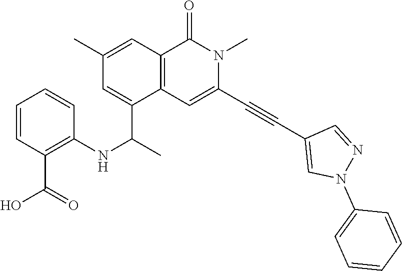 [SMILES: Cc1cc(C(C)Nc2ccccc2C(=O)O)c2cc(C#Cc3cnn(-c4ccccc4)c3)n(C)c(=O)c2c1]

### Step 1: 4-iodo-1-phenyl-1H-pyrazole

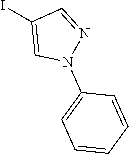 [SMILES: Ic1cnn(-c2ccccc2)c1]

To a solution of 4-iodo-1H-pyrazole (3 g, 15 mmol), phenylboronic acid (3.7 g, 30 mmol) and pyridine (5.9 g, 75 mmol) in DCM (10 ml) was added Cu(OAc)₂ (2.8 g, 15 mmol). The resulting solution was stirred at 25° C. for 36 h. The mixture was diluted with DCM, washed with H₂O and brine. The organic layer was dried over Na₂SO₄, filtered, and concentrated under vacuum. The residue was purified by CombiFlash chromatography on silica gel to give the desired product (2.8 g, 69%). MS (ESI) m/e [M+1]⁺=271.

### Step 2: 1-phenyl-4-((trimethylsilyl)ethynyl)-1H-pyrazole

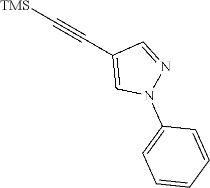 [SMILES: C[Si](C)(C)C#Cc1cnn(-c2ccccc2)c1]

A mixture of 4-iodo-1-phenyl-1H-pyrazole (2.8 g, 10.3 mmol), ethynyltrimethylsilane (1.5 g, 15.5 mmol), Pd(PPh₂)Cl₂ (702 mg, 1.0 mmol) in TEA (5 mL) and DMF (20 mL) was stirred at 80° C. for 15 h under N₂. The mixture was poured into H₂O and extracted with EtOAc. The organic layer was dried over Na₂SO₄, filtered, and concentrated under vacuum. The residue was purified CombiFlash chromatography on silica gel to give the desired product (2.3 g, 92%). MS (ESI) m/e [M+1]⁺=241.

### Step 3: 4-ethynyl-1-phenyl-1H-pyrazole

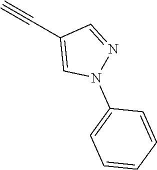

To a solution of 1-phenyl-4-((trimethylsilyl)ethynyl)-1H-pyrazole (2.3 g, 9.5 mmol) in MeOH (30 mL) was added K₂CO₃ (2.6 g, 19 mmol). The resulting mixture was stirred at 25° C. for 15 h. The mixture was concentrated under vacuum, diluted with EtOAc, washed with H₂O and brine. The organic layer was dried over Na₂SO₄, filtered, and concentrated under vacuum. The residue was purified CombiFlash chromatography on silica gel to give the desired product (1.1 g, 69%). MS (ESI) m/e [M+1]⁺=169.

### Step 4: methyl 2-((1-(2,7-dimethyl-1-oxo-3-((1-phenyl-1H-pyrazol-4-yl)ethynyl)-1,2-dihydroisoquinolin-5-yl)ethyl)amino)benzoate

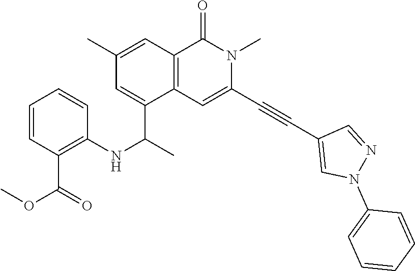 [SMILES: COC(=O)c1ccccc1NC(C)c1cc(C)cc2c(=O)n(C)c(C#Cc3cnn(-c4ccccc4)c3)cc12]

A mixture of methyl 2-((1-(3-chloro-2,7-dimethyl-1-oxo-1,2-dihydroisoquinolin-5-yl)ethyl)amino)benzoate (50 mg, 0.13 mmol), 4-ethynyl-1-phenyl-1H-pyrazole (50 mg, 0.3 mmol), Pd(PPh₂)Cl₂ (8 mg, 0.01 mmol) in TEA (1 mL) and DMF (2 mL) was stirred at 100° C. for 15 h under N₂. The mixture was poured into H₂O and extracted with EtOAc. The organic layer was dried over Na₂SO₄, filtered, and concentrated under vacuum. The residue was purified by Prep-TLC to give the desired product (30 mg, 45%). MS (ESI) m/e [M+1]⁺=517.

### Step 5: 2-((1-(2,7-dimethyl-1-oxo-3-((1-phenyl-1H-pyrazol-4-yl)ethynyl)-1,2-dihydroisoquinolin-5-yl)ethyl)amino)benzoic acid

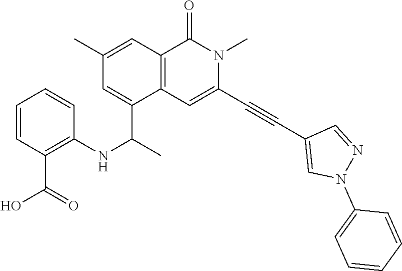 [SMILES: Cc1cc(C(C)Nc2ccccc2C(=O)O)c2cc(C#Cc3cnn(-c4ccccc4)c3)n(C)c(=O)c2c1]

To a solution of methyl 2-((1-(2,7-dimethyl-1-oxo-3-((1-phenyl-1H-pyrazol-4-yl)ethynyl)-1,2-dihydroisoquinolin-5-yl)ethyl)amino)benzoate (30 mg, 0.06 mmol) in methanol (3 ml) was added NaOH (3 M, 1 mL). The resulting solution was stirred for 5 h at 50° C. The solution was concentrated under vacuum. The residue was redissolved in H₂O and acidized to pH 5˜6 with HCl (2 M in water). The mixture was filtered and the solid was collected, purified by Prep-HPLC to give the desired product (19 mg, 63%). ¹H NMR (400 MHz, DMSO-d₆) δ ppm 9.06 (s, 1H), 8.55 (s, 1H), 8.14 (s, 1H), 7.96 (s, 1H), 7.91-7.84 (m, 2H), 7.83-7.76 (m, 1H), 7.59-7.47 (m, 3H), 7.45-7.32 (m, 2H), 7.21-7.09 (m, 1H), 6.51 (m, 1H), 6.30 (d, J=8.5 Hz, 1H), 5.30-5.14 (m, 1H), 3.71 (s, 3H), 2.35 (s, 3H), 1.51 (d, J=6.5 Hz, 3H). MS (ESI) m/e [M+1]⁺=503.

### Example 95: 2-((1-(3-(4-fluorophenyl)-7-methyl-2-(oxetan-3-yl)-1-oxo-1,2-dihydroisoquinolin-5-yl)ethyl)amino)benzoic acid

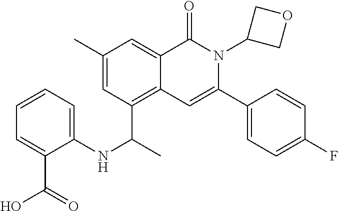 [SMILES: Cc1cc(C(C)Nc2ccccc2C(=O)O)c2cc(-c3ccc(F)cc3)n(C3COC3)c(=O)c2c1]

The desired product (29 mg) was obtained following the similar procedure as Example 63. ¹H NMR (400 MHz, DMSO-d₆) δ ppm 8.72 (s, 1H), 8.29-8.19 (m, 3H), 7.96 (s, 1H), 7.86-7.77 (m, 1H), 7.58 (s, 1H), 7.39-7.26 (m, 2H), 7.17-7.05 (m, 1H), 6.55-6.45 (m, 1H), 6.37 (d, J=8.5 Hz, 1H), 5.95-5.84 (m, 1H), 5.63-5.51 (m, 1H), 5.07 (t, J=6.9 Hz, 2H), 4.82-4.71 (m, 2H), 2.43 (s, 3H), 1.58 (d, J=6.5 Hz, 3H). MS (ESI) m/e [M+1]⁺=473.

### Example 96: 2-((1-(3-(4-fluorophenyl)-7-methyl-1-oxo-2-(tetrahydro-2H-pyran-4-yl)-1,2-dihydroisoquinolin-5-yl)ethyl)amino)benzoic acid

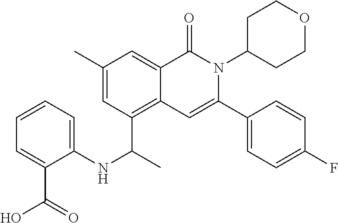 [SMILES: Cc1cc(C(C)Nc2ccccc2C(=O)O)c2cc(-c3ccc(F)cc3)n(C3CCOCC3)c(=O)c2c1]

The desired product (55 mg) was obtained following the similar procedure as Example 63. ¹H NMR (400 MHz, DMSO-d₆) δ ppm 12.76 (s, 1H), 8.54-8.41 (m, 1H), 8.33-8.24 (m, 2H), 8.19 (s, 1H), 7.91 (s, 1H), 7.81 (dd, J=7.9, 1.5 Hz, 1H), 7.59-7.49 (m, 1H), 7.39-7.27 (m, 2H), 7.21-7.10 (m, 1H), 6.51 (m, 1H), 6.40 (d, J=8.5 Hz, 1H), 5.67-5.51 (m, 2H), 4.01-3.89 (m, 2H), 3.72-3.57 (m, 2H), 2.42 (s, 3H), 2.22-2.10 (m, 2H), 1.91-1.75 (m, 2H), 1.59 (d, J=6.5 Hz, 3H). MS (ESI) m/e [M+1]⁺=501.

### Example 97: 4-((1-(3-(4-fluorophenyl)-2,7-dimethyl-1-oxo-1,2-dihydroisoquinolin-5-yl)ethyl)amino)nicotinic acid

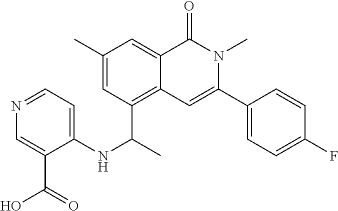 [SMILES: Cc1cc(C(C)Nc2ccncc2C(=O)O)c2cc(-c3ccc(F)cc3)n(C)c(=O)c2c1]

### Step 1: methyl 4-((1-(3-(4-fluorophenyl)-2,7-dimethyl-1-oxo-1,2-dihydroisoquinolin-5-yl)ethyl)amino)nicotinate

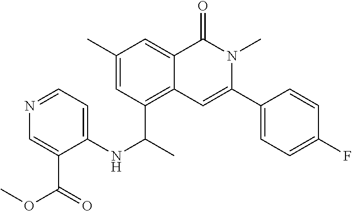 [SMILES: COC(=O)c1cnccc1NC(C)c1cc(C)cc2c(=O)n(C)c(-c3ccc(F)cc3)cc12]

A mixture of 5-(1-bromoethyl)-3-(4-fluorophenyl)-2,7-dimethylisoquinolin-1(2H)-one (40 mg, 0.11 mmol), methyl 4-aminonicotinate (24 mg, 0.16 mmol), DIEA (41 mg, 0.32 mmol) in DMF (4 ml) was stirred at 90° C. for 2 h. The mixture was concentrated under vacuum. The residue was purified by Prep-TLC to give the desired product (30 mg, 63%). MS (ESI) m/e [M+1]⁺=446.

### Step 2: 4-((1-(3-(4-fluorophenyl)-2,7-dimethyl-1-oxo-1,2-dihydroisoquinolin-5-yl)ethyl)amino)nicotinic acid

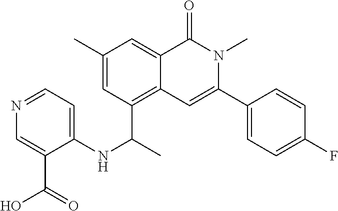 [SMILES: Cc1cc(C(C)Nc2ccncc2C(=O)O)c2cc(-c3ccc(F)cc3)n(C)c(=O)c2c1]

To a solution of methyl 4-((1-(3-(4-fluorophenyl)-2,7-dimethyl-1-oxo-1,2-dihydroisoquinolin-5-yl)ethyl)amino)nicotinate (20 mg, 0.04 mmol) in MeOH (1 ml) and THF (0.5 ml) was added 4 N NaOH (0.2 mL). The reaction mixture was stirred at rt for 1 h. The mixture was adjusted to PH 5 by 1 N HCl and concentrated under vacuum. The residue was purified by Prep-HPLC to give the desired product (6.88 mg, 35%). ¹H NMR (400 MHz, DMSO-d₆) δ ppm 10.53-10.51 (m, 1H), 8.64 (s, 1H), 8.44 (d, J=8.0 Hz, 1H), 8.14 (s, 1H), 8.09 (d, J=5.4 Hz, 1H), 7.74 (s, 1H), 7.56-7.52 (m, 2H), 7.36-7.32 (m, 2H), 6.79 (d, J=8.0 Hz, 1H), 6.61 (s, 1H), 6.38 (d, J=8.0 Hz, 1H), 3.27 (s, 3H), 2.51 (s, 3H), 1.86 (d, J=6.7 Hz, 3H). MS (ESI) m/e [M+1]⁺=432.

### Example 98: (R)-2-((1-(3-(4-(2-hydroxypropan-2-yl)phenyl)-2,7-dimethyl-1-oxo-1,2-dihydroisoquinolin-5-yl)ethyl)amino)benzoic acid

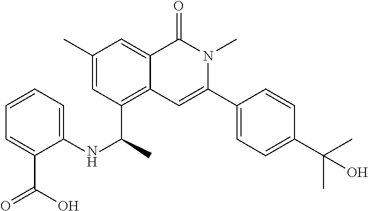 [SMILES: Cc1cc([C@@H](C)Nc2ccccc2C(=O)O)c2cc(-c3ccc(C(C)(C)O)cc3)n(C)c(=O)c2c1]

### Step 1: (S)-3-chloro-5-(1-hydroxyethyl)-2,7-dimethylisoquinolin-1(2H)-one

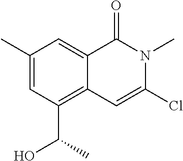

A N₂ filled and dried 100 mL screw-cap Schlenk bottle containing a magnetic stir bar was charged with Cu(OAc)₂ (36 mg, 0.2 mmol), S-DTBM-SEGPHOS (259 mg, 0.22 mol). Anhydrous THF (20 mL) was added, and the mixture was stirred for 30 min. The resulting solution was added PhSiH₃ (648 mg, 6 mmol) and the resulting solution was stirred for another 30 min. After cooled to −25° C. A solution of 5-acetyl-3-chloro-2,7-dimethylisoquinolin-1(2H)-one (1 g, 4 mmol) in 20 mL THF was added. After stirring for 2 h, saturated NH₄F solution in methanol was added and stirred for 10 min. Saturated NH₄Cl (aqueous) was then added. The resulting mixture was extracted with EA. The combined organic layer was washed with brine, dried over anhydrous Na₂SO₄, and concentrated under vacuum. The residue was subjected to column chromatography on silica gel to afford the product (820 mg, 96:4 e.r.), which was then recrystallized in organic solvent to afford the desired product (650 mg, 65%, 99:1 e.r.). MS (ESI) m/e [M+1]⁺=252.

### Step 2: (R)-1-(1-(3-chloro-2,7-dimethyl-1-oxo-1,2-dihydroisoquinolin-5-yl)ethyl)-2H-benzo[d][1,3]oxazine-2,4(1H)-dione

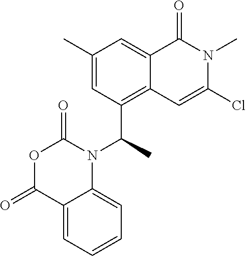 [SMILES: Cc1cc([C@@H](C)n2c(=O)oc(=O)c3ccccc32)c2cc(Cl)n(C)c(=O)c2c1]

A solution of (S)-3-chloro-5-(1-hydroxyethyl)-2,7-dimethylisoquinolin-1(2H)-one (150 mg, 0.60 mmol), PPh₃ (237 mg, 0.90 mmol) and 2H-benzo[d][1,3]oxazine-2,4(1H)-dione (147 mg, 0.90 mmol) in THF (1 mL) was added DIAD (180 mg, 0.90 mmol) at 0° C. The mixture was stirred at rt for 2 h. The reaction was quenched by adding H₂O, and extracted with EA. The combined organic layer was dried over anhydrous Na₂SO₄, and concentrated under vacuum. The residue (150 mg, crude) was directly used in the next step without further purification. MS (ESI) m/e [M+1]⁺=367.

### Step 3: (R)-2-((1-(3-(4-(2-hydroxypropan-2-yl)phenyl)-2,7-dimethyl-1-oxo-1,2-dihydroisoquinolin-5-yl)ethyl)amino)benzoic acid

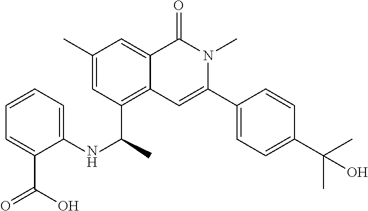 [SMILES: Cc1cc([C@@H](C)Nc2ccccc2C(=O)O)c2cc(-c3ccc(C(C)(C)O)cc3)n(C)c(=O)c2c1]

A mixture of (R)-1-(1-(3-chloro-2,7-dimethyl-1-oxo-1,2-dihydroisoquinolin-5-yl)ethyl)-2H-benzo[d][1,3]oxazine-2,4(1H)-dione (150 mg crude, 0.4 mmol), (4-(2-hydroxypropan-2-yl)phenyl)boronic acid (140 mg, 0.8 mmol), Pd(PPh₃)₄ (46 mg, 0.04 mmol) and K₃PO₄ (169 mg, 0.8 mmol) in dioxane (24 mL) and H₂O (6 mL) was stirred at 90° C. under N₂ for 5 h. The solvent was removed, and the residue was purified by Prep-HPLC followed by Chiral SFC to give desired product (73 mg, 26% yield over 2 steps, 99.6:0.4 e.r.). ¹H NMR (400 MHz, DMSO-d₆) δ ppm 12.80 (brs, 1H), 8.41 (s, 1H), 7.99 (s, 1H), 7.81 (dd, J=7.9, 1.5 Hz, 1H), 7.62 (d, J=8.3 Hz, 2H), 7.56-7.45 (m, 3H), 7.25-7.08 (m, 1H), 6.83 (s, 1H), 6.58-6.46 (m, 1H), 6.33 (d, J=8.5 Hz, 1H), 5.25-5.16 (s, 1H), 5.15 (brs, 1H), 3.34 (s, 3H), 2.37 (s, 3H), 1.56-1.41 (m, 9H). MS (ESI) m/e [M+Na]⁺=493.

### Biochemical Assays

PI3Kα (p110/p85) kinase ADP-Glo Assay. Certain compounds disclosed herein were tested for inhibition of PI3Kα (p110/p85) kinase (wild type, “WT”) in assays based on the ADP-Glo™ Kinase Assay methodology. The assays were carried out in 384-well white plates in a reaction mixture containing PI3Kα (p110/p85) kinase, 25 M ATP, 5.35 g/mL PI(4,5)P2:PS Lipid Kinase Substrate and 0-100 M compound in buffer containing 50 mM HEPES pH7.5, 50 mM NaCl, 3 mM MgCl2, 0.0025% BSA and 2 mM DTT. PI3Kα (p110/p85) kinase was incubated with Lipid Kinase Substrate and compound for 30 minutes at room temperature and the reaction was initiated by the addition of ATP. After reaction at room temperature for 120 minutes, ADP-Glo™ reagents were added to terminate the kinase reaction and deplete the remaining ATP according to the manufacture's instruction (Promega Corporation). After incubation at room temperature for 1 hour, Kinase Detection Reagent (prepared by reconstituting the lyophilized Kinase Detection Substrate with Kinase Detection Buffer of the kit) was added to convert the ADP to ATP and to allow for the newly synthesized ATP to be measured using a luciferase/luciferin reaction. After incubation at room temperature for 0.5 hour, chemiluminescence signals were recorded on a PHERAstar FSX plate reader (BMG Labtech). The chemiluminescence generated in this assay correlates to the amount of ADP generated in the kinase assay, indicating kinase activity. Inhibition of PI3Kα (p110/p85) kinase activity resulted in decrease of the chemiluminescence signal. The IC₅₀ for each compound was derived from fitting the data to the four-parameter logistic equation by Dotmatics.

PI3Kα (p110[H1047R]/p85) kinase ADP-Glo Assay. Certain compounds disclosed herein were tested for inhibition of PI3Kα (p110[H1047R]/p85) kinase in assays based on the ADP-Glo™ Kinase Assay methodology. The assays were carried out in 384-well white plates in a reaction mixture containing PI3Kα (p110[H1047R]/p85) kinase, 25 M ATP, 5.35 g/ml PI(4,5)P2:PS Lipid Kinase Substrate and 0-100 M compound in buffer containing 50 mM HEPES pH7.5, 50 mM NaCl, 3 mM MgCl₂, 0.0025% BSA and 2 mM DTT. PI3Kα (p110[H1047R]/p85) kinase was incubated with Lipid Kinase Substrate and compound for 30 minutes at room temperature and the reaction was initiated by the addition of ATP.

After reaction at room temperature for 120 minutes, ADP-Glo™ reagents were added to terminate the kinase reaction and deplete the remaining ATP according to the manufacture's instruction (Promega Corporation). After incubation at room temperature for 1 hour, Kinase Detection Reagent (prepared by reconstituting the lyophilized Kinase Detection Substrate with Kinase Detection Buffer of the kit) was added to convert the ADP to ATP and to allow for the newly synthesized ATP to be measured using a luciferase/luciferin reaction. After incubation at room temperature for 0.5 hour, chemiluminescence signals were recorded on a PHERAstar FSX plate reader (BMG Labtech). The chemiluminescence generated in this assay correlates to the amount of ADP generated in the kinase assay, indicating kinase activity. Inhibition of PI3Kα (p110[H1047R]/p85) kinase activity resulted in decrease of the chemiluminescence signal. The IC₅₀ for each compound was derived from fitting the data to the four-parameter logistic equation by Dotmatics.

TABLE 1
Enzyme Activity IC₅₀ for certain compounds disclosed herein

| | ADP-Glo Assay IC₅₀ (nM), 25 uM ATP | | |
|---|---|---|---|
| | | selectivity: | |
| Example NO. | H1047R | WT/H1047R | WT |
| 1 | 170.0 | 16.3 | 2769 |
| 3 | 671.0 | 14.7 | 9847 |
| 4 | 443.0 | 8.7 | 3868 |
| 5 | 375.0 | 14.1 | 5279 |
| 6 | 162.0 | 17.8 | 2884 |
| 7 | 164.0 | 15.3 | 2516 |
| 8 | 183.0 | 32.6 | 5962 |
| 9 | 187.0 | 14.5 | 2710 |
| 10 | 224.0 | 36.1 | 8082 |
| 11 | 210.0 | 100.2 | 21052 |
| 12 | 175.0 | 38.2 | 6693 |
| 13 | 192.0 | 30.3 | 5823 |
| 15 | 858.0 | 8.0 | 6858 |
| 20 | 93.0 | 108.4 | 10077 |
| 21 | 121.0 | 11.1 | 1345 |
| 22 | 1725.0 | 30.3 | 52274 |
| 23 | 223.0 | 9.1 | 2035 |
| 25 | 229.0 | 104.0 | 23818 |
| 26 | 158 | 40 | 6251 |
| 27 | 762 | 79 | 60236 |
| 28 | 505 | >198 | >100000 |
| 29 | 260 | 15 | 3796 |
| 2 | 161.0 | 139.3 | 22427.0 |
| 14 | 156.0 | 23.9 | 3722.0 |
| 16 | 240.0 | 15.3 | 3666.0 |
| 17 | 255.0 | 30.5 | 7777.0 |
| 18 | 272.0 | 16.1 | 4367.0 |
| 19 | 311.0 | 21.1 | 6559.0 |
| 24 | 1027.0 | 38.2 | 39240.0 |
| 30 | 104.0 | 24.1 | 2502.0 |
| 31 | >100000 | NA | >100000 |
| 32 | 332.0 | 184.0 | 61090.0 |
| 33 | 1048.0 | 52.6 | 55127.0 |
| 34 | 107.0 | 39.0 | 4177.0 |
| 35 | >100000 | NA | >100000 |
| 36 | 603.0 | 84.2 | 50765.0 |
| 37 | 309.0 | 40.6 | 12541.0 |
| 38 | 267.0 | 36.7 | 9805.0 |
| 39 | 922.0 | 11.5 | 10629.0 |
| 40 | 55.0 | 93.1 | 5123.0 |
| 41 | 7180.0 | 8.5 | 61036.0 |
| 42 | 971.0 | 73.8 | 71645.0 |
| 43 | 235.0 | 10.1 | 2363.0 |
| 44 | 795.0 | 2.3 | 1838.0 |
| 45 | 273.0 | 26.4 | 7212.0 |
| 46 | 480.0 | 23.0 | 11063.0 |
| 47 | 525.0 | 27.3 | 14316.0 |
| 48 | 829.0 | 6.2 | 5164.0 |
| 49 | 457.0 | 20.8 | 9526.0 |
| 50 | 376.0 | 27.6 | 10386.0 |
| 51 | 247.0 | 42.1 | 10411.0 |
| 52 | 375.0 | 25.2 | 9447.0 |
| 53 | 261.0 | 18.5 | 4837.0 |
| 54 | 9289.0 | >10 | >100000 |
| 55 | 725.0 | 35.9 | 26004.0 |
| 56 | 1047 | 16.3 | 17075 |
| 57 | >100000 | <0.9 | 88364 |
| 58 | 336.0 | 29.7 | 9992.0 |
| 59 | 281.0 | 15.3 | 4311.0 |
| 60 | 16392.0 | 1.5 | 24113.0 |
| 61 | 790.0 | 24.6 | 19466.0 |
| 62 | 230.0 | 20.6 | 4727.0 |
| 63 | 92.0 | 66.8 | 6143.0 |
| 64 | 113.0 | 21.9 | 2475.0 |
| 65 | >100000 | NA | >100000 |
| 66 | 1113.0 | 19.3 | 21512.0 |
| 67 | 1214.0 | 11.2 | 13588.0 |
| 68 | 21695.0 | 1.6 | 34288.0 |
| 69 | 416.0 | 45.3 | 18830.0 |
| 70 | 872.0 | 24.2 | 21132.0 |
| 71 | 312.0 | 45.7 | 14247.0 |
| 72 | 1072.0 | 19.2 | 20535.0 |
| 73 | 1027.0 | 25.4 | 26108.0 |
| 74 | 729.0 | 18.0 | 13111.0 |
| 75 | 974.0 | 32.3 | 31419.0 |
| 76 | 516.0 | 22.8 | 11780.0 |
| 77 | 325.0 | 21.4 | 6954.0 |
| 78 | 2214.0 | 29.7 | 65859.0 |
| 79 | 2422 | 24.4 | 59191 |
| 80 | 304.0 | 10.8 | 3279.0 |
| 81 | 215.0 | 17.6 | 3781.0 |
| 82 | 549.0 | 6.0 | 3313.0 |
| 83 | 485.0 | 114.5 | 55541.0 |
| 84 | 242.0 | 60.4 | 14623.0 |
| 85 | 1101.0 | 24.3 | 26809.0 |
| 86 | 575.0 | 26.3 | 15141.0 |
| 87 | 1503.0 | 23.5 | 35360.0 |
| 88 | 152.0 | 11.5 | 1746.0 |
| 89 | 158.0 | 43.3 | 6844.0 |
| 90 | 1931.0 | 46.0 | 88785.0 |
| 91 | 240.0 | 40.9 | 9821.0 |
| 92 | 887.0 | >113 | >100000 |
| 93 | >100000 | NA | >100000 |
| 94 | 387.0 | >258 | >100000 |
| 95 | 91.0 | 47.6 | 4335.0 |
| 96 | 910.0 | 79.1 | 71962.0 |
| 97 | >100000 | NA | >100000 |
| 98 | 432.0 | 17.0 | 7355.0 |

### Cell Assay

Cell Culture Maintenance. The T-47D (ATCC, HTB-133) cell line was obtained from the American Type Culture Collection. Cells were maintained in RPMI1640 medium (Gibco, REF #22400105) supplemented with 10% Fetal Bovine Serum (Gibco, REF #10091148). Cultures were maintained in a humidified incubator at 37° C. under 5% CO₂/95% air.

The SK-BR-3 (ATCC, HTB-30) cell line was obtained from the American Type Culture Collection. Cells were maintained in McCoy' 5A medium (Gibco, REF #16600108) supplemented with 10% Fetal Bovine Serum. Cultures were maintained in a humidified incubator at 37° C. under 5% CO₂/95% air.

Phospho-AKT Inhibition Assay. HTRF Phospho-AKT (Ser473) detection kit (Cisbio, REF #64AKSPEH) was used in phosphor-AKT inhibition assay. For compound screening, PI3Kα H1047R mutated cell line T-47D and WT cell line SK-BR-3 cells were seeded at a density of 3×10⁴ cells per well in 96 well plate (Costar, REF #3599) in 95 μL of assay medium (MEM medium (Gibco, REF #12561-056) with 1×NEAA (Gibco, REF #11140-050), 1 mM sodium pyruvate (Gibco, REF #11360-070), and 1 g/ml human insulin (Topscience, REF #T8221)), overnight. Compounds dissolved in 10 mM stock solutions in DMSO were serially diluted 1:3 in DMSO to generate an 11-point dilution series and plated using an INTEGRA ASSIST PLUS pipetting robot. A 20× intermediate compound dilution plate in RPMI1640 medium (200 M starting compound concentration in 2% DMSO) was then prepared. Five μL of intermediate serially diluted compounds were added to the cell plate to final concentrations ranging from 10 M to 0.1 nM in 0.1% DMSO. 0.1% DMSO alone was used to establish the maximum signal and GDC0077 at final concentration of 10 μM was used as a reference compound for minimum signal. After 1 hour treatment, removed medium and added 50 μL of supplemented lysis buffer (1×) immediately. Incubated for at least 30 minutes at room temperature under shaking. Transferred 16 μL of cell lysate from the 96-well cell-culture plate to a 384-white detection plate (PerkinElmer, Optiplate 384, 6007299). Added 4 μL of premixed antibody solutions prepared in the detection buffer. Incubated 4 hours at room temperature in dark. The plates were read on PHERAstar FSX Microplate reader (BMG LABTECH, Inc.) using standard HTRF settings.

Relative IC₅₀ values were determined using luminescence units by calculating percent inhibition with respect to the in-plate “MIN” (GDC0077 reference control) and “MAX” (DMSO) controls. The data were analyzed using a 4-parameter nonlinear logistic equation (four-parameter logistic concentration-response curve): Y=bottom+[(top−bottom)/1+(X/IC₅₀) slope]
where Y=% inhibition, X=concentration of inhibitor, bottom=minimum value of y attained by curve-fit, top=maximum value of y attained by curve-fit and slope=steepness of curve at the IC₅₀.

$$Selectivity = {IC}_{50} ( PI 3 K α WT ) / {IC}_{50} ( PI 3 K α H 1047 R )$$

TABLE 2
Phospho-AKT Inhibition Assay IC₅₀ for certain compounds disclosed herein

| | p-AKT Inhibition assay (S473) IC₅₀ (nM) | | |
|---|---|---|---|
| | | Selectivity: | |
| Example NO. | T-47D | SK-BR-3/T-47D | SK-BR-3 |
| 1 | 43.5 | 48.6 | 2110.4 |
| 3 | 167.5 | 26.0 | 4354.0 |
| 4 | 144.4 | >69 | >10000.0 |
| 5 | 134.6 | 34.3 | 4617.5 |
| 6 | 62.4 | 51.5 | 3213.9 |
| 7 | 53.9 | 34.5 | 1857.5 |
| 8 | 66.4 | 37.7 | 2501.1 |
| 9 | 70.9 | 51.4 | 3642.4 |
| 10 | 81.98 | 33.3 | 2730.5 |
| 11 | 80.21 | >125 | >10000.0 |
| 12 | 55.27 | 39.0 | 2157.5 |
| 13 | 64.0 | 56.1 | 3588.0 |
| 15 | 217.0 | 38.5 | 8364.4 |
| 20 | 24.1 | >416 | >10000 |
| 21 | 35.55 | 165.3 | 5877.1 |
| 22 | 1753.8 | >5.7 | >10000.0 |
| 23 | 102.6 | 16.6 | 1699.1 |
| 25 | 299.1 | >33.4 | >10000.0 |
| 26 | 50 | >198 | >10000 |
| 27 | 245 | >21 | >10000 |
| 28 | 472 | 17 | 6431.2 |
| 29 | 107 | 25 | 2704.3 |
| 2 | 66.3 | 43.0 | 2848.8 |
| 14 | 63.2 | 32.5 | 2055.4 |
| 16 | 1579.6 | >6.3 | >10000.0 |
| 17 | 58.8 | 13.5 | 795.3 |
| 18 | 85.1 | 27.3 | 2322.3 |
| 19 | 165.8 | 13.8 | 2285.1 |
| 24 | 3135.9 | >3.1 | >10000.0 |
| 30 | 22.02 | 113.7 | 2503.3 |
| 31 | >10000.0 | NA | >10000.0 |
| 32 | 263.7 | >38 | >10000.0 |
| 33 | 271.1 | 36.6 | 9920.2 |
| 34 | 20.23 | >494 | >10000.0 |
| 35 | >10000.0 | NA | >10000.0 |
| 36 | 466.9 | >21 | >10000.0 |
| 37 | 381.9 | >26 | >10000.0 |
| 38 | 284.6 | >35 | >10000.0 |
| 39 | 1110.2 | 5.6 | 6167.2 |
| 40 | 24.3 | 33.5 | 813.8 |
| 41 | >10000.0 | NA | 3230.9 |
| 42 | 297.8 | | |
| 43 | 408.5 | 4.0 | 1643.3 |
| 44 | 559.5 | 3.8 | 2129.7 |
| 45 | 63.92 | 44.0 | 2815.3 |
| 46 | 92.37 | 54.8 | 5065.9 |
| 47 | 57.17 | >175 | >10000.0 |
| 48 | 631.5 | 6.0 | 3794.8 |
| 49 | 89.22 | 50.7 | 4521.7 |
| 50 | 86.58 | 79.1 | 6850.2 |
| 51 | 166.9 | 38.7 | 6452.9 |
| 52 | 75.6 | 95.1 | 7186.4 |
| 53 | 44.66 | 56.6 | 2526.4 |
| 54 | | | |
| 55 | 1563.0 | >6.4 | >10000.0 |
| 56 | 511.6 | >19.55 | >10000.0 |
| 57 | >10000.0 | NA | >10000.0 |
| 58 | 220.2 | 14.3 | 3158.4 |
| 59 | 81.53 | >123 | >10000.0 |
| 60 | | | |
| 61 | 1397.9 | >7.15 | >10000.0 |
| 62 | 129.8 | 22.9 | 2973.3 |
| 63 | 57.28 | 83.3 | 4771.8 |
| 64 | 32.42 | >308 | >10000.0 |
| 65 | | | |
| 66 | 2799.0 | >3.57 | >10000.0 |
| 67 | 1537.2 | >6.51 | >10000.0 |
| 68 | | | |
| 69 | 102.9 | >97 | >10000.0 |
| 70 | 1098.1 | >9.11 | >10000.0 |
| 71 | 101.6 | 55.0 | 5589.8 |
| 72 | 633.4 | >15.79 | >10000.0 |
| 73 | 923.9 | >10.82 | >10000.0 |
| 74 | 307.1 | 14.3 | 4387.7 |
| 75 | 546.6 | >18.29 | >10000.0 |
| 76 | 122.7 | 43.5 | 5338.7 |
| 77 | 84.29 | 24.2 | 2039.2 |
| 78 | 1954.0 | >5.12 | >10000.0 |
| 79 | >10000.0 | NA | >10000.0 |
| 80 | 69.75 | 44.1 | 3077.9 |
| 81 | 40.92 | 45.6 | 1865.5 |
| 82 | 155.0 | 13.0 | 2020.1 |
| 83 | 6754.0 | >1.48 | >10000.0 |
| 84 | 59.96 | 52.0 | 3115.9 |
| 85 | >10000.0 | NA | >10000.0 |
| 86 | 372.0 | >27 | >10000.0 |
| 87 | 432.2 | 6.6 | 2835.4 |
| 88 | 68.48 | 28.3 | 1940.0 |
| 89 | 183.6 | 37.2 | 6837.9 |
| 90 | | | |
| 91 | 409.7 | 11.6 | 4733.1 |
| 92 | 109.4 | >91 | >10000.0 |
| 93 | | | |
| 94 | 3275.5 | >3.05 | >10000.0 |
| 95 | 25.64 | 52.9 | 1356.4 |
| 96 | 330.3 | 28.8 | 9521.5 |
| 97 | >10000.0 | NA | >10000.0 |

Cell Proliferation Assay. PI3Kα H1047R mutated cell line T-47D cells and WT cell line SK-BR-3 were seeded at a density of 3×10³ cells per well in 96 well plate (Xinyou Biotechnology, 062096) in 95 μL of culture medium, overnight. Compounds dissolved in 10 mM stock solutions in DMSO were serially diluted 1:3 in DMSO to generate a 9-point dilution series and plated using an INTEGRA ASSIST Plus pipetting robot. A 20× intermediate compound dilution plate in RPMI1640 medium (200 M starting compound concentration in 2% DMSO) was then prepared. Five μL of intermediate serially diluted compounds were added to the cell plate to final concentrations ranging from 10 M to 1 nM in 0.1% DMSO. 0.1% DMSO alone was used to establish the maximum signal and medium without cells was for minimum signal. After 3 days treatment, equilibrated the plate and its contents at room temperature for approximately 30 minutes. Added 30 μL CellTiter-Glo® Reagent (Promega, REF #G7571) to each well. Then mixed contents for 5 min on plate shaker to induce cell lysis. Incubated at room temperature for 10 minutes to stabilize luminescent signal. The plates were read on PHERAstar FSX Microplate reader using standard luminescence settings.

Relative IC₅₀ values were determined using luminescence units by calculating percent inhibition with respect to the in-plate “MIN” (medium only) and “MAX” (0.1% DMSO) controls. The data were analyzed using a 4-parameter nonlinear logistic equation (four-parameter logistic concentration-response curve): Y=bottom+[(top−bottom)/1+(X/IC₅₀) slope]
where Y=% inhibition, X=concentration of inhibitor, bottom=minimum value of y attained by curve-fit, top=maximum value of y attained by curve-fit and slope=steepness of curve at the IC₅₀.

$$Selectivity = {IC}_{50} ( PI 3 K α WT ) / {IC}_{50} ( PI 3 K α H 1047 R )$$

TABLE 3
Cellular Activity IC₅₀ for certain compounds disclosed herein

| | Cell proliferation IC₅₀ (nM) | | |
|---|---|---|---|
| | | Selectivity: | |
| Example NO. | T-47D | SK-BR-3/T-47D | SK-BR-3 |
| 1 | 84.58 | >118 | >10000.0 |
| 3 | 142.7 | >70 | >10000.0 |
| 4 | 112.4 | >89 | >10000.0 |
| 5 | 520.2 | >19 | >10000.0 |
| 6 | 326.5 | >31 | >10000.0 |
| 7 | 101.2 | >98.8 | >10000.0 |
| 8 | 158.0 | >63 | >10000.0 |
| 9 | 143.4 | >70 | >10000.0 |
| 10 | 128.5 | >78 | >10000.0 |
| 11 | 489.8 | >20.42 | >10000.0 |
| 12 | 236.9 | >42 | >10000.0 |
| 13 | 233.2 | >43 | >10000.0 |
| 15 | 112.1 | >89 | >10000.0 |
| 20 | 933.8 | >10.7 | >10000.0 |
| 21 | 2542.4 | >3.93 | >10000.0 |
| 22 | >10000.0 | NA | >10000.0 |
| 23 | 147.2 | >68 | >10000.0 |
| 25 | >10000.0 | NA | >10000.0 |
| 2 | 1225.9 | >8.2 | >10000.0 |
| 14 | 232.4 | >43 | >10000.0 |
| 16 | 590.7 | >17 | >10000.0 |
| 17 | 745.5 | >13 | >10000.0 |
| 18 | 222.4 | >45 | >10000.0 |
| 19 | 115.7 | >86 | >10000.0 |
| 24 | 9340.4 | >1.07 | >10000.0 |
| 30 | 177.0 | >57 | >10000.0 |
| 31 | >10000.0 | NA | >10000.0 |
| 32 | 2458.4 | >4.07 | >10000.0 |
| 33 | 1593.2 | >6.28 | >10000.0 |
| 34 | 175.1 | >57 | >10000.0 |
| 35 | >10000.0 | NA | >10000.0 |
| 36 | 1420.1 | >7.04 | >10000.0 |
| 37 | 171.5 | >58.3 | >10000.0 |
| 38 | 215.6 | >46 | >10000.0 |
| 39 | 495.7 | >20 | >10000.0 |
| 40 | 52.66 | >190 | >10000.0 |
| 41 | | | |
| 42 | 3533.6 | | |
| 43 | 619.5 | >16 | >10000.0 |
| 44 | 4766.0 | >2.1 | >10000.0 |
| 45 | 840.8 | >12 | >10000.0 |
| 46 | 210.2 | >48 | >10000.0 |
| 47 | >10000.0 | NA | >10000.0 |
| 48 | >10000.0 | NA | >10000.0 |
| 49 | 985.9 | >10 | >10000.0 |
| 50 | 3226.3 | >3.1 | >10000.0 |
| 51 | 94.52 | >106 | >10000.0 |
| 52 | 706.5 | >14 | >10000.0 |
| 53 | 815.2 | >12 | >10000.0 |
| 54 | | | |
| 55 | 295.6 | >34 | >10000.0 |
| 56 | 96.25 | >104 | >10000.0 |
| 57 | >10000.0 | NA | >10000.0 |
| 58 | 350.9 | >29 | >10000.0 |
| 59 | 93.14 | >107 | >10000.0 |
| 60 | | | |
| 61 | 340.5 | >29 | >10000.0 |
| 62 | 118.1 | >85 | >10000.0 |
| 63 | 205.2 | >49 | >10000.0 |
| 64 | 70.0 | >143 | >10000.0 |
| 65 | | | |
| 66 | 908.2 | >11 | >10000.0 |
| 67 | 279.2 | >36 | >10000.0 |
| 68 | | | |
| 69 | 1689.1 | >5.9 | >10000.0 |
| 70 | 245.1 | >41 | >10000.0 |
| 71 | 266.9 | >34 | >10000.0 |
| 72 | 174.7 | >57 | >10000.0 |
| 73 | 170.7 | >59 | >10000.0 |
| 74 | 86.9 | >115 | >10000.0 |
| 75 | 269.9 | >37 | >10000.0 |
| 76 | 674.2 | >14.8 | >10000.0 |
| 77 | 1396.3 | >7.1 | >10000.0 |
| 78 | 328.8 | >30 | >10000.0 |
| 79 | >10000.0 | NA | >10000.0 |
| 80 | 253.8 | >39.4 | >10000.0 |
| 81 | 769.7 | >13 | >10000.0 |
| 82 | >10000.0 | NA | >10000.0 |
| 83 | | | |
| 84 | 464.8 | >22 | >10000.0 |
| 85 | | | |
| 86 | 375.2 | >26.6 | >10000.0 |
| 87 | 889.9 | >11.2 | >10000.0 |
| 88 | 695.4 | >14 | >10000.0 |
| 89 | 937.6 | >11 | >10000.0 |
| 90 | | | |
| 91 | 287.4 | >35 | >10000.0 |
| 92 | >10000.0 | NA | >10000.0 |
| 93 | | | |
| 94 | >10000.0 | NA | >10000.0 |
| 95 | 284.4 | >35 | >10000.0 |
| 96 | 992.6 | >10 | >10000.0 |
| 97 | >10000.0 | NA | >10000.0 |

Mouse Pharmacokinetics (PK) methods. Male CD-1 mice (n=3/study) were used for pharmacokinetics (cassette and discrete) investigation of PI3Kα inhibitors. A mixture of five test compounds (cassette, p.o., 2 mg/kg for each compound, formulation: 10% EtOH/30% PEG-400/60% phosal 50 PG) or a single test compound (discrete) at dose of 1 mg/kg (i.v. formulation: 20% DMA+20% (30% solutol HS-15)+60% Saline) or 5 mg/kg (p.o. formulation: 0.5% MC) were designed for PK investigation.

In the oral dosing group, mice were fasted overnight with free access to water, and they were fed four hours after dosing. In the intravenous injection group, mice had free access to food and water. At the time points after dosing (cassette oral dosing group: 0.25 h, 1 h, 4 h, 8 h; discrete oral dosing group: 0.25 h, 0.5 h, 1 h, 2 h, 4 h, 8 h, 24 h; intravenous dosing group: 0.083, 0.25 h, 0.5 h, 1 h, 2 h, 4 h, 8 h, 24 h), mice were anesthetized by isoflurane, blood samples were collected into 1.5 mL EDTA·K2 coated tube from orbital bleeding, then centrifuge at 5600 rpm 7 min at 4° C. to obtain plasma. Samples were analyzed using LC-MS/MS method. Non-compartment model (Phoenix Winnolin software 8.3) was used for pharmacokinetics parameters calculation.

It is to be understood that, if any prior art publication is referred to herein, such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

1. A compound of formula (IV): 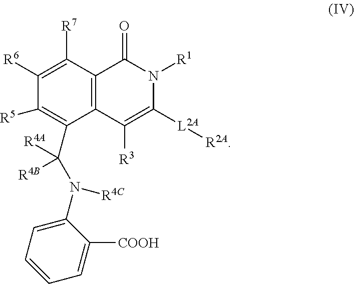
or a stereoisomer, tautomer, or pharmaceutically acceptable salt thereof, wherein:
R¹ is hydrogen, deuterium, —C₁₋₆alkyl, deuterated —C₁₋₆alkyl, heterocyclyl, heteroaryl, or aryl; wherein said —C₁₋₆alkyl, deuterated —C₁₋₆alkyl, heterocyclyl, heteroaryl, or aryl are each independently optionally substituted with at least one substituent R^{11a};
each R^{11a} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, aryl, heteroaryl, —CN, —NO₂, —OR^{1a}, —COR^{1a}, —CO₂R^{1a}, —CONR^{1a}R^{1b}, —SO₂R^{1a}, —NR^{1a}R^{1b}, —NR^{1a}COR^{1b}, —NR^{1a}CONR^{1b}R^{1c} or —NR^{1a}CO₂R^{1b};
wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{1d};
R^{1a}, R^{1b}, and R^{1c} are each independently hydrogen, —C₁₋₆alkyl, —C₂₋₆alkenyl, —C₂₋₆alkynyl, cycloalkyl, cycloalkenyl, cycloalkynyl, heterocyclyl, aryl, or heteroaryl; wherein said —C₁₋₆alkyl, —C₂₋₆alkenyl, —C₂₋₆alkynyl, cycloalkyl, cycloalkenyl, cycloalkynyl, heterocyclyl, aryl, or heteroaryl are each independently optionally substituted with at least one substituent R^{1d};
each R^{1d} is independently hydrogen, halogen, oxo, —CN, —NO₂, —C₁₋₆alkyl, —C₂₋₆alkenyl, —C₂₋₆alkynyl, —C₁₋₆haloalkyl, cycloalkyl, cycloalkenyl, cycloalkynyl, heterocyclyl, aryl, heteroaryl, —OC₁₋₆alkyl, —COC₁₋₆alkyl, or —CO₂C₁₋₆alkyl.
R³ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
R^{4A} and R^{4B}, which may be the same or different, are each independently hydrogen, deuterium, halogen, —C₁₋₆alkyl, or deuterated —C₁₋₆alkyl; wherein said —C₁₋₆alkyl or deuterated —C₁₋₆alkyl is each independently optionally substituted with at least one substituent selected from halogen, —C₁₋₆alkyl, —C₁₋₆haloalkyl, —CN, —NO₂, —OC₁₋₆alkyl, —COC₁₋₆alkyl, and —CO₂C₁₋₆alkyl;
R^{4C} is hydrogen, halogen, —C₁₋₆alkyl or deuterated —C₁₋₆alkyl; wherein said —C₁₋₆alkyl or deuterated —C₁₋₆alkyl is optionally substituted with at least one substituent selected from hydrogen, halogen, —C₁₋₆alkyl, —C₁₋₆haloalkyl, —C₂₋₆alkenyl, —C₂₋₆alkynyl, cycloalkyl, cycloalkenyl, cycloalkynyl, heterocyclyl, aryl, heteroaryl, —CN, —NO₂, —OC₁₋₆alkyl, —COC₁₋₆alkyl, and —CO₂C₁₋₆alkyl;
R⁵ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
R⁶ is hydrogen, halogen, —C₁₋₆alkyl, —OC₁₋₆alkyl, —CN, or —NO₂; wherein said —C₁₋₆alkyl or —OC₁₋₆alkyl is each independently optionally substituted with at least one substituent selected from hydrogen, deuterium, halogen, —C₁₋₆alkyl, —CN, and —NO₂;
R⁷ is hydrogen, halogen, —C₁₋₆alkyl, —OC₁₋₆alkyl, —CN, or —NO₂; wherein said —C₁₋₆alkyl or —OC₁₋₆alkyl is optionally substituted with at least one substituent selected from hydrogen, halogen, —C₁₋₆alkyl, —CN, and —NO₂;
-L^{2A}- is a covalent bond, *—N(R^{21a})C(O)—**, **—N(R^{21a})C(O)—*, *—(CR^{22a}R^{23a})ₘ—C(R^{22a})═C(R^{23a})—(CR^{22a}R^{23a})ₙ—**, or 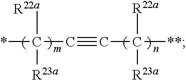
* refers to the position attached to the isoquinolin-1(2H)-one;
** refers to the position attached to R^{2A};
m and n are each independently 0, 1, 2, or 3;
R^{21a} is hydrogen or —C₁₋₆alkyl; wherein said —C₁₋₆alkyl is optionally substituted with at least one substituent R^{211b};
each R^{211b} is independently hydrogen or halogen;
R^{22a} and R^{23a} are each independently hydrogen, halogen, or —C₁₋₆alkyl; wherein said —C₁₋₆alkyl is each independently optionally substituted with at least one substituent R^{223a};
each R^{223a} is independently hydrogen or halogen;
R^{2A} is —C₁₋₆alkyl, 3- to 15-membered cycloalkyl, unsaturated 3- to 15-membered heterocyclyl comprising at least one carbon-carbon double bond, 5- to 12-membered aryl, or 5- to 15-membered heteroaryl; wherein each of said —C₁₋₆alkyl, 3- to 15-membered cycloalkyl, unsaturated 3- to 15-membered heterocyclyl comprising at least one carbon-carbon double bond, 5- to 12-membered aryl, or 5- to 15-membered heteroaryl is optionally substituted with at least one R^{Y1};
each R^{Y1} is independently hydrogen, halogen, oxo, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl, or —SC₁₋₆alkyl are each independently optionally substituted with at least one substituent R^{Y1a}; and
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂.

2. The compound according to claim 1, wherein:
L^{2A} is a covalent bond, *—NHC(O)—**, **—NHC(O)—*, *—CH═CH—**, or *—C≡C—**.

3. The compound according to claim 1, wherein L^{2A} is a covalent bond.

4. The compound according to claim 1, wherein:
R^{2A} is selected from methyl, 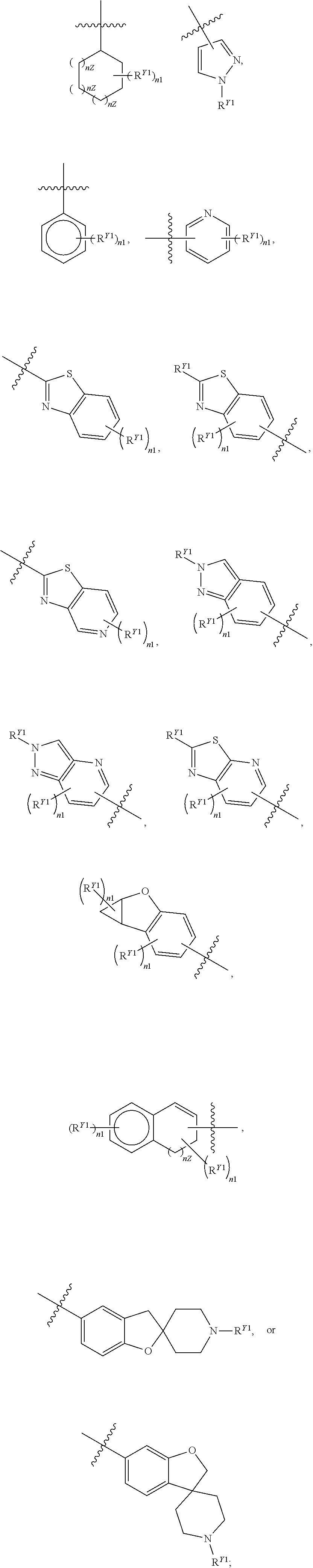
n^{Z} is each independently selected from 0, 1, 2, or 3; and
n1 is each independently selected from 0, 1, 2, or 3.

5. The compound according to claim 1, wherein:
R^{2A} is 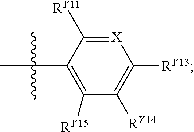
X is C(R^{Y12}) or N;
R^{Y11}, R^{Y12}, R^{Y13}, R^{Y14}, and R^{Y15} are each independently hydrogen, halogen, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl or —SC₁₋₆alkyl are each independently optionally substituted with at least one substituent R^{Y1a}; or
(R^{Y11} and R^{Y12}), (R^{Y12} and R^{Y13}), (R^{Y13} and R^{Y14}), or (R^{Y14} and R^{Y15}), together with the atoms to which they are attached, form a C₃₋₁₀ cycloalkyl, C₃₋₁₀ cycloalkenyl, 4- to 12-membered heterocyclyl ring containing 1, 2 or 3 heteroatoms independently selected from nitrogen, oxygen and optionally oxidized sulfur, 6- to 12-membered aryl, or 4- to 12-membered heteroaryl ring containing 1, 2 or 3 heteroatoms independently selected from nitrogen, oxygen and optionally oxidized sulfur, and optionally substituted with at least one substituent R^{Y1a}; and
each R^{Y1a} is independently hydrogen, halogen, —C₁₋₆alkyl, —NHC₁₋₆alkyl, —C₂₋₆alkenyl, —C₂₋₆alkynyl, cycloalkyl, cycloalkenyl, cycloalkynyl, heterocyclyl, aryl, heteroaryl, —OH, —CN, —CONH₂, —COCH₃, —OC₁₋₆alkyl, or —NO₂.

6. The compound according to claim 5, wherein R^{Y11} and R^{Y15} are hydrogen; and 1, 2, or 3 of R^{Y12}, R^{Y13}, and R^{Y14} is hydrogen.

7. (canceled)

8. The compound according to claim 6, wherein:
R^{Y12} and R^{Y14} are hydrogen; and
R^{Y13} is halogen, —C₁₋₆alkyl, —C₂₋₆alkenyl, —C₂₋₆alkynyl, cycloalkyl, cycloalkenyl, cycloalkynyl, heterocyclyl, —O-heterocyclyl, —CO-heterocyclyl, aryl, heteroaryl, —P(O)(C₁₋₆alkyl)₂, —OH, —CN, —OC₁₋₆alkyl, —SC₁₋₆alkyl, or —NO₂, wherein said —C₁₋₆alkyl, —C₂₋₆alkenyl, —C₂₋₆alkynyl, cycloalkyl, cycloalkenyl, cycloalkynyl, heterocyclyl, aryl, heteroaryl, —OC₁₋₆alkyl or —SC₁₋₆alkyl are each independently optionally substituted with at least one substituent R^{Y1a}.

9. The compound according to claim 6, wherein R^{Y12}, R^{Y13}, and R^{Y14} are selected from hydrogen, halogen, —C₁₋₆alkyl, —P(O)(C₁₋₆alkyl)₂, —OH, —CN, —OC₁₋₆alkyl, —SC₁₋₆alkyl, or —NO₂.

10. The compound according to claim 6, wherein R^{Y12} and R^{Y13}, together with the atoms to which they are attached, form a 4- to 12-membered heterocyclyl ring containing 1, 2 or 3 heteroatoms independently selected from nitrogen, oxygen and optionally oxidized sulfur, or 4- to 12-membered heteroaryl ring containing 1, 2 or 3 heteroatoms independently selected from nitrogen, oxygen and optionally oxidized sulfur, and optionally substituted with at least one substituent R^{Y1a}.

11-17. (canceled)

18. The compound according to claim 1, wherein:
R^{2A} is methyl,

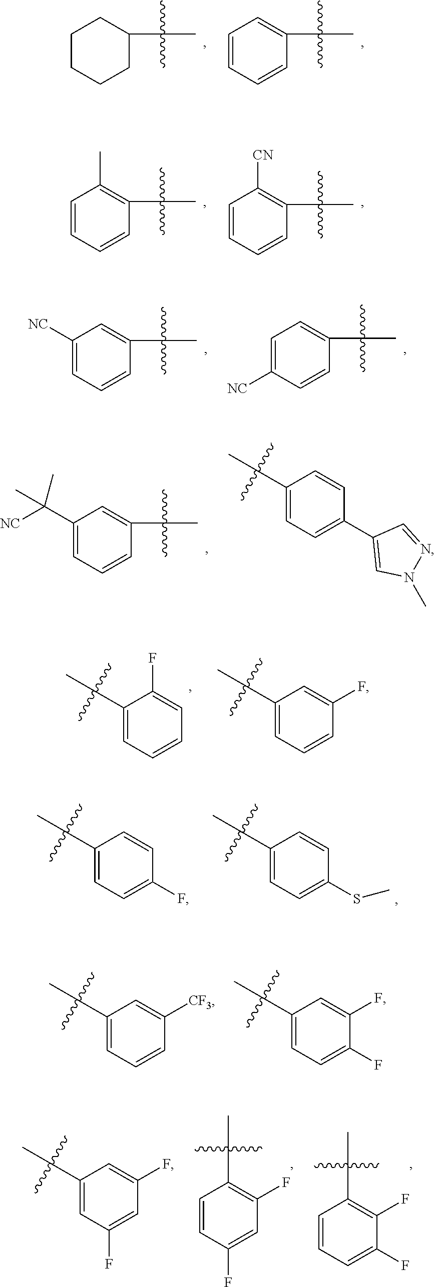

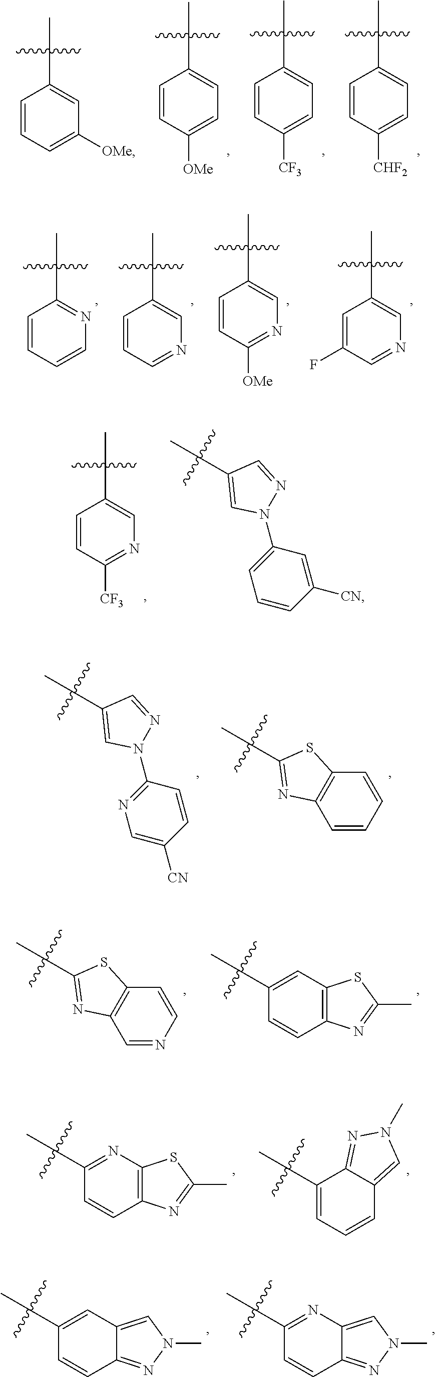

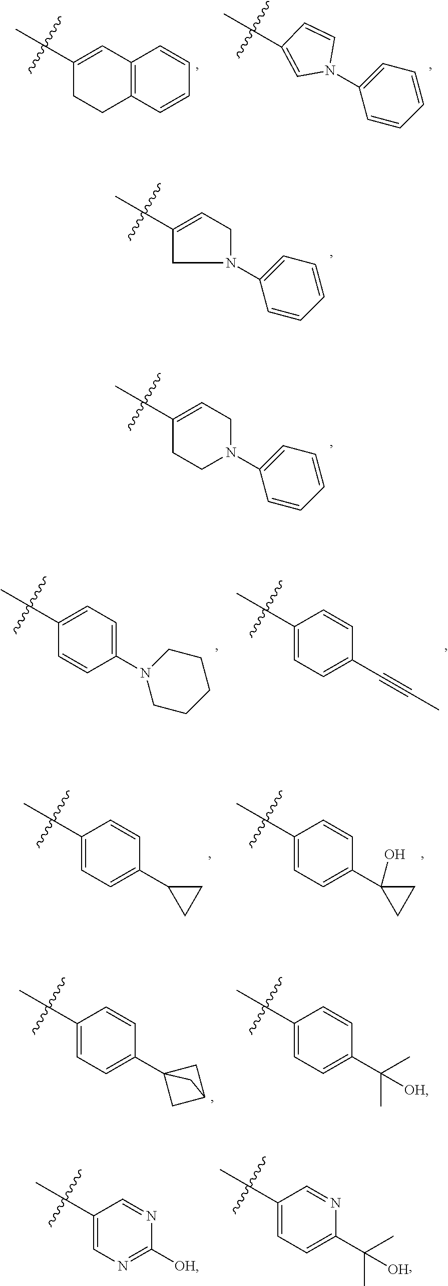

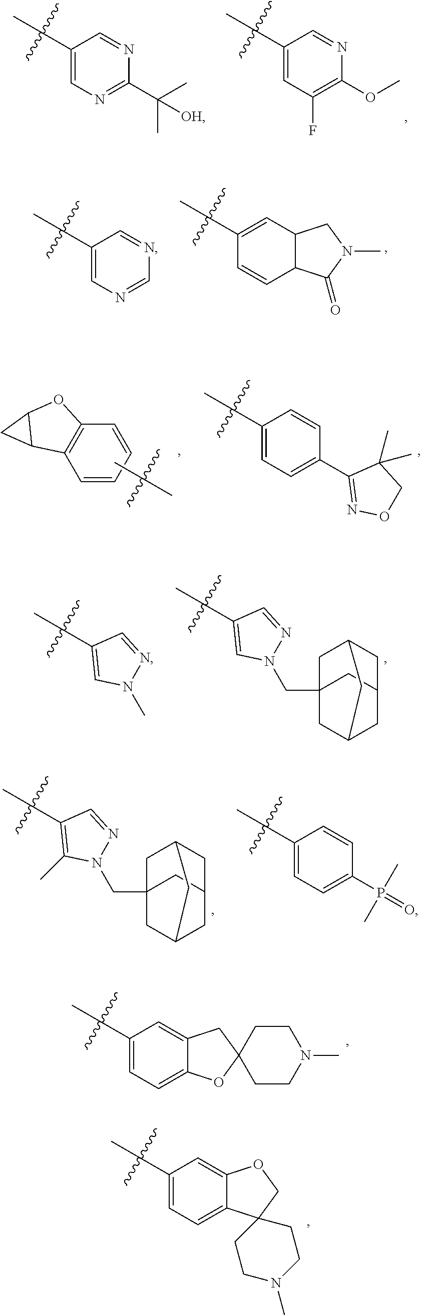

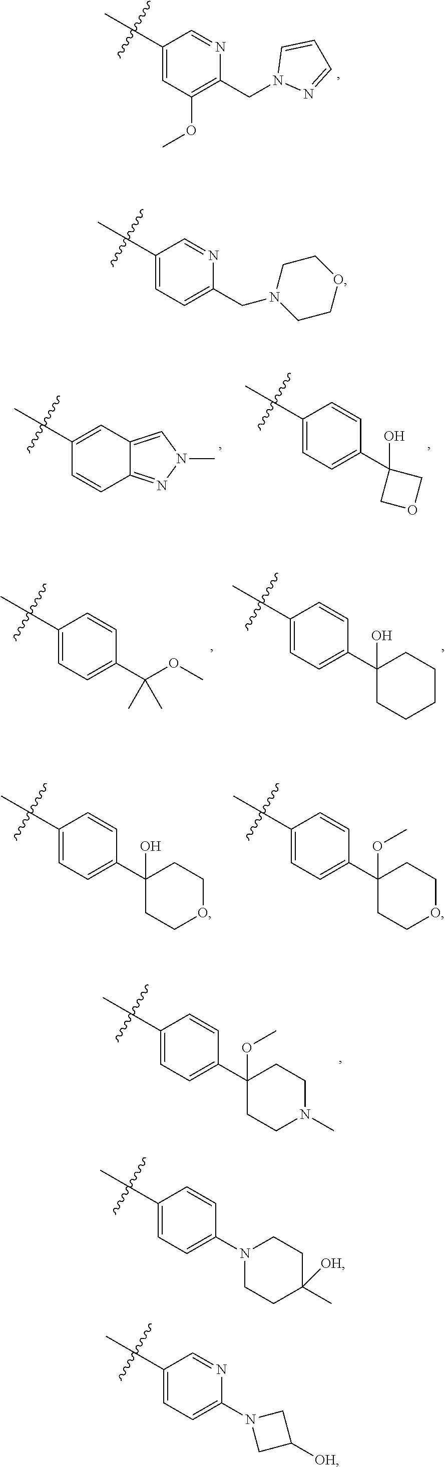

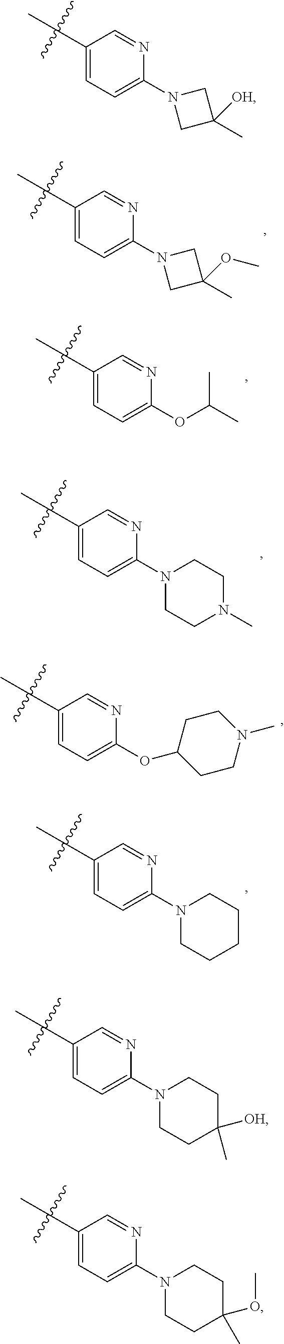

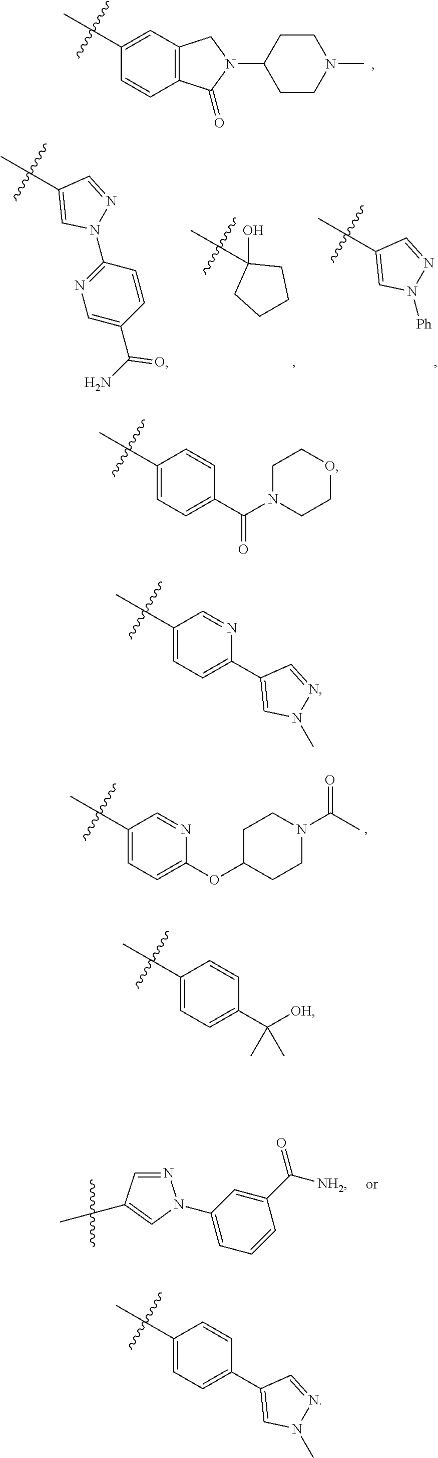

19. The compound according to claim 1, wherein:
R^{4A} and R^{4B} are different, and are each independently hydrogen, deuterium, halogen, —C₁₋₆alkyl, or deuterated —C₁₋₆alkyl, and wherein the carbon atom to which R^{4A} and R^{4B} are attached is S or R configuration.

20-23. (canceled)

24. The compound according to claim 1, the compound having formula (IV-1) 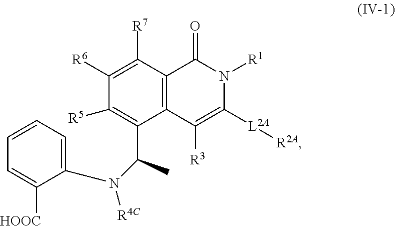
or a tautomer or pharmaceutically acceptable salt thereof.

25. The compound according to claim 1, wherein R^{4C} is hydrogen.

26. (canceled)

27. The compound according to claim 1, wherein R¹ is hydrogen, methyl, —CH₂CN, —CH₂OCH₃, —CH₂CH₃, —CH₂CH₃CN, —CH₂SO₂CH₃, —COCH₃, —CH₂OH, —CH₂CH₂OCH₃,

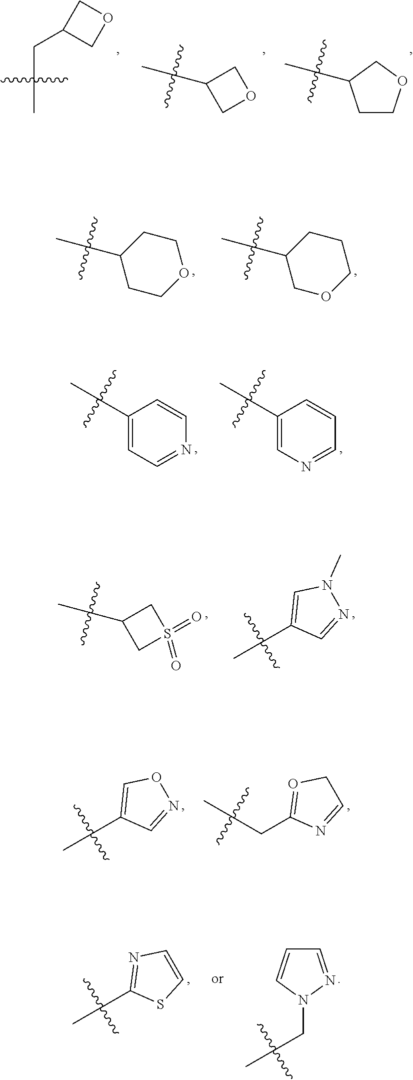

28. The compound according to claim 1, wherein R¹ is methyl or —CD₃.

29. (canceled)

30. The compound according to claim 1, wherein R³ is hydrogen; R₅ is hydrogen; R⁶ is hydrogen, F, methyl, —CD₃, —CF₃, —CHF₂ or —CH₂F, and R⁷ is hydrogen.

31-35. (canceled)

36. A compound selected from: 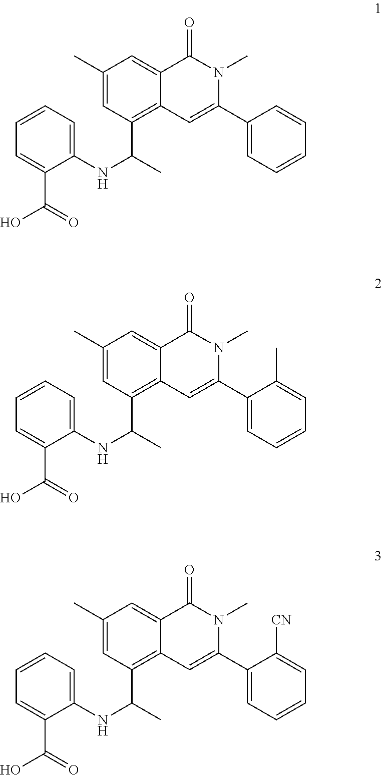 [SMILES: Cc1cc(C(C)Nc2ccccc2C(=O)O)c2cc(-c3ccccc3)n(C)c(=O)c2c1 ; Cc1cc(C(C)Nc2ccccc2C(=O)O)c2cc(-c3ccccc3C)n(C)c(=O)c2c1 ; Cc1cc(C(C)Nc2ccccc2C(=O)O)c2cc(-c3ccccc3C#N)n(C)c(=O)c2c1] 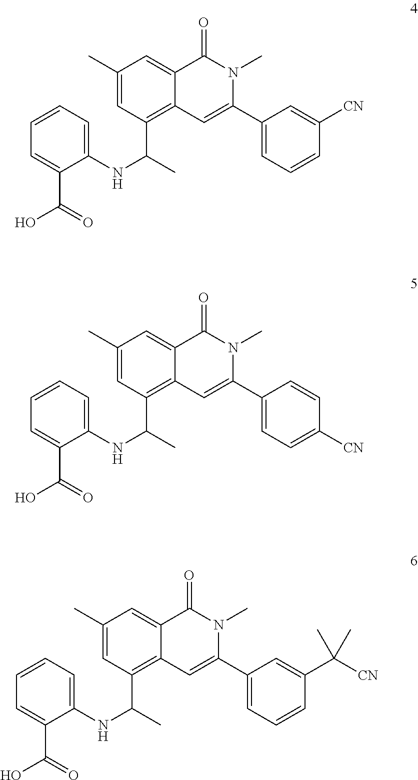 [SMILES: Cc1cc(C(C)Nc2ccccc2C(=O)O)c2cc(-c3cccc(C#N)c3)n(C)c(=O)c2c1 ; Cc1cc(C(C)Nc2ccccc2C(=O)O)c2cc(-c3ccc(C#N)cc3)n(C)c(=O)c2c1 ; Cc1cc(C(C)Nc2ccccc2C(=O)O)c2cc(-c3cccc(C(C)(C)C#N)c3)n(C)c(=O)c2c1] 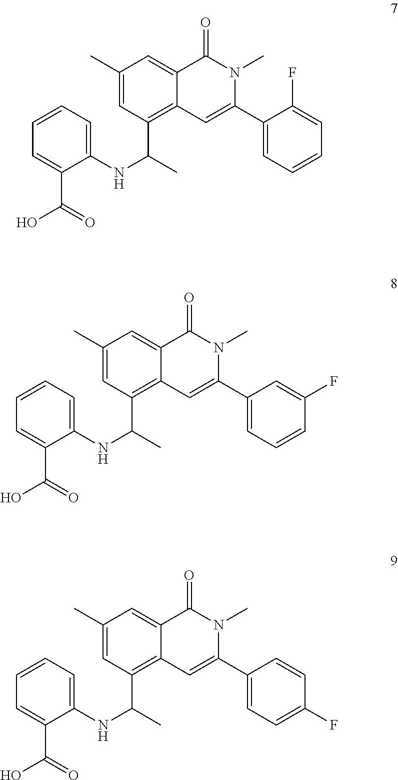 [SMILES: Cc1cc(C(C)Nc2ccccc2C(=O)O)c2cc(-c3ccccc3F)n(C)c(=O)c2c1 ; Cc1cc(C(C)Nc2ccccc2C(=O)O)c2cc(-c3cccc(F)c3)n(C)c(=O)c2c1 ; Cc1cc(C(C)Nc2ccccc2C(=O)O)c2cc(-c3ccc(F)cc3)n(C)c(=O)c2c1] 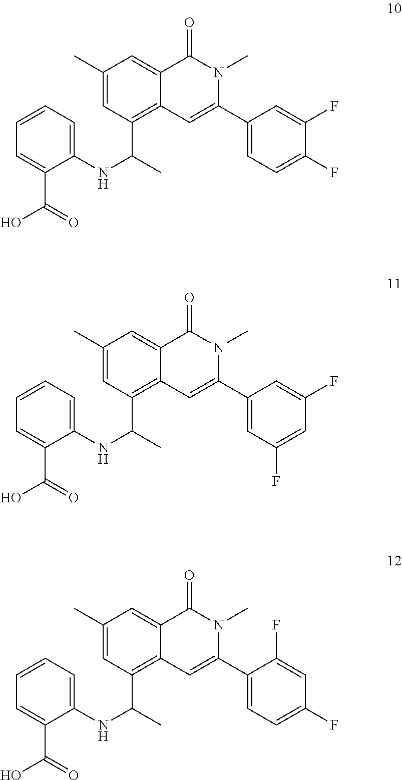 [SMILES: Cc1cc(C(C)Nc2ccccc2C(=O)O)c2cc(-c3ccc(F)c(F)c3)n(C)c(=O)c2c1 ; Cc1cc(C(C)Nc2ccccc2C(=O)O)c2cc(-c3cc(F)cc(F)c3)n(C)c(=O)c2c1 ; Cc1cc(C(C)Nc2ccccc2C(=O)O)c2cc(-c3ccc(F)cc3F)n(C)c(=O)c2c1] 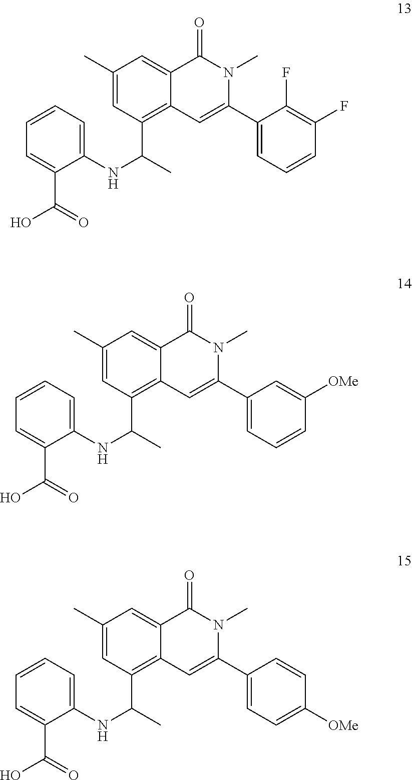 [SMILES: Cc1cc(C(C)Nc2ccccc2C(=O)O)c2cc(-c3cccc(F)c3F)n(C)c(=O)c2c1 ; COc1cccc(-c2cc3c(C(C)Nc4ccccc4C(=O)O)cc(C)cc3c(=O)n2C)c1 ; COc1ccc(-c2cc3c(C(C)Nc4ccccc4C(=O)O)cc(C)cc3c(=O)n2C)cc1] 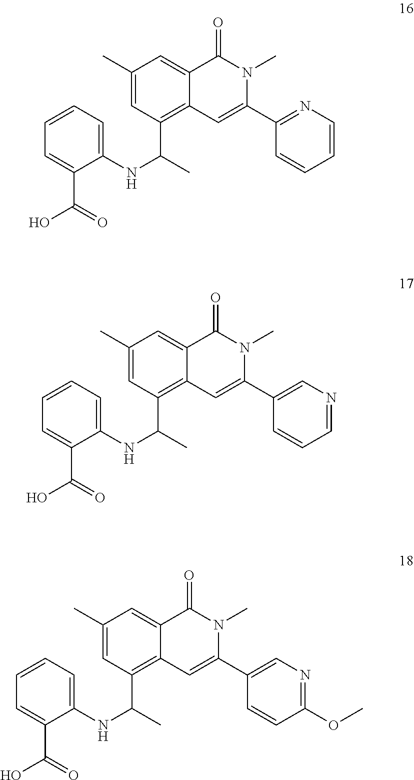 [SMILES: Cc1cc(C(C)Nc2ccccc2C(=O)O)c2cc(-c3ccccn3)n(C)c(=O)c2c1 ; Cc1cc(C(C)Nc2ccccc2C(=O)O)c2cc(-c3cccnc3)n(C)c(=O)c2c1 ; COc1ccc(-c2cc3c(C(C)Nc4ccccc4C(=O)O)cc(C)cc3c(=O)n2C)cn1] 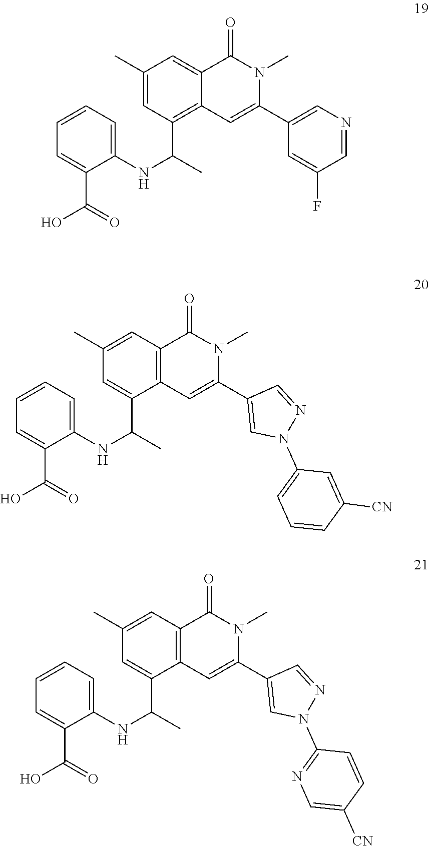 [SMILES: Cc1cc(C(C)Nc2ccccc2C(=O)O)c2cc(-c3cncc(F)c3)n(C)c(=O)c2c1 ; Cc1cc(C(C)Nc2ccccc2C(=O)O)c2cc(-c3cnn(-c4cccc(C#N)c4)c3)n(C)c(=O)c2c1 ; Cc1cc(C(C)Nc2ccccc2C(=O)O)c2cc(-c3cnn(-c4ccc(C#N)cn4)c3)n(C)c(=O)c2c1] 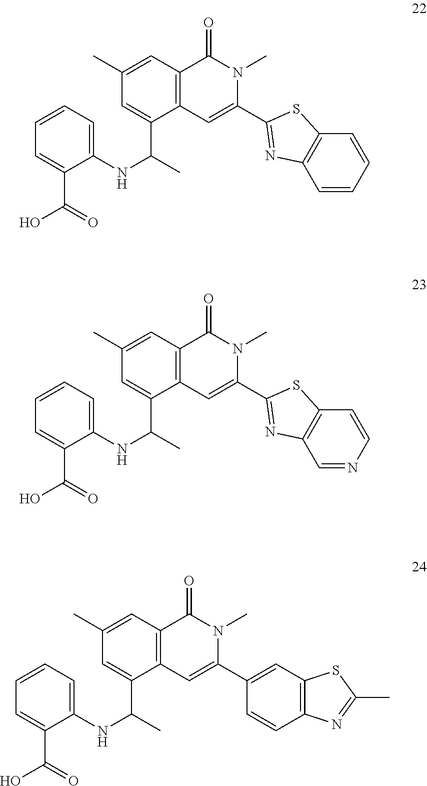 [SMILES: Cc1cc(C(C)Nc2ccccc2C(=O)O)c2cc(-c3nc4ccccc4s3)n(C)c(=O)c2c1 ; Cc1cc(C(C)Nc2ccccc2C(=O)O)c2cc(-c3nc4cnccc4s3)n(C)c(=O)c2c1 ; Cc1cc(C(C)Nc2ccccc2C(=O)O)c2cc(-c3ccc4nc(C)sc4c3)n(C)c(=O)c2c1] 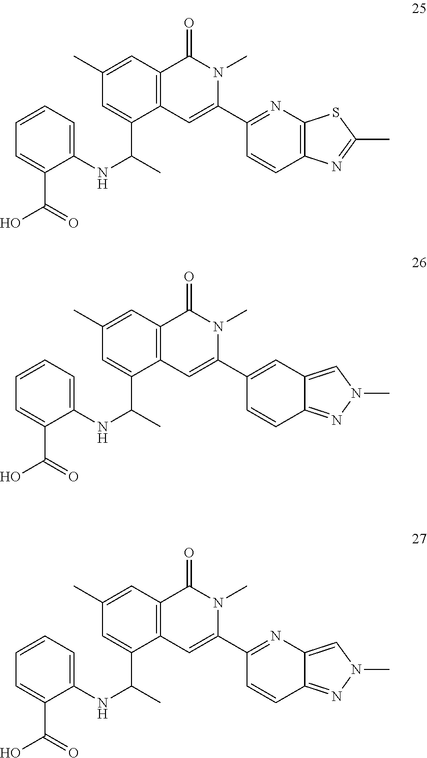 [SMILES: Cc1cc(C(C)Nc2ccccc2C(=O)O)c2cc(-c3ccc4nc(C)sc4n3)n(C)c(=O)c2c1 ; Cc1cc(C(C)Nc2ccccc2C(=O)O)c2cc(-c3ccc4nn(C)cc4c3)n(C)c(=O)c2c1 ; Cc1cc(C(C)Nc2ccccc2C(=O)O)c2cc(-c3ccc4nn(C)cc4n3)n(C)c(=O)c2c1] 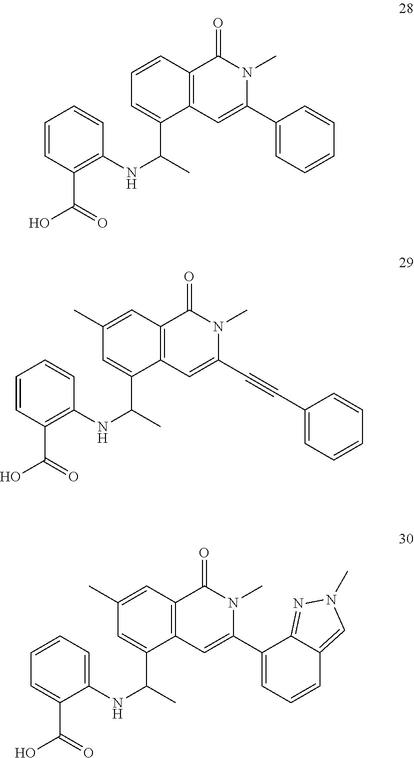 [SMILES: CC(Nc1ccccc1C(=O)O)c1cccc2c(=O)n(C)c(-c3ccccc3)cc12 ; Cc1cc(C(C)Nc2ccccc2C(=O)O)c2cc(C#Cc3ccccc3)n(C)c(=O)c2c1 ; Cc1cc(C(C)Nc2ccccc2C(=O)O)c2cc(-c3cccc4cn(C)nc34)n(C)c(=O)c2c1] 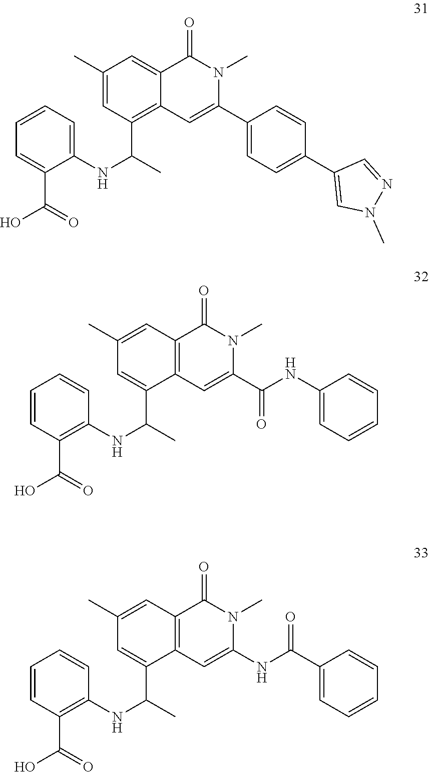 [SMILES: Cc1cc(C(C)Nc2ccccc2C(=O)O)c2cc(-c3ccc(-c4cnn(C)c4)cc3)n(C)c(=O)c2c1 ; Cc1cc(C(C)Nc2ccccc2C(=O)O)c2cc(C(=O)Nc3ccccc3)n(C)c(=O)c2c1 ; Cc1cc(C(C)Nc2ccccc2C(=O)O)c2cc(NC(=O)c3ccccc3)n(C)c(=O)c2c1] 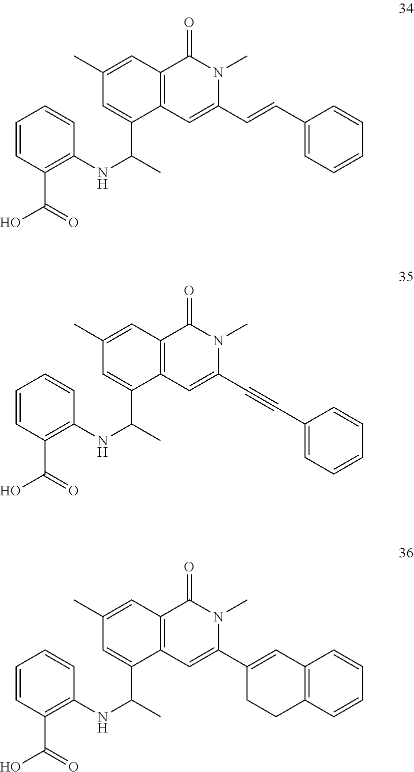 [SMILES: Cc1cc(C(C)Nc2ccccc2C(=O)O)c2cc(/C=C/c3ccccc3)n(C)c(=O)c2c1 ; Cc1cc(C(C)Nc2ccccc2C(=O)O)c2cc(C#Cc3ccccc3)n(C)c(=O)c2c1 ; Cc1cc(C(C)Nc2ccccc2C(=O)O)c2cc(C3=Cc4ccccc4CC3)n(C)c(=O)c2c1] 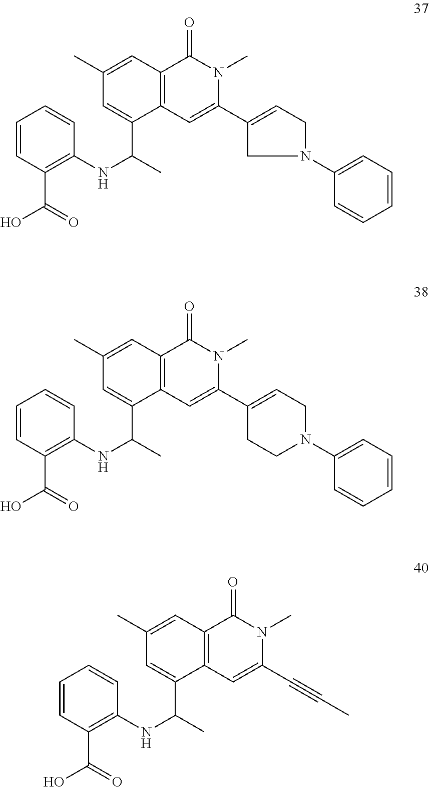 [SMILES: Cc1cc(C(C)Nc2ccccc2C(=O)O)c2cc(C3=CCN(c4ccccc4)C3)n(C)c(=O)c2c1 ; Cc1cc(C(C)Nc2ccccc2C(=O)O)c2cc(C3=CCN(c4ccccc4)CC3)n(C)c(=O)c2c1 ; CC#Cc1cc2c(C(C)Nc3ccccc3C(=O)O)cc(C)cc2c(=O)n1C] 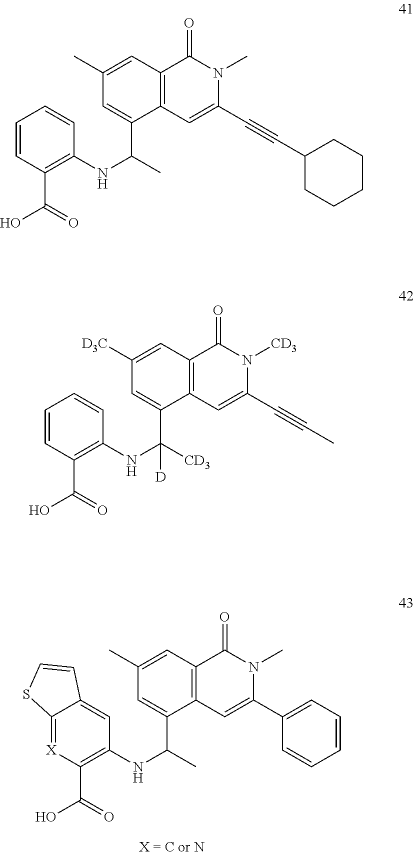 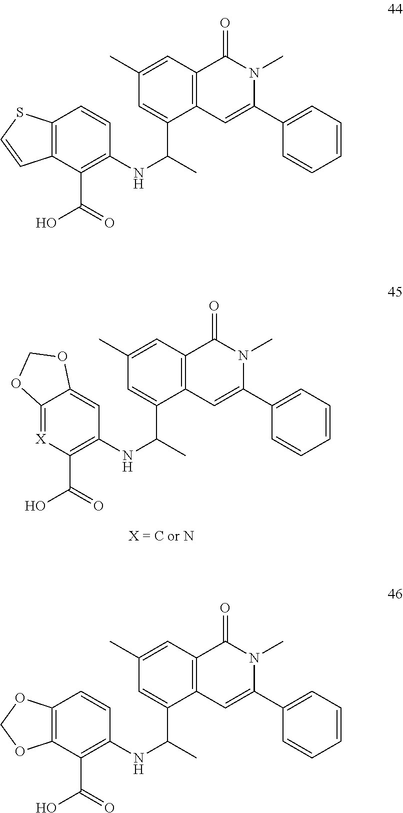 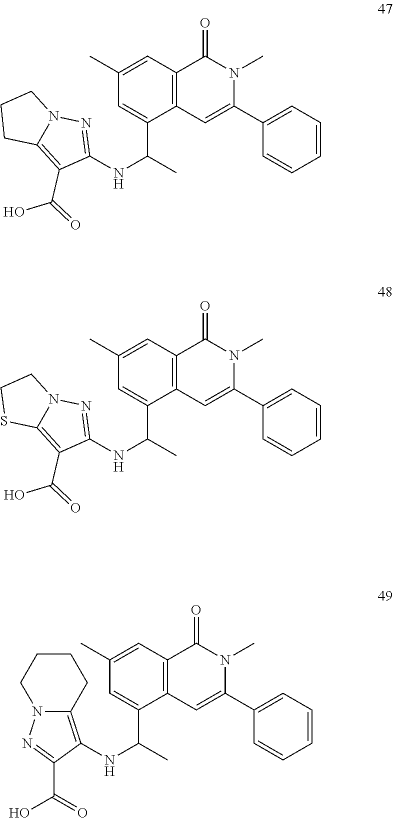 [SMILES: Cc1cc(C(C)Nc2nn3c(c2C(=O)O)CCC3)c2cc(-c3ccccc3)n(C)c(=O)c2c1 ; Cc1cc(C(C)Nc2nn3c(c2C(=O)O)SCC3)c2cc(-c3ccccc3)n(C)c(=O)c2c1 ; Cc1cc(C(C)Nc2c(C(=O)O)nn3c2CCCC3)c2cc(-c3ccccc3)n(C)c(=O)c2c1] 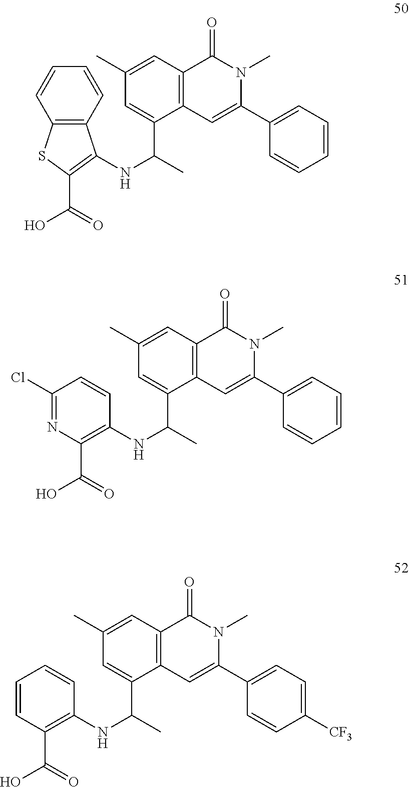 [SMILES: Cc1cc(C(C)Nc2c(C(=O)O)sc3ccccc23)c2cc(-c3ccccc3)n(C)c(=O)c2c1 ; Cc1cc(C(C)Nc2ccc(Cl)nc2C(=O)O)c2cc(-c3ccccc3)n(C)c(=O)c2c1 ; Cc1cc(C(C)Nc2ccccc2C(=O)O)c2cc(-c3ccc(C(F)(F)F)cc3)n(C)c(=O)c2c1] 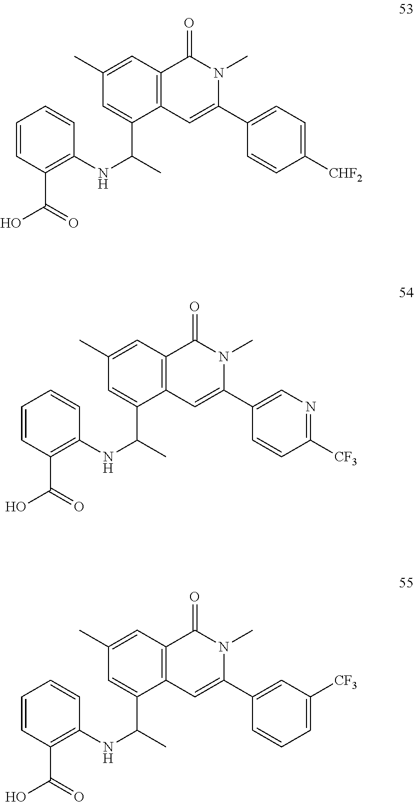 [SMILES: Cc1cc(C(C)Nc2ccccc2C(=O)O)c2cc(-c3ccc(C(F)F)cc3)n(C)c(=O)c2c1 ; Cc1cc(C(C)Nc2ccccc2C(=O)O)c2cc(-c3ccc(C(F)(F)F)nc3)n(C)c(=O)c2c1 ; Cc1cc(C(C)Nc2ccccc2C(=O)O)c2cc(-c3cccc(C(F)(F)F)c3)n(C)c(=O)c2c1] 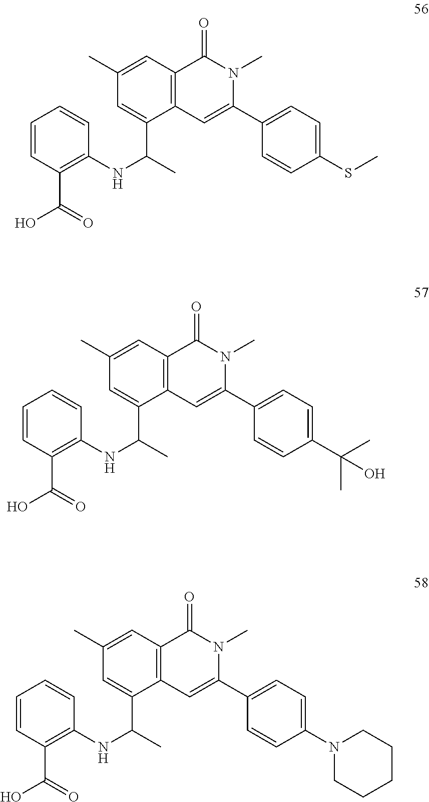 [SMILES: CSc1ccc(-c2cc3c(C(C)Nc4ccccc4C(=O)O)cc(C)cc3c(=O)n2C)cc1 ; Cc1cc(C(C)Nc2ccccc2C(=O)O)c2cc(-c3ccc(C(C)(C)O)cc3)n(C)c(=O)c2c1 ; Cc1cc(C(C)Nc2ccccc2C(=O)O)c2cc(-c3ccc(N4CCCCC4)cc3)n(C)c(=O)c2c1] 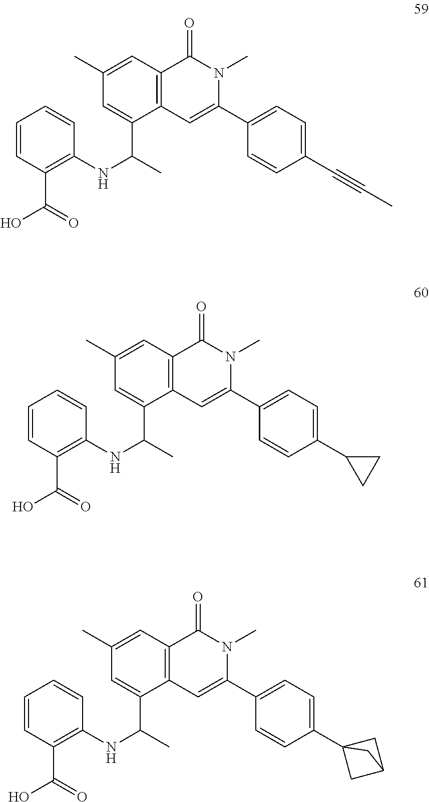 [SMILES: CC#Cc1ccc(-c2cc3c(C(C)Nc4ccccc4C(=O)O)cc(C)cc3c(=O)n2C)cc1 ; Cc1cc(C(C)Nc2ccccc2C(=O)O)c2cc(-c3ccc(C4CC4)cc3)n(C)c(=O)c2c1 ; Cc1cc(C(C)Nc2ccccc2C(=O)O)c2cc(-c3ccc(C45CC(C4)C5)cc3)n(C)c(=O)c2c1] 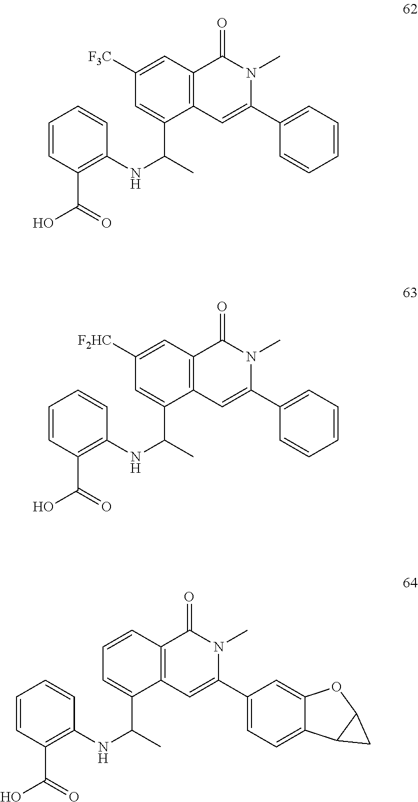 [SMILES: CC(Nc1ccccc1C(=O)O)c1cc(C(F)(F)F)cc2c(=O)n(C)c(-c3ccccc3)cc12 ; CC(Nc1ccccc1C(=O)O)c1cc(C(F)F)cc2c(=O)n(C)c(-c3ccccc3)cc12 ; CC(Nc1ccccc1C(=O)O)c1cccc2c(=O)n(C)c(-c3ccc4c(c3)OC3CC43)cc12] 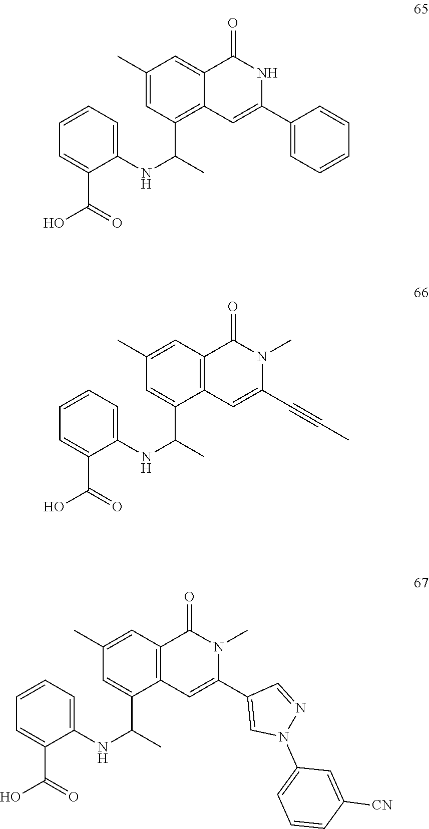 [SMILES: Cc1cc(C(C)Nc2ccccc2C(=O)O)c2cc(-c3ccccc3)[nH]c(=O)c2c1 ; CC#Cc1cc2c(C(C)Nc3ccccc3C(=O)O)cc(C)cc2c(=O)n1C ; Cc1cc(C(C)Nc2ccccc2C(=O)O)c2cc(-c3cnn(-c4cccc(C#N)c4)c3)n(C)c(=O)c2c1] 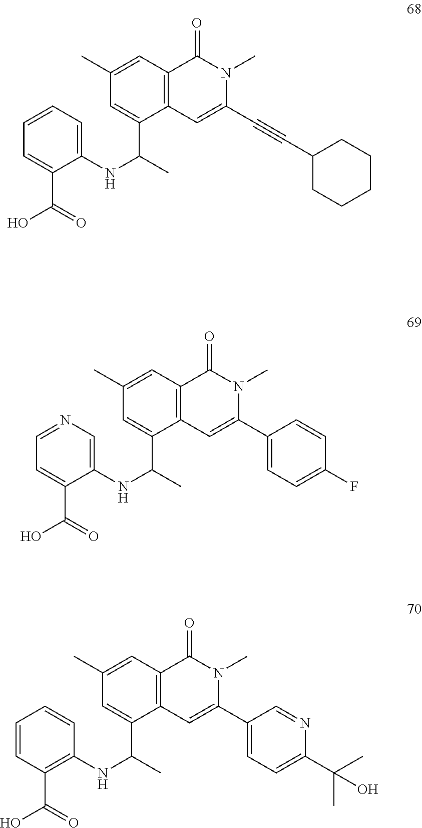 [SMILES: Cc1cc(C(C)Nc2ccccc2C(=O)O)c2cc(C#CC3CCCCC3)n(C)c(=O)c2c1 ; Cc1cc(C(C)Nc2cnccc2C(=O)O)c2cc(-c3ccc(F)cc3)n(C)c(=O)c2c1 ; Cc1cc(C(C)Nc2ccccc2C(=O)O)c2cc(-c3ccc(C(C)(C)O)nc3)n(C)c(=O)c2c1] 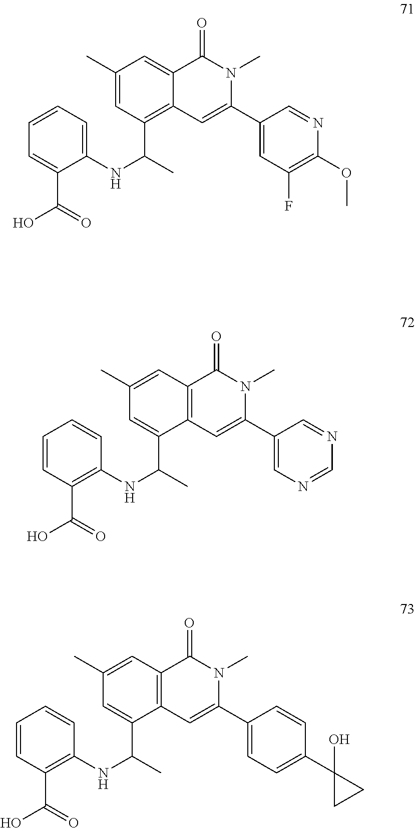 [SMILES: COc1ncc(-c2cc3c(C(C)Nc4ccccc4C(=O)O)cc(C)cc3c(=O)n2C)cc1F ; Cc1cc(C(C)Nc2ccccc2C(=O)O)c2cc(-c3cncnc3)n(C)c(=O)c2c1 ; Cc1cc(C(C)Nc2ccccc2C(=O)O)c2cc(-c3ccc(C4(O)CC4)cc3)n(C)c(=O)c2c1] 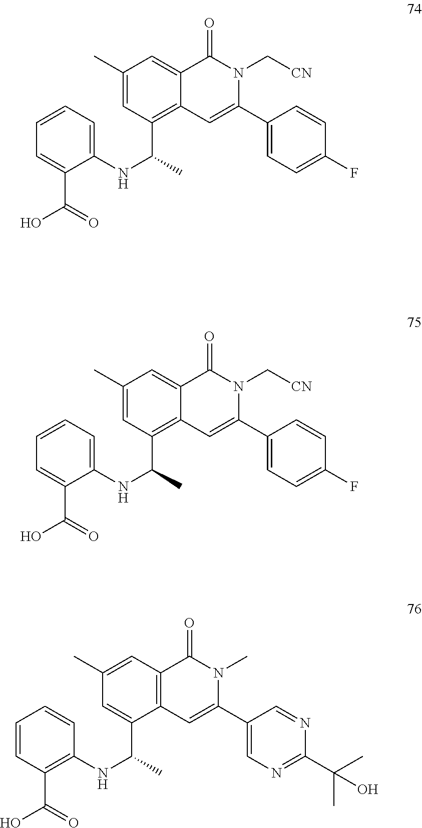 [SMILES: Cc1cc([C@H](C)Nc2ccccc2C(=O)O)c2cc(-c3ccc(F)cc3)n(CC#N)c(=O)c2c1 ; Cc1cc([C@@H](C)Nc2ccccc2C(=O)O)c2cc(-c3ccc(F)cc3)n(CC#N)c(=O)c2c1 ; Cc1cc([C@H](C)Nc2ccccc2C(=O)O)c2cc(-c3cnc(C(C)(C)O)nc3)n(C)c(=O)c2c1] 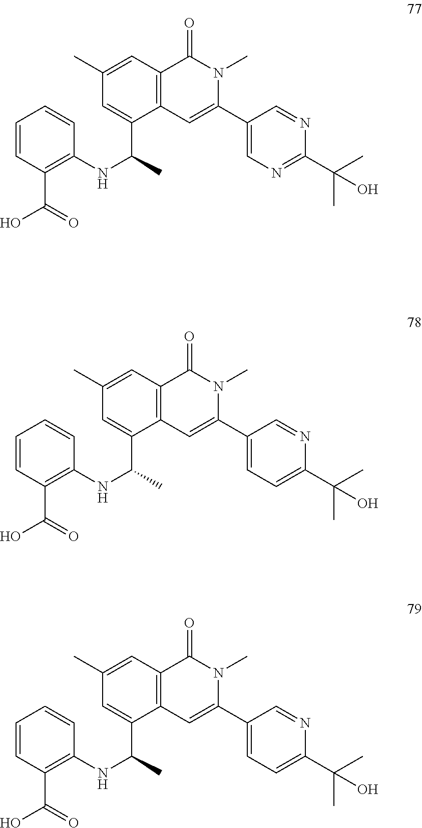 [SMILES: Cc1cc([C@@H](C)Nc2ccccc2C(=O)O)c2cc(-c3cnc(C(C)(C)O)nc3)n(C)c(=O)c2c1 ; Cc1cc([C@H](C)Nc2ccccc2C(=O)O)c2cc(-c3ccc(C(C)(C)O)nc3)n(C)c(=O)c2c1 ; Cc1cc([C@@H](C)Nc2ccccc2C(=O)O)c2cc(-c3ccc(C(C)(C)O)nc3)n(C)c(=O)c2c1] 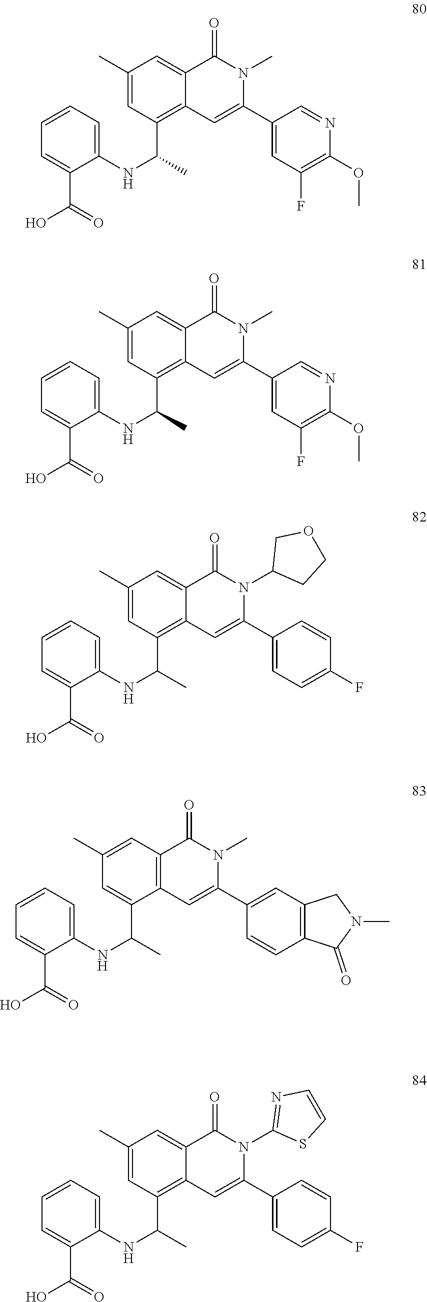 [SMILES: COc1ncc(-c2cc3c([C@H](C)Nc4ccccc4C(=O)O)cc(C)cc3c(=O)n2C)cc1F ; COc1ncc(-c2cc3c([C@@H](C)Nc4ccccc4C(=O)O)cc(C)cc3c(=O)n2C)cc1F ; Cc1cc(C(C)Nc2ccccc2C(=O)O)c2cc(-c3ccc(F)cc3)n(C3CCOC3)c(=O)c2c1 ; Cc1cc(C(C)Nc2ccccc2C(=O)O)c2cc(-c3ccc4c(c3)CN(C)C4=O)n(C)c(=O)c2c1 ; Cc1cc(C(C)Nc2ccccc2C(=O)O)c2cc(-c3ccc(F)cc3)n(-c3nccs3)c(=O)c2c1] 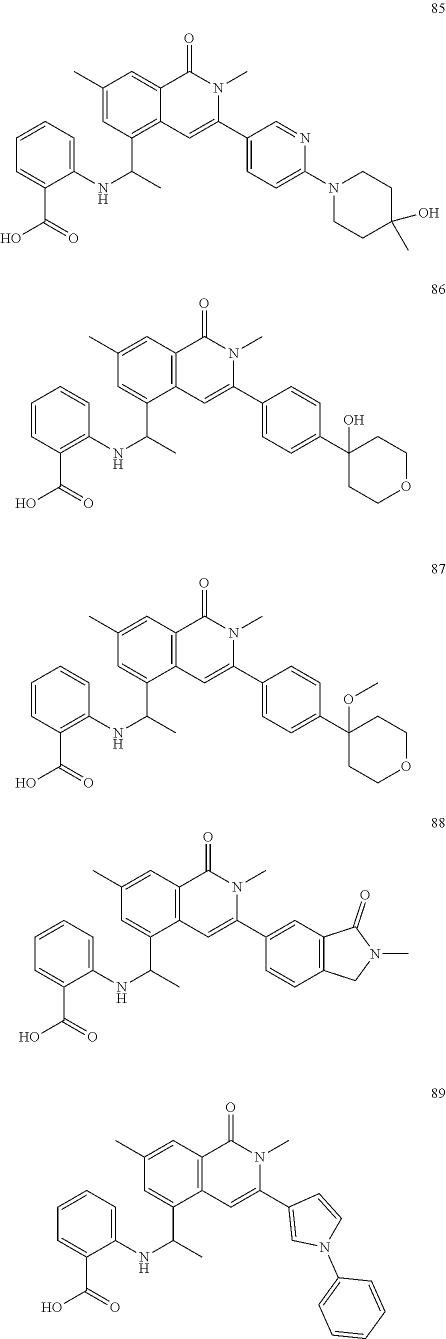 [SMILES: Cc1cc(C(C)Nc2ccccc2C(=O)O)c2cc(-c3ccc(N4CCC(C)(O)CC4)nc3)n(C)c(=O)c2c1 ; Cc1cc(C(C)Nc2ccccc2C(=O)O)c2cc(-c3ccc(C4(O)CCOCC4)cc3)n(C)c(=O)c2c1 ; COC1(c2ccc(-c3cc4c(C(C)Nc5ccccc5C(=O)O)cc(C)cc4c(=O)n3C)cc2)CCOCC1 ; Cc1cc(C(C)Nc2ccccc2C(=O)O)c2cc(-c3ccc4c(c3)C(=O)N(C)C4)n(C)c(=O)c2c1 ; Cc1cc(C(C)Nc2ccccc2C(=O)O)c2cc(-c3ccn(-c4ccccc4)c3)n(C)c(=O)c2c1] 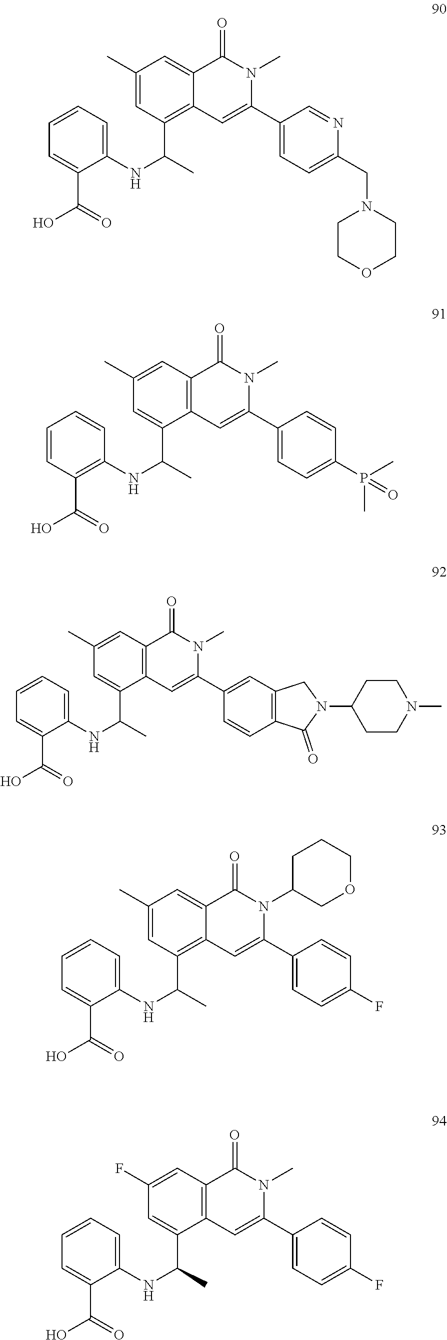 [SMILES: Cc1cc(C(C)Nc2ccccc2C(=O)O)c2cc(-c3ccc(CN4CCOCC4)nc3)n(C)c(=O)c2c1 ; Cc1cc(C(C)Nc2ccccc2C(=O)O)c2cc(-c3ccc(P(C)(C)=O)cc3)n(C)c(=O)c2c1 ; Cc1cc(C(C)Nc2ccccc2C(=O)O)c2cc(-c3ccc4c(c3)CN(C3CCN(C)CC3)C4=O)n(C)c(=O)c2c1 ; Cc1cc(C(C)Nc2ccccc2C(=O)O)c2cc(-c3ccc(F)cc3)n(C3CCCOC3)c(=O)c2c1 ; C[C@@H](Nc1ccccc1C(=O)O)c1cc(F)cc2c(=O)n(C)c(-c3ccc(F)cc3)cc12] 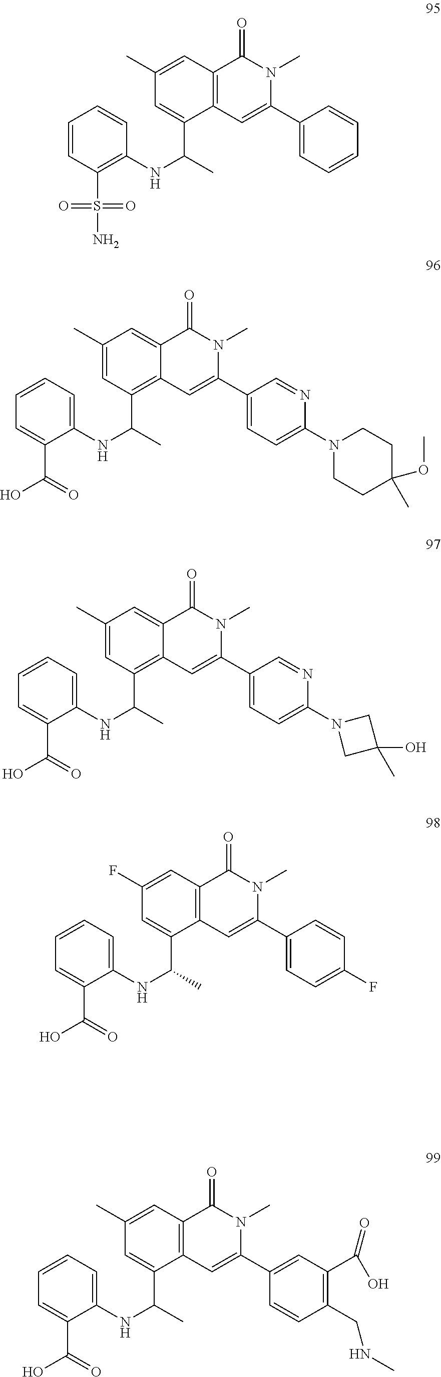 [SMILES: Cc1cc(C(C)Nc2ccccc2S(N)(=O)=O)c2cc(-c3ccccc3)n(C)c(=O)c2c1 ; COC1(C)CCN(c2ccc(-c3cc4c(C(C)Nc5ccccc5C(=O)O)cc(C)cc4c(=O)n3C)cn2)CC1 ; Cc1cc(C(C)Nc2ccccc2C(=O)O)c2cc(-c3ccc(N4CC(C)(O)C4)nc3)n(C)c(=O)c2c1 ; C[C@H](Nc1ccccc1C(=O)O)c1cc(F)cc2c(=O)n(C)c(-c3ccc(F)cc3)cc12 ; CNCc1ccc(-c2cc3c(C(C)Nc4ccccc4C(=O)O)cc(C)cc3c(=O)n2C)cc1C(=O)O] 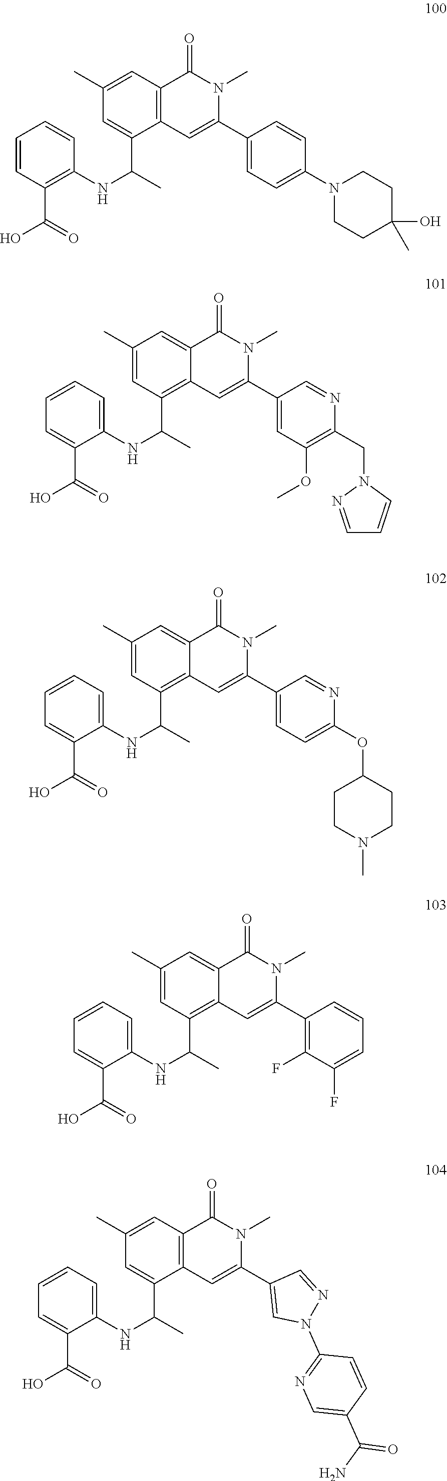 [SMILES: Cc1cc(C(C)Nc2ccccc2C(=O)O)c2cc(-c3ccc(N4CCC(C)(O)CC4)cc3)n(C)c(=O)c2c1 ; COc1cc(-c2cc3c(C(C)Nc4ccccc4C(=O)O)cc(C)cc3c(=O)n2C)cnc1Cn1cccn1 ; Cc1cc(C(C)Nc2ccccc2C(=O)O)c2cc(-c3ccc(OC4CCN(C)CC4)nc3)n(C)c(=O)c2c1 ; Cc1cc(C(C)Nc2ccccc2C(=O)O)c2cc(-c3cccc(F)c3F)n(C)c(=O)c2c1 ; Cc1cc(C(C)Nc2ccccc2C(=O)O)c2cc(-c3cnn(-c4ccc(C(N)=O)cn4)c3)n(C)c(=O)c2c1] 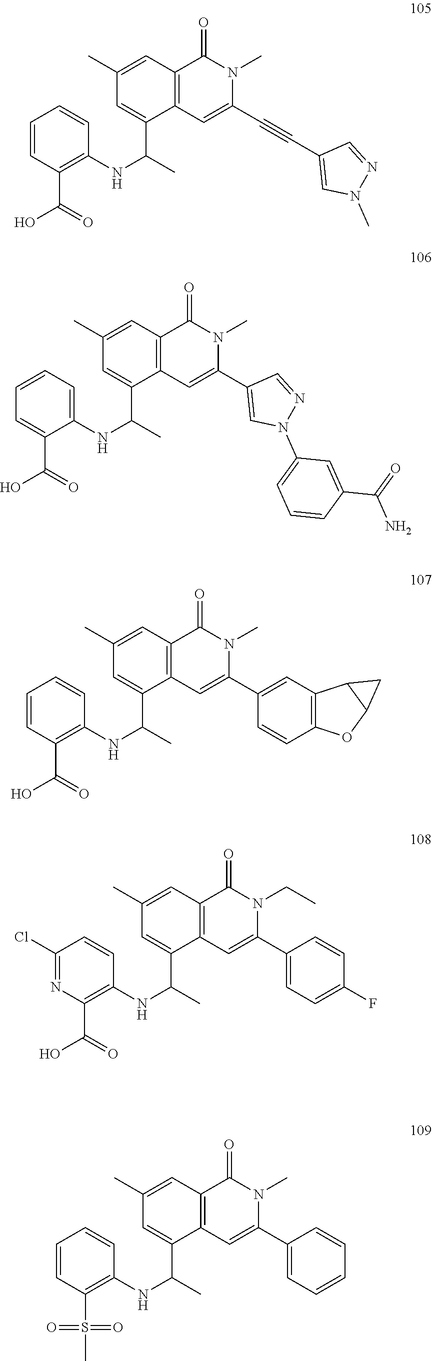 [SMILES: Cc1cc(C(C)Nc2ccccc2C(=O)O)c2cc(C#Cc3cnn(C)c3)n(C)c(=O)c2c1 ; Cc1cc(C(C)Nc2ccccc2C(=O)O)c2cc(-c3cnn(-c4cccc(C(N)=O)c4)c3)n(C)c(=O)c2c1 ; Cc1cc(C(C)Nc2ccccc2C(=O)O)c2cc(-c3ccc4c(c3)C3CC3O4)n(C)c(=O)c2c1 ; CCn1c(-c2ccc(F)cc2)cc2c(C(C)Nc3ccc(Cl)nc3C(=O)O)cc(C)cc2c1=O ; Cc1cc(C(C)Nc2ccccc2S(C)(=O)=O)c2cc(-c3ccccc3)n(C)c(=O)c2c1] 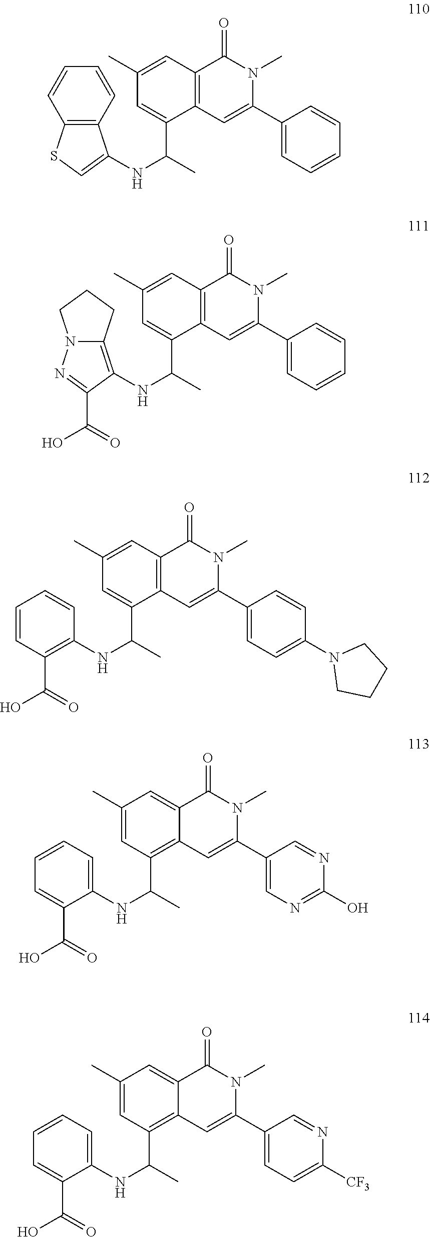 [SMILES: Cc1cc(C(C)Nc2csc3ccccc23)c2cc(-c3ccccc3)n(C)c(=O)c2c1 ; Cc1cc(C(C)Nc2c(C(=O)O)nn3c2CCC3)c2cc(-c3ccccc3)n(C)c(=O)c2c1 ; Cc1cc(C(C)Nc2ccccc2C(=O)O)c2cc(-c3ccc(N4CCCC4)cc3)n(C)c(=O)c2c1 ; Cc1cc(C(C)Nc2ccccc2C(=O)O)c2cc(-c3cnc(O)nc3)n(C)c(=O)c2c1 ; Cc1cc(C(C)Nc2ccccc2C(=O)O)c2cc(-c3ccc(C(F)(F)F)nc3)n(C)c(=O)c2c1] 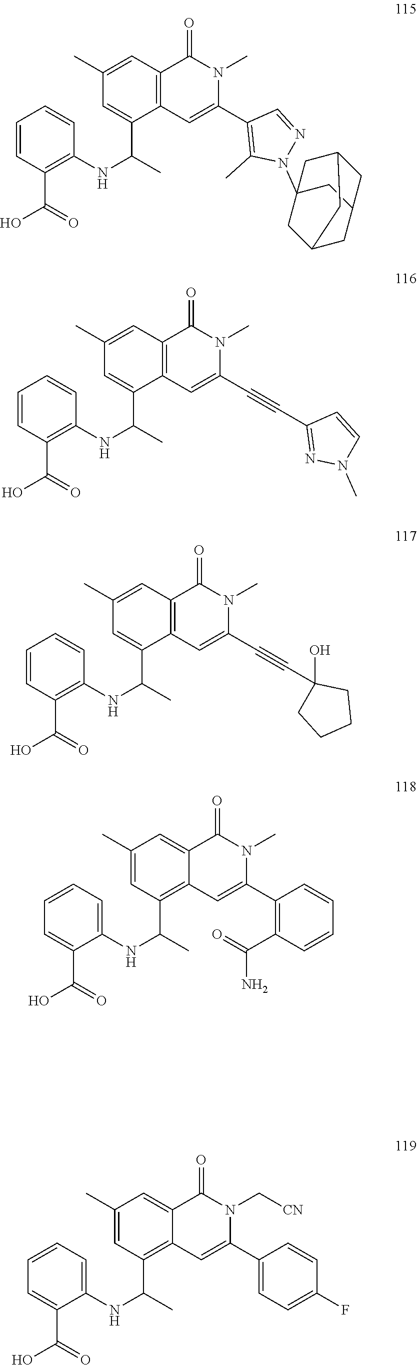 [SMILES: Cc1cc(C(C)Nc2ccccc2C(=O)O)c2cc(-c3cnn(C45CC6CC(CC(C6)C4)C5)c3C)n(C)c(=O)c2c1 ; Cc1cc(C(C)Nc2ccccc2C(=O)O)c2cc(C#Cc3ccn(C)n3)n(C)c(=O)c2c1 ; Cc1cc(C(C)Nc2ccccc2C(=O)O)c2cc(C#CC3(O)CCCC3)n(C)c(=O)c2c1 ; Cc1cc(C(C)Nc2ccccc2C(=O)O)c2cc(-c3ccccc3C(N)=O)n(C)c(=O)c2c1 ; Cc1cc(C(C)Nc2ccccc2C(=O)O)c2cc(-c3ccc(F)cc3)n(CC#N)c(=O)c2c1] 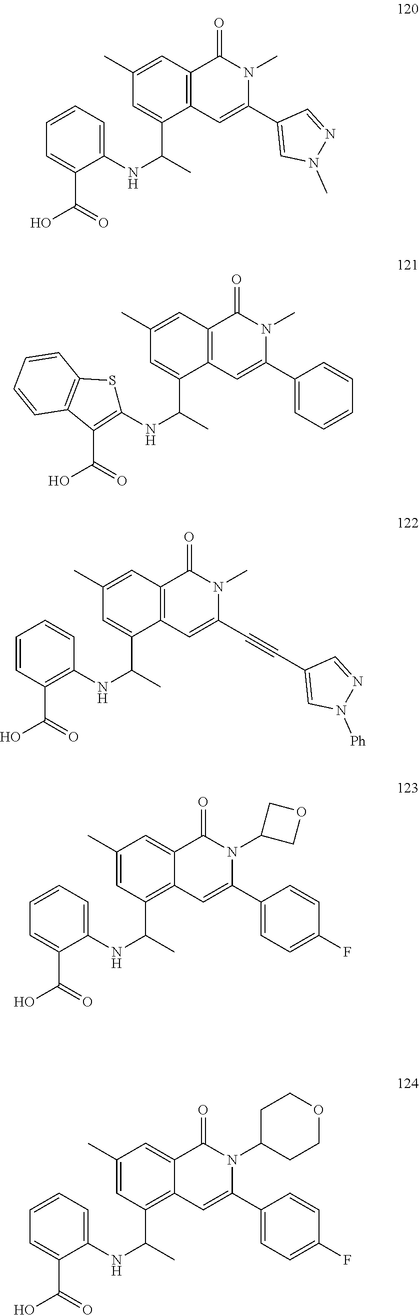 [SMILES: Cc1cc(C(C)Nc2ccccc2C(=O)O)c2cc(-c3cnn(C)c3)n(C)c(=O)c2c1 ; Cc1cc(C(C)Nc2sc3ccccc3c2C(=O)O)c2cc(-c3ccccc3)n(C)c(=O)c2c1 ; Cc1cc(C(C)Nc2ccccc2C(=O)O)c2cc(C#Cc3cnn(-c4ccccc4)c3)n(C)c(=O)c2c1 ; Cc1cc(C(C)Nc2ccccc2C(=O)O)c2cc(-c3ccc(F)cc3)n(C3COC3)c(=O)c2c1 ; Cc1cc(C(C)Nc2ccccc2C(=O)O)c2cc(-c3ccc(F)cc3)n(C3CCOCC3)c(=O)c2c1] 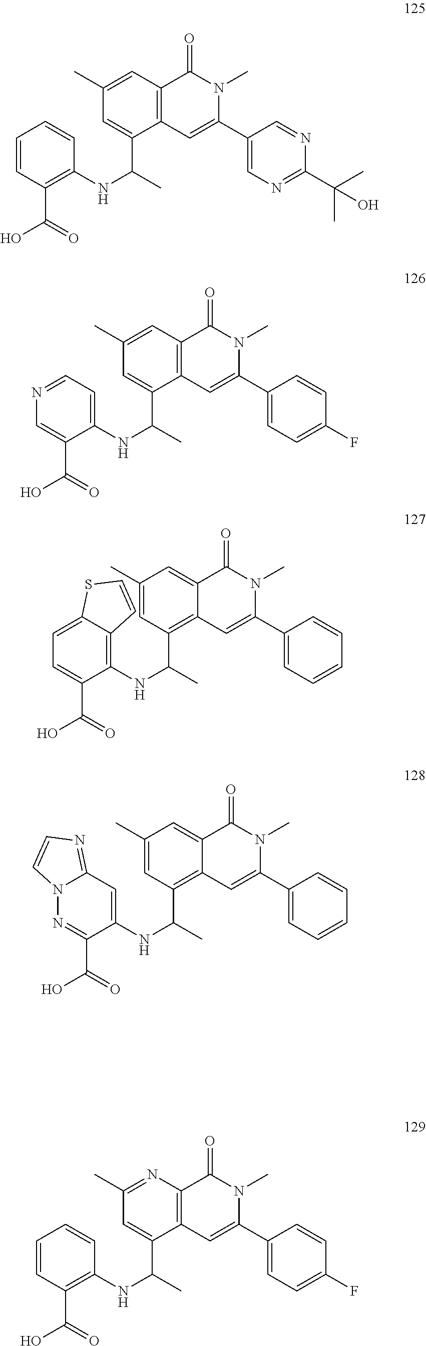 [SMILES: Cc1cc(C(C)Nc2ccccc2C(=O)O)c2cc(-c3cnc(C(C)(C)O)nc3)n(C)c(=O)c2c1 ; Cc1cc(C(C)Nc2ccncc2C(=O)O)c2cc(-c3ccc(F)cc3)n(C)c(=O)c2c1 ; Cc1cc(C(C)Nc2c(C(=O)O)ccc3sccc23)c2cc(-c3ccccc3)n(C)c(=O)c2c1 ; Cc1cc(C(C)Nc2cc3nccn3nc2C(=O)O)c2cc(-c3ccccc3)n(C)c(=O)c2c1 ; Cc1cc(C(C)Nc2ccccc2C(=O)O)c2cc(-c3ccc(F)cc3)n(C)c(=O)c2n1] 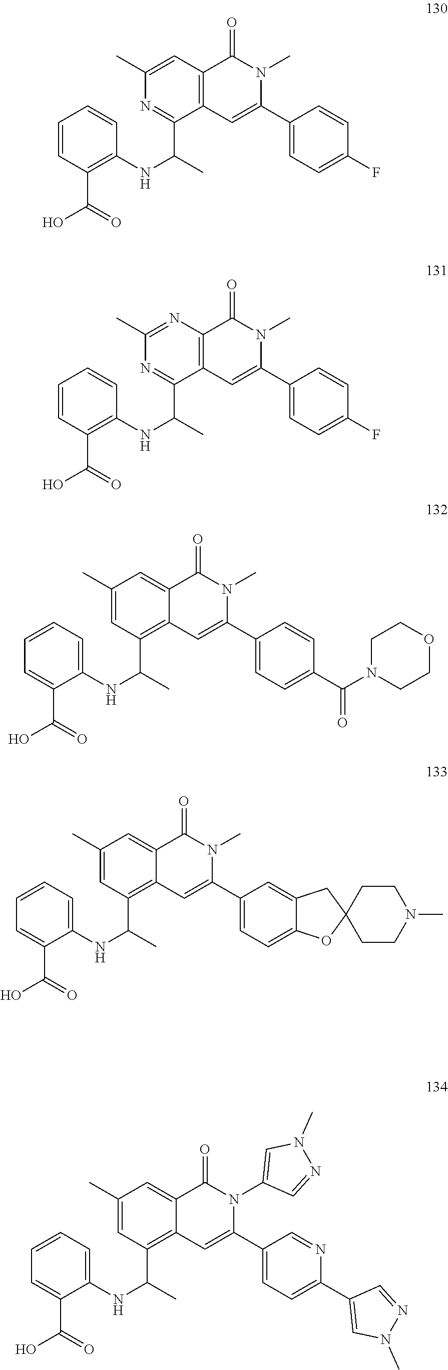 [SMILES: Cc1cc2c(=O)n(C)c(-c3ccc(F)cc3)cc2c(C(C)Nc2ccccc2C(=O)O)n1 ; Cc1nc(C(C)Nc2ccccc2C(=O)O)c2cc(-c3ccc(F)cc3)n(C)c(=O)c2n1 ; Cc1cc(C(C)Nc2ccccc2C(=O)O)c2cc(-c3ccc(C(=O)N4CCOCC4)cc3)n(C)c(=O)c2c1 ; Cc1cc(C(C)Nc2ccccc2C(=O)O)c2cc(-c3ccc4c(c3)CC3(CCN(C)CC3)O4)n(C)c(=O)c2c1 ; Cc1cc(C(C)Nc2ccccc2C(=O)O)c2cc(-c3ccc(-c4cnn(C)c4)nc3)n(-c3cnn(C)c3)c(=O)c2c1] 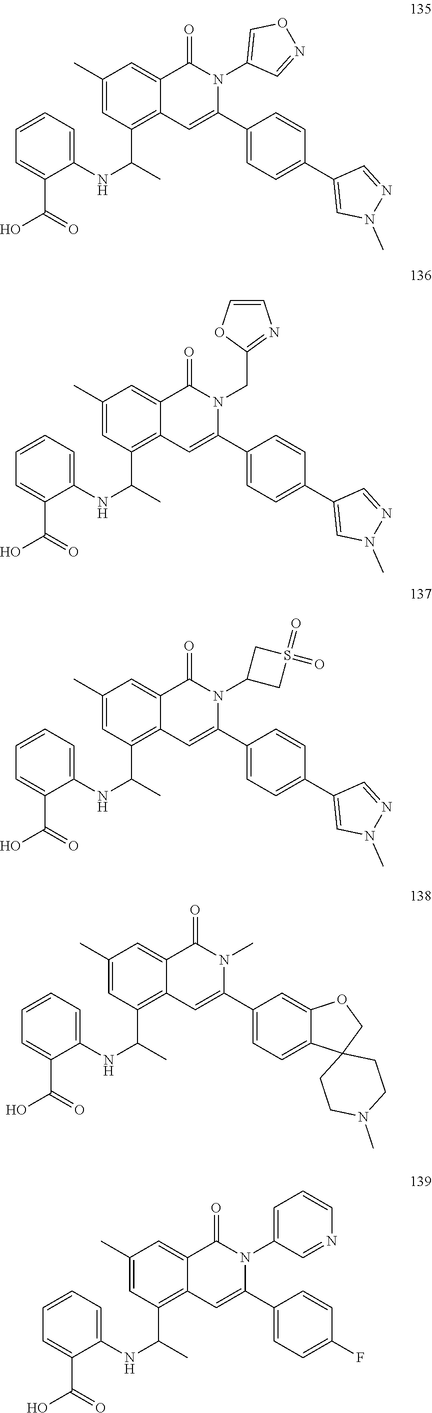 [SMILES: Cc1cc(C(C)Nc2ccccc2C(=O)O)c2cc(-c3ccc(-c4cnn(C)c4)cc3)n(-c3cnoc3)c(=O)c2c1 ; Cc1cc(C(C)Nc2ccccc2C(=O)O)c2cc(-c3ccc(-c4cnn(C)c4)cc3)n(Cc3ncco3)c(=O)c2c1 ; Cc1cc(C(C)Nc2ccccc2C(=O)O)c2cc(-c3ccc(-c4cnn(C)c4)cc3)n(C3CS(=O)(=O)C3)c(=O)c2c1 ; Cc1cc(C(C)Nc2ccccc2C(=O)O)c2cc(-c3ccc4c(c3)OCC43CCN(C)CC3)n(C)c(=O)c2c1 ; Cc1cc(C(C)Nc2ccccc2C(=O)O)c2cc(-c3ccc(F)cc3)n(-c3cccnc3)c(=O)c2c1] 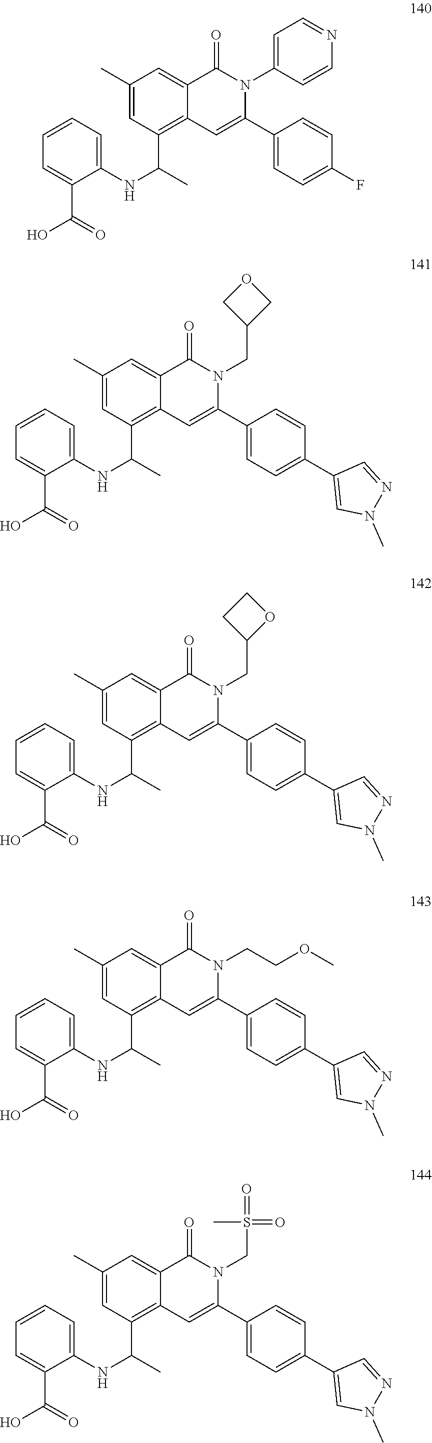 [SMILES: Cc1cc(C(C)Nc2ccccc2C(=O)O)c2cc(-c3ccc(F)cc3)n(-c3ccncc3)c(=O)c2c1 ; Cc1cc(C(C)Nc2ccccc2C(=O)O)c2cc(-c3ccc(-c4cnn(C)c4)cc3)n(CC3COC3)c(=O)c2c1 ; Cc1cc(C(C)Nc2ccccc2C(=O)O)c2cc(-c3ccc(-c4cnn(C)c4)cc3)n(CC3CCO3)c(=O)c2c1 ; COCCn1c(-c2ccc(-c3cnn(C)c3)cc2)cc2c(C(C)Nc3ccccc3C(=O)O)cc(C)cc2c1=O ; Cc1cc(C(C)Nc2ccccc2C(=O)O)c2cc(-c3ccc(-c4cnn(C)c4)cc3)n(CS(C)(=O)=O)c(=O)c2c1] 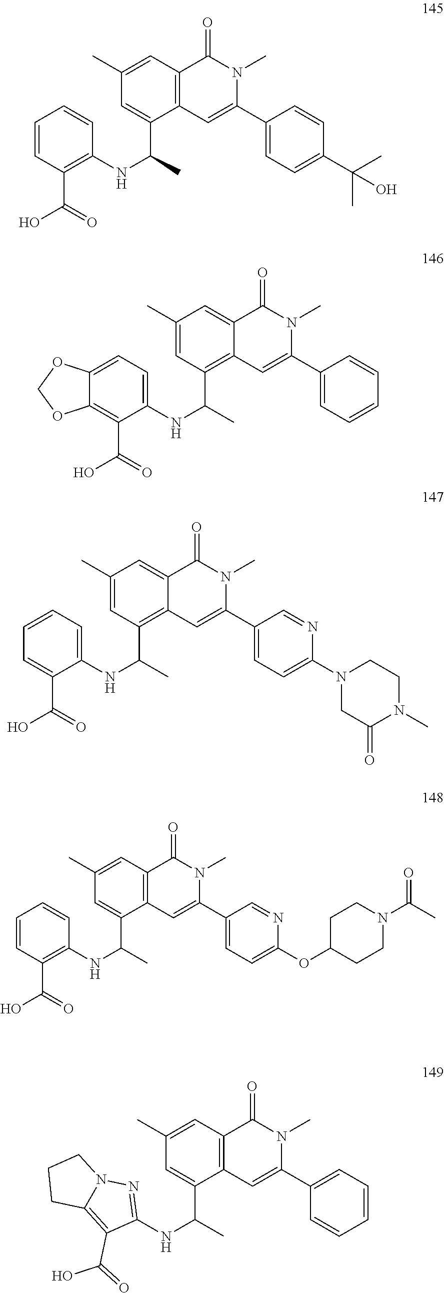 [SMILES: Cc1cc([C@@H](C)Nc2ccccc2C(=O)O)c2cc(-c3ccc(C(C)(C)O)cc3)n(C)c(=O)c2c1 ; Cc1cc(C(C)Nc2ccc3c(c2C(=O)O)OCO3)c2cc(-c3ccccc3)n(C)c(=O)c2c1 ; Cc1cc(C(C)Nc2ccccc2C(=O)O)c2cc(-c3ccc(N4CCN(C)C(=O)C4)nc3)n(C)c(=O)c2c1 ; CC(=O)N1CCC(Oc2ccc(-c3cc4c(C(C)Nc5ccccc5C(=O)O)cc(C)cc4c(=O)n3C)cn2)CC1 ; Cc1cc(C(C)Nc2nn3c(c2C(=O)O)CCC3)c2cc(-c3ccccc3)n(C)c(=O)c2c1] 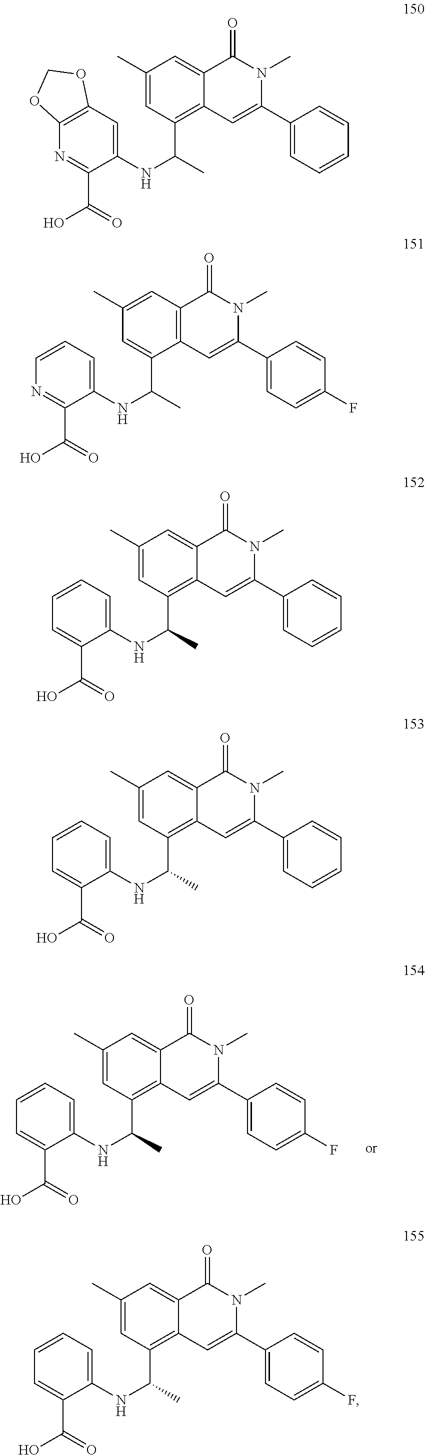 [SMILES: Cc1cc(C(C)Nc2cc3c(nc2C(=O)O)OCO3)c2cc(-c3ccccc3)n(C)c(=O)c2c1 ; Cc1cc(C(C)Nc2cccnc2C(=O)O)c2cc(-c3ccc(F)cc3)n(C)c(=O)c2c1 ; Cc1cc([C@@H](C)Nc2ccccc2C(=O)O)c2cc(-c3ccccc3)n(C)c(=O)c2c1 ; Cc1cc([C@H](C)Nc2ccccc2C(=O)O)c2cc(-c3ccccc3)n(C)c(=O)c2c1 ; Cc1cc([C@@H](C)Nc2ccccc2C(=O)O)c2cc(-c3ccc(F)cc3)n(C)c(=O)c2c1 ; Cc1cc([C@H](C)Nc2ccccc2C(=O)O)c2cc(-c3ccc(F)cc3)n(C)c(=O)c2c1]
or a pharmaceutically acceptable salt thereof.

37. A compound selected from: 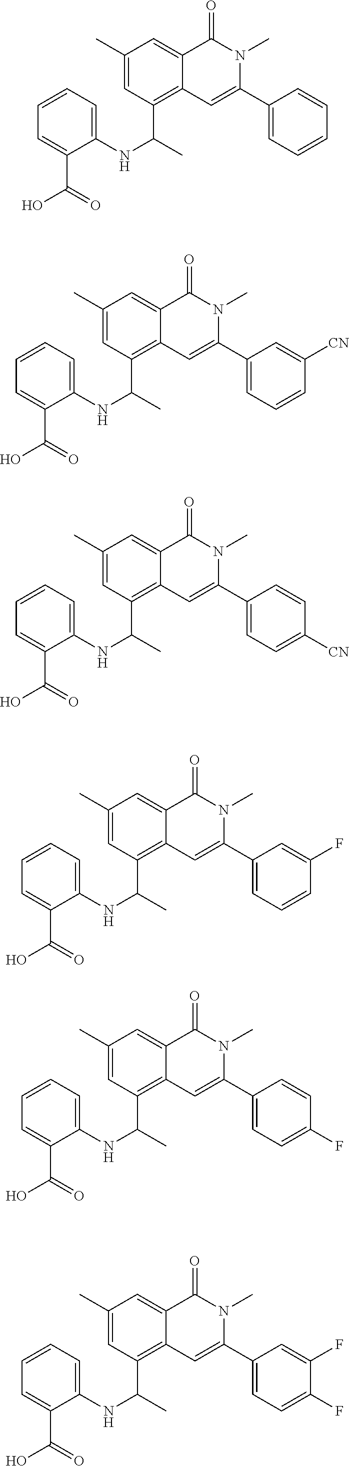 [SMILES: Cc1cc(C(C)Nc2ccccc2C(=O)O)c2cc(-c3ccccc3)n(C)c(=O)c2c1 ; Cc1cc(C(C)Nc2ccccc2C(=O)O)c2cc(-c3cccc(C#N)c3)n(C)c(=O)c2c1 ; Cc1cc(C(C)Nc2ccccc2C(=O)O)c2cc(-c3ccc(C#N)cc3)n(C)c(=O)c2c1 ; Cc1cc(C(C)Nc2ccccc2C(=O)O)c2cc(-c3cccc(F)c3)n(C)c(=O)c2c1 ; Cc1cc(C(C)Nc2ccccc2C(=O)O)c2cc(-c3ccc(F)cc3)n(C)c(=O)c2c1 ; Cc1cc(C(C)Nc2ccccc2C(=O)O)c2cc(-c3ccc(F)c(F)c3)n(C)c(=O)c2c1] 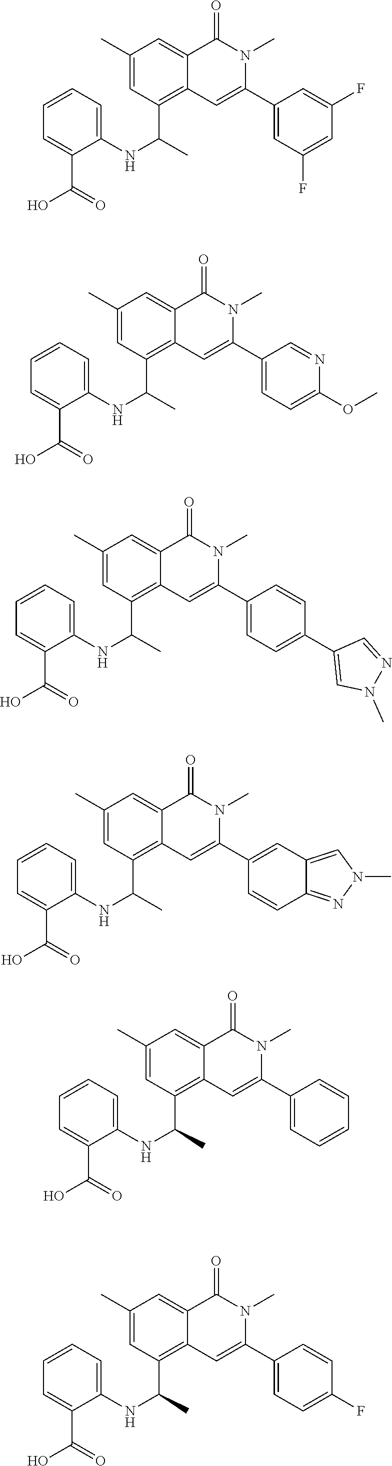 [SMILES: Cc1cc(C(C)Nc2ccccc2C(=O)O)c2cc(-c3cc(F)cc(F)c3)n(C)c(=O)c2c1 ; COc1ccc(-c2cc3c(C(C)Nc4ccccc4C(=O)O)cc(C)cc3c(=O)n2C)cn1 ; Cc1cc(C(C)Nc2ccccc2C(=O)O)c2cc(-c3ccc(-c4cnn(C)c4)cc3)n(C)c(=O)c2c1 ; Cc1cc(C(C)Nc2ccccc2C(=O)O)c2cc(-c3ccc4nn(C)cc4c3)n(C)c(=O)c2c1 ; Cc1cc([C@@H](C)Nc2ccccc2C(=O)O)c2cc(-c3ccccc3)n(C)c(=O)c2c1 ; Cc1cc([C@@H](C)Nc2ccccc2C(=O)O)c2cc(-c3ccc(F)cc3)n(C)c(=O)c2c1] 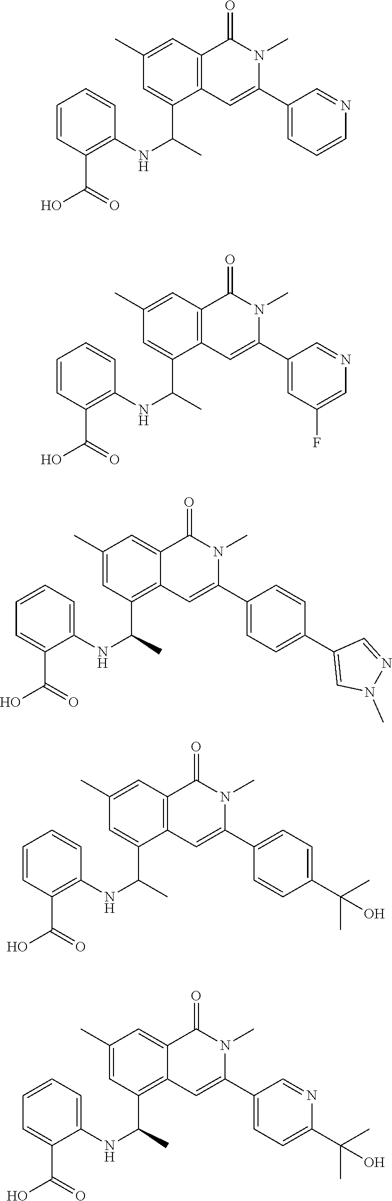 [SMILES: Cc1cc(C(C)Nc2ccccc2C(=O)O)c2cc(-c3cccnc3)n(C)c(=O)c2c1 ; Cc1cc(C(C)Nc2ccccc2C(=O)O)c2cc(-c3cncc(F)c3)n(C)c(=O)c2c1 ; Cc1cc([C@@H](C)Nc2ccccc2C(=O)O)c2cc(-c3ccc(-c4cnn(C)c4)cc3)n(C)c(=O)c2c1 ; Cc1cc(C(C)Nc2ccccc2C(=O)O)c2cc(-c3ccc(C(C)(C)O)cc3)n(C)c(=O)c2c1 ; Cc1cc([C@@H](C)Nc2ccccc2C(=O)O)c2cc(-c3ccc(C(C)(C)O)nc3)n(C)c(=O)c2c1] 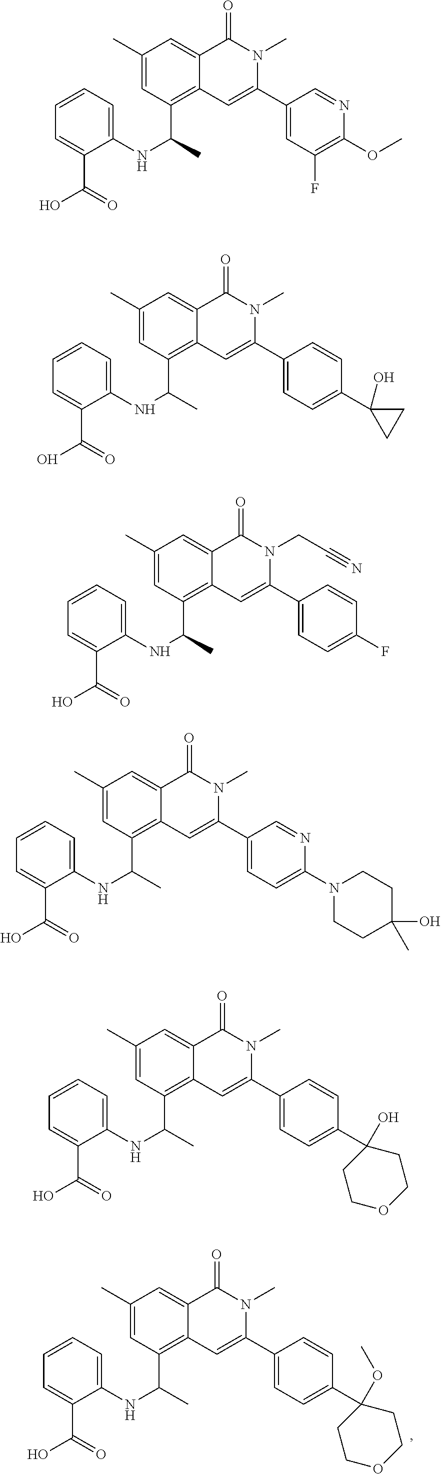 [SMILES: COc1ncc(-c2cc3c([C@@H](C)Nc4ccccc4C(=O)O)cc(C)cc3c(=O)n2C)cc1F ; Cc1cc(C(C)Nc2ccccc2C(=O)O)c2cc(-c3ccc(C4(O)CC4)cc3)n(C)c(=O)c2c1 ; Cc1cc([C@@H](C)Nc2ccccc2C(=O)O)c2cc(-c3ccc(F)cc3)n(CC#N)c(=O)c2c1 ; Cc1cc(C(C)Nc2ccccc2C(=O)O)c2cc(-c3ccc(N4CCC(C)(O)CC4)nc3)n(C)c(=O)c2c1 ; Cc1cc(C(C)Nc2ccccc2C(=O)O)c2cc(-c3ccc(C4(O)CCOCC4)cc3)n(C)c(=O)c2c1 ; COC1(c2ccc(-c3cc4c(C(C)Nc5ccccc5C(=O)O)cc(C)cc4c(=O)n3C)cc2)CCOCC1]
or a pharmaceutically acceptable salt thereof.

38. A pharmaceutical composition comprising a compound or stereoisomer, tautomer, or pharmaceutically acceptable salt thereof of claim 1, and a pharmaceutically acceptable excipient.

39. A method for treating a disorder or a disease responsive to the inhibition of PI3Kα activity in a subject in need thereof, comprising administering to the subject a therapeutically effective amount of a compound or stereoisomer, tautomer, or pharmaceutically acceptable salt thereof of claim 1.

40-43. (canceled)
